(19) United States
(12) Patent Application Publication
(10) Pub. No.: US 2024/0184199 A1
(43) Pub. Date: Jun. 6, 2024

(54) Onium Salt, Resist Composition, And Patterning Process

(71) Applicant: SHIN-ETSU CHEMICAL CO., LTD., Tokyo (JP)

(72) Inventor: Masahiro FUKUSHIMA, Joetsu-shi (JP)

(73) Assignee: SHIN-ETSU CHEMICAL CO., LTD., Tokyo (JP)

(21) Appl. No.: 18/377,117

(22) Filed: Oct. 5, 2023

(30) Foreign Application Priority Data
Oct. 12, 2022 (JP) 2022164317

(51) Int. Cl. G03F 7/004; C07C 25/18; C07C 211/63; C07C 233/54; C07C 233/63; C07C 233/81; C07C 311/08; C07C 311/21; C07C 381/12; C07D 327/08; C07D 333/54; C07D 333/76

(52) U.S. Cl. CPC G03F 7/0045; C07C 25/18; C07C 211/63; C07C 233/54; C07C 233/63; C07C 233/81; C07C 311/08; C07C 311/21; C07C 381/12; C07D 327/08; C07D 333/54; C07D 333/76; C07C 2601/14; C07C 2603/74

(57) ABSTRACT

The present invention is an onium salt represented by the following general formula (1), where n1 represents an integer of 0 or 1; n2 represents an integer of 0 to 3; R^{1a} represents a hydrocarbyl group having 1 to 20 carbon atoms and optionally containing a heteroatom; n3 represents an integer of 0 to 3; R^{1b} represents a hydrocarbyl group having 1 to 36 carbon atoms and optionally containing a heteroatom; X^{A} represents a carbonyl group that forms an amide bond together with an adjacent —NH or represents a sulfonyl group that forms a sulfonamide bond together with the adjacent —NH; n4 represents an integer of 1 or 2; and Z⁺ represents an onium cation. This provides: a resist composition in lithography that has high sensitivity and excellent resolution, can improve LWR and CDU, and can also suppress collapse of a resist pattern; and a novel onium salt to be used in the resist composition.

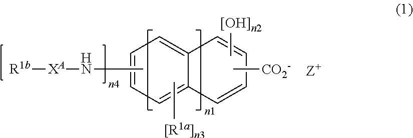

### TECHNICAL FIELD

The present invention relates to: an onium salt; a resist composition containing an acid diffusion controller including the onium salt; and a patterning process using the resist composition.

### BACKGROUND ART

As LSIs advance toward higher integration and higher processing speed, miniaturization of pattern rule is progressing rapidly. This is because the spread of high-speed communication of 5 G and artificial intelligence (AI) has progressed, and high-performance devices for processing these are needed. As a cutting-edge technology for miniaturization, 5-nm node devices have been mass-produced by extreme ultraviolet ray (EUV) lithography at a wavelength of 13.5 nm. Furthermore, studies are also in progress on employing EUV lithography in next-generation 3-nm node and the following-generation 2-nm node devices.

As the miniaturization progresses, image blurs due to acid diffusion become a problem. To ensure resolution for fine patterns with dimensional sizes of 45 nm and smaller, there is a proposal that it is important to not only improve dissolution contrast as previously reported, but also control acid diffusion (Non Patent Document 1). Nevertheless, since chemically amplified resist materials (compositions) enhance the sensitivity and contrast through acid diffusion, an attempt to minimize acid diffusion by reducing the temperature and/or time of post-exposure bake (PEB) results in significant reductions of sensitivity and contrast.

A triangular tradeoff relationship among sensitivity, resolution, and edge roughness has been pointed out. Specifically, resolution improvement requires suppression of acid diffusion, whereas shortening acid diffusion distance lowers sensitivity.

The addition of an acid generator capable of generating a bulky acid is effective in suppressing acid diffusion. Hence, it has been proposed to incorporate in a polymer a repeating unit derived from an onium salt having a polymerizable unsaturated bond. In this case, the polymer also functions as an acid generator (polymer-bound acid generator). Patent Document 1 proposes a sulfonium and iodonium salt having a polymerizable unsaturated bond that generates a particular sulfonic acid. Patent Document 2 proposes a sulfonium salt having a sulfonate acid moiety directly bonded to the main chain.

In an acid-labile group used for a (meth)acrylate polymer for an ArF resist material, a deprotection reaction progresses by the use of a photo-acid generator that generates a sulfonic acid having a fluorine atom substituted at a position. However, a deprotection reaction does not progress when using an acid generator that generates a sulfonic acid not having fluorine substituted at a position or generates carboxylic acid. When a sulfonium salt or iodonium salt that generates a sulfonic acid having fluorine substituted at a position is mixed with a sulfonium salt or iodonium salt that generates a sulfonic acid not having fluorine substituted at a position, the sulfonium salt or iodonium salt that generates the sulfonic acid not having fluorine substituted at a position undergoes ion exchange with the sulfonic acid having fluorine substituted at a position. A sulfonic acid having fluorine substituted at a position generated by light returns to being a sulfonium salt or iodonium salt by ion exchange. Therefore, a sulfonium salt or iodonium salt of a sulfonic acid not having fluorine substituted at a position or of carboxylic acid functions as a quencher (acid diffusion controller). A resist material in which a sulfonium salt or iodonium salt that generates carboxylic acid is used as a quencher is proposed (Patent Document 3).

Various sulfonium salt quenchers that generate a carboxylic acid have been proposed. In particular, disclosed are sulfonium salts of salicylic acid, β-hydroxycarboxylic acid (Patent Document 4), salicylic acid derivatives (Patent Documents 5 and 6), fluorosalicylic acid (Patent Document 7), and hydroxynaphthoic acid (Patent Document 8).

Meanwhile, it is pointed out that cohesion of a quencher reduces the critical dimension uniformity of a resist pattern. There are expectations that the critical dimension uniformity of a pattern after development can be improved by preventing the cohesion of the quencher in a resist film to achieve a uniform distribution. Regarding the above-mentioned salicylic-acid-type sulfonium salt quenchers, there are also proposals for structures having a plurality of hydroxy groups in an aromatic ring (Patent Documents 6, 9, 10, and 11). However, having a plurality of hydroxy groups causes low solvent solubility, and there is concern for precipitation.

There is a problem against requirements for further miniaturization that swelling caused by a developer occurs during alkaline development particularly in a positive resist, resulting in pattern collapse during formation of a fine pattern. To deal with such a problem in miniaturization, it is important to develop a novel resist material. Development is desired for an onium salt quencher that has excellent sensitivity, sufficiently controlled acid diffusion, and excellent solvent solubility and is effective for suppressing pattern collapse.

### CITATION LIST

### Patent Literature

Patent Document 1: JP 2006-045311 A
Patent Document 2: JP 2006-178317 A
Patent Document 3: JP 2007-114431 A
Patent Document 4: WO 2018/159560 A1
Patent Document 5: JP 2020-203984 A
Patent Document 6: JP 2020-91404 A
Patent Document 7: JP 2020-91312 A
Patent Document 8: JP 2019-120760 A
Patent Document 9: WO 2020/195428 A1
Patent Document 10: JP 2022-91525 A
Patent Document 11: JP 2020-152721 A

### Non Patent Literature

Non Patent Document 1: SPIE Vol. 6520 65203L-1 (2007)

### SUMMARY OF INVENTION

### Technical Problem

The present invention has been made in view of the above-described circumstances. An object of the present invention is to provide a novel onium salt to be contained in a resist composition that has high sensitivity and excellent resolution, can improve LWR (roughness) and CDU (critical dimension uniformity), and can suppress resist pattern collapse in lithography such as deep ultraviolet lithography and EUV lithography, in both positive and negative types.

### Solution to Problem

To achieve the object, the present invention provides an onium salt represented by the following general formula (1),

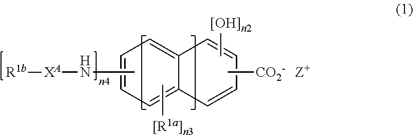

wherein n1 represents an integer of 0 or 1; n2 represents an integer of 0 to 3; R^{1a} represents a hydrocarbyl group having 1 to 20 carbon atoms and optionally containing a heteroatom; n3 represents an integer of 0 to 3; R^{1b} represents a hydrocarbyl group having 1 to 36 carbon atoms and optionally containing a heteroatom; X^{A} represents a carbonyl group that forms an amide bond together with an adjacent —NH or represents a sulfonyl group that forms a sulfonamide bond together with the adjacent —NH; n4 represents an integer of 1 or 2; and Z⁺ represents an onium cation.

Such an onium salt is useful as a novel onium salt to be contained in a resist composition in lithography with a positive or negative resist, and the onium salt has high sensitivity and excellent resolution, can improve LWR and CDU, and can also suppress collapse of a resist pattern.

Furthermore, the general formula (1) is preferably represented by the following formula (1-A),

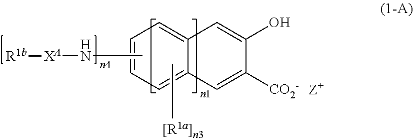

wherein R^{1a}, R^{1b}, X^{A}, n1, n3, n4, and Z⁺ are as defined above.

Such an onium salt functions more favorably as an acid diffusion controller contained in a resist composition.

Furthermore, the general formula (1) is preferably represented by the following formula (1-B),

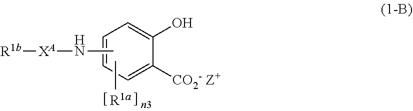

wherein R^{1a}, R^{1b}, X^{A}, n3, and Z⁺ are as defined above.

Such an onium salt functions even more favorably as an acid diffusion controller contained in a resist composition.

The Z⁺ in the general formula (1) preferably further represents an onium cation represented by one of the following general formulae (Cation-1) to (Cation-3),

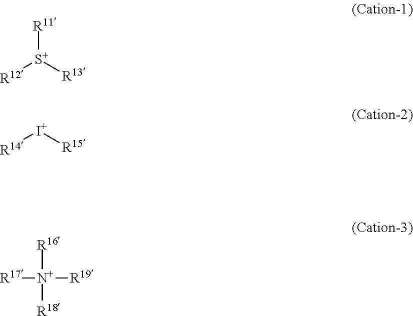

wherein R^{11′} to R^{19′} each independently represent a saturated or unsaturated, linear, branched, or cyclic hydrocarbyl group having 1 to 30 carbon atoms and optionally containing a heteroatom.

Such an onium salt functions particularly favorably as an acid diffusion controller contained in a resist composition.

In addition, the present invention provides an acid diffusion controller comprising the above-described onium salt.

The inventive onium salt is useful as an acid diffusion controller.

In addition, the present invention provides a resist composition comprising the above-described acid diffusion controller.

An excellent resist composition can be achieved when the above-described acid diffusion controller is contained.

The resist composition preferably further comprises an acid generator to generate an acid.

In such a resist composition, the above-described onium salt functions as an acid diffusion controller, and the inventive resist composition functions.

The acid generator preferably generates a sulfonic acid, imide acid, or methide acid.

Such an acid generator is more suitable as an acid generator.

The resist composition preferably further comprises an organic solvent.

Such a solvent can dissolve each component, and the coating property of the composition can be improved.

The resist composition preferably further comprises a base polymer.

Such a resist composition is suitable as a resist composition.

The base polymer preferably contains a repeating unit represented by the following general formula (a1) and/or a repeating unit represented by the following general formula (a2),

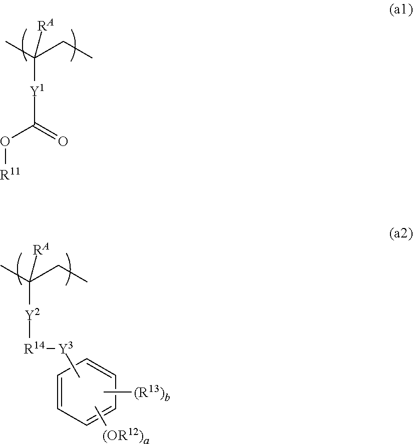

wherein each R^{A} independently represents a hydrogen atom or a methyl group; Y¹ represents a single bond or a linking group having 1 to 12 carbon atoms containing at least one selected from a phenylene group, a naphthylene group, an ester bond, and a lactone ring; Y² represents a single bond or an ester bond; Y³ represents a single bond, an ether bond, or an ester bond; R¹¹ and R¹² each independently represent an acid-labile group; R¹³ represents a fluorine atom, a trifluoromethyl group, a cyano group, or a saturated hydrocarbyl group having 1 to 6 carbon atoms; R¹⁴ represents a single bond or an alkanediyl group having 1 to 6 carbon atoms, and some of the carbon atoms are optionally substituted with an ether bond or an ester bond; and “a” represents 1 or 2 and “b” represents an integer of 0 to 4, provided that 1≤a+b≤5.

Such a resist composition contains an acid-labile group, and is suitable as a positive resist composition.

The resist composition is preferably a chemically amplified positive resist composition.

The inventive resist composition can function as a chemically amplified positive resist composition.

Furthermore, the base polymer preferably contains no acid-labile group.

Such a resist composition contains no acid-labile group, and is suitable as a negative resist composition.

The resist composition is preferably a chemically amplified negative resist composition.

The inventive resist composition can function as a chemically amplified negative resist composition.

The base polymer preferably further contains at least one repeating unit selected from repeating units represented by the following general formulae (f1) to (f3),

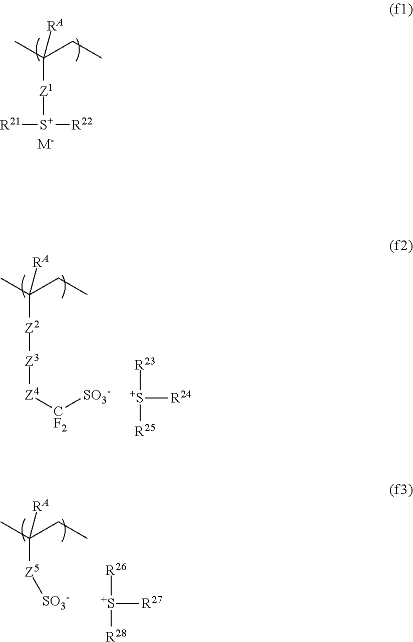

wherein each R^{A} independently represents a hydrogen atom or a methyl group; Z¹ represents a single bond, an aliphatic hydrocarbylene group having 1 to 6 carbon atoms, a phenylene group, a naphthylene group, an ester bond, a group having 7 to 18 carbon atoms derived from a combination of these groups, —O—Z¹¹—, —C(═O)—O—Z¹¹—, or —C(═O)—NH—Z¹¹—; Z″ represents an aliphatic hydrocarbylene group having 1 to 6 carbon atoms, a phenylene group, a naphthylene group, or a group having 7 to 18 carbon atoms derived from a combination of these groups, Z¹¹ optionally containing a carbonyl group, an ester bond, an ether bond, or a hydroxy group; Z² represents a single bond or an ester bond; Z³ represents a single bond, —Z³¹ —C(═O)—O—, —Z³¹ —O—, or —Z³¹ —O—C(═O)—; Z³¹ represents a hydrocarbylene group having 1 to 12 carbon atoms, a phenylene group, or a group having 7 to 18 carbon atoms derived from a combination of these groups, Z³¹ optionally containing a carbonyl group, an ester bond, an ether bond, an iodine atom, or a bromine atom; Z⁴ represents a methylene group, a 2,2,2-trifluoro-1,1-ethanediyl group, or a carbonyl group; Z⁵ represents a single bond, a methylene group, an ethylene group, a phenylene group, a fluorinated phenylene group, a phenylene group substituted with a trifluoromethyl group, —O—Z⁵¹ —, —C(═O)—O—Z⁵¹ —, or —C(═O)—NH—Z⁵¹ —; Z⁵¹ represents an aliphatic hydrocarbylene group having 1 to 6 carbon atoms, a phenylene group, a fluorinated phenylene group, a phenylene group substituted with a trifluoromethyl group, or a combination of these groups, Z⁵¹ optionally containing any of a carbonyl group, an ester bond, an ether bond, a halogen atom, and a hydroxy group; R²¹ to R²⁸ each independently represent a hydrocarbyl group having 1 to 20 carbon atoms and optionally containing a heteroatom; R²³ and R²⁴ or R²⁶ and R²⁷ are optionally bonded to each other to form a ring together with a sulfur atom bonded to R²³ and R²⁴ or R²⁶ and R²⁷; and M⁻ represents a non-nucleophilic counterion.

Such repeating units function as an acid generator in the base polymer.

The resist composition preferably further comprises a surfactant.

In this manner, coating properties of the resist composition can be improved and controlled.

In addition, the present invention provides a patterning process comprising the steps of:
forming a resist film on a substrate by using the above-described resist composition;
exposing the resist film to a high-energy beam; and
developing the exposed resist film by using a developer.

According to such a patterning process, an excellent pattern can be formed.

The high-energy beam can be a KrF excimer laser beam, an ArF excimer laser beam, an electron beam, or an extreme ultraviolet ray having a wavelength of 3 to 15 nm.

When such a high-energy beam is used, a better pattern can be formed.

### Advantageous Effects of Invention

The inventive novel onium salt functions favorably as an acid diffusion controller (quencher) in a resist composition and has high sensitivity and excellent dissolution contrast, so that it is possible to construct a pattern profile having low LWR, low CDU, excellent rectangularity, and high resolution as a result. In addition, it is possible to provide: a resist composition that suppresses swelling of a resist pattern during alkaline development, can form a pattern that is resistant to collapse, and contains the inventive novel onium salt excellent in fine pattern formation; and a patterning process using the resist composition.

### DESCRIPTION OF EMBODIMENTS

As stated above, it has been desired to develop an onium salt quencher that has excellent sensitivity, can control acid diffusion sufficiently, and also has excellent solvent solubility and is effective for suppressing pattern collapse.

To achieve the object, the present inventor has studied earnestly and found out that a resist composition containing, as an acid diffusion controller, an onium salt having a particular structure allows excellent sensitivity and resolution of a resist film, low LWR of a line pattern, low CDU of a hole pattern, and furthermore, suppresses swelling during development and is extremely effective for accurate fine processing. Thus, the present inventor has conceived the present invention.

That is, the present invention is an onium salt represented by the following general formula (1),

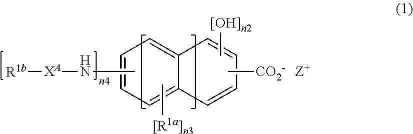

wherein n1 represents an integer of 0 or 1; n2 represents an integer of 0 to 3; R^{1a} represents a hydrocarbyl group having 1 to 20 carbon atoms and optionally containing a heteroatom; n3 represents an integer of 0 to 3; R^{1b} represents a hydrocarbyl group having 1 to 36 carbon atoms and optionally containing a heteroatom; X^{A} represents a carbonyl group that forms an amide bond together with an adjacent —NH or represents a sulfonyl group that forms a sulfonamide bond together with the adjacent —NH; n4 represents an integer of 1 or 2; and Z⁺ represents an onium cation.

Hereinafter, the present invention will be described in detail, but the present invention is not limited thereto.

### [Onium Salt]

The inventive onium salt is represented by the following general formula (1).

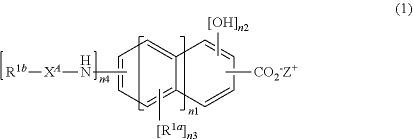

In the general formula (1), n1 represents an integer of 0 or 1. When n1=0, a benzene ring is indicated, and when n1=1, a naphthalene ring is indicated. From the viewpoint of solvent solubility, a benzene ring, where n1 is 0, is preferable.

In the general formula (1), n2 represents an integer of 0 to 3. When n2 is 1 or more, at least one OH group is preferably bonded to a carbon atom adjacent to the carbon atom to which a carboxylate group (CO₂⁻ group) is bonded. The substituent represented by [OH]n2 indicates that n2 hydrogen atoms of the benzene ring or naphthalene ring are each substituted with an OH group.

In the general formula (1), R^{1a} represents a hydrocarbyl group having 1 to 20 carbon atoms and optionally containing a heteroatom such as a halogen atom, an oxygen atom, a sulfur atom, and a nitrogen atom. Part or all of the hydrogen atoms in the hydrocarbyl group may be substituted with a halogen atom, and —CH₂— constituting the hydrocarbyl group may be substituted with —O— or —C(═O)—. The hydrocarbyl group may be saturated or unsaturated, and may be linear, branched, or cyclic. Specific examples thereof include: alkyl groups having 1 to 20 carbon atoms, such as a methyl group, an ethyl group, an n-propyl group, an isopropyl group, an n-butyl group, and a tert-butyl group; cyclic saturated hydrocarbyl groups having 3 to 20 carbon atoms, such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups having 2 to 20 carbon atoms, such as a vinyl group, an allyl group, a propenyl group, a butenyl group, and a hexenyl group; cyclic unsaturated hydrocarbyl groups having 3 to 20 carbon atoms, such as a cyclohexenyl group; aryl groups having 6 to 20 carbon atoms, such as a phenyl group and a naphthyl group; aralkyl groups having 7 to 20 carbon atoms, such as a benzyl group, a 1-phenylethyl group, and a 2-phenylethyl group; and groups derived from combinations of these groups. Furthermore, part or all of the hydrogen atoms of the hydrocarbyl group may be substituted with a group containing a heteroatom, such as an oxygen atom, a sulfur atom, a nitrogen atom, or a halogen atom, and part of the —CH₂— constituting the hydrocarbyl group may be substituted with a group containing a heteroatom, such as an oxygen atom, a sulfur atom, or a nitrogen atom. The resulting hydrocarbyl group may contain a hydroxy group, a cyano group, a fluorine atom, a chlorine atom, a bromine atom, an iodine atom, a carbonyl group, an ether bond, an ester bond, a sulfonic acid ester bond, a carbonate bond, a lactone ring, a sultone ring, a carboxylic anhydride, a haloalkyl group, etc. Examples of the halogen atom include a fluorine atom, a chlorine atom, a bromine atom, and an iodine atom. In particular, a fluorine atom and an iodine atom are preferable.

In the general formula (1), n3 represents an integer of 0 to 3. When n3≥2, the plurality of R^{1a}s may be bonded to each other to form a ring structure together with the carbon atoms bonded to the R^{1a}s. When a ring structure is formed, specific examples include a five-membered ring and six-membered ring structures, but the structure is not limited thereto.

In the general formula (1), R^{1b} represents a hydrocarbyl group having 1 to 36 carbon atoms and optionally containing a heteroatom. Part or all of the hydrogen atoms in the hydrocarbyl group may be substituted with a heteroatom such as a halogen atom, and —CH₂— constituting the hydrocarbyl group may be substituted with —O— or —C(═O)—. The hydrocarbyl group may be saturated or unsaturated, and may be linear, branched, or cyclic. Specific examples thereof include the groups given for R^{1a}.

In the general formula (1), X^{A} represents a carbonyl group that forms an amide bond together with an adjacent —NH or represents a sulfonyl group that forms a sulfonamide bond together with the adjacent —NH. In view of acid diffusion control and acidity of the conjugate carboxylic acid, a sulfonyl group is preferable.

In the general formula (1), n4 represents an integer of 1 or 2, and from the viewpoint of availability of starting materials, n4 is preferably 1.

Furthermore, the general formula (1) is preferably represented by the following formula (1-A). In particular, salicylic acid has an effect of suppressing acid diffusion by an intramolecular hydrogen bond between the carboxylic acid and the hydroxy group, and therefore, the general formula (1) is more preferably represented by the following general formula (1-B).

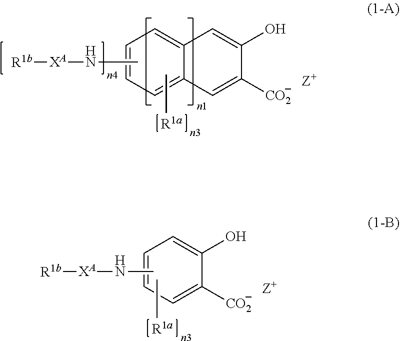

In the formulae, R^{1a}, R^{1b}, X^{A}, n1, n3, n4, and Z⁺ are as defined above.

When the structure represented by the general formula (1) is represented by the formula (1-A), the onium salt functions more favorably as an acid diffusion controller contained in a resist composition. When the structure represented by the general formula (1-A) is represented by the general formula (1-B), the onium salt functions further favorably.

Examples of the anion of the onium salt represented by the general formula (1) include the following, but are not limited thereto.

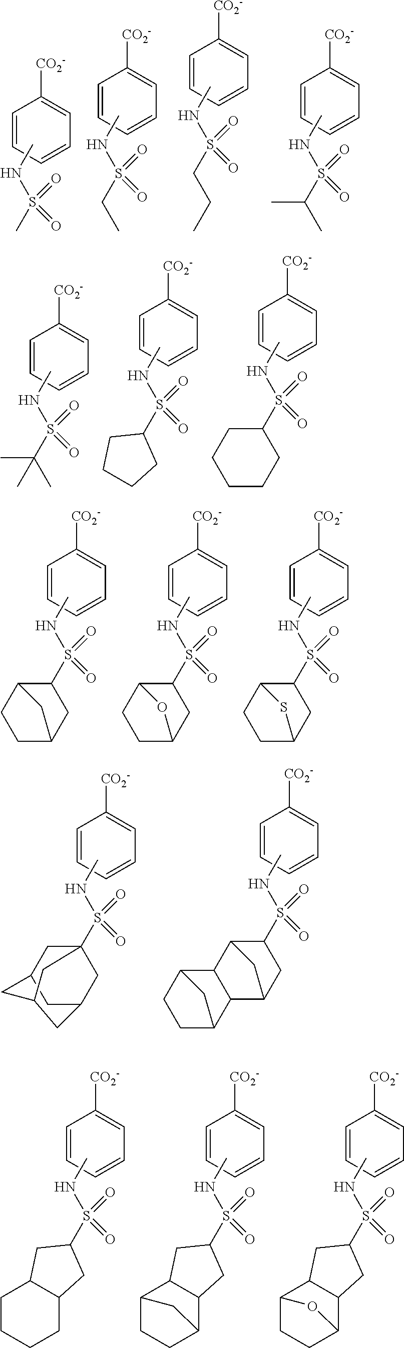

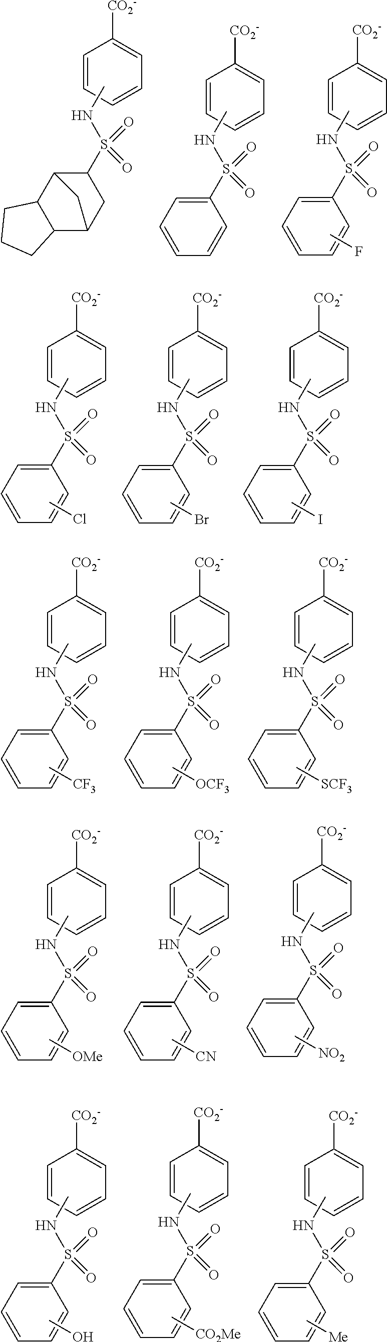

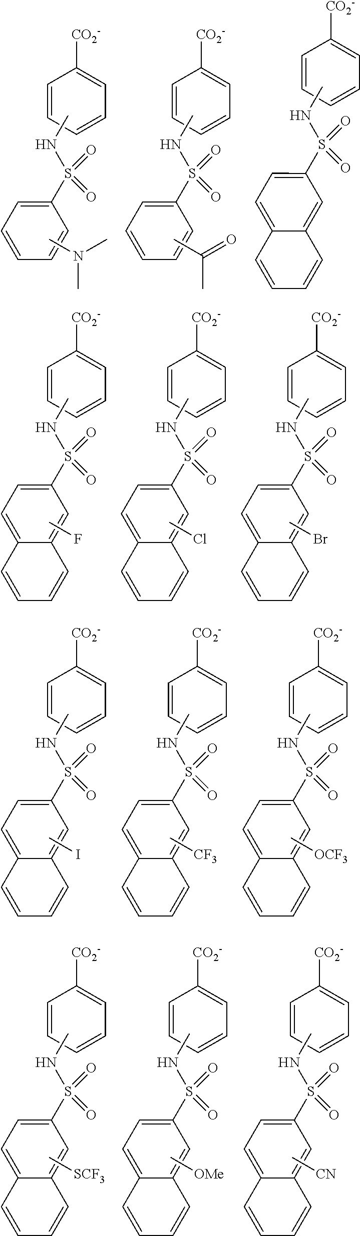

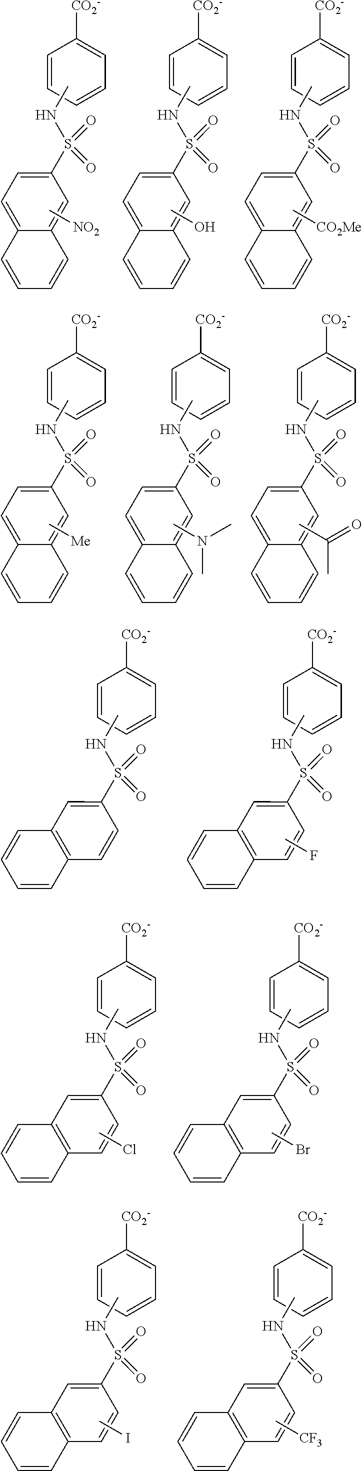

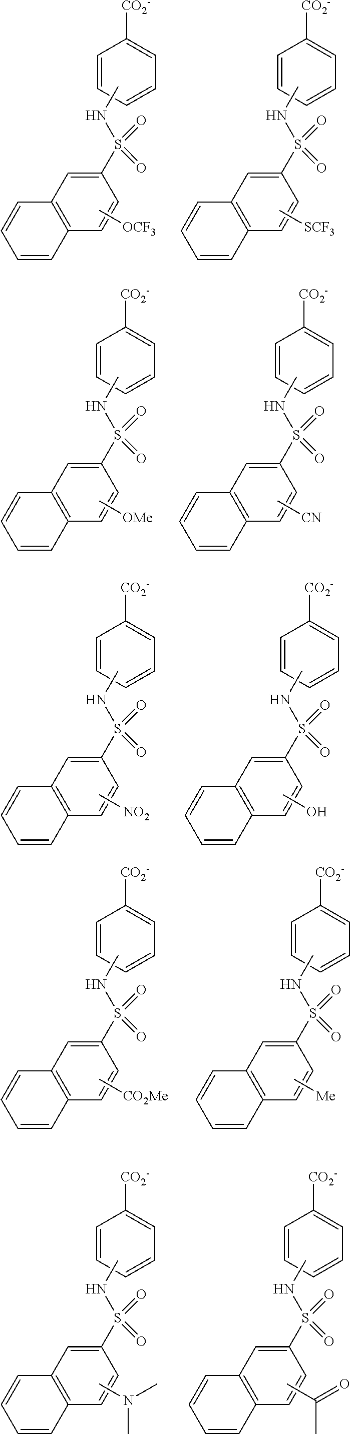

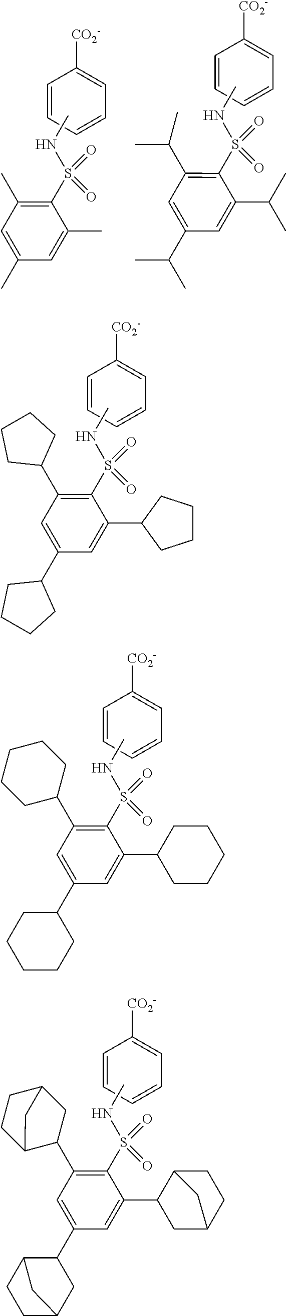

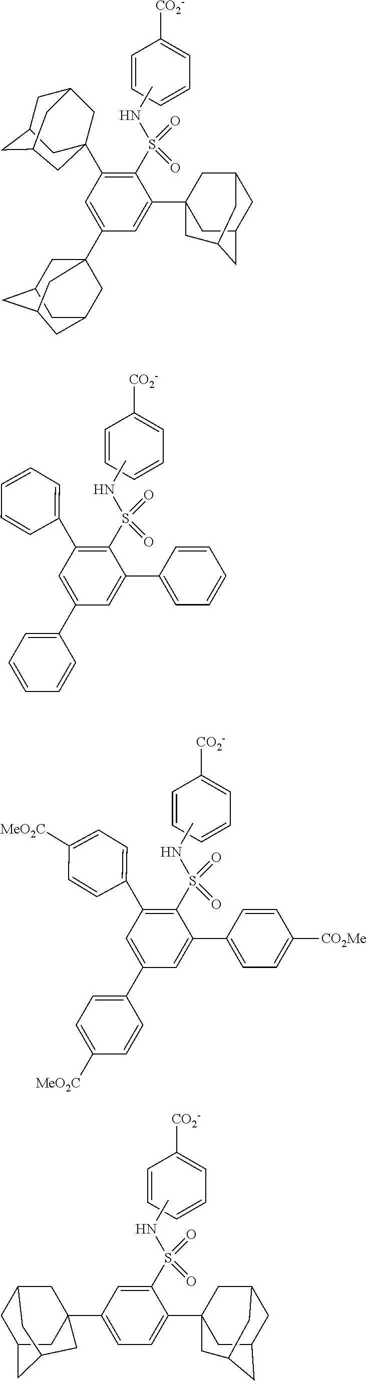

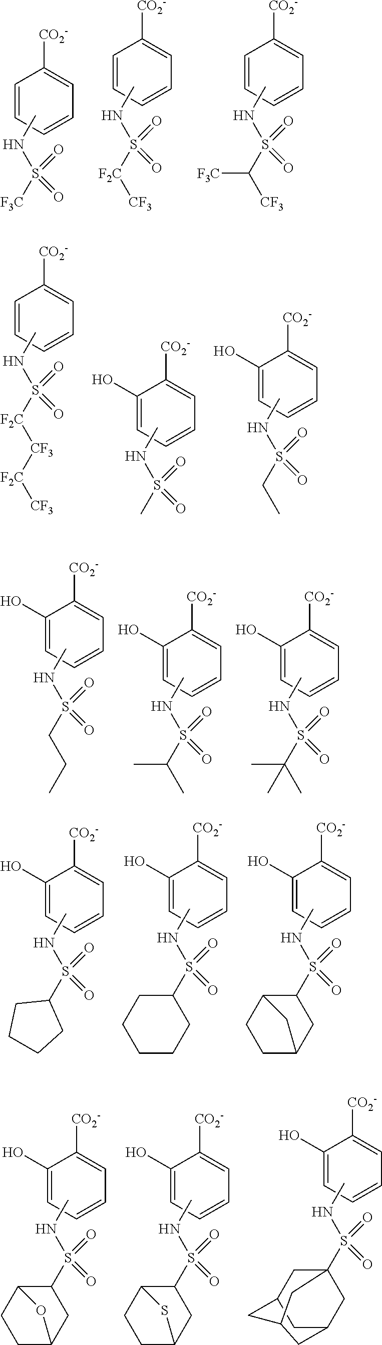

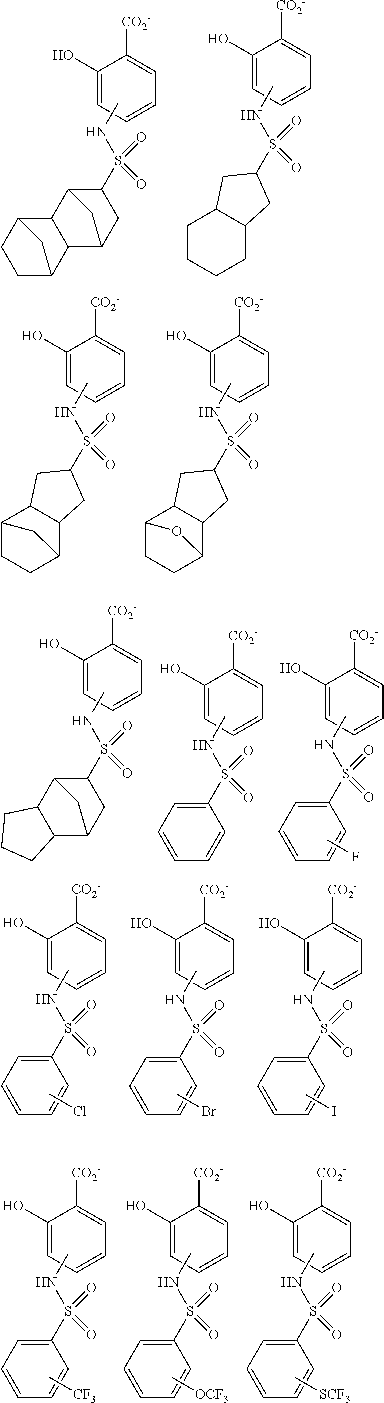

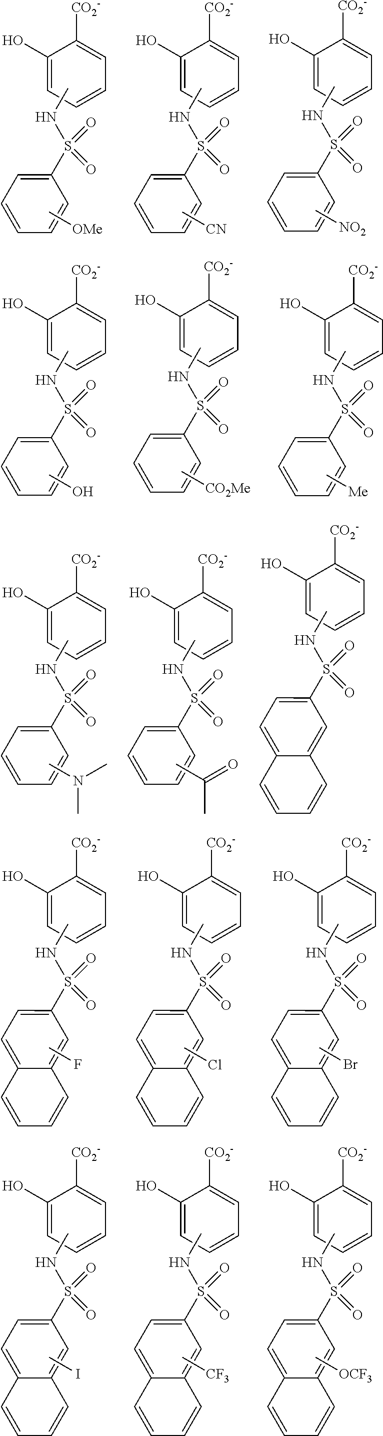

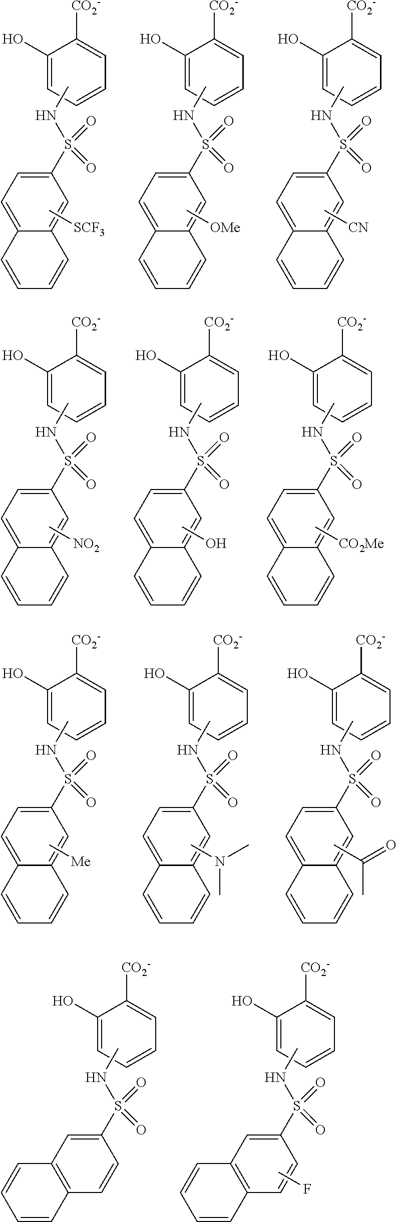

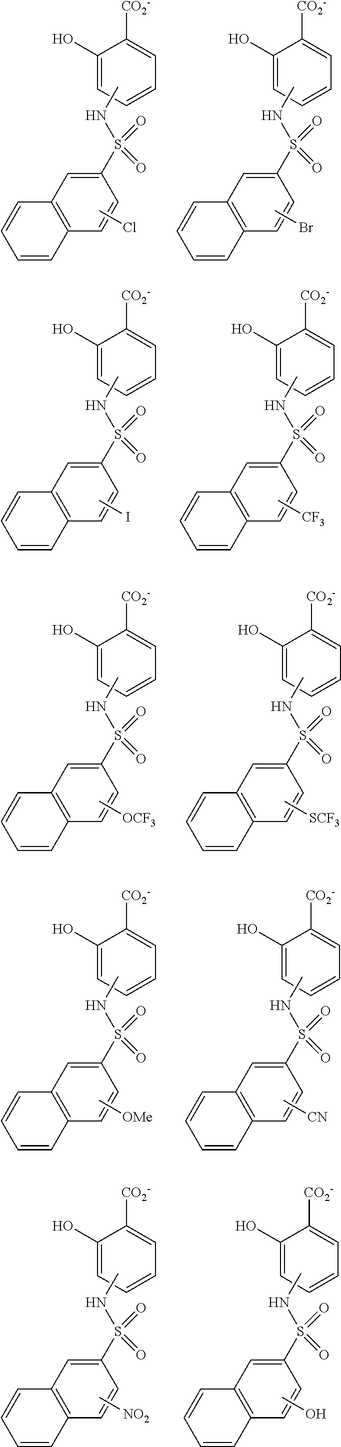

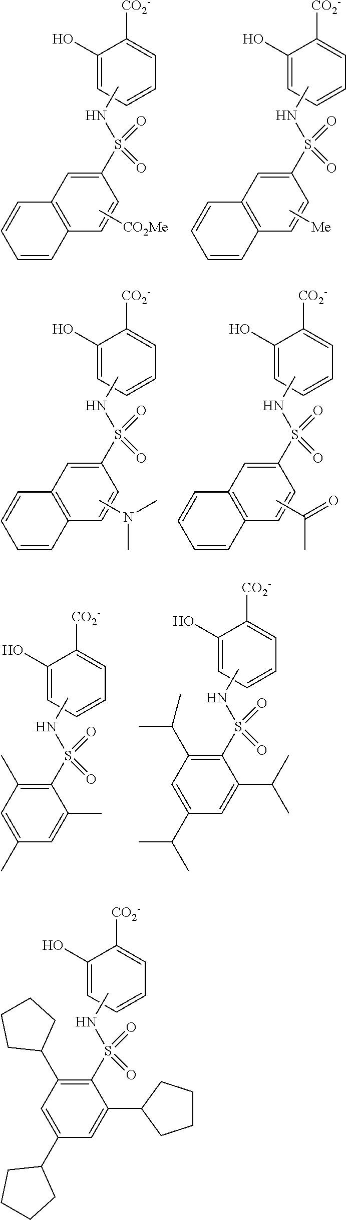

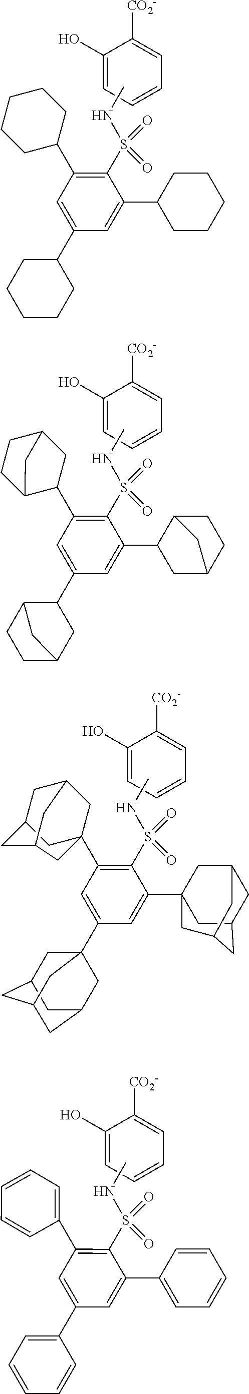

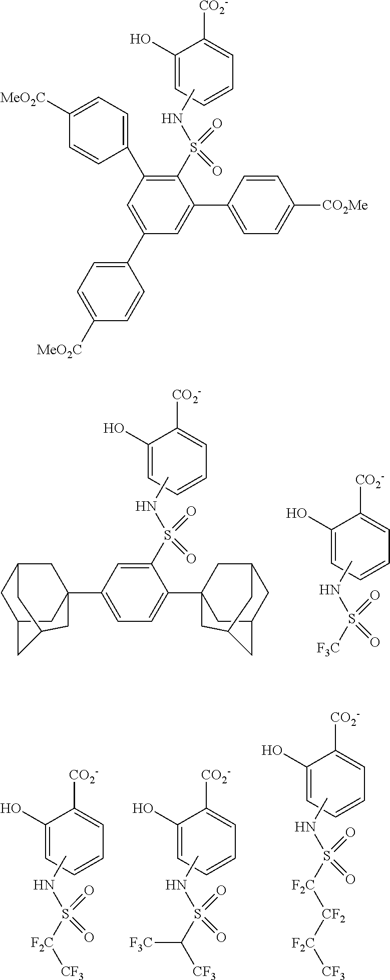

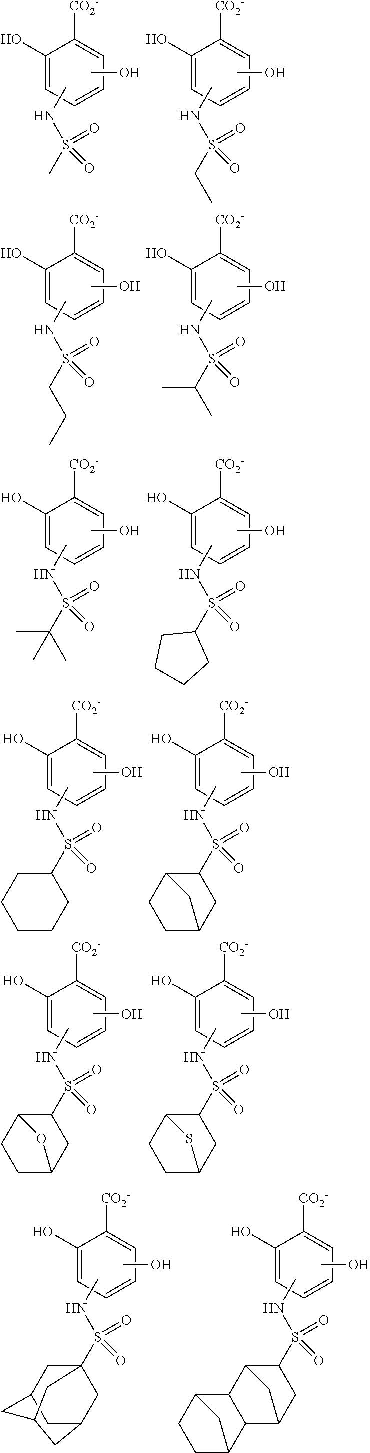

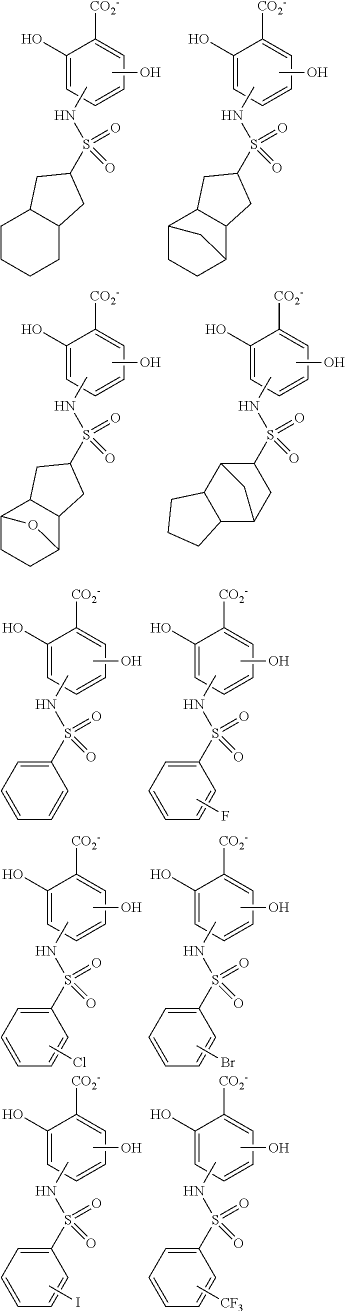

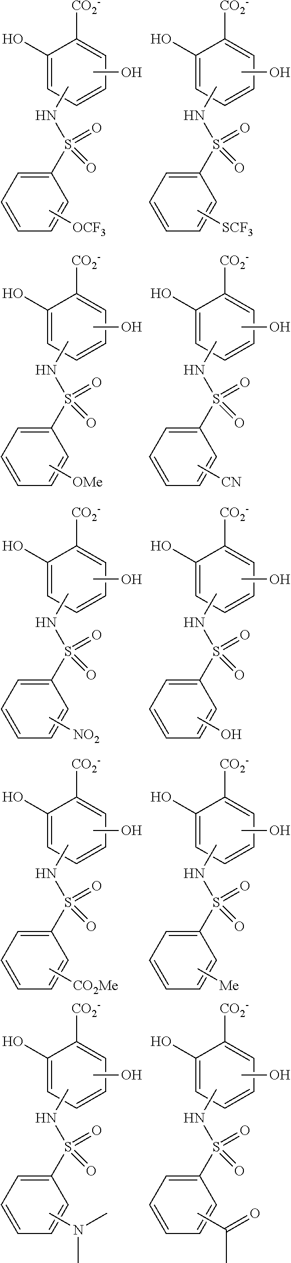

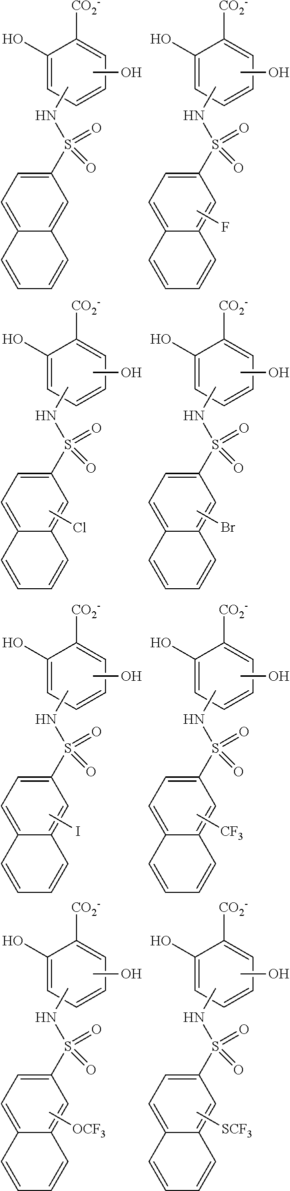

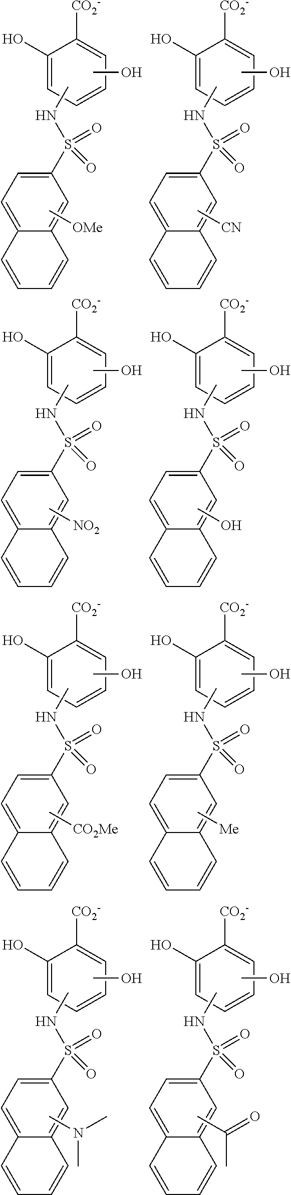

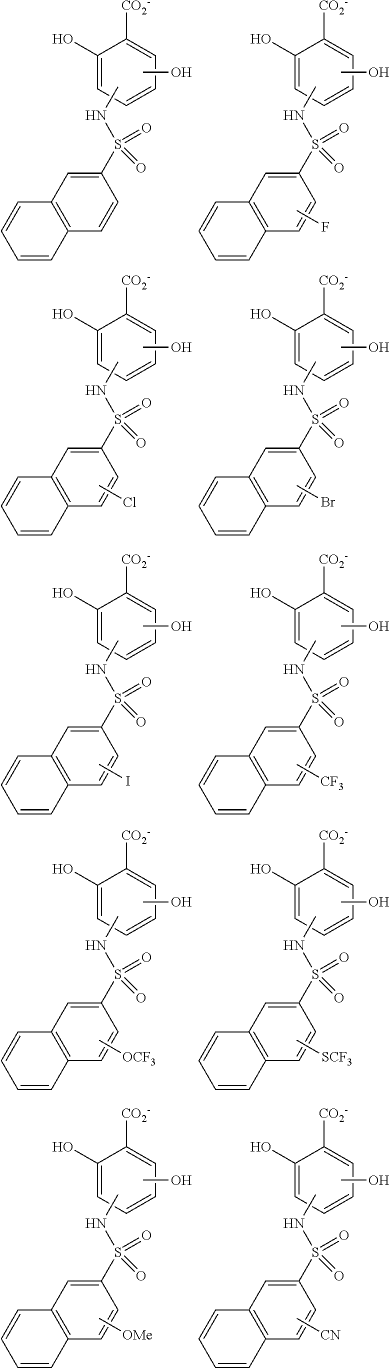

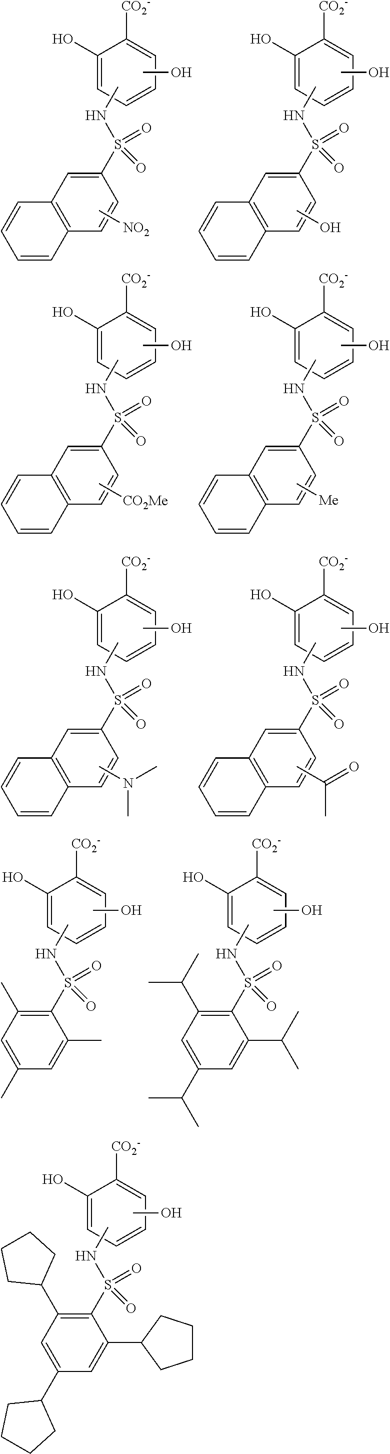

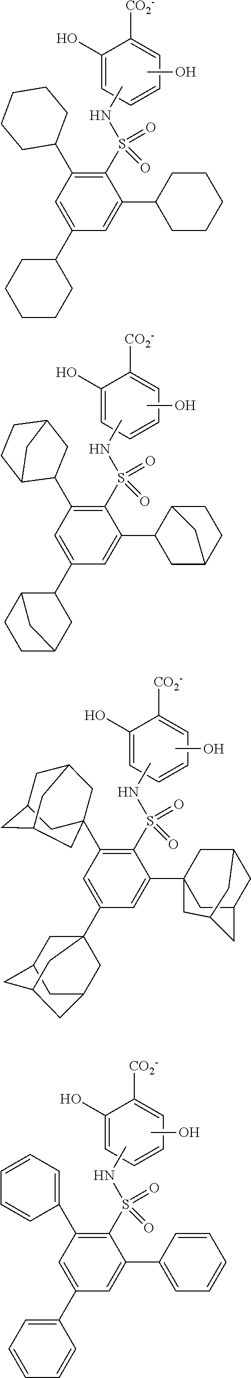

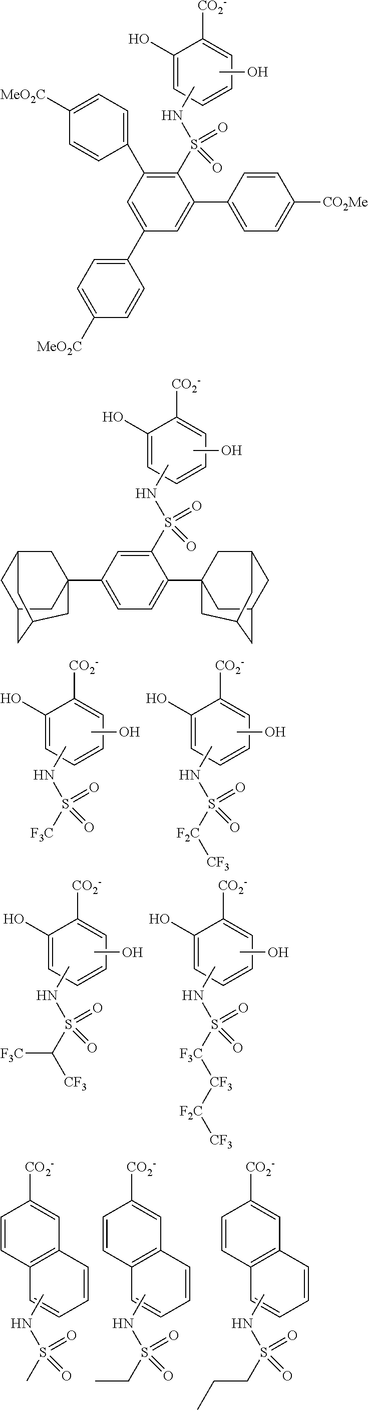

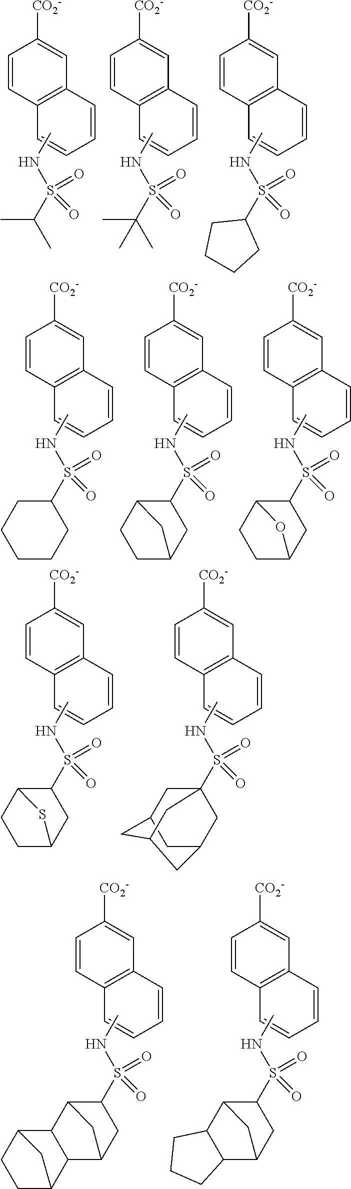

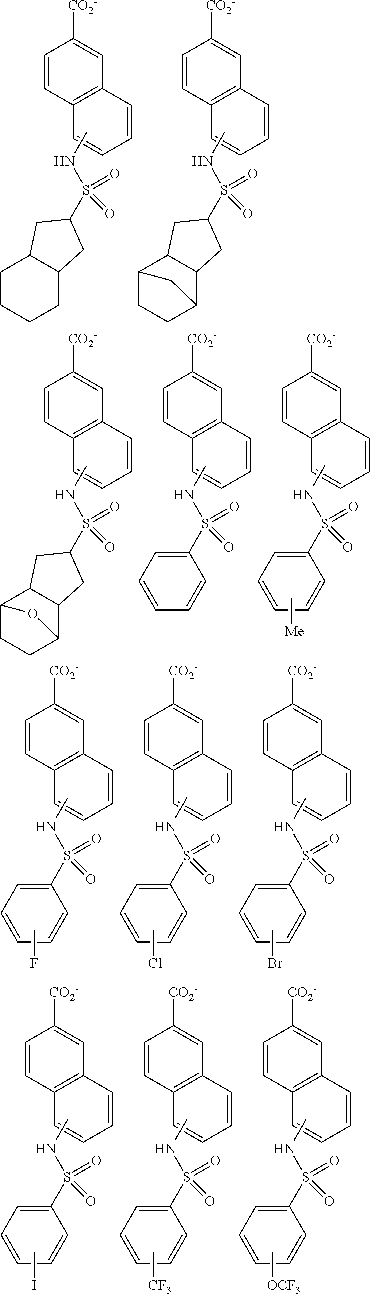

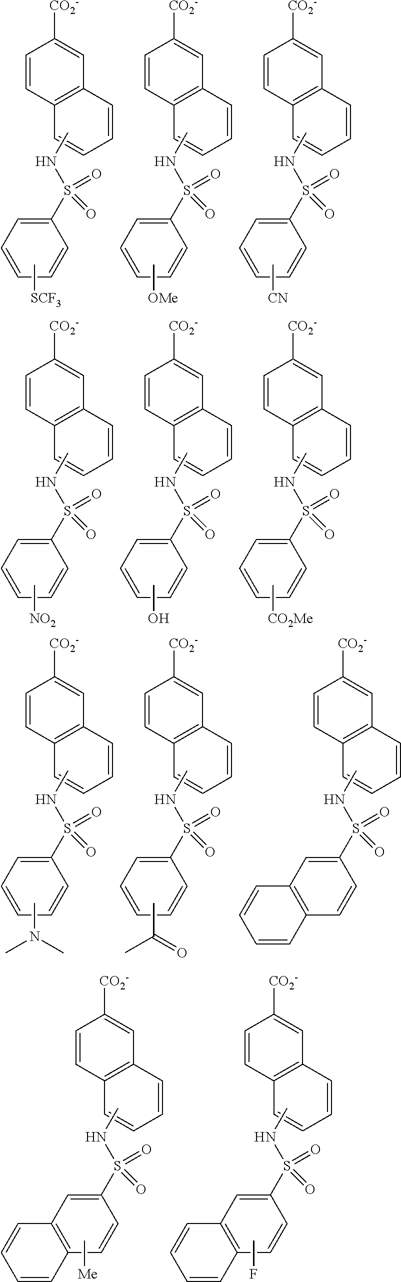

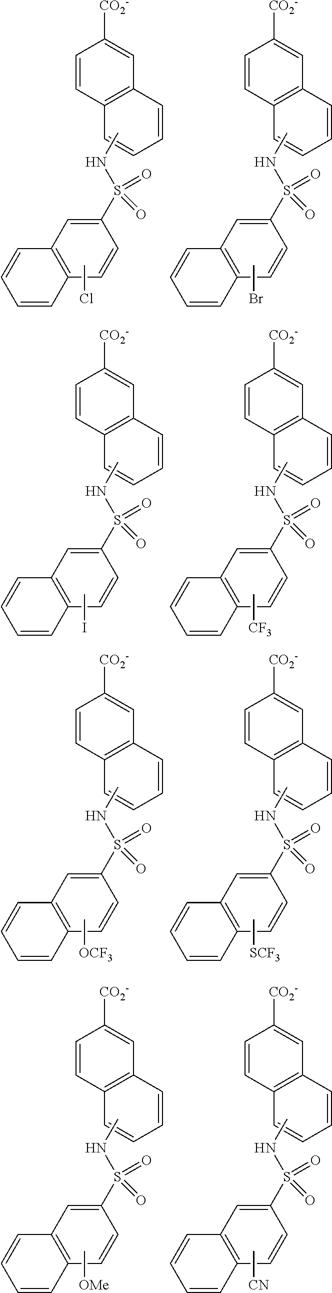

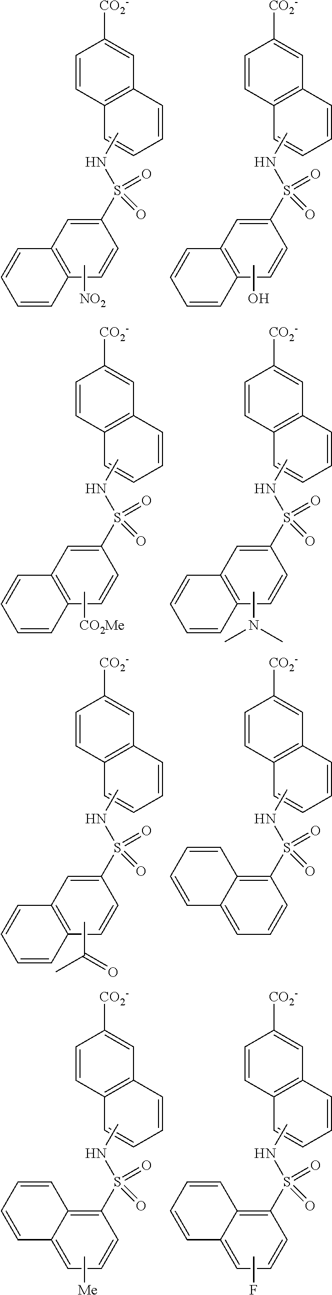

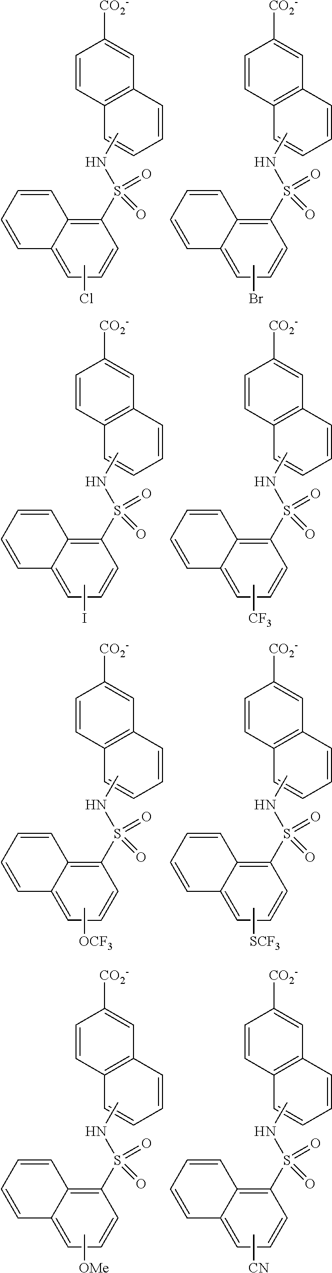

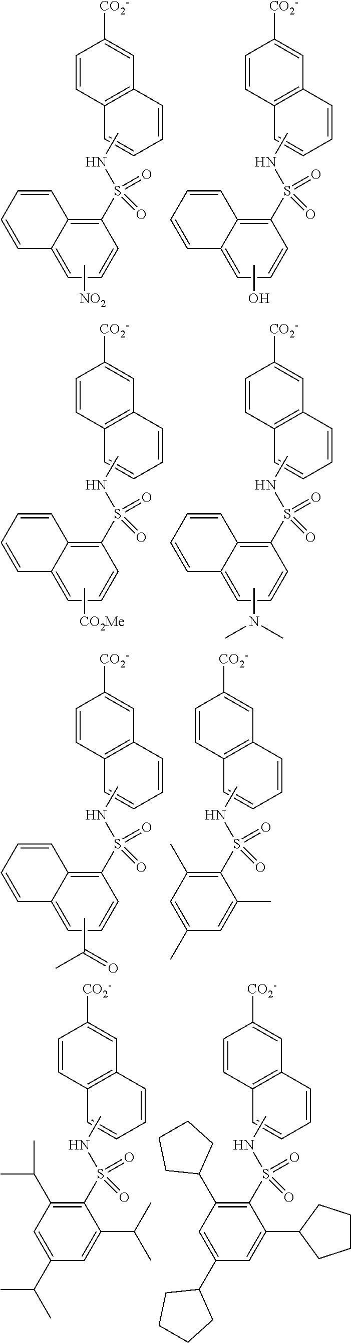

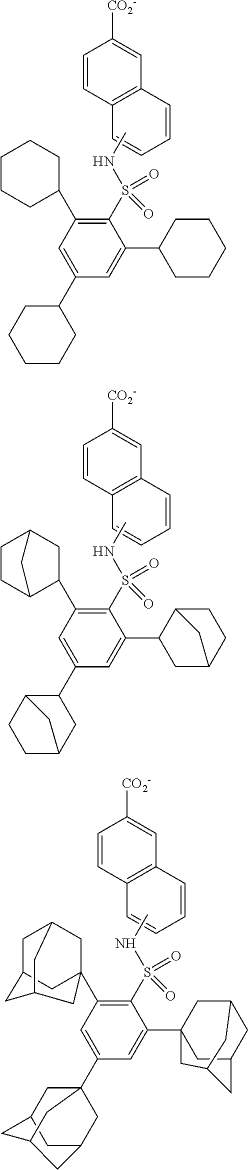

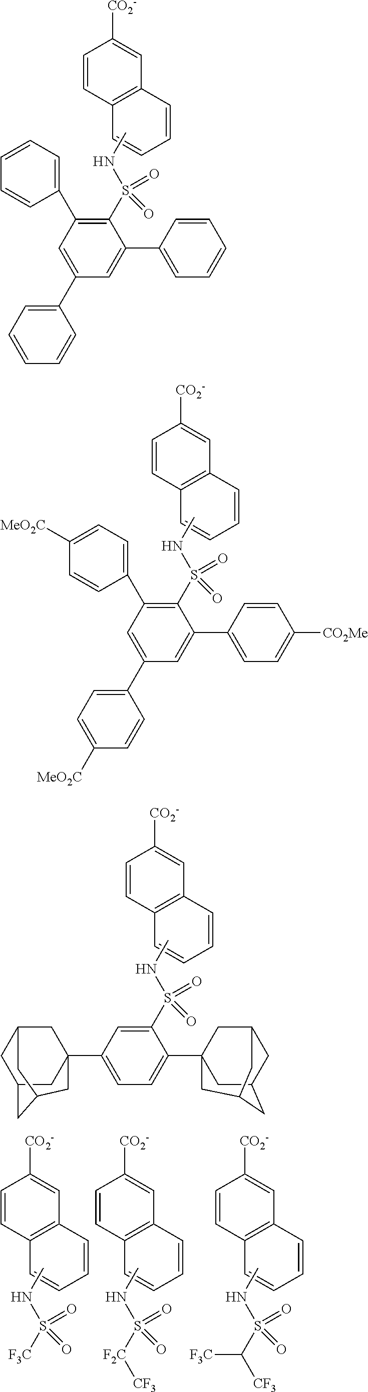

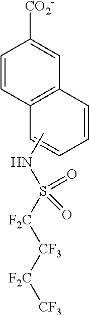

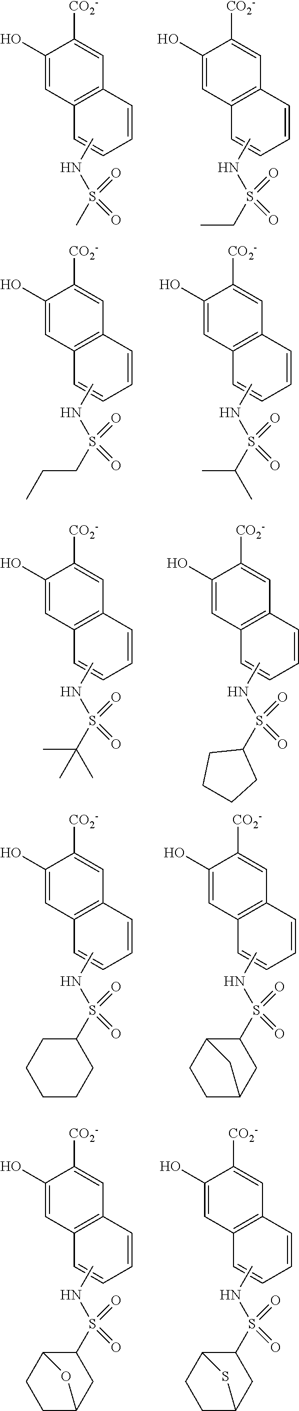

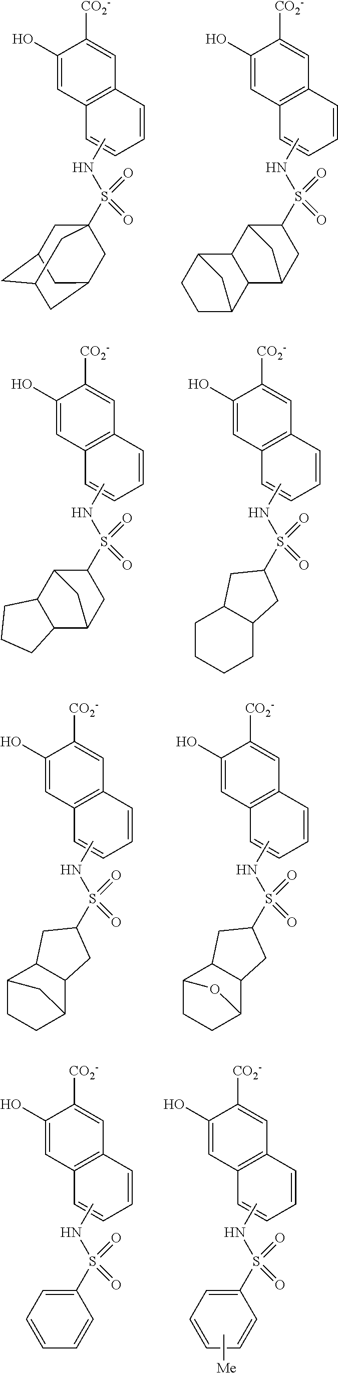

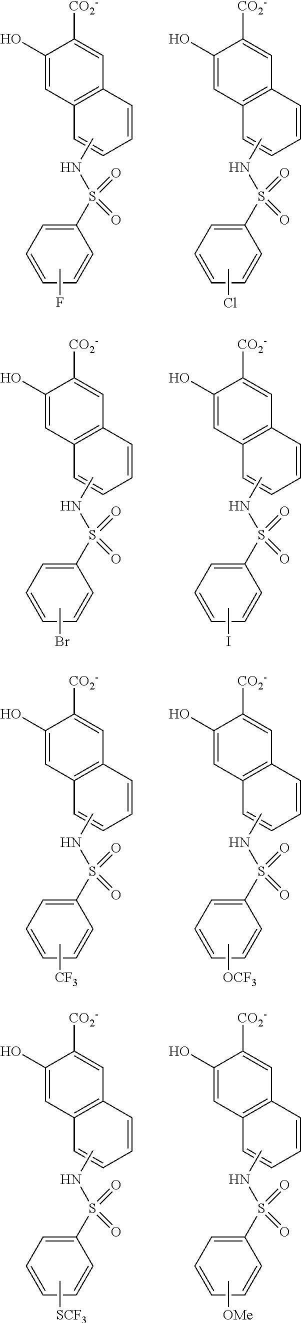

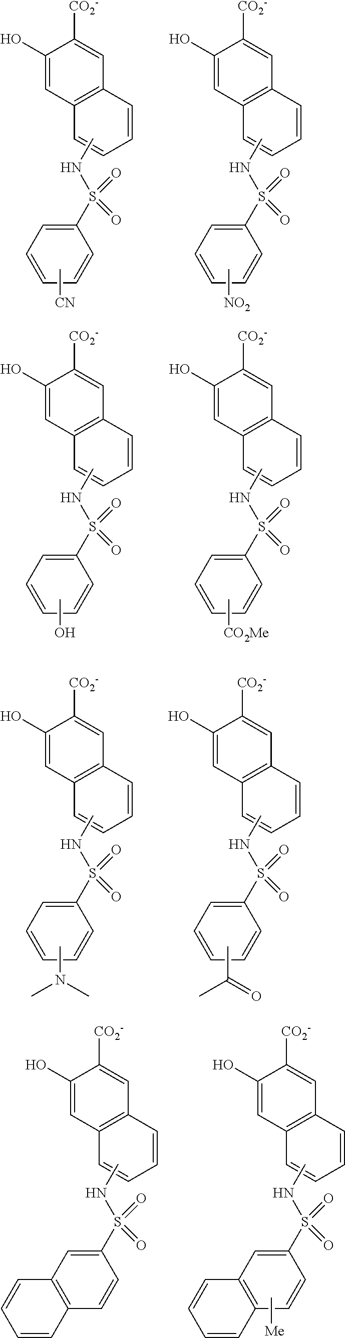

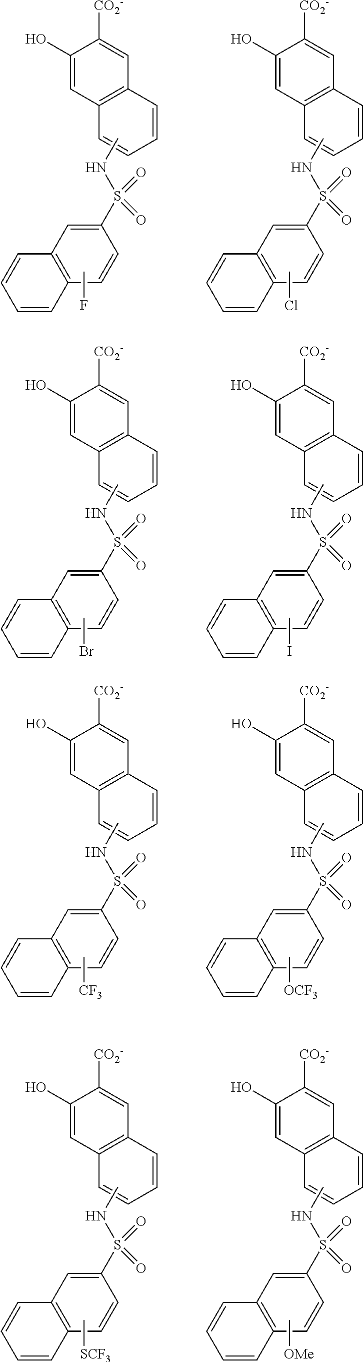

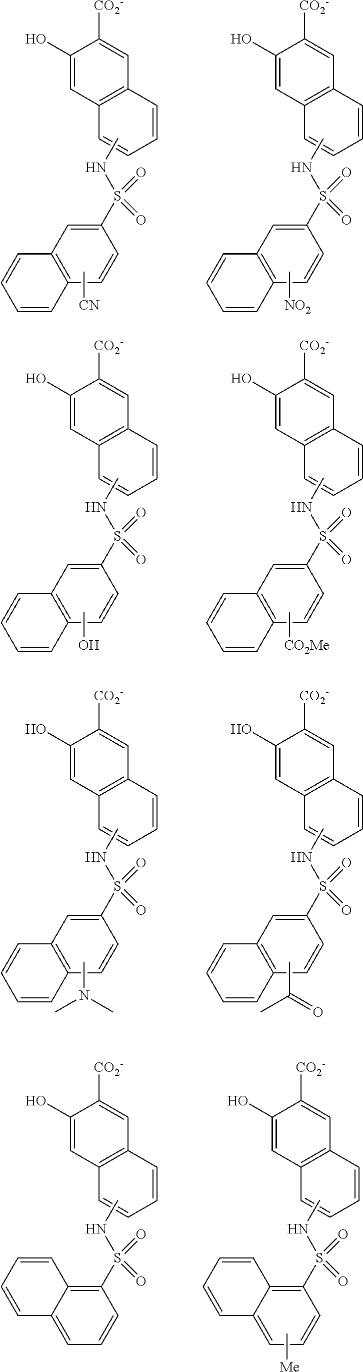

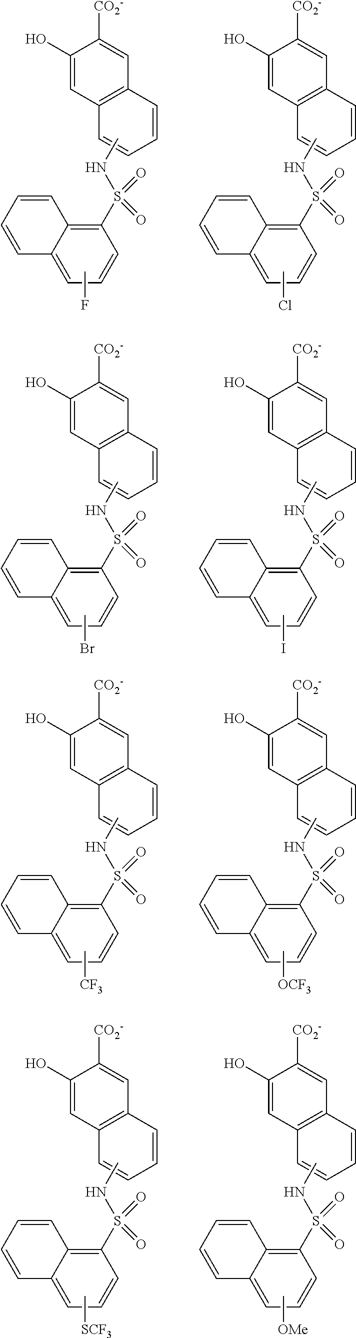

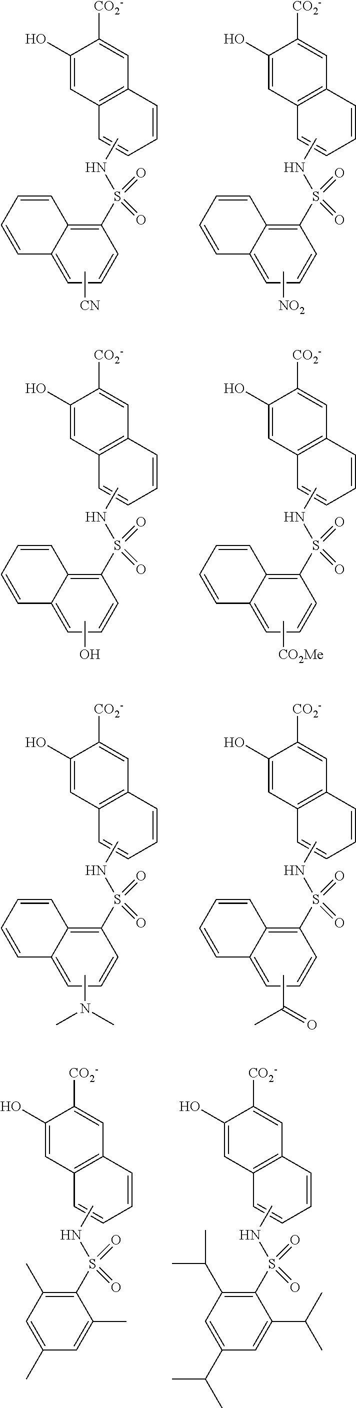

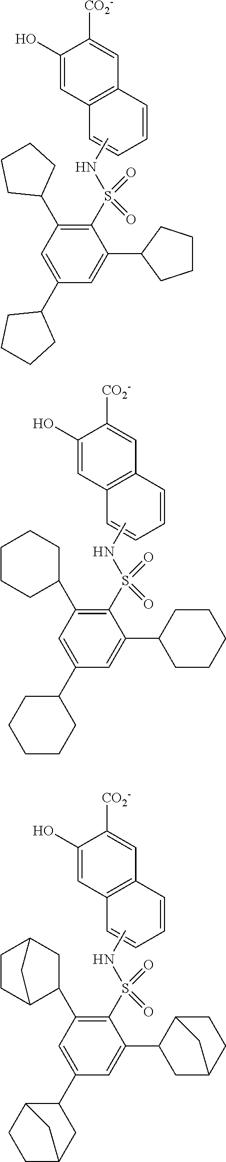

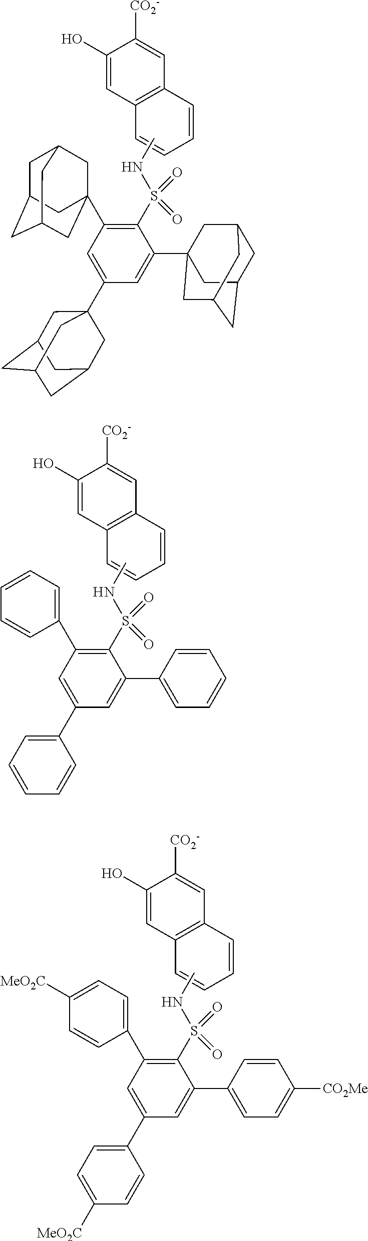

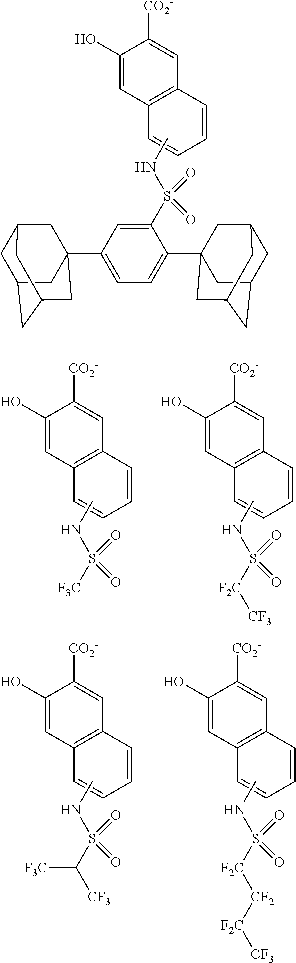

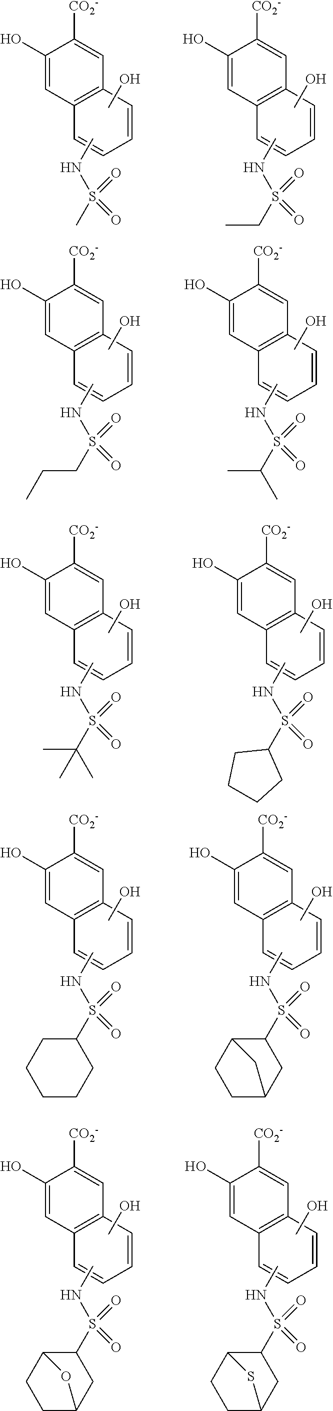

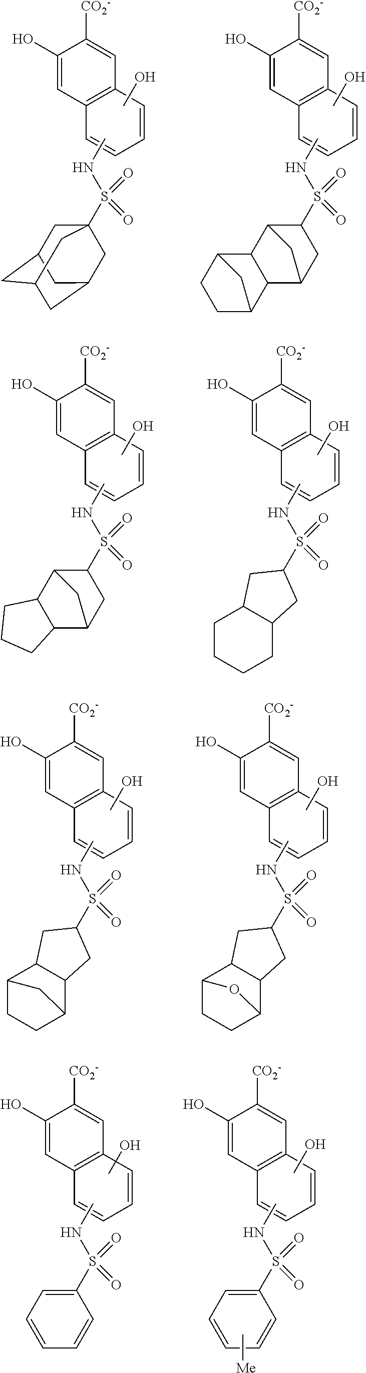

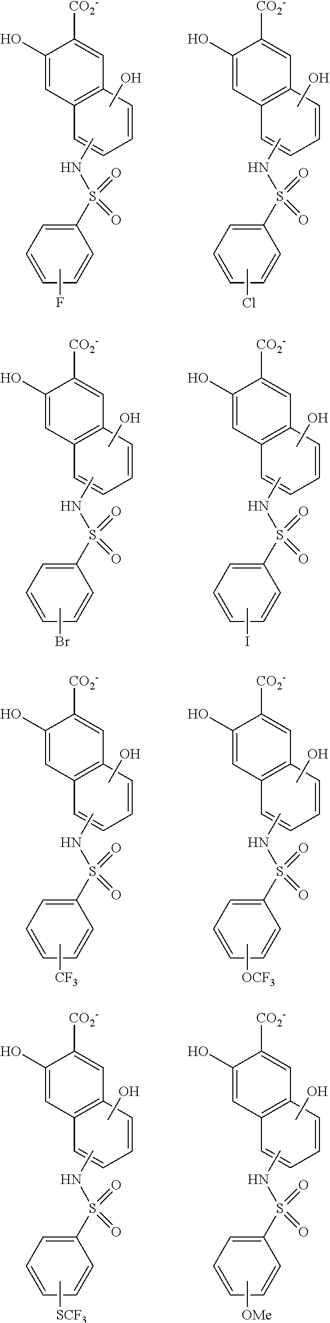

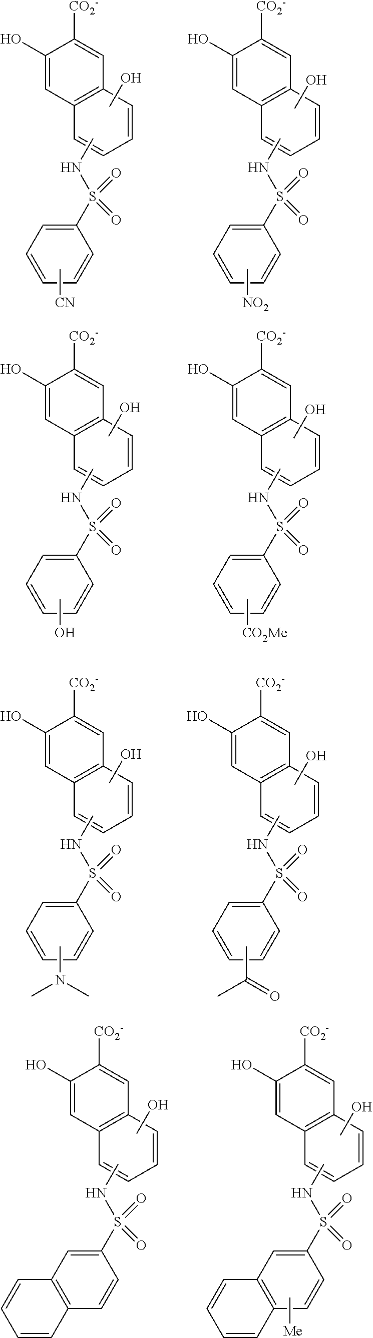

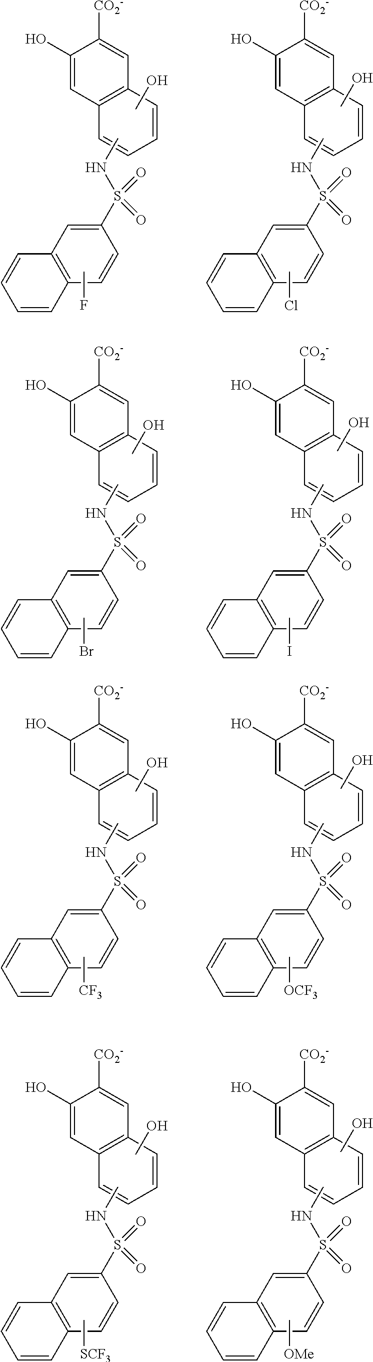

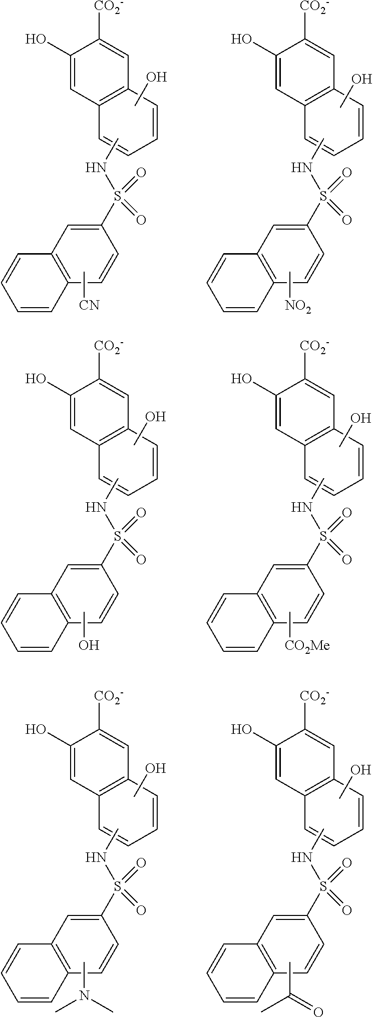

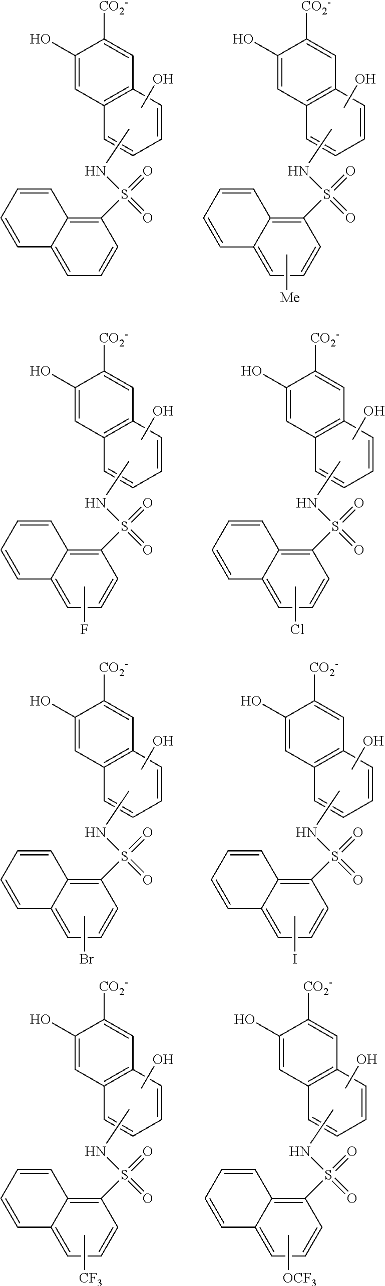

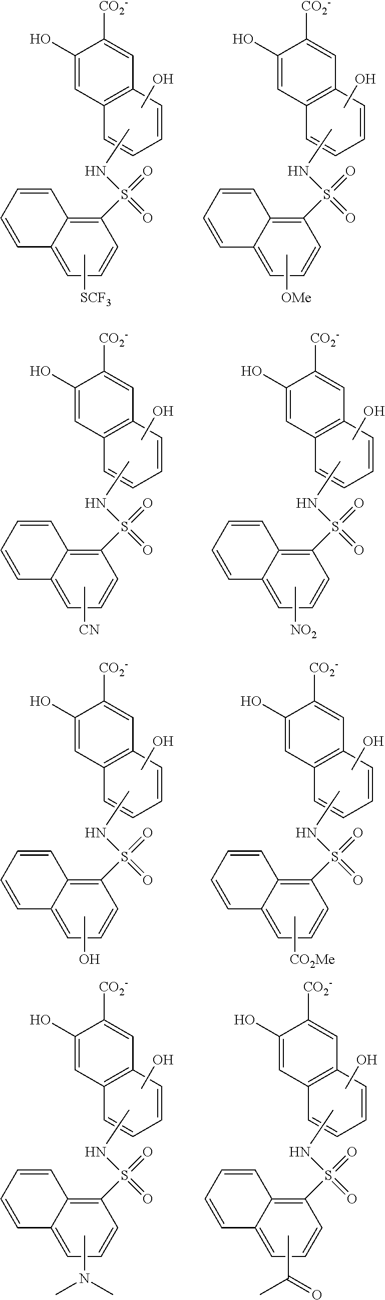

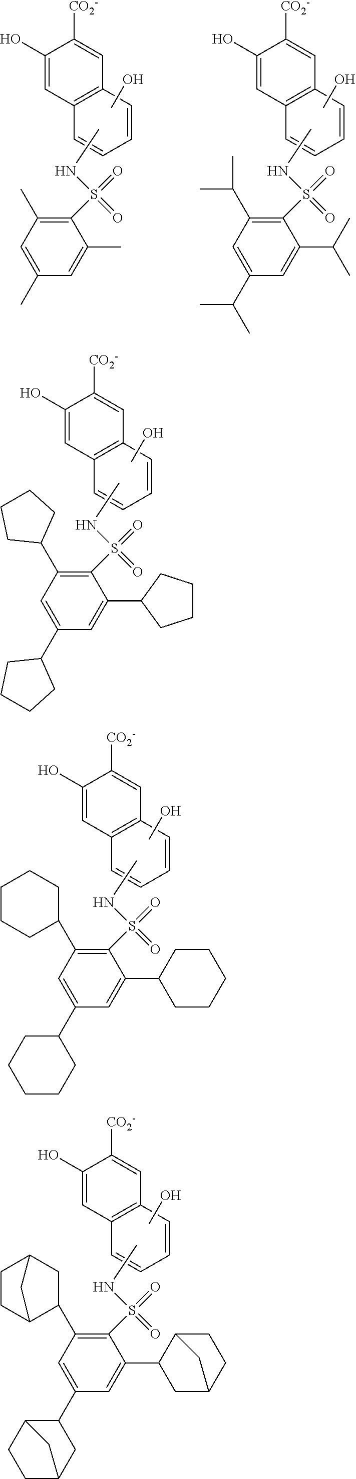

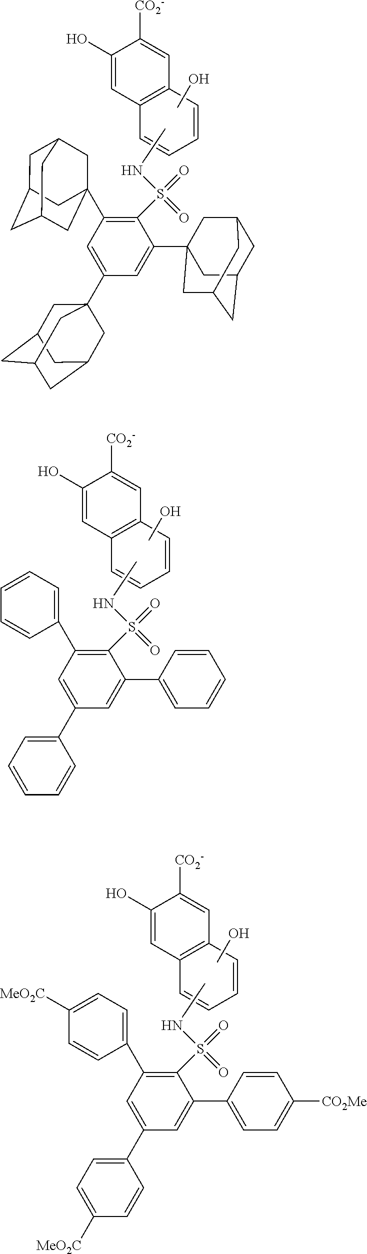

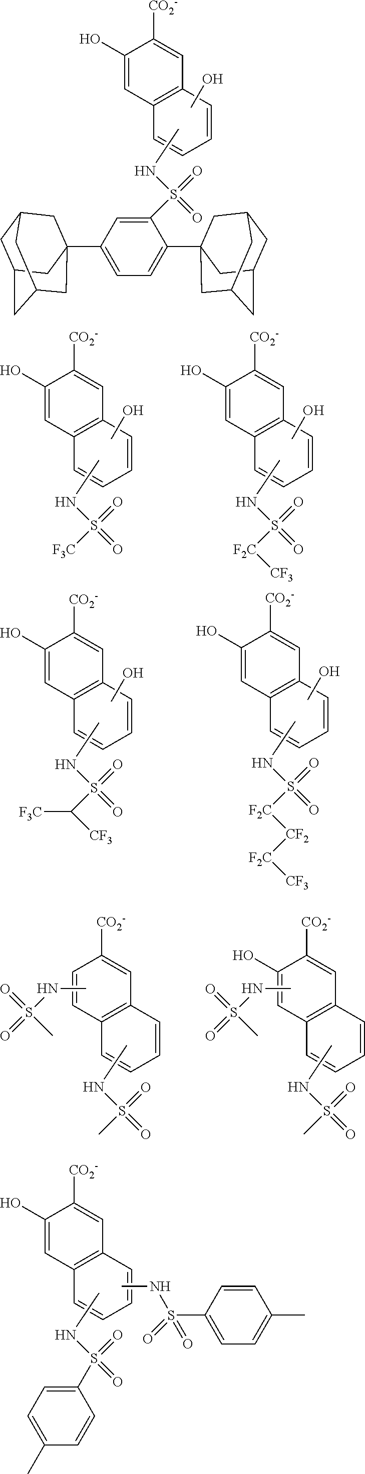

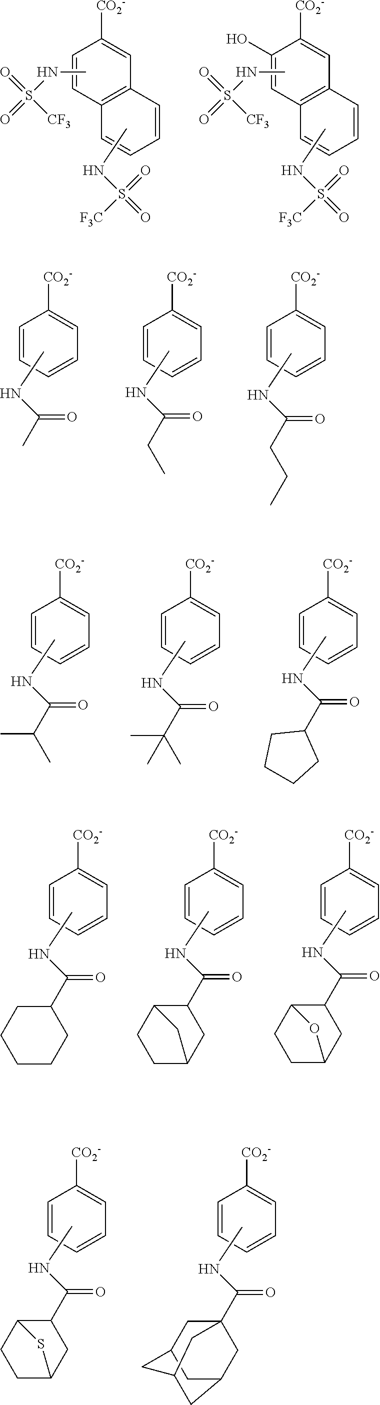

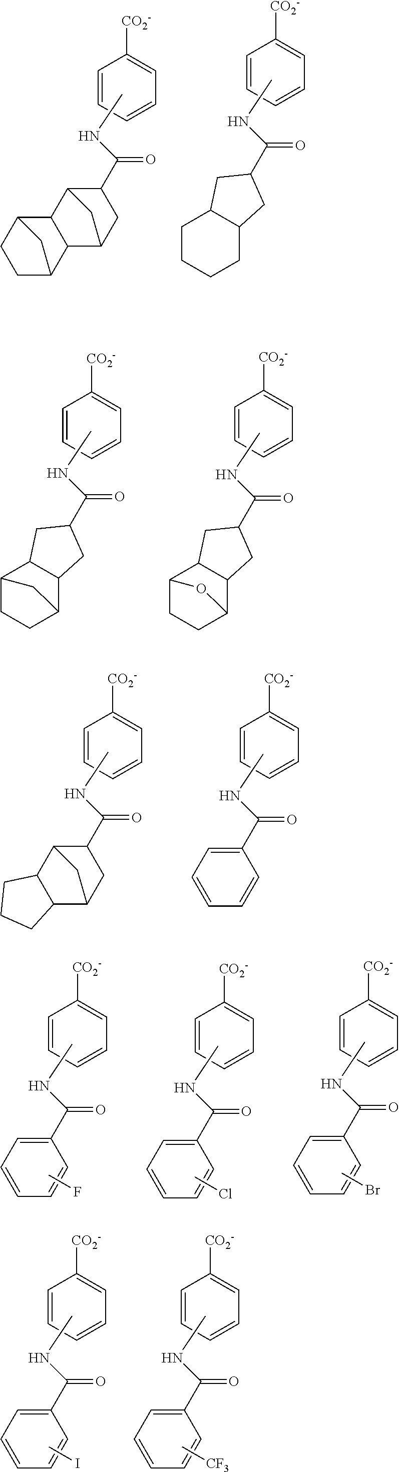

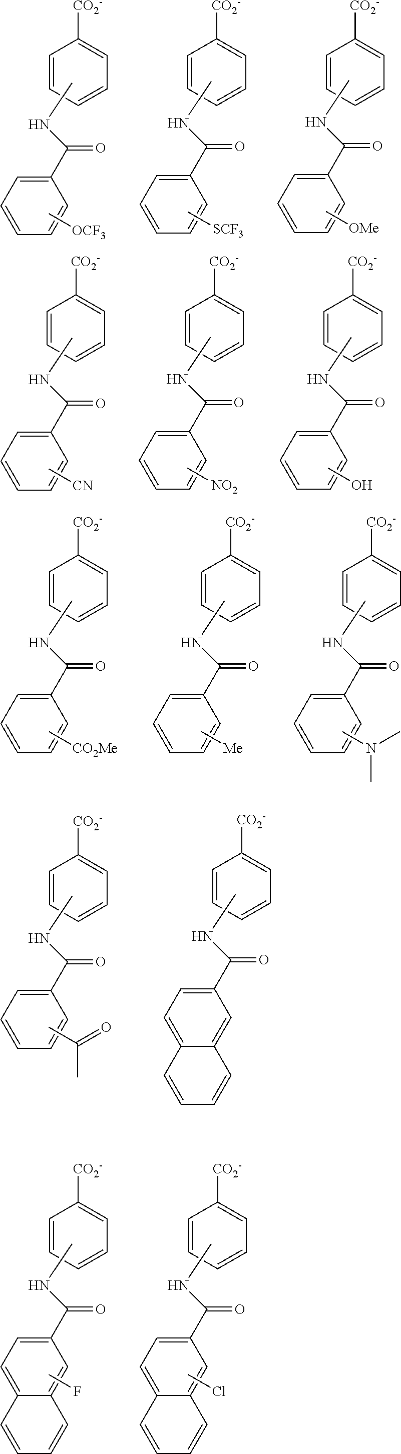

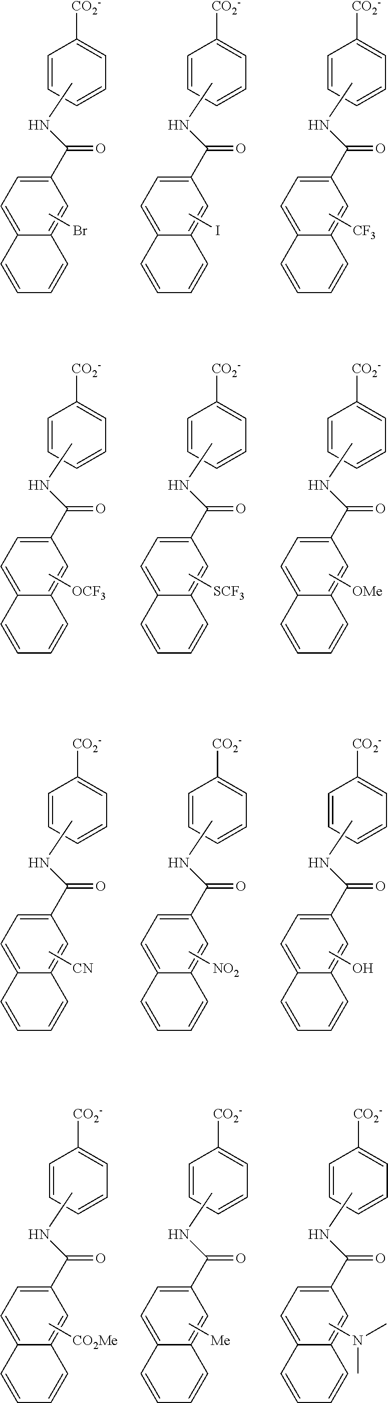

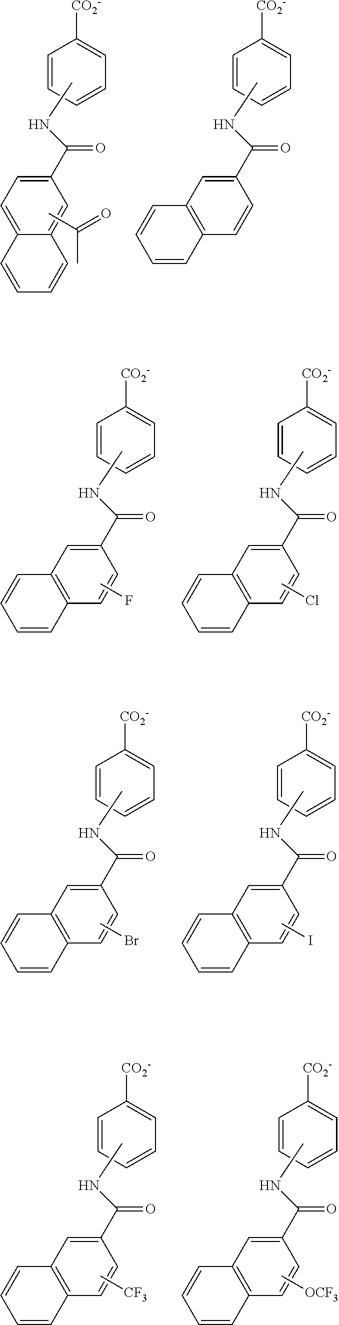

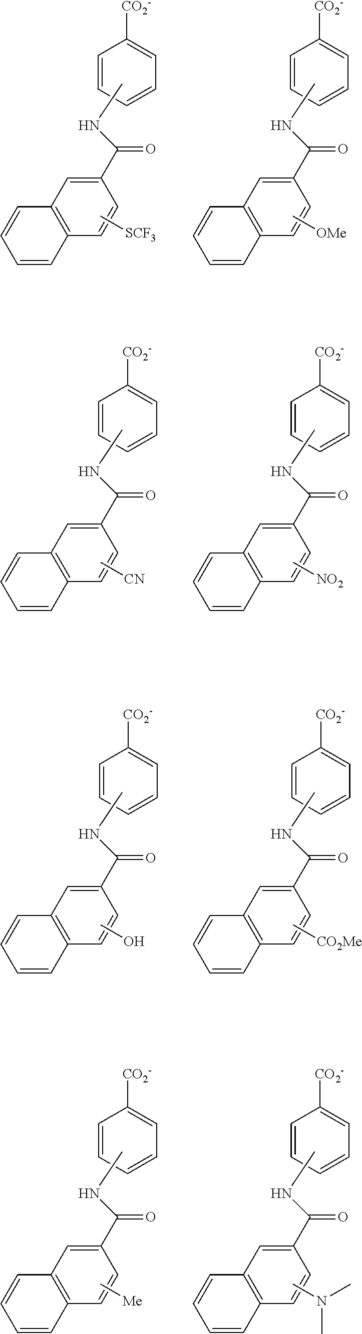

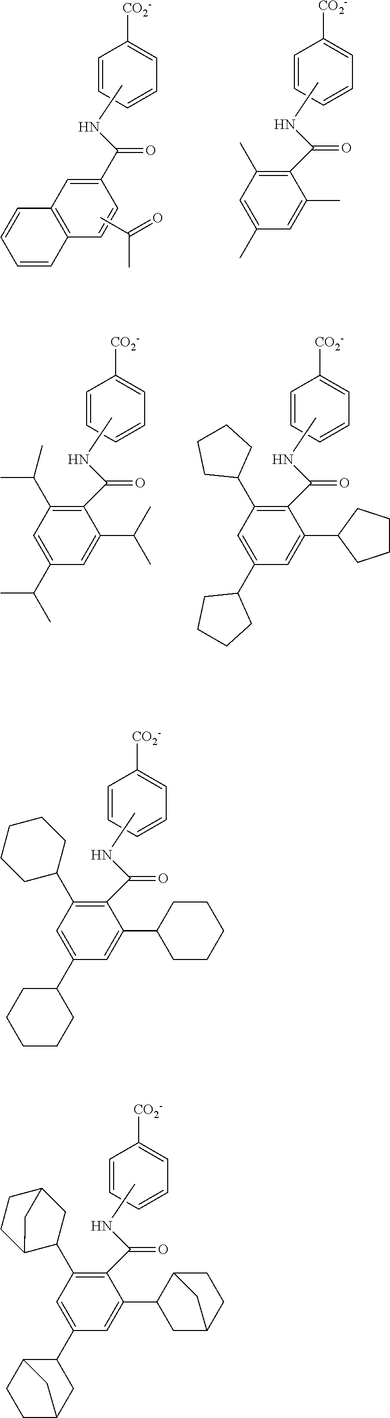

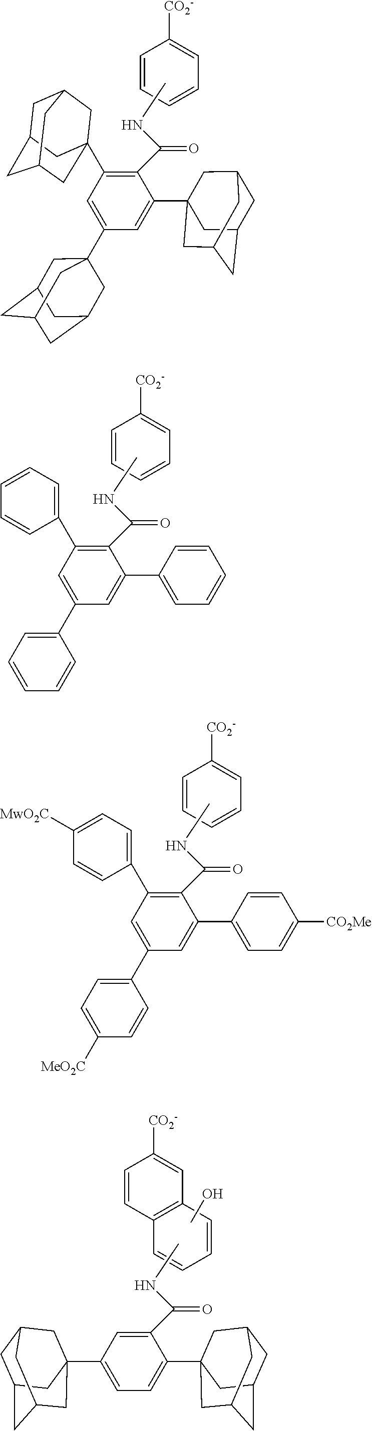

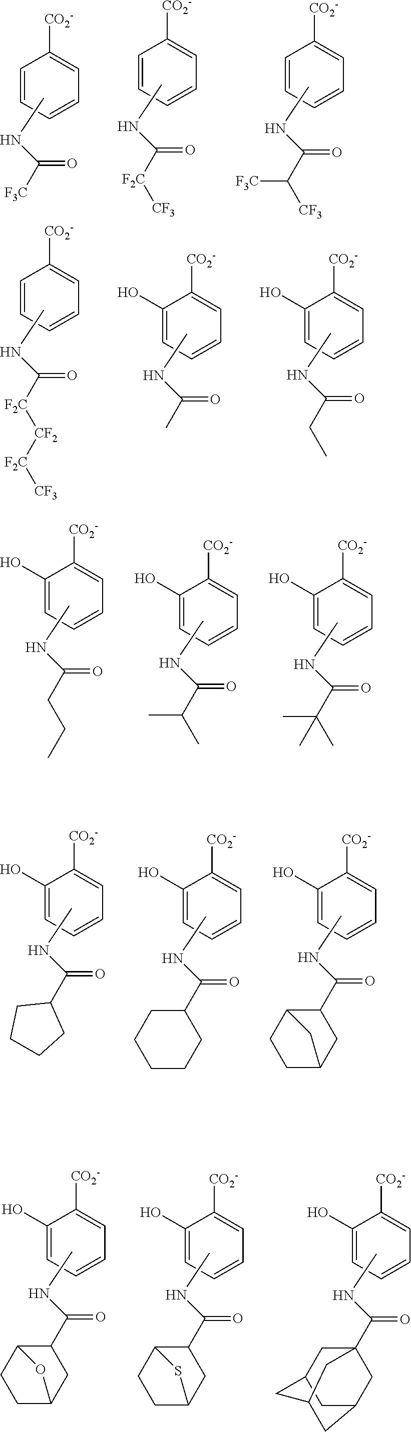

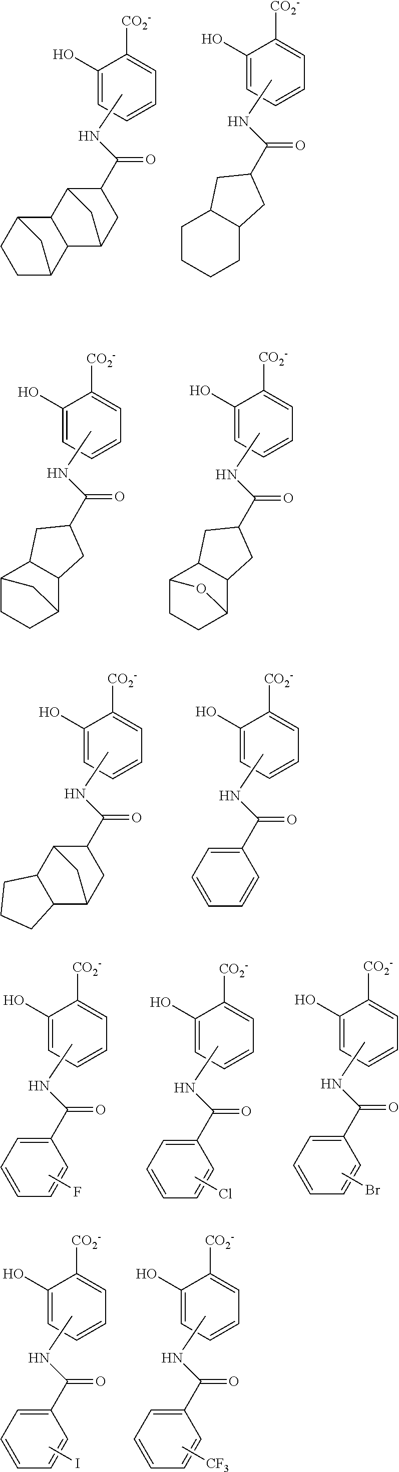

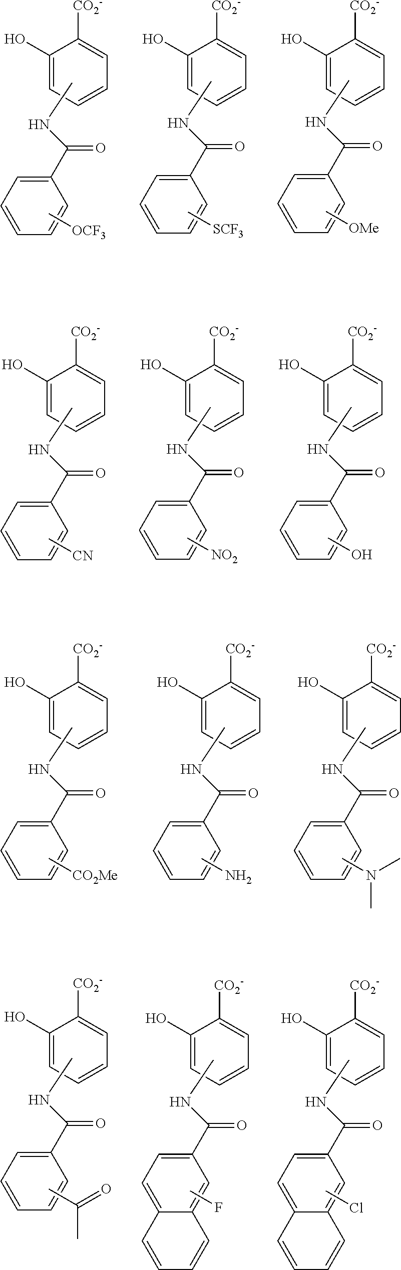

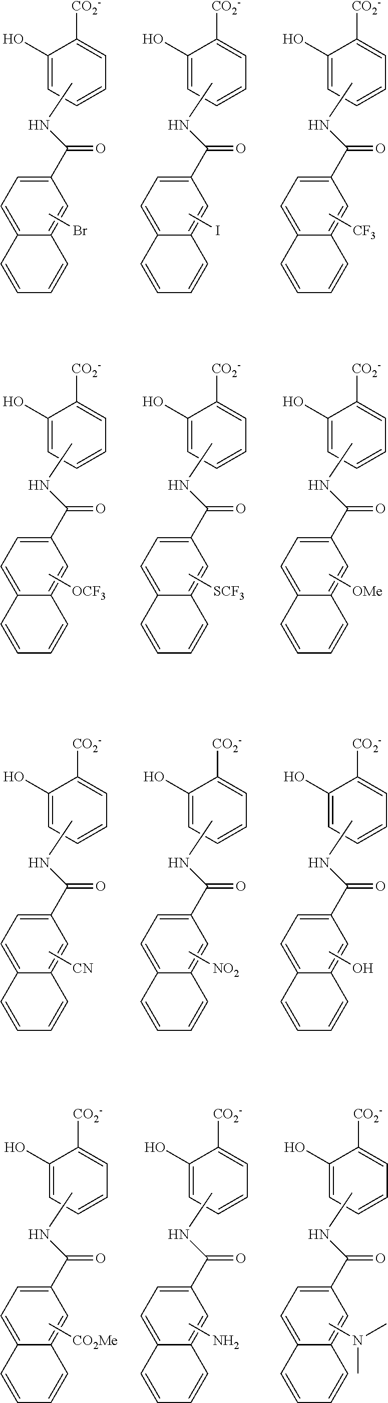

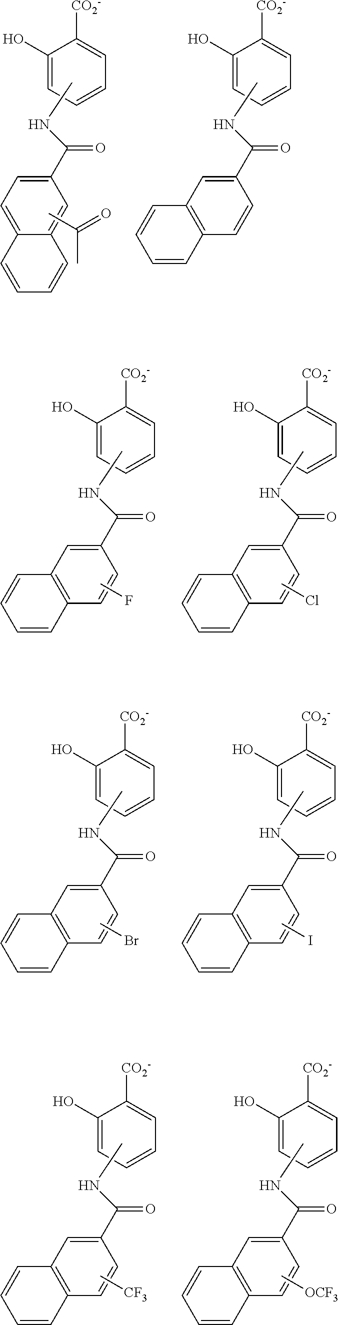

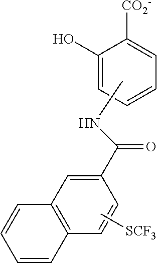

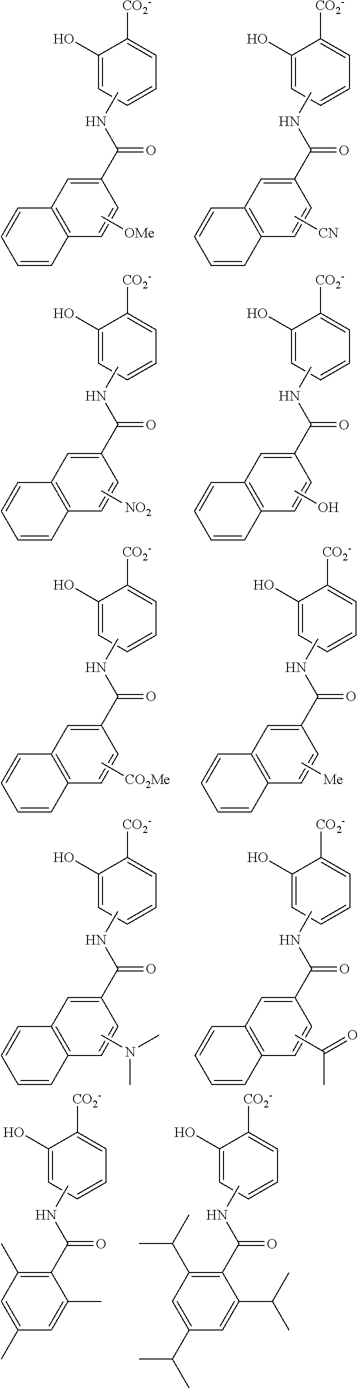

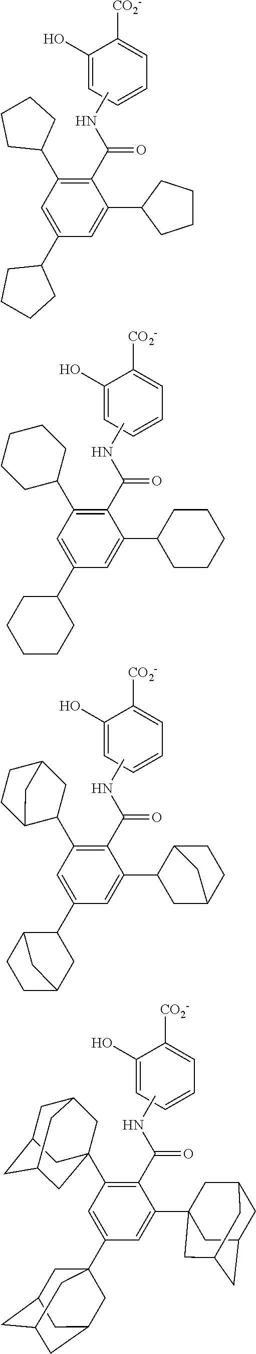

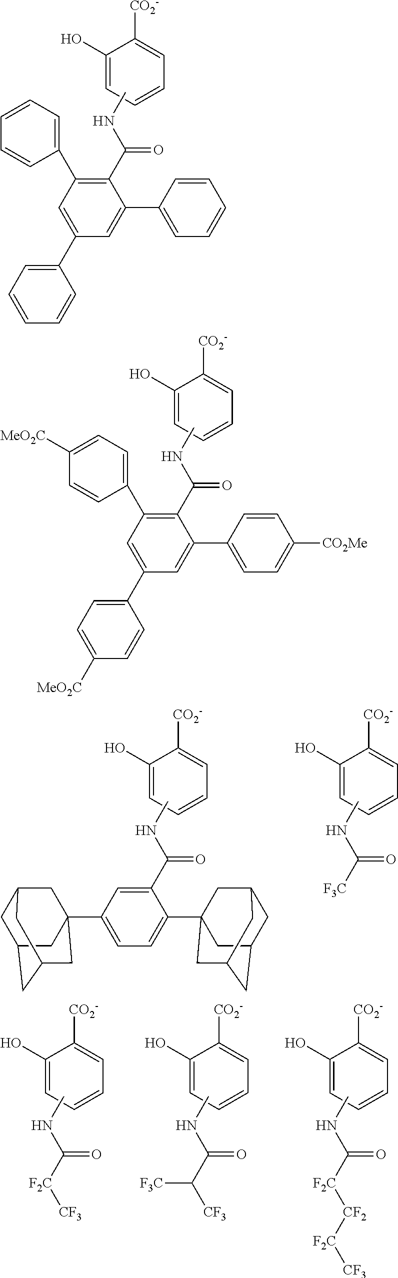

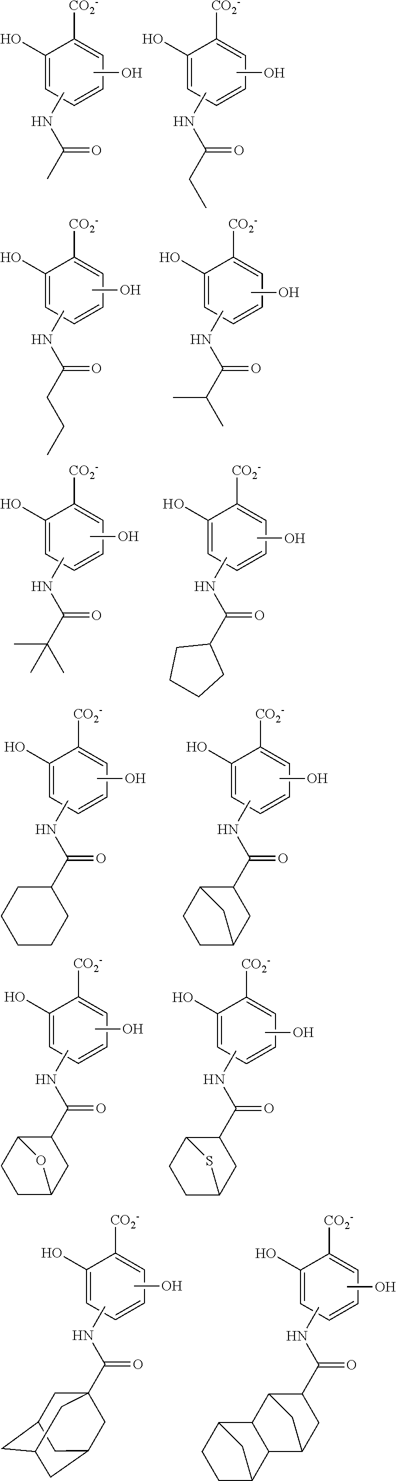

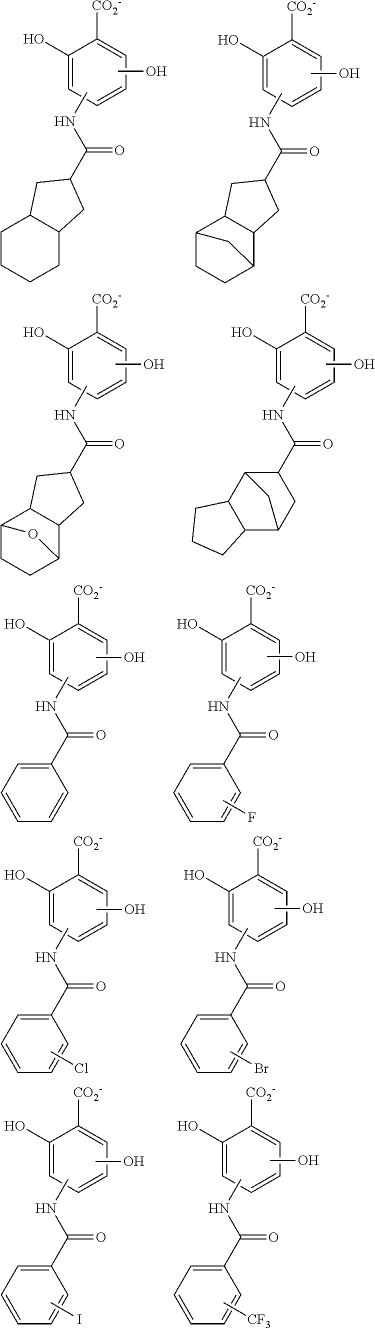

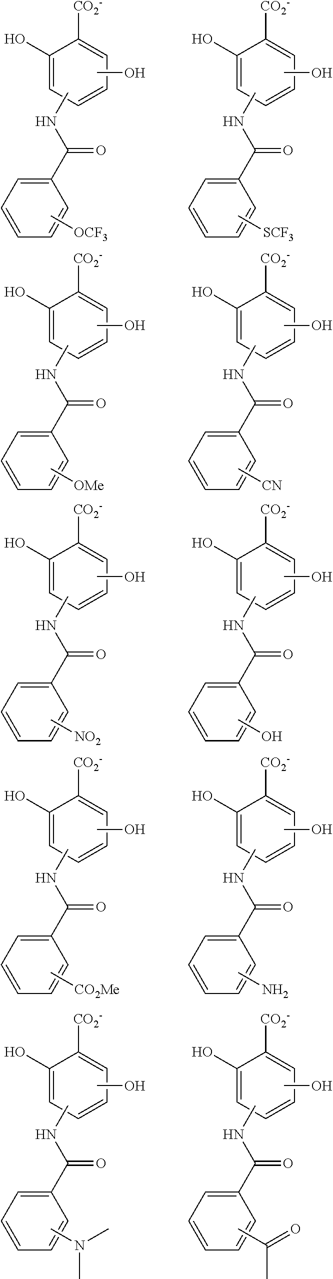

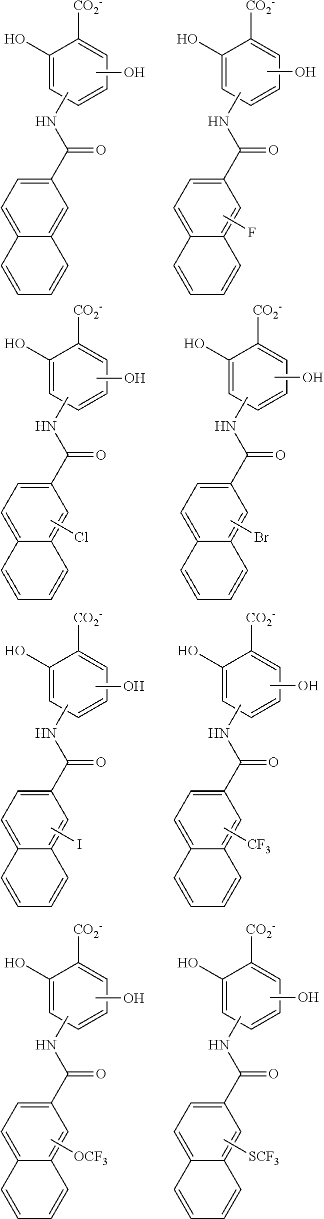

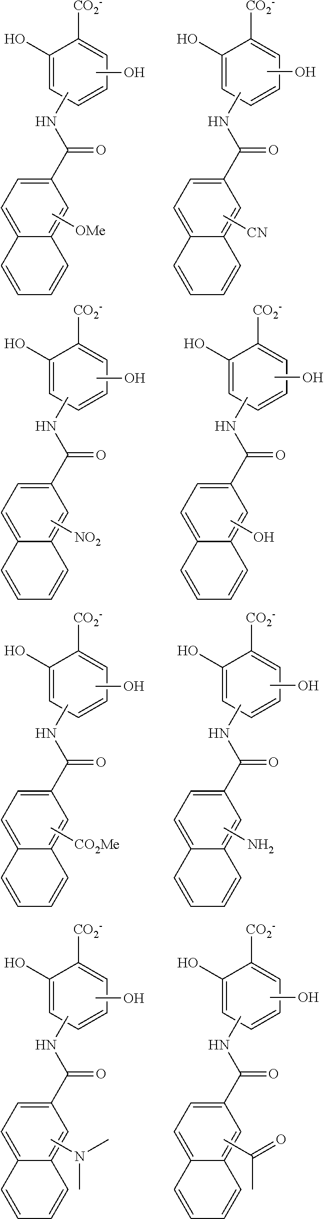

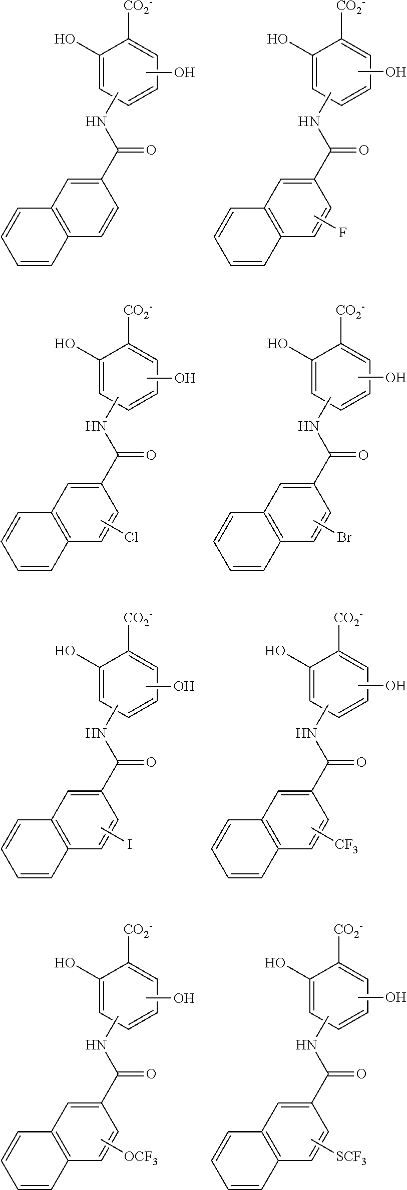

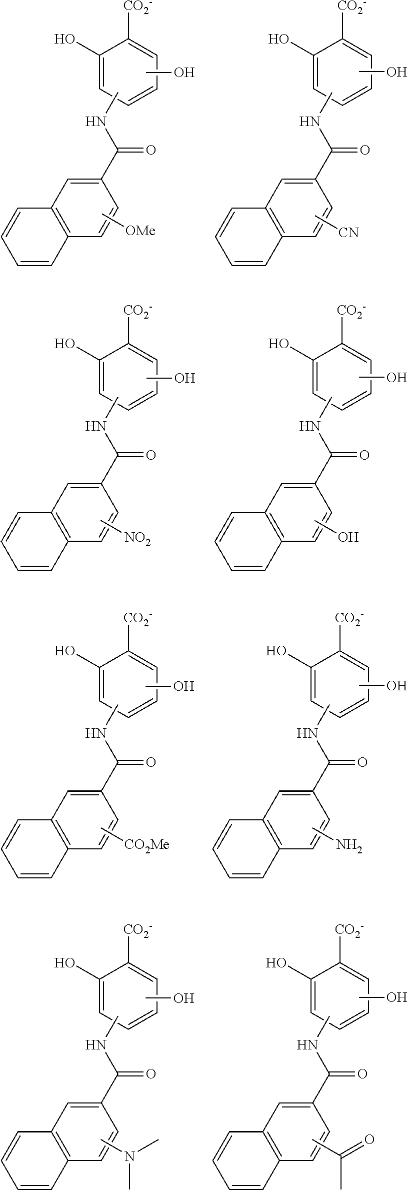

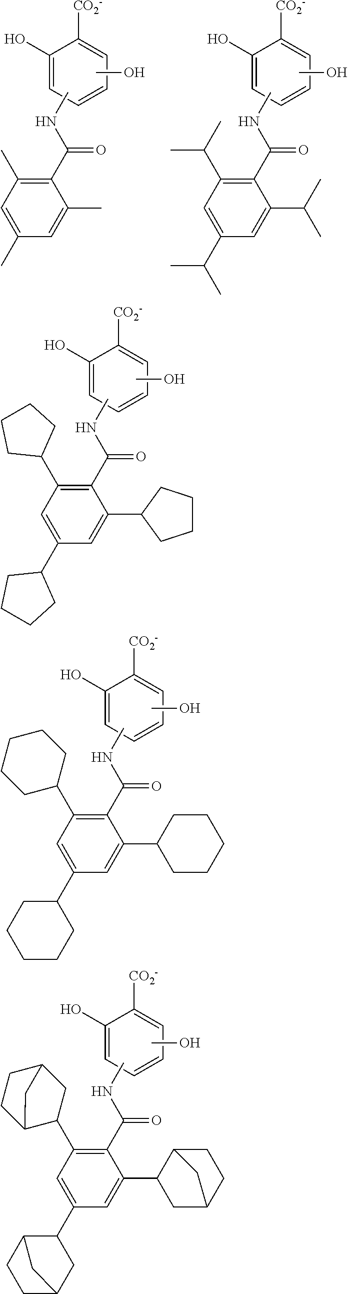

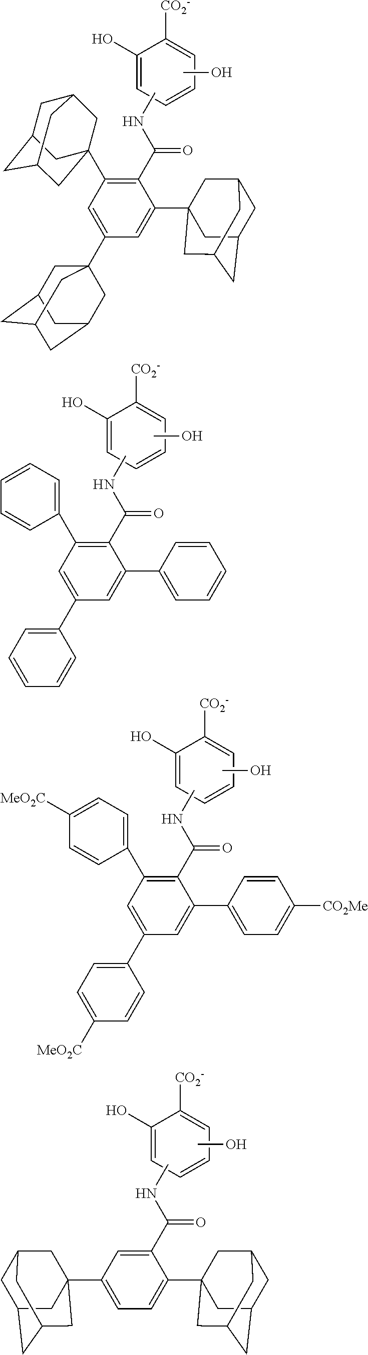

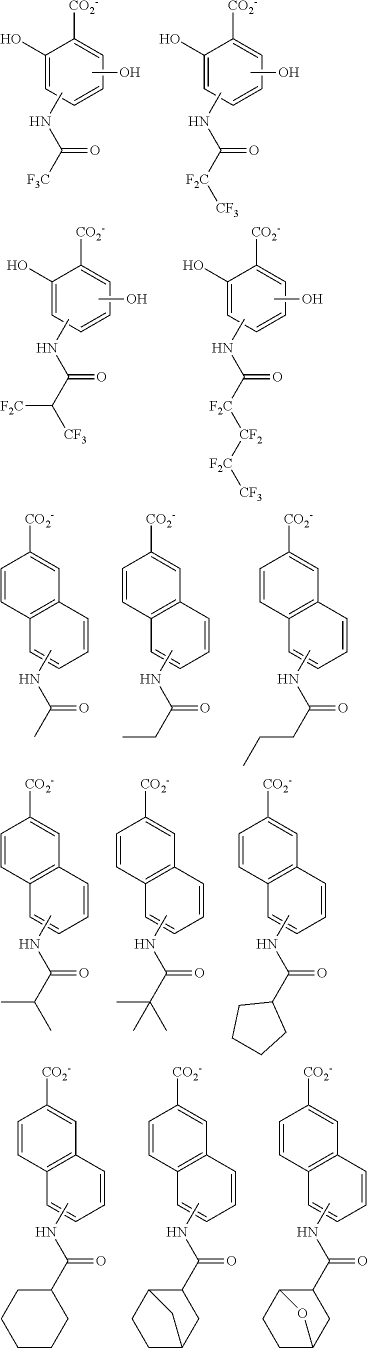

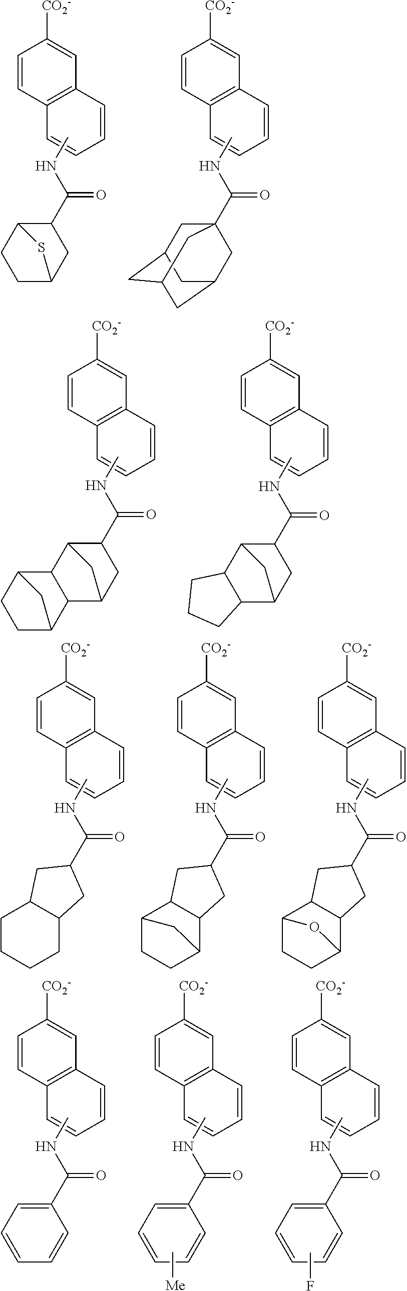

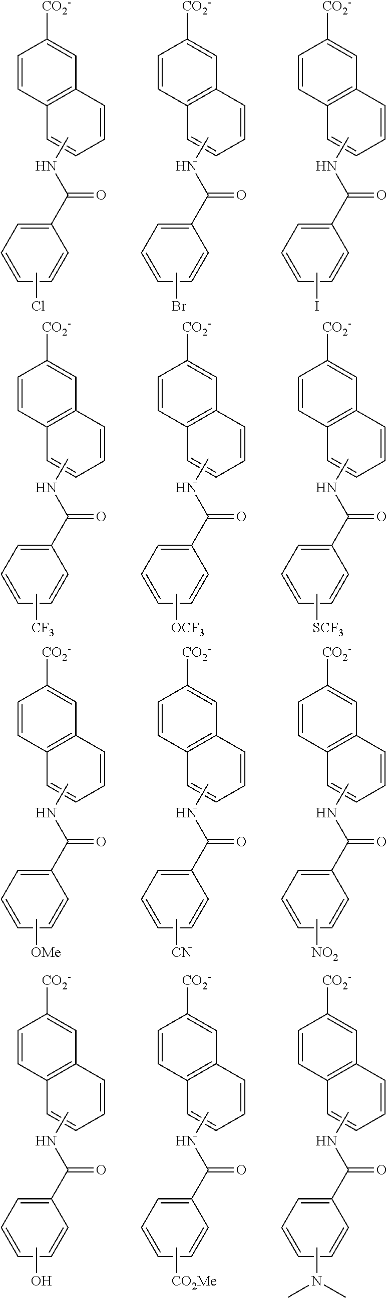

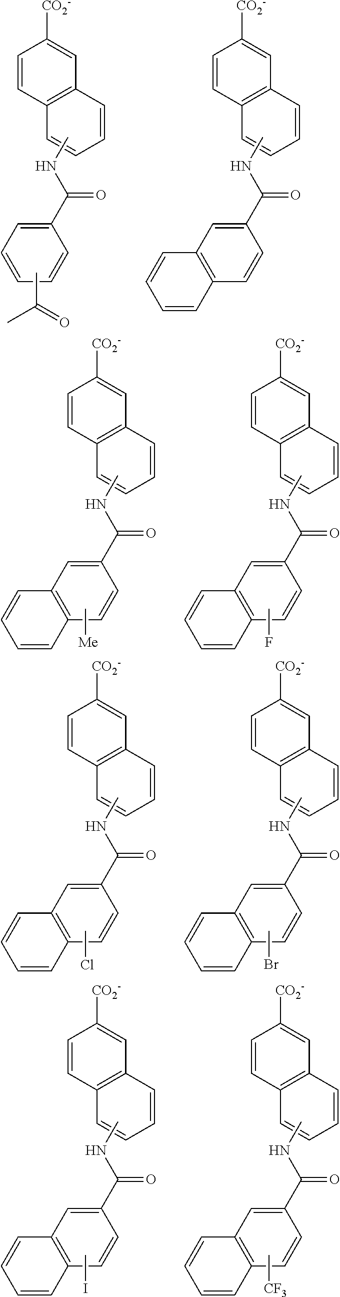

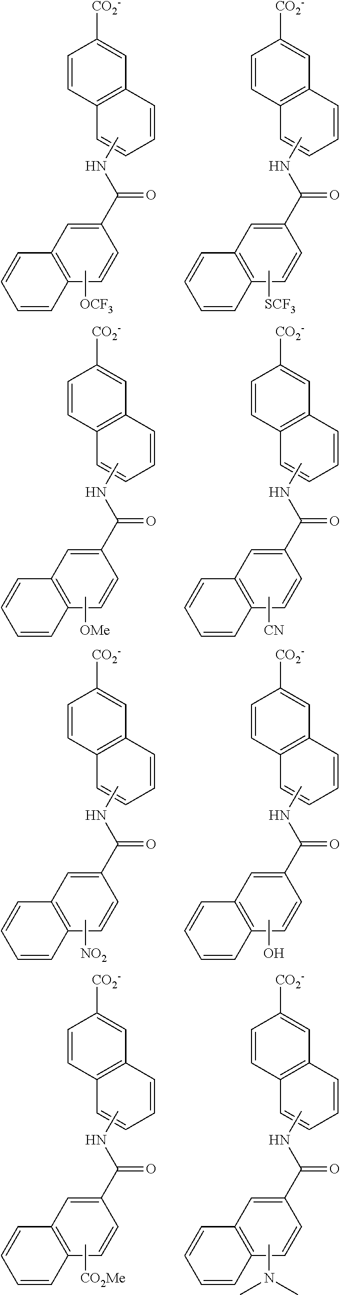

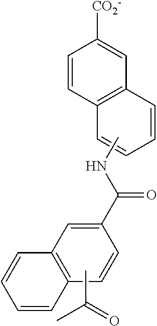

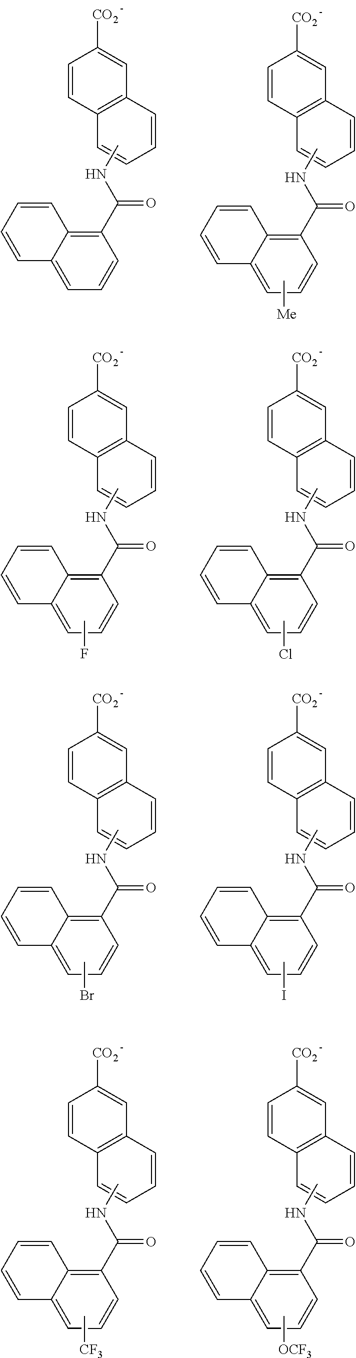

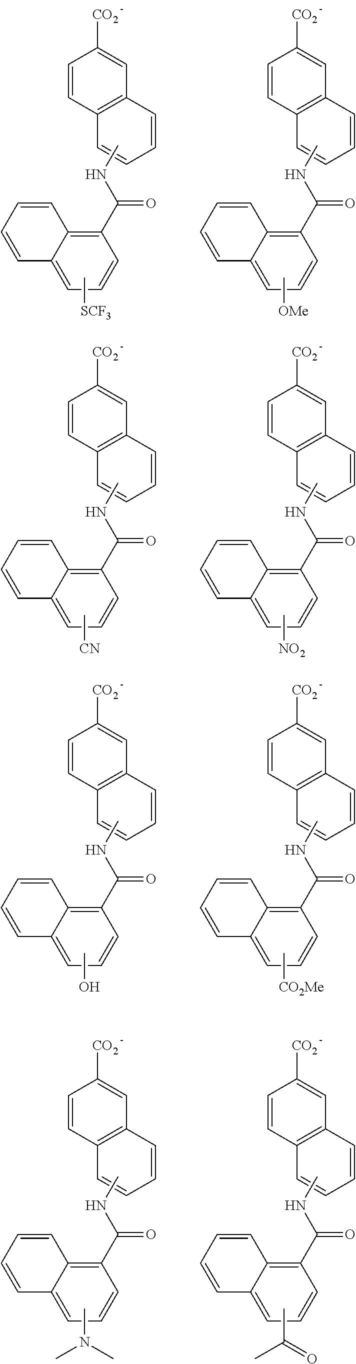

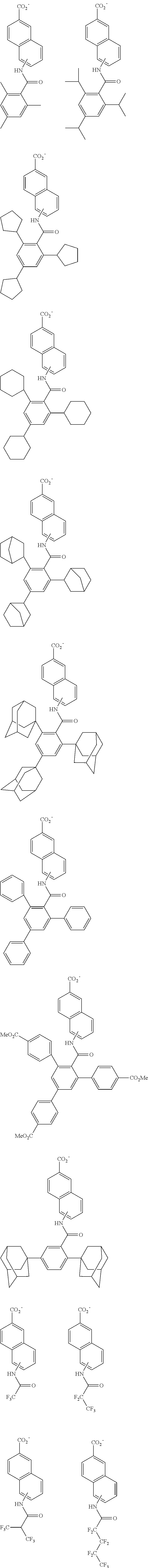

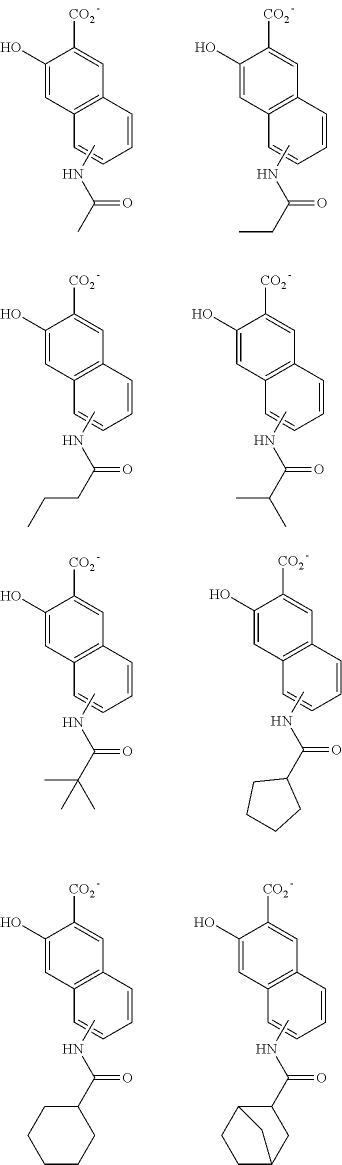

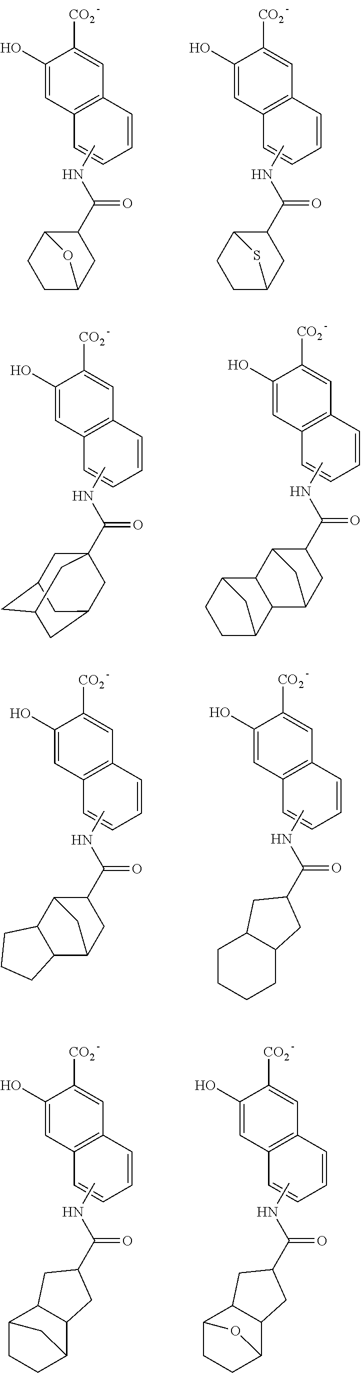

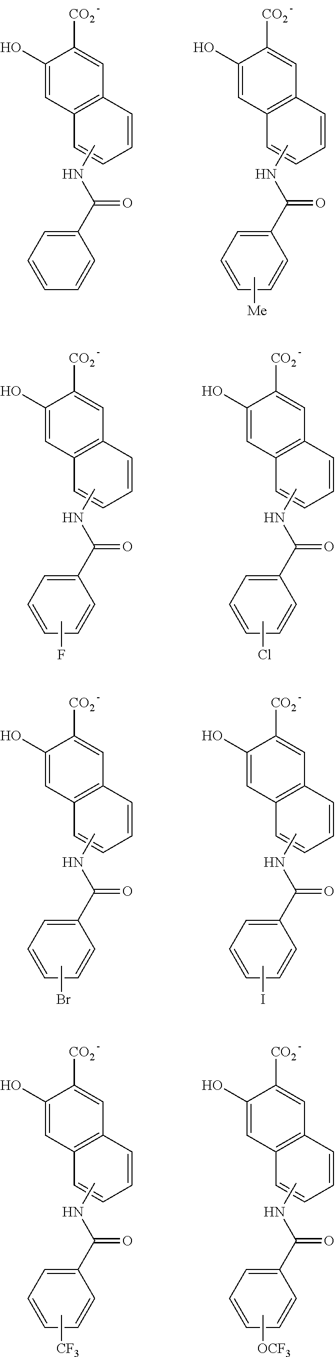

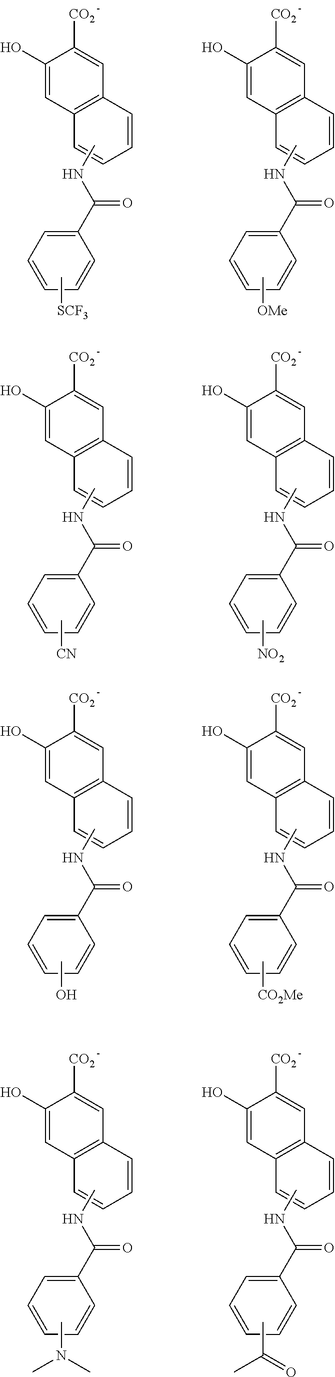

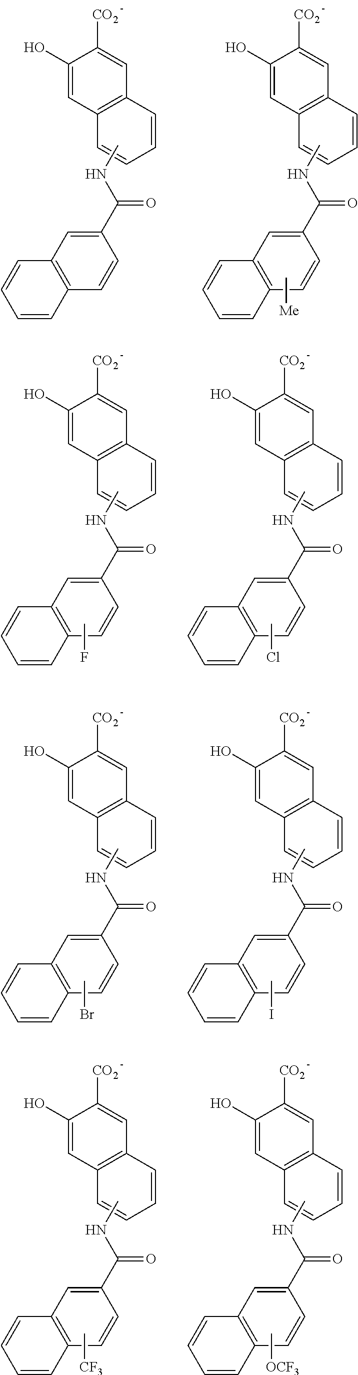

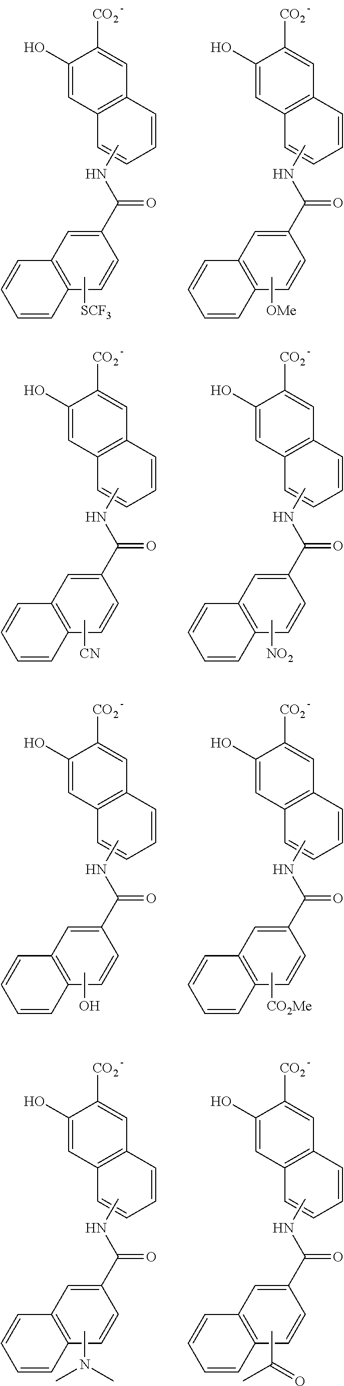

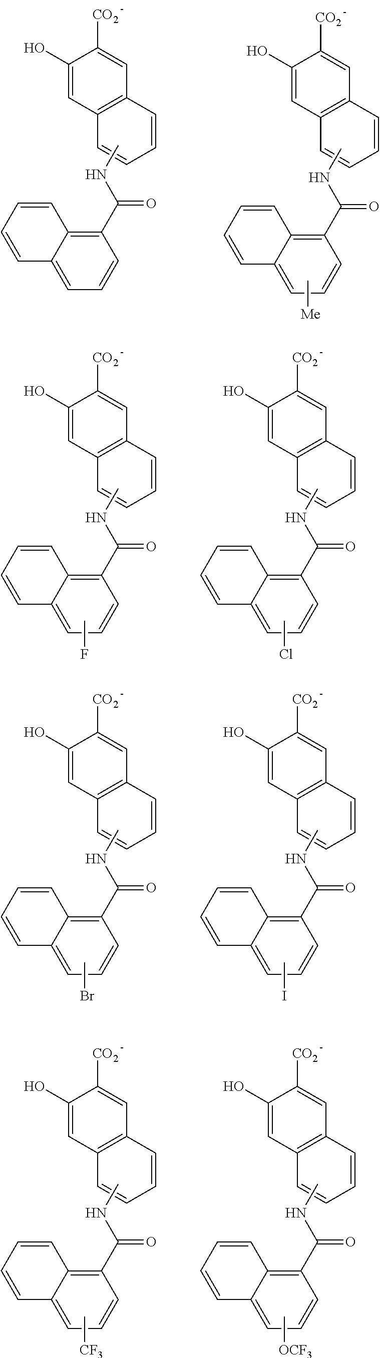

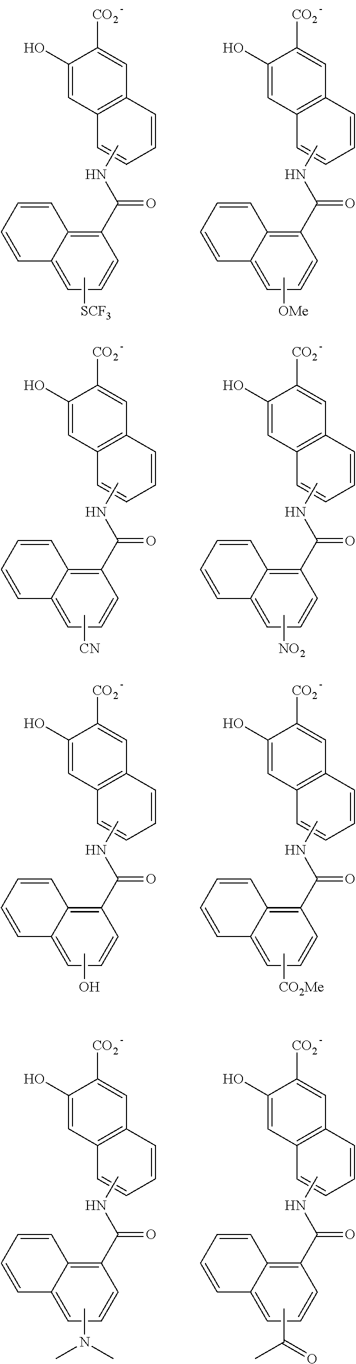

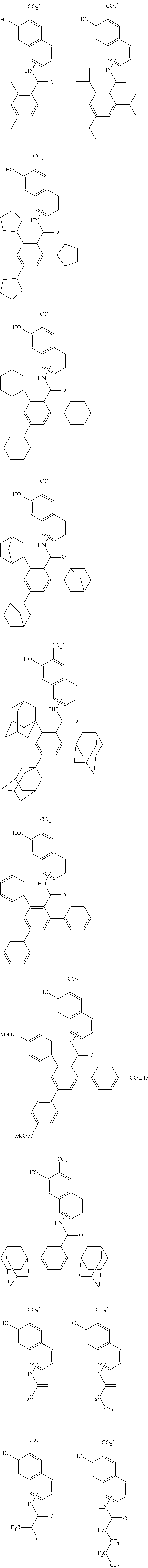

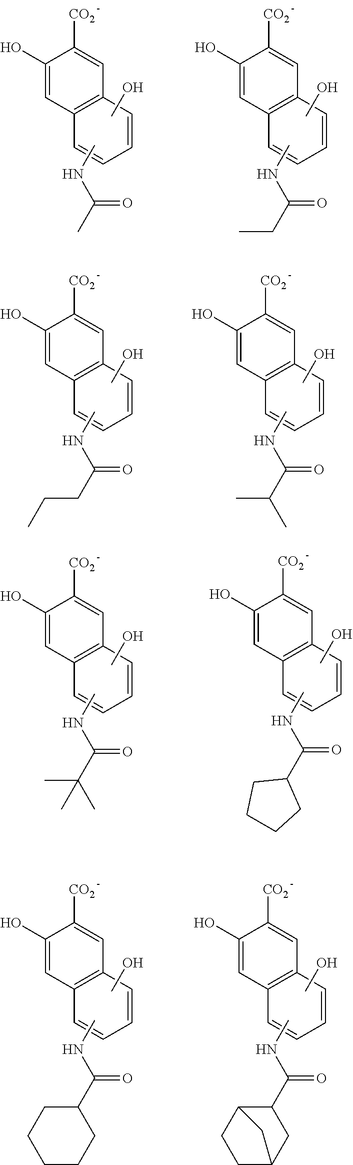

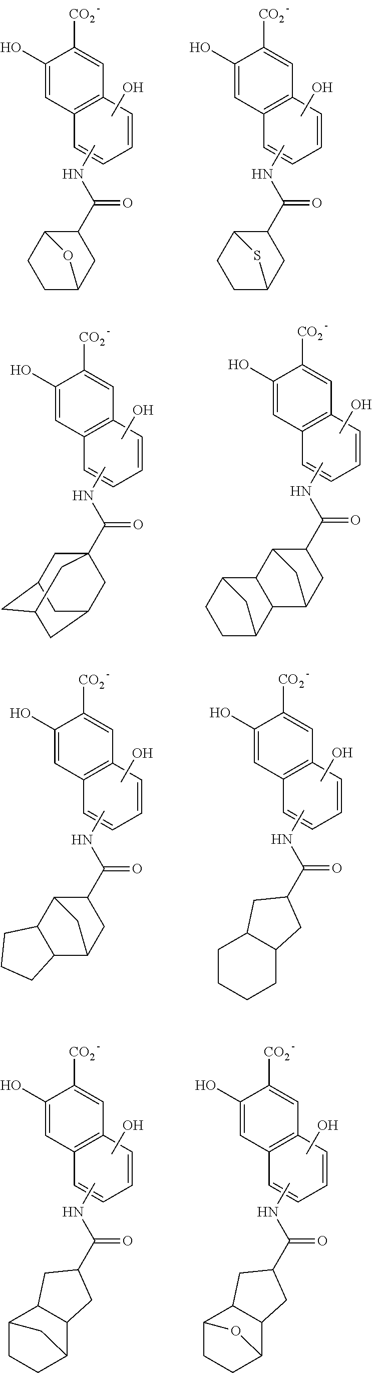

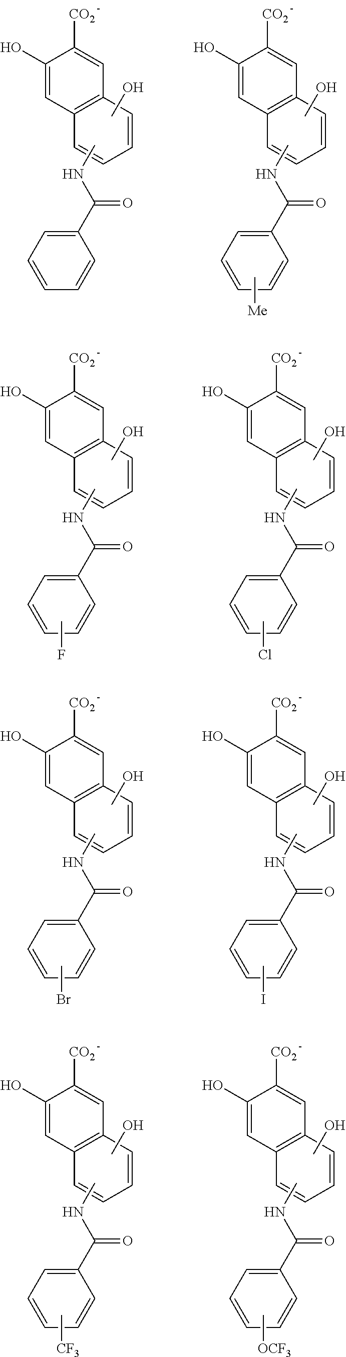

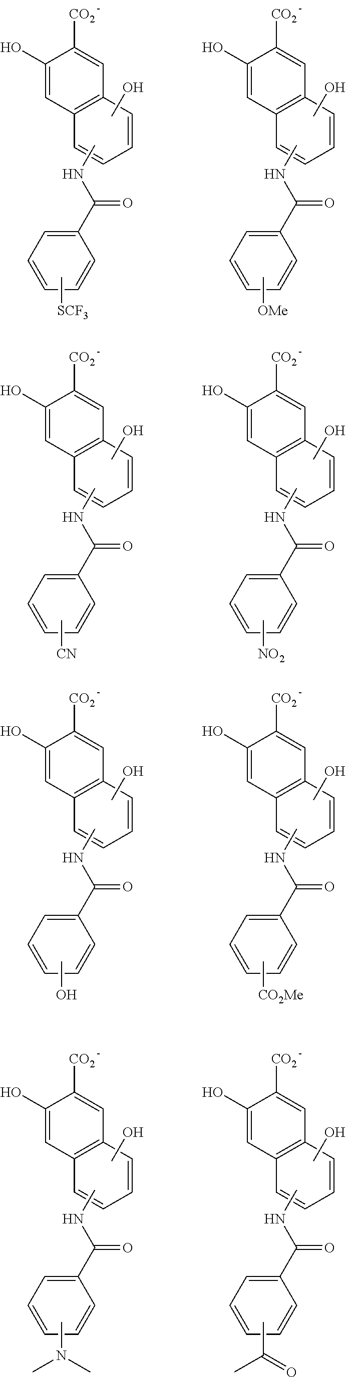

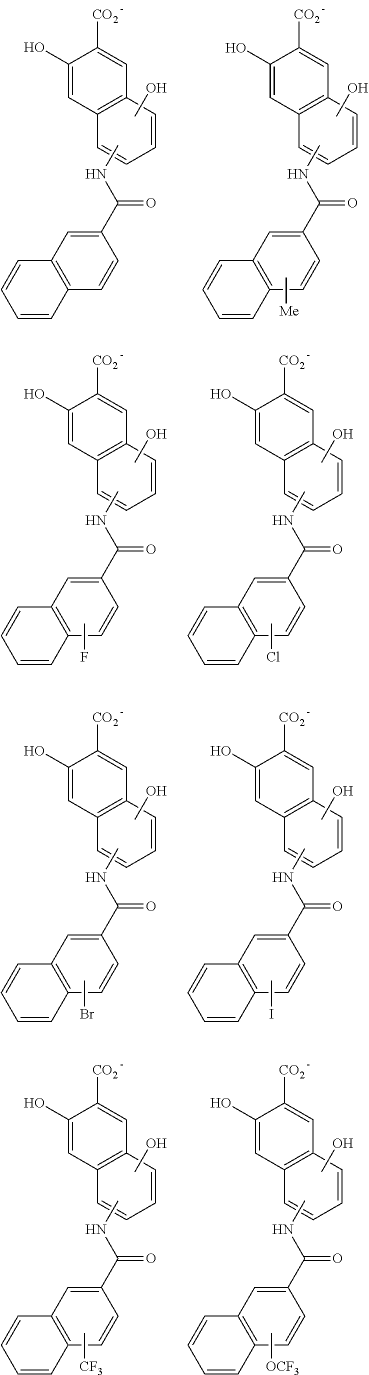

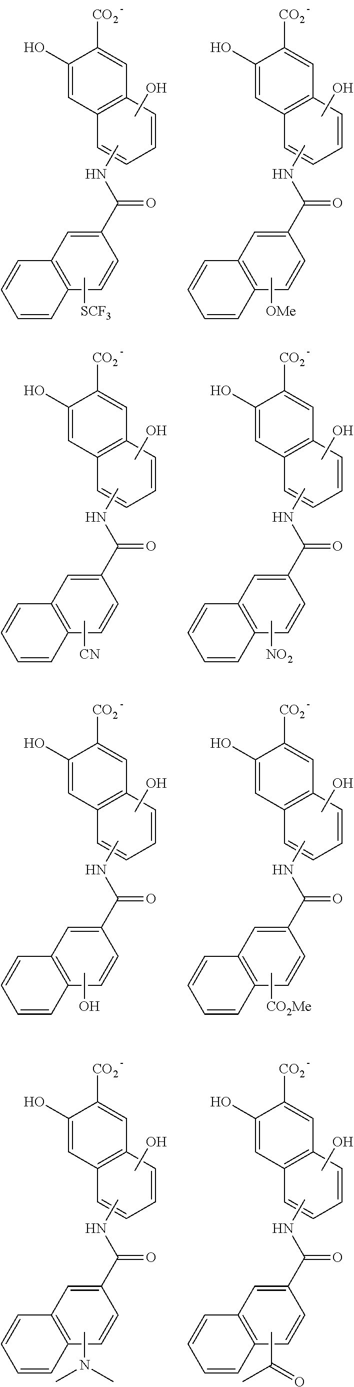

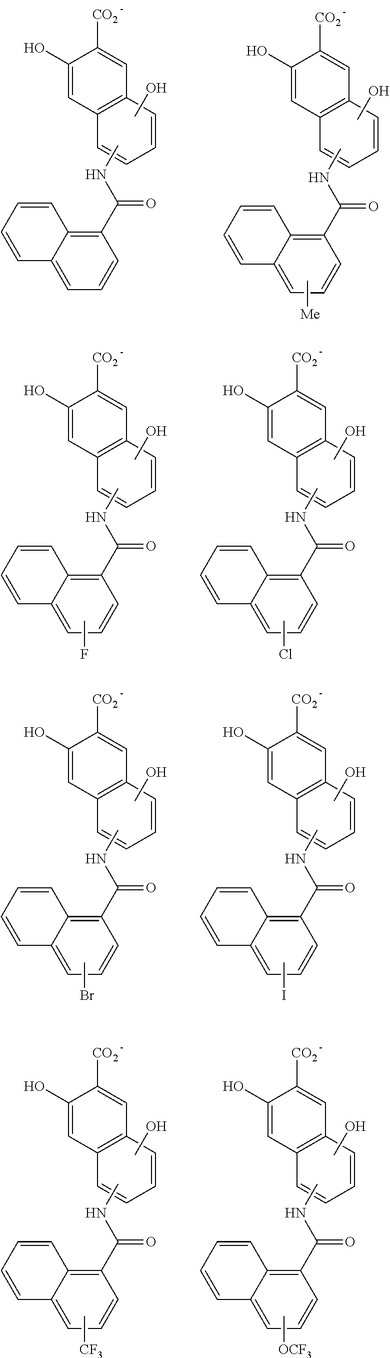

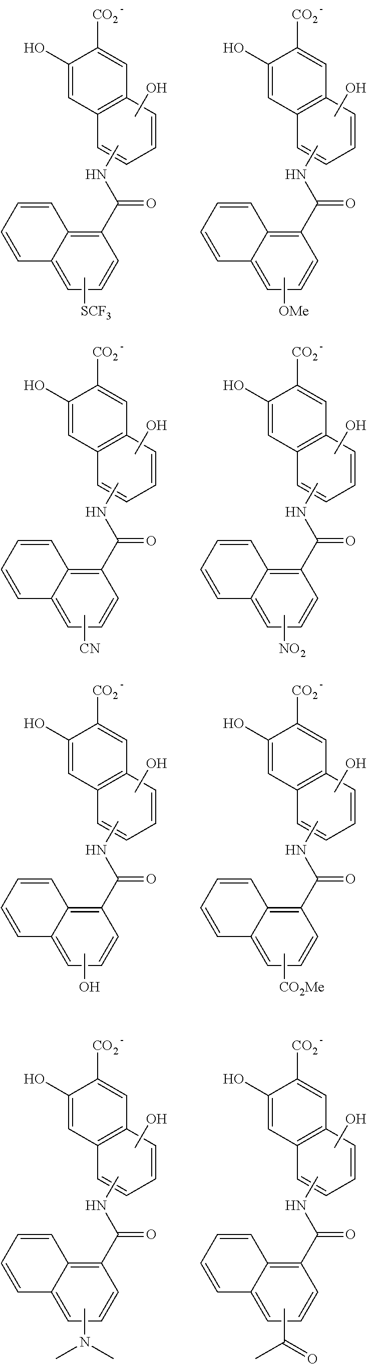

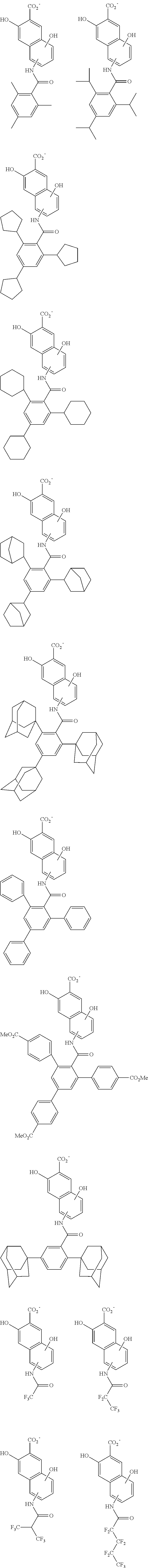

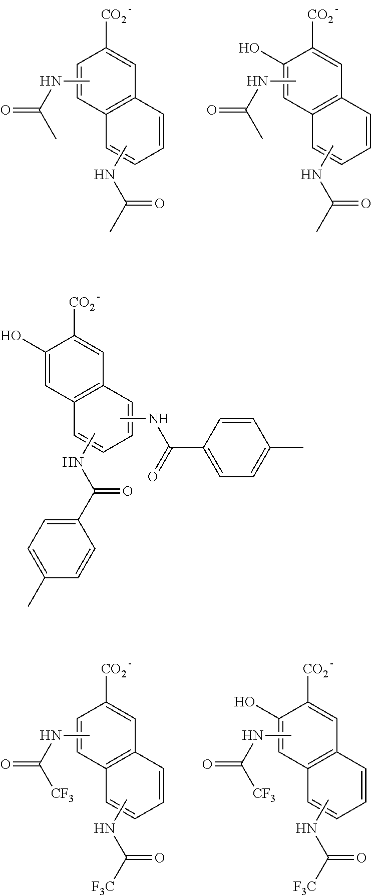

In the general formula (1), Z⁺ represents an onium cation. Specific examples include sulfonium cations, iodonium cations, ammonium cations, phosphonium cations, etc., and the sulfonium cations, iodonium cations, and ammonium cations shown below are preferable.

In the general formula (1), Z⁺ is preferably represented by one of the following general formulae (Cation-1) to (Cation-3).

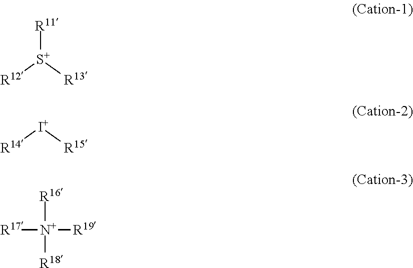

In the general formulae (Cation-1) to (Cation-3), R^{11′} to R^{19′} each independently represent a hydrocarbyl group having 1 to 30 carbon atoms and optionally containing a heteroatom. The hydrocarbyl group may be saturated or unsaturated, and may be linear, branched, or cyclic. Specific examples thereof include: alkyl groups, such as a methyl group, an ethyl group, an n-propyl group, an isopropyl group, an n-butyl group, and a tert-butyl group; cyclic saturated hydrocarbyl groups, such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups, such as a vinyl group, an allyl group, a propenyl group, a butenyl group, and a hexenyl group; cyclic unsaturated hydrocarbyl groups, such as a cyclohexenyl group; aryl groups, such as a phenyl group, a naphthyl group, and a thienyl group; aralkyl groups, such as a benzyl group, a 1-phenylethyl group, and a 2-phenylethyl group; and groups derived from combinations of these groups. Aryl groups are preferable. In addition, part of the hydrogen atoms of the hydrocarbyl group may be substituted with a group containing a heteroatom, such as an oxygen atom, a sulfur atom, a nitrogen atom, or a halogen atom, and the hydrocarbyl group may have an intervening group having a heteroatom, such as an oxygen atom, a sulfur atom, or a nitrogen atom, between the carbon atoms of these groups. The resulting hydrocarbyl group may contain a hydroxy group, a cyano group, a carbonyl group, an ether bond, an ester bond, a sulfonic acid ester bond, a carbonate bond, a lactone ring, a sultone ring, a carboxylic anhydride, a haloalkyl group, etc.

R^{11′} and R^{12′} may also be bonded to each other to form a ring together with the sulfur atom bonded to R^{11′} and R^{12′}. In this event, examples of the sulfonium cation represented by the formula (Cation-1) include those represented by the following formulae.

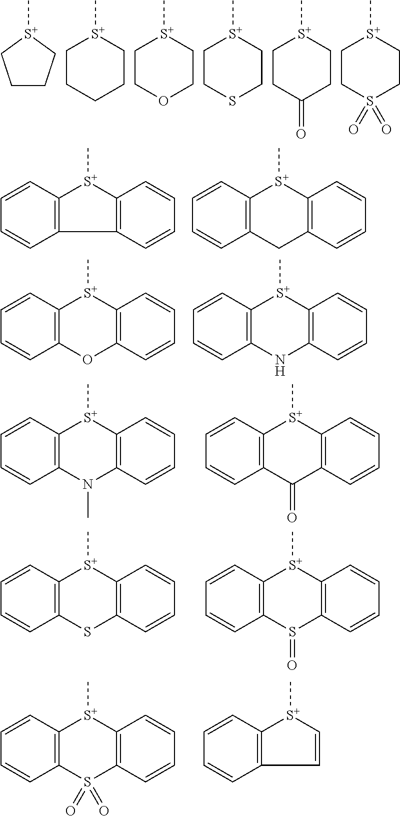

In the formulae, a broken line represents an attachment point to R¹³.

Examples of the cation of a sulfonium salt represented by the formula (Cation-1) include the following, but are not limited thereto.

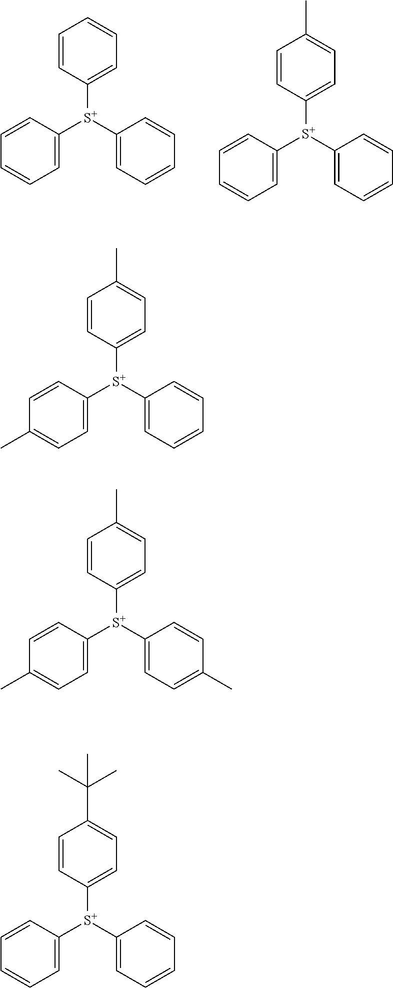

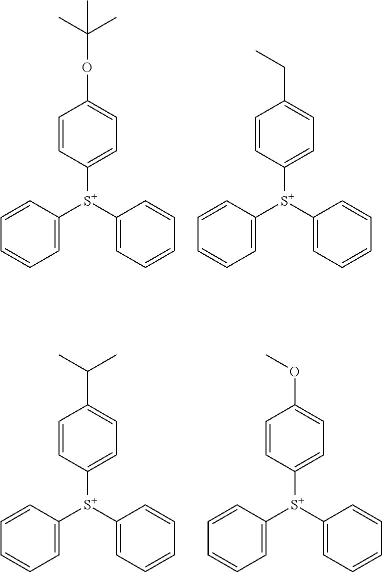 [SMILES: CC(C)(C)Oc1ccc([S+](c2ccccc2)c2ccccc2)cc1 ; CCc1ccc([S+](c2ccccc2)c2ccccc2)cc1 ; CC(C)c1ccc([S+](c2ccccc2)c2ccccc2)cc1 ; COc1ccc([S+](c2ccccc2)c2ccccc2)cc1]

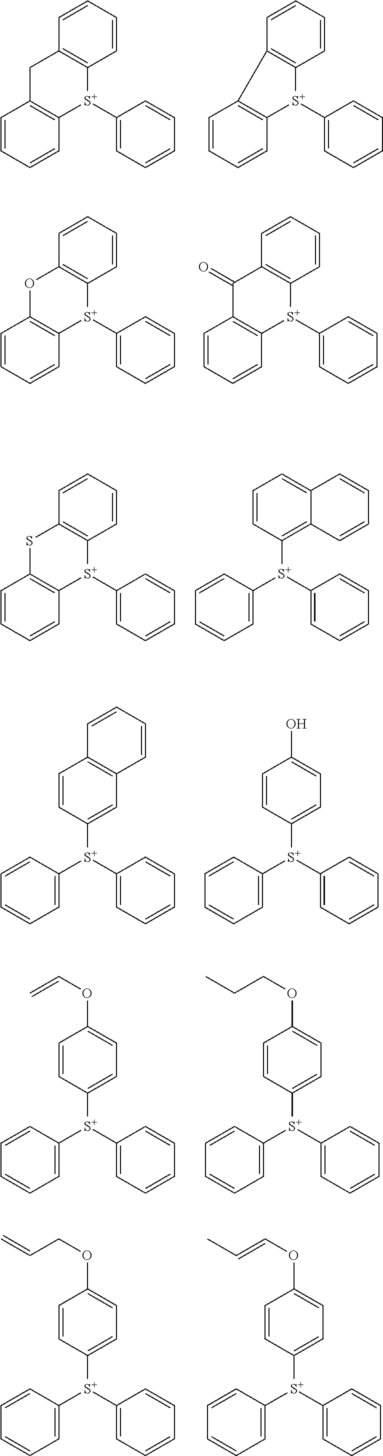

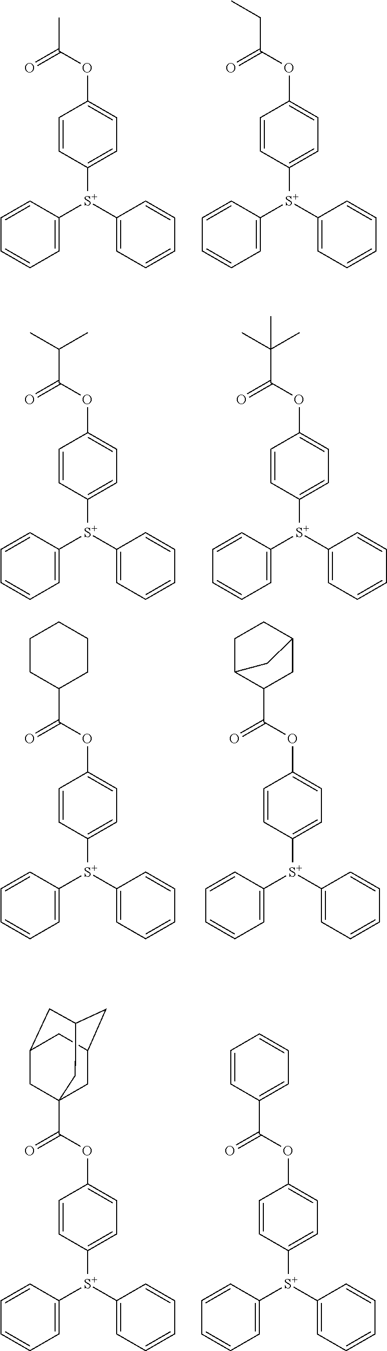

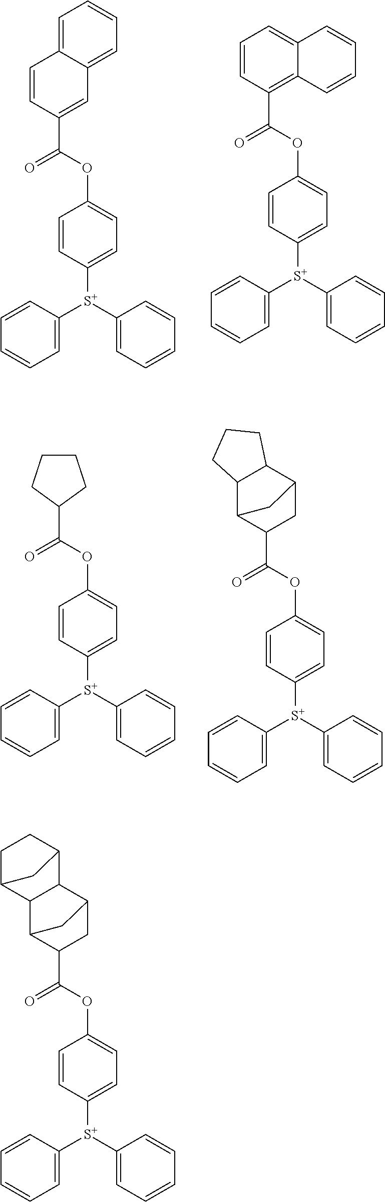 [SMILES: O=C(Oc1ccc([S+](c2ccccc2)c2ccccc2)cc1)c1ccc2ccccc2c1 ; O=C(Oc1ccc([S+](c2ccccc2)c2ccccc2)cc1)c1cccc2ccccc12 ; O=C(Oc1ccc([S+](c2ccccc2)c2ccccc2)cc1)C1CCCC1 ; O=C(Oc1ccc([S+](c2ccccc2)c2ccccc2)cc1)C1CC2CC1C1CCCC21 ; O=C(Oc1ccc([S+](c2ccccc2)c2ccccc2)cc1)C1CC2CC1C1C3CCC(C3)C21]

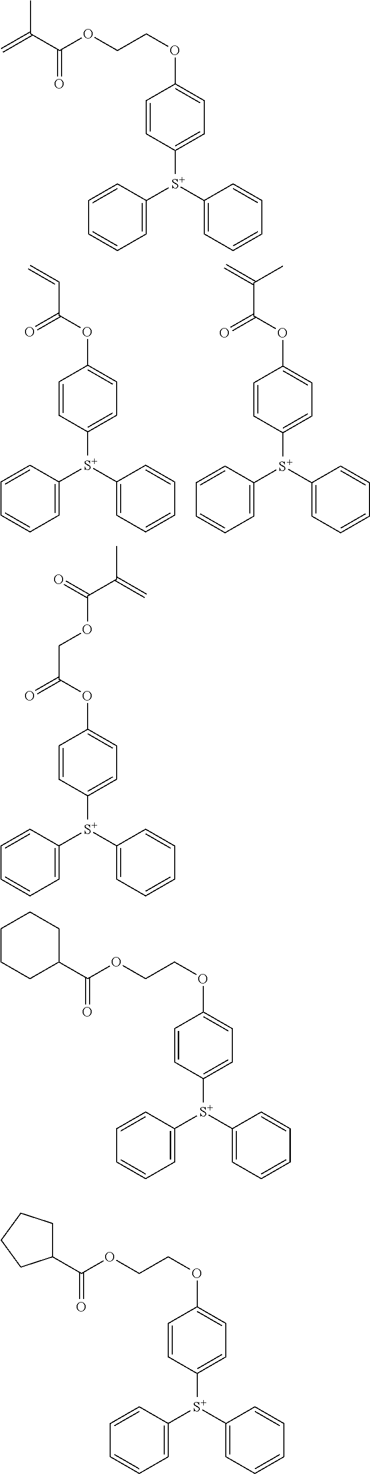 [SMILES: C=C(C)C(=O)OCCOc1ccc([S+](c2ccccc2)c2ccccc2)cc1 ; C=CC(=O)Oc1ccc([S+](c2ccccc2)c2ccccc2)cc1 ; C=C(C)C(=O)Oc1ccc([S+](c2ccccc2)c2ccccc2)cc1 ; C=C(C)C(=O)OCC(=O)Oc1ccc([S+](c2ccccc2)c2ccccc2)cc1 ; O=C(OCCOc1ccc([S+](c2ccccc2)c2ccccc2)cc1)C1CCCCC1 ; O=C(OCCOc1ccc([S+](c2ccccc2)c2ccccc2)cc1)C1CCCC1]

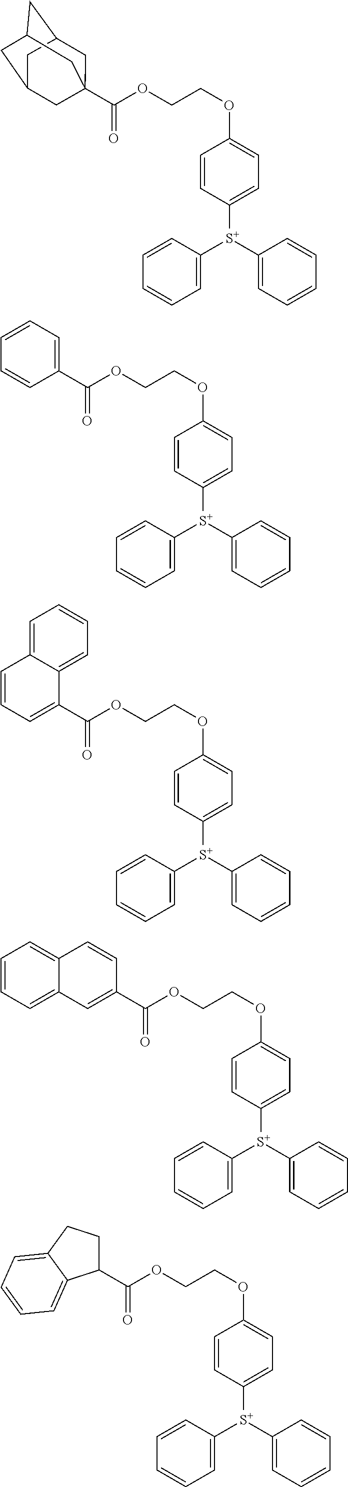 [SMILES: O=C(OCCOc1ccc([S+](c2ccccc2)c2ccccc2)cc1)C12CC3CC(CC(C3)C1)C2 ; O=C(OCCOc1ccc([S+](c2ccccc2)c2ccccc2)cc1)c1ccccc1 ; O=C(OCCOc1ccc([S+](c2ccccc2)c2ccccc2)cc1)c1cccc2ccccc12 ; O=C(OCCOc1ccc([S+](c2ccccc2)c2ccccc2)cc1)c1ccc2ccccc2c1 ; O=C(OCCOc1ccc([S+](c2ccccc2)c2ccccc2)cc1)C1CCc2ccccc21]

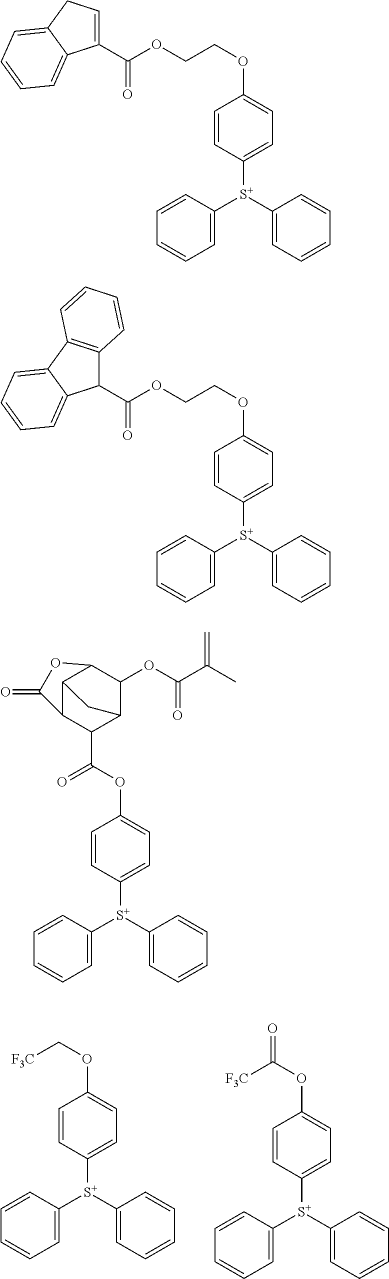 [SMILES: O=C(OCCOc1ccc([S+](c2ccccc2)c2ccccc2)cc1)C1=CCc2ccccc21 ; O=C(OCCOc1ccc([S+](c2ccccc2)c2ccccc2)cc1)C1c2ccccc2-c2ccccc21 ; C=C(C)C(=O)OC1C2CC3C1OC(=O)C3C2C(=O)Oc1ccc([S+](c2ccccc2)c2ccccc2)cc1 ; FC(F)(F)COc1ccc([S+](c2ccccc2)c2ccccc2)cc1 ; O=C(Oc1ccc([S+](c2ccccc2)c2ccccc2)cc1)C(F)(F)F]

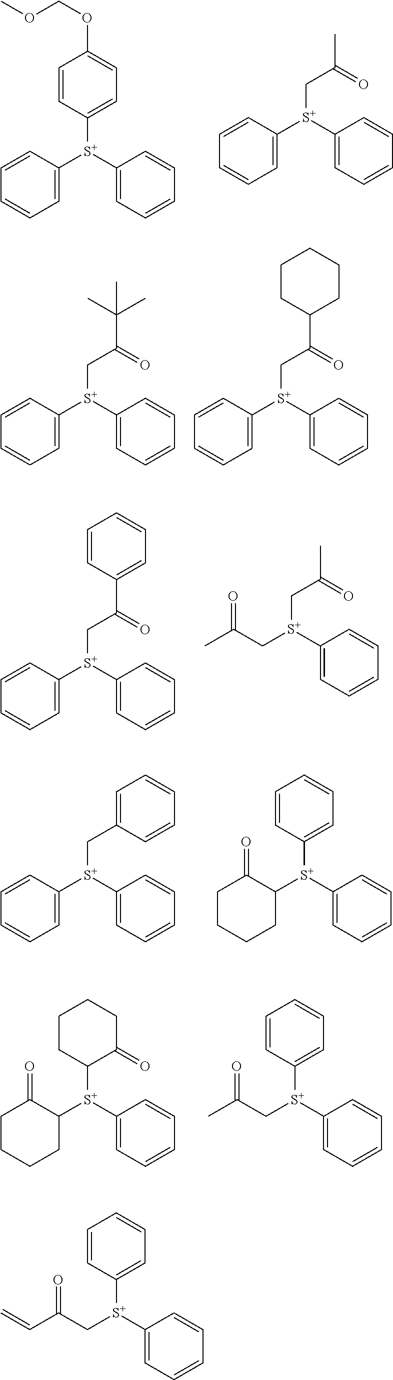

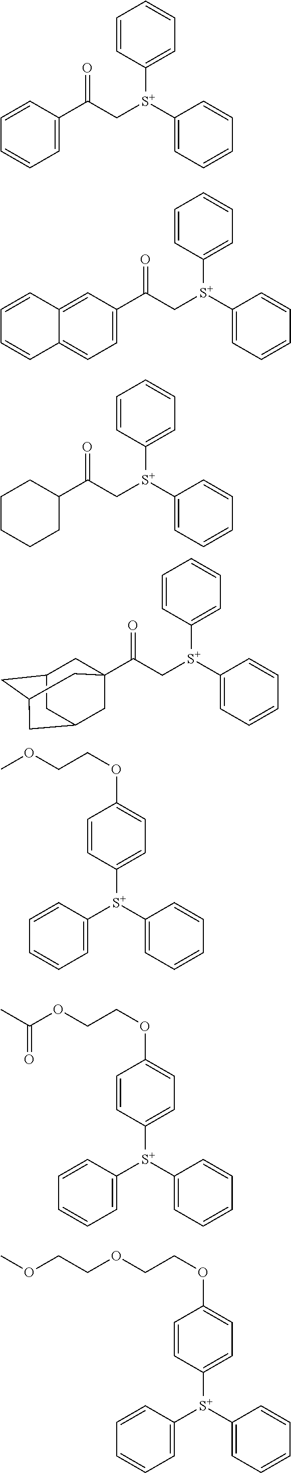

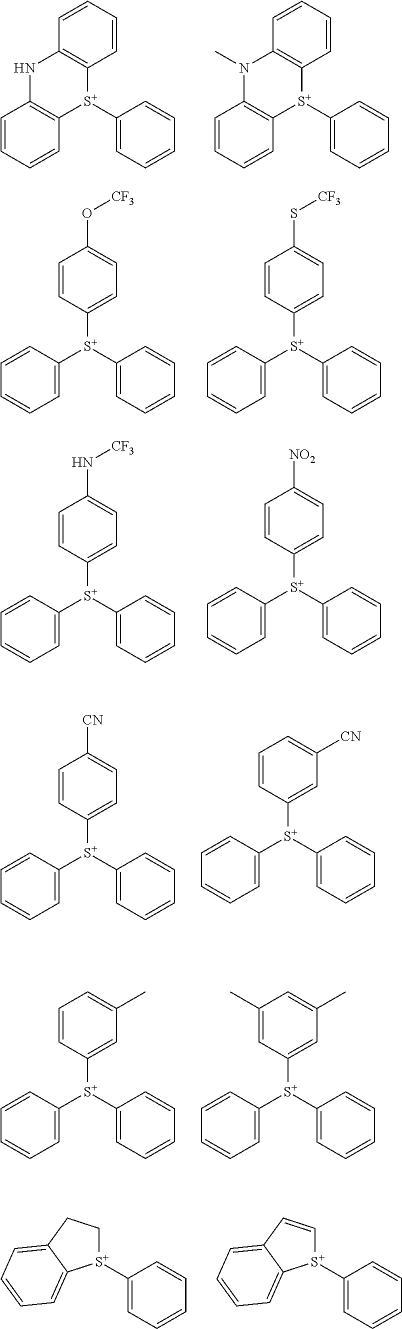

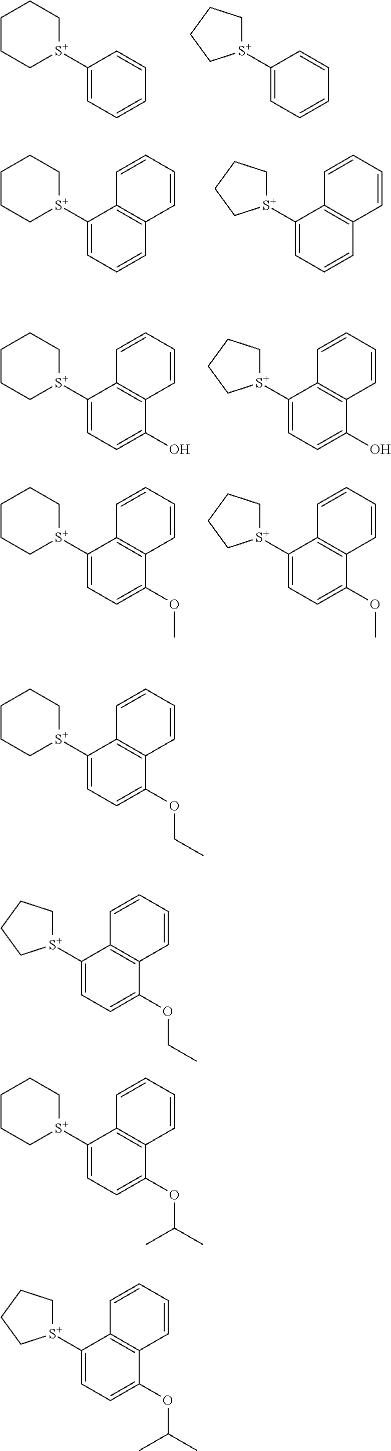

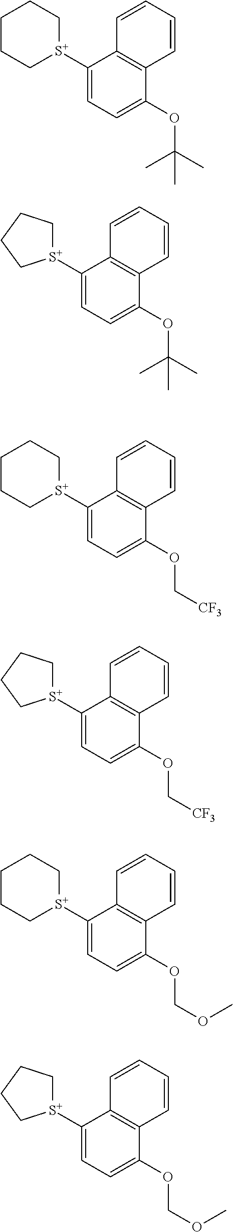 [SMILES: CC(C)(C)Oc1ccc([S+]2CCCCC2)c2ccccc12 ; CC(C)(C)Oc1ccc([S+]2CCCC2)c2ccccc12 ; FC(F)(F)COc1ccc([S+]2CCCCC2)c2ccccc12 ; FC(F)(F)COc1ccc([S+]2CCCC2)c2ccccc12 ; COCOc1ccc([S+]2CCCCC2)c2ccccc12 ; COCOc1ccc([S+]2CCCC2)c2ccccc12]

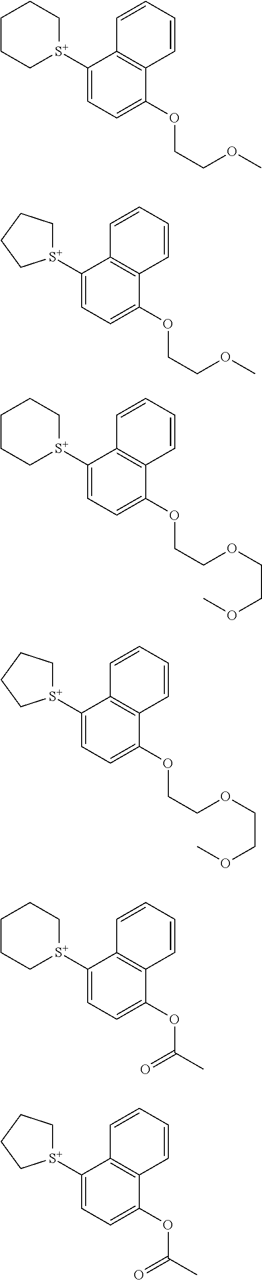 [SMILES: COCCOc1ccc([S+]2CCCCC2)c2ccccc12 ; COCCOc1ccc([S+]2CCCC2)c2ccccc12 ; COCCOCCOc1ccc([S+]2CCCCC2)c2ccccc12 ; COCCOCCOc1ccc([S+]2CCCC2)c2ccccc12 ; CC(=O)Oc1ccc([S+]2CCCCC2)c2ccccc12 ; CC(=O)Oc1ccc([S+]2CCCC2)c2ccccc12]

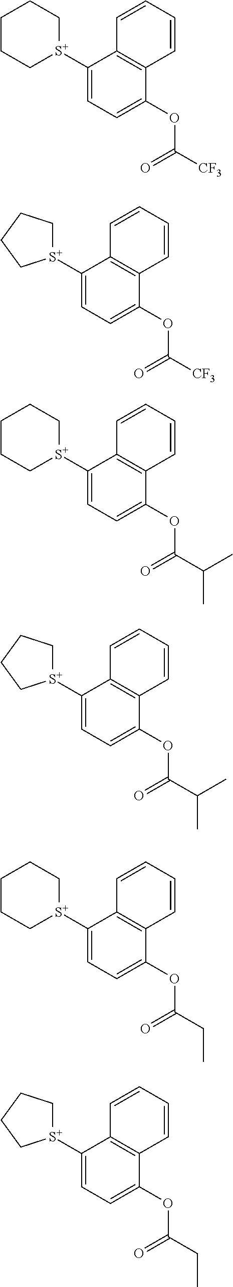 [SMILES: O=C(Oc1ccc([S+]2CCCCC2)c2ccccc12)C(F)(F)F ; O=C(Oc1ccc([S+]2CCCC2)c2ccccc12)C(F)(F)F ; CC(C)C(=O)Oc1ccc([S+]2CCCCC2)c2ccccc12 ; CC(C)C(=O)Oc1ccc([S+]2CCCC2)c2ccccc12 ; CCC(=O)Oc1ccc([S+]2CCCCC2)c2ccccc12 ; CCC(=O)Oc1ccc([S+]2CCCC2)c2ccccc12]

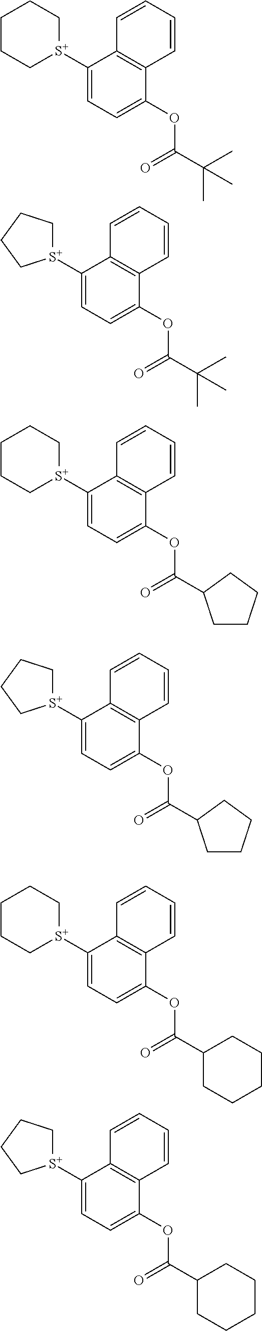 [SMILES: CC(C)(C)C(=O)Oc1ccc([S+]2CCCCC2)c2ccccc12 ; CC(C)(C)C(=O)Oc1ccc([S+]2CCCC2)c2ccccc12 ; O=C(Oc1ccc([S+]2CCCCC2)c2ccccc12)C1CCCC1 ; O=C(Oc1ccc([S+]2CCCC2)c2ccccc12)C1CCCC1 ; O=C(Oc1ccc([S+]2CCCCC2)c2ccccc12)C1CCCCC1 ; O=C(Oc1ccc([S+]2CCCC2)c2ccccc12)C1CCCCC1]

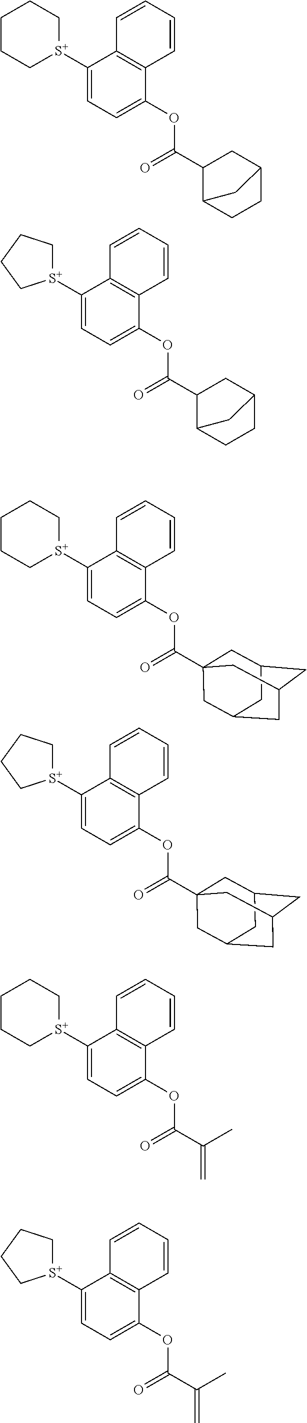 [SMILES: O=C(Oc1ccc([S+]2CCCCC2)c2ccccc12)C1CC2CCC1C2 ; O=C(Oc1ccc([S+]2CCCC2)c2ccccc12)C1CC2CCC1C2 ; O=C(Oc1ccc([S+]2CCCCC2)c2ccccc12)C12CC3CC(CC(C3)C1)C2 ; O=C(Oc1ccc([S+]2CCCC2)c2ccccc12)C12CC3CC(CC(C3)C1)C2 ; C=C(C)C(=O)Oc1ccc([S+]2CCCCC2)c2ccccc12 ; C=C(C)C(=O)Oc1ccc([S+]2CCCC2)c2ccccc12]

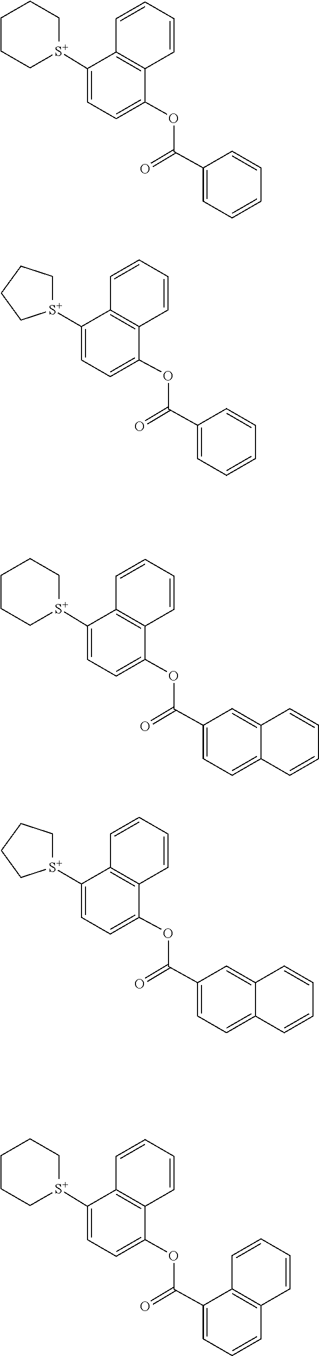 [SMILES: O=C(Oc1ccc([S+]2CCCCC2)c2ccccc12)c1ccccc1 ; O=C(Oc1ccc([S+]2CCCC2)c2ccccc12)c1ccccc1 ; O=C(Oc1ccc([S+]2CCCCC2)c2ccccc12)c1ccc2ccccc2c1 ; O=C(Oc1ccc([S+]2CCCC2)c2ccccc12)c1ccc2ccccc2c1 ; O=C(Oc1ccc([S+]2CCCCC2)c2ccccc12)c1cccc2ccccc12]

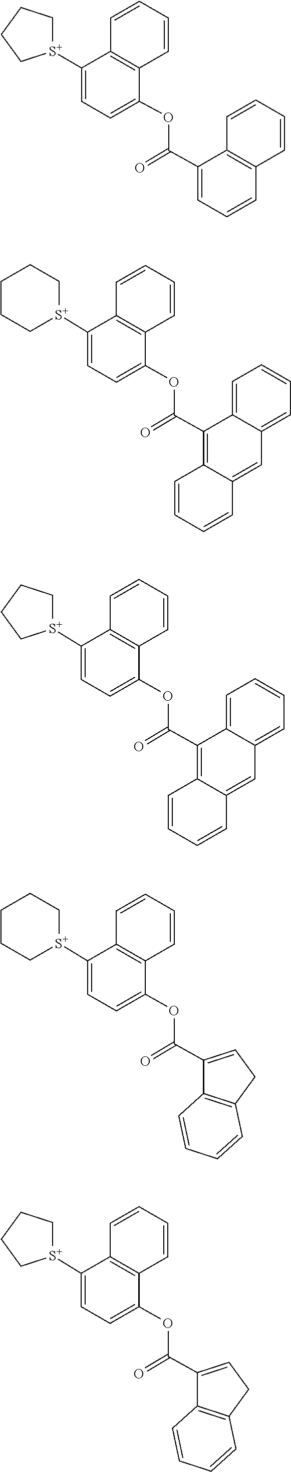 [SMILES: O=C(Oc1ccc([S+]2CCCC2)c2ccccc12)c1cccc2ccccc12 ; O=C(Oc1ccc([S+]2CCCCC2)c2ccccc12)c1c2ccccc2cc2ccccc12 ; O=C(Oc1ccc([S+]2CCCC2)c2ccccc12)c1c2ccccc2cc2ccccc12 ; O=C(Oc1ccc([S+]2CCCCC2)c2ccccc12)C1=CCc2ccccc21 ; O=C(Oc1ccc([S+]2CCCC2)c2ccccc12)C1=CCc2ccccc21]

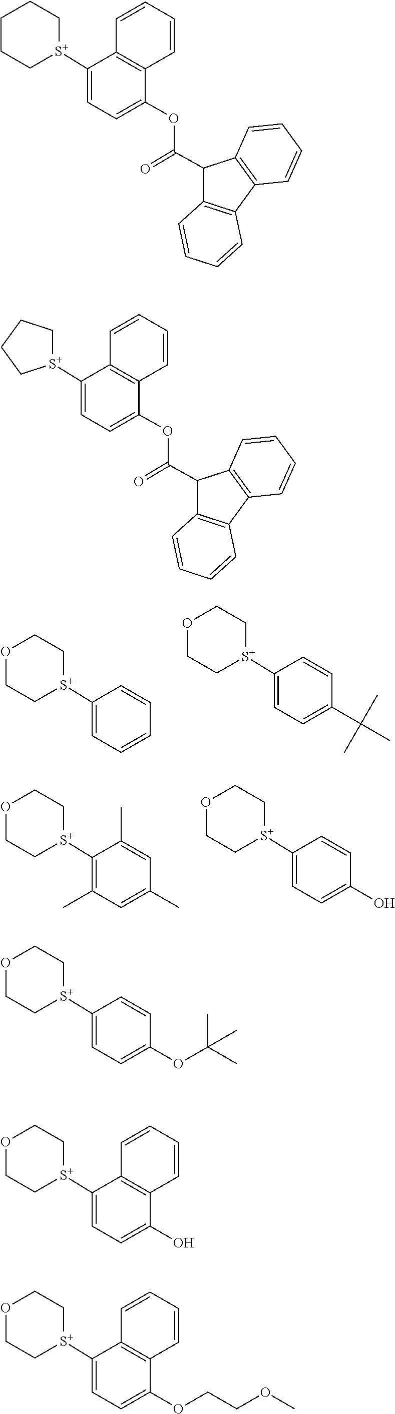

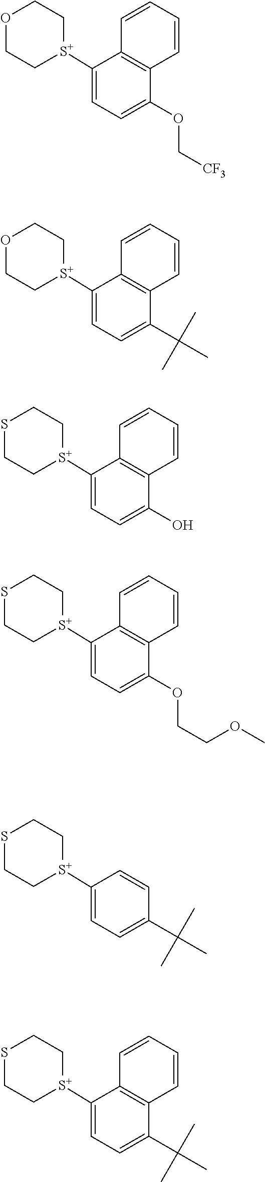 [SMILES: FC(F)(F)COc1ccc([S+]2CCOCC2)c2ccccc12 ; CC(C)(C)c1ccc([S+]2CCOCC2)c2ccccc12 ; Oc1ccc([S+]2CCSCC2)c2ccccc12 ; COCCOc1ccc([S+]2CCSCC2)c2ccccc12 ; CC(C)(C)c1ccc([S+]2CCSCC2)cc1 ; CC(C)(C)c1ccc([S+]2CCSCC2)c2ccccc12]

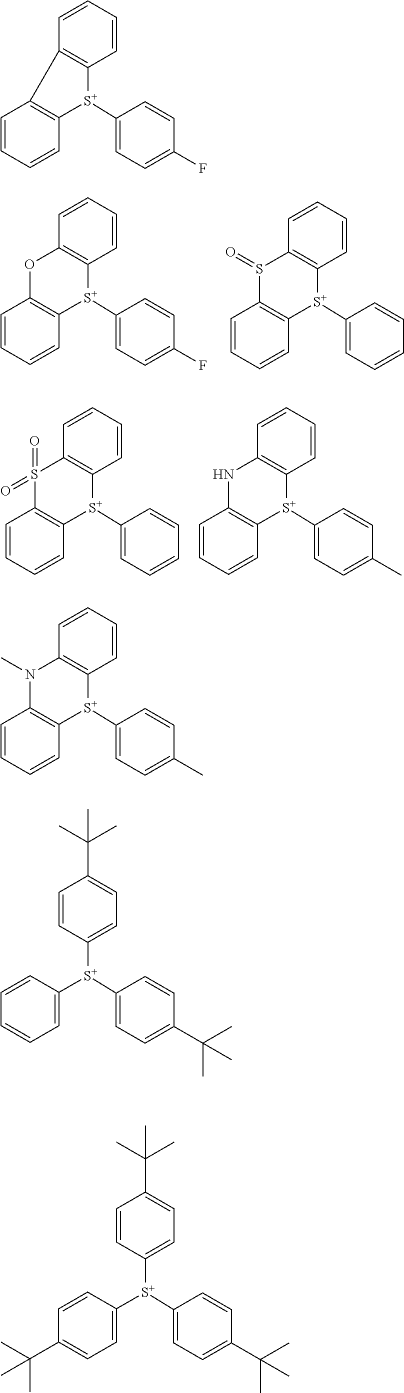

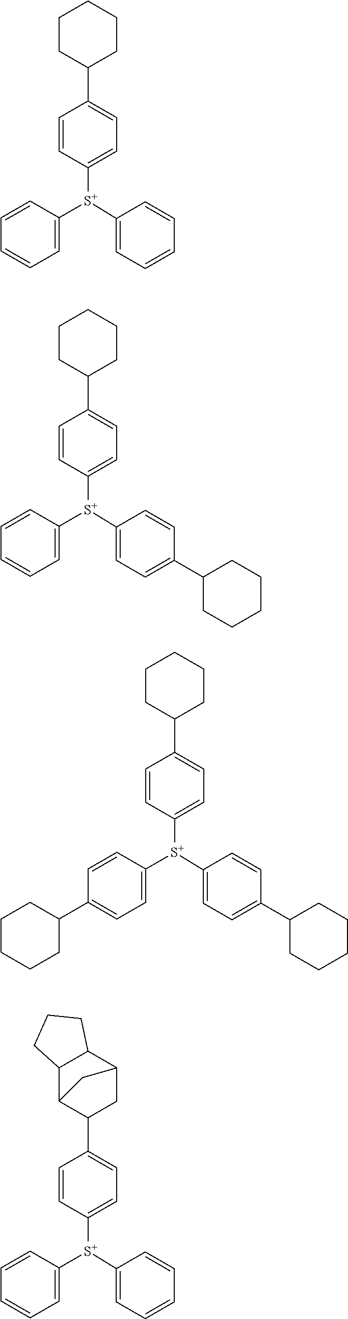 [SMILES: c1ccc([S+](c2ccccc2)c2ccc(C3CCCCC3)cc2)cc1 ; c1ccc([S+](c2ccc(C3CCCCC3)cc2)c2ccc(C3CCCCC3)cc2)cc1 ; c1cc([S+](c2ccc(C3CCCCC3)cc2)c2ccc(C3CCCCC3)cc2)ccc1C1CCCCC1 ; c1ccc([S+](c2ccccc2)c2ccc(C3CC4CC3C3CCCC43)cc2)cc1]

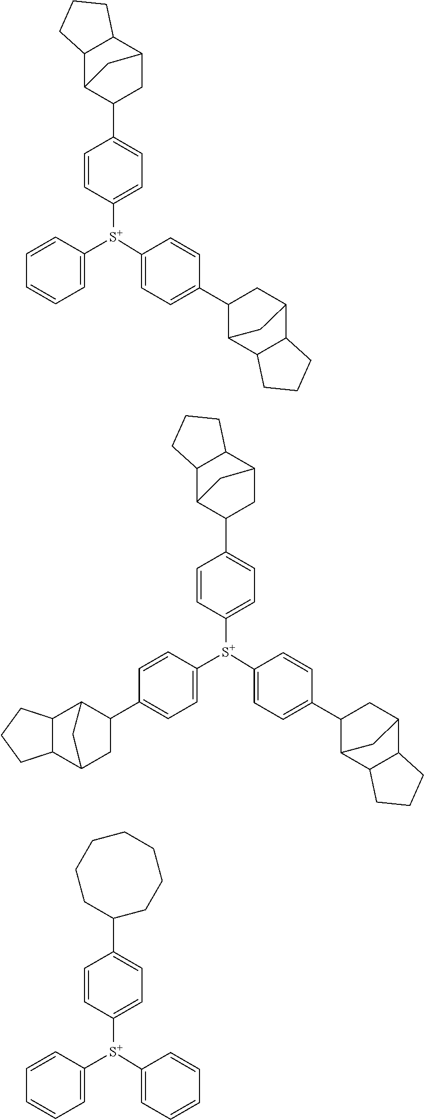 [SMILES: c1ccc([S+](c2ccc(C3CC4CC3C3CCCC43)cc2)c2ccc(C3CC4CC3C3CCCC43)cc2)cc1 ; c1cc([S+](c2ccc(C3CC4CC3C3CCCC43)cc2)c2ccc(C3CC4CC3C3CCCC43)cc2)ccc1C1CC2CC1C1CCCC21 ; c1ccc([S+](c2ccccc2)c2ccc(C3CCCCCCC3)cc2)cc1]

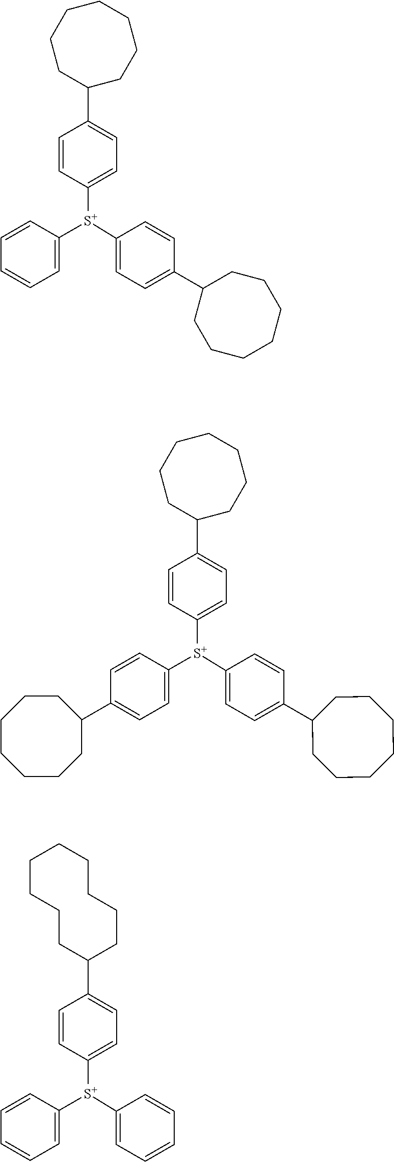 [SMILES: c1ccc([S+](c2ccc(C3CCCCCCC3)cc2)c2ccc(C3CCCCCCC3)cc2)cc1 ; c1cc([S+](c2ccc(C3CCCCCCC3)cc2)c2ccc(C3CCCCCCC3)cc2)ccc1C1CCCCCCC1 ; c1ccc([S+](c2ccccc2)c2ccc(C3CCCCCCCCC3)cc2)cc1]

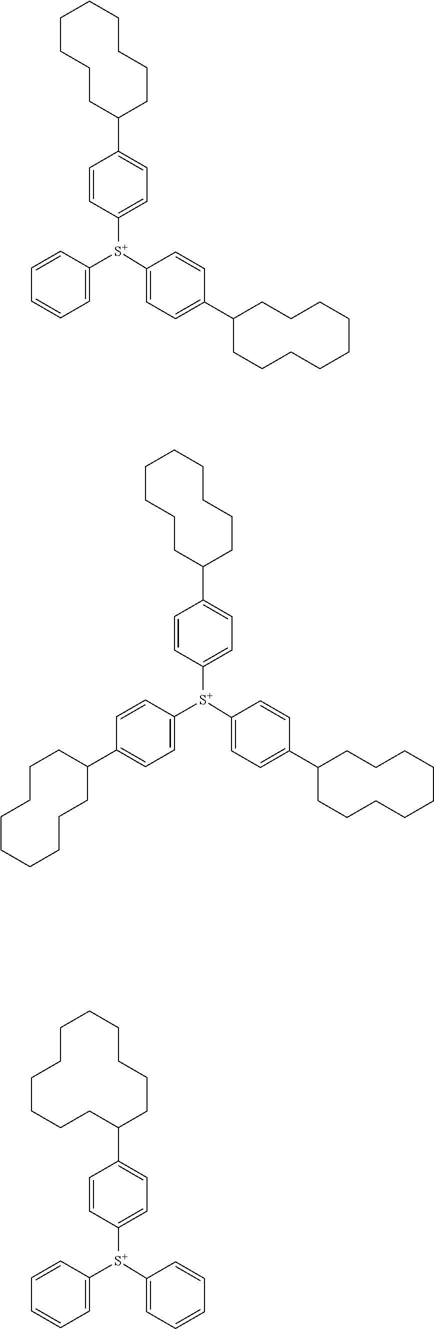 [SMILES: c1ccc([S+](c2ccc(C3CCCCCCCCC3)cc2)c2ccc(C3CCCCCCCCC3)cc2)cc1 ; c1cc([S+](c2ccc(C3CCCCCCCCC3)cc2)c2ccc(C3CCCCCCCCC3)cc2)ccc1C1CCCCCCCCC1 ; c1ccc([S+](c2ccccc2)c2ccc(C3CCCCCCCCCCC3)cc2)cc1]

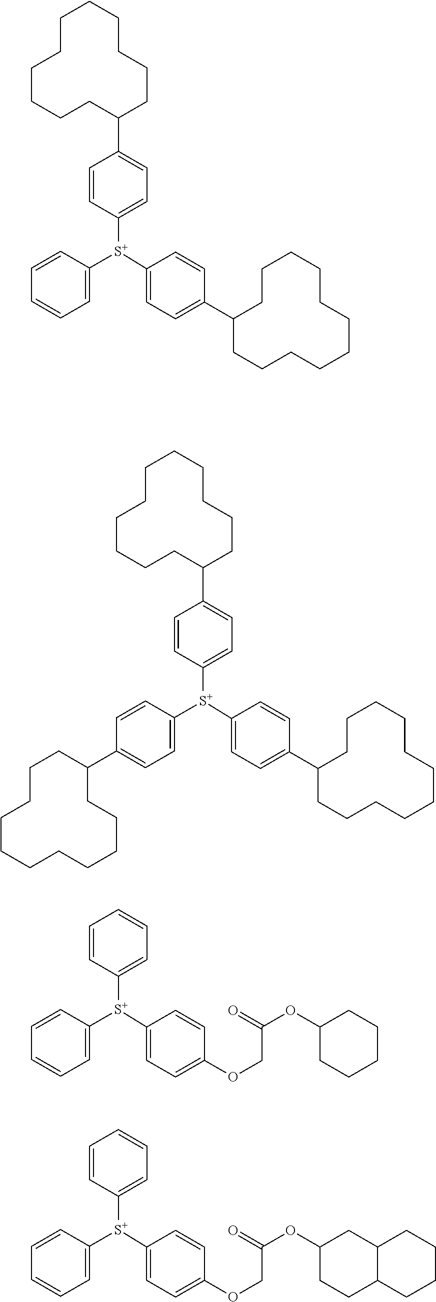 [SMILES: c1ccc([S+](c2ccc(C3CCCCCCCCCCC3)cc2)c2ccc(C3CCCCCCCCCCC3)cc2)cc1 ; c1cc([S+](c2ccc(C3CCCCCCCCCCC3)cc2)c2ccc(C3CCCCCCCCCCC3)cc2)ccc1C1CCCCCCCCCCC1 ; O=C(COc1ccc([S+](c2ccccc2)c2ccccc2)cc1)OC1CCCCC1 ; O=C(COc1ccc([S+](c2ccccc2)c2ccccc2)cc1)OC1CCC2CCCCC2C1]

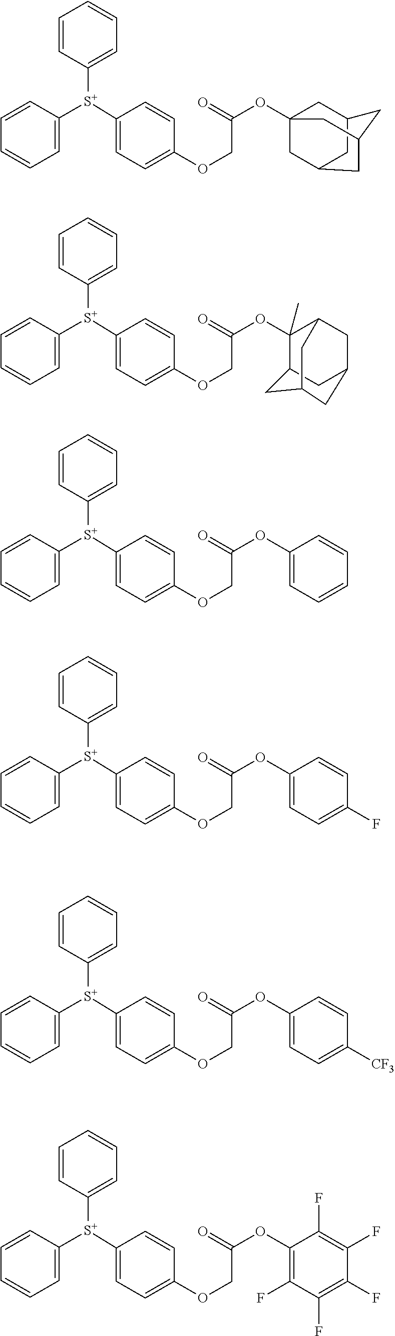 [SMILES: O=C(COc1ccc([S+](c2ccccc2)c2ccccc2)cc1)OC12CC3CC(CC(C3)C1)C2 ; CC1(OC(=O)COc2ccc([S+](c3ccccc3)c3ccccc3)cc2)C2CC3CC(C2)CC1C3 ; O=C(COc1ccc([S+](c2ccccc2)c2ccccc2)cc1)Oc1ccccc1 ; O=C(COc1ccc([S+](c2ccccc2)c2ccccc2)cc1)Oc1ccc(F)cc1 ; O=C(COc1ccc([S+](c2ccccc2)c2ccccc2)cc1)Oc1ccc(C(F)(F)F)cc1 ; O=C(COc1ccc([S+](c2ccccc2)c2ccccc2)cc1)Oc1c(F)c(F)c(F)c(F)c1F]

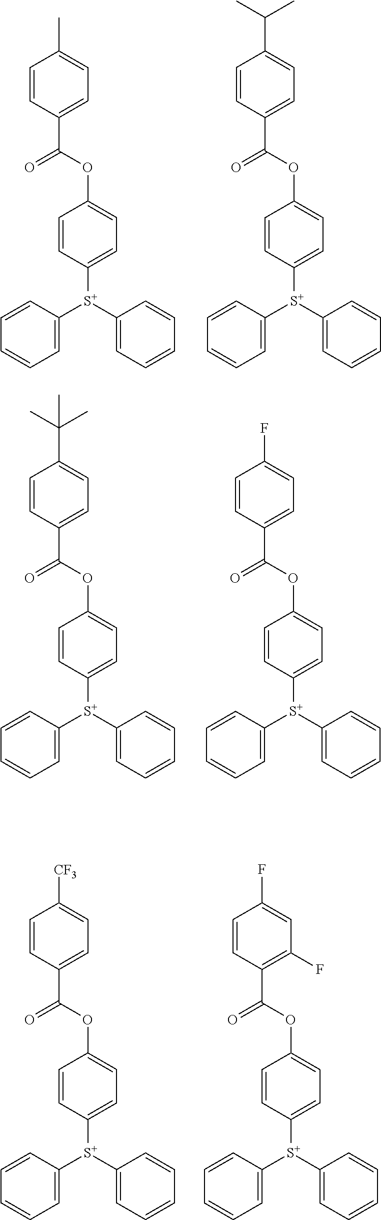 [SMILES: Cc1ccc(C(=O)Oc2ccc([S+](c3ccccc3)c3ccccc3)cc2)cc1 ; CC(C)c1ccc(C(=O)Oc2ccc([S+](c3ccccc3)c3ccccc3)cc2)cc1 ; CC(C)(C)c1ccc(C(=O)Oc2ccc([S+](c3ccccc3)c3ccccc3)cc2)cc1 ; O=C(Oc1ccc([S+](c2ccccc2)c2ccccc2)cc1)c1ccc(F)cc1 ; O=C(Oc1ccc([S+](c2ccccc2)c2ccccc2)cc1)c1ccc(C(F)(F)F)cc1 ; O=C(Oc1ccc([S+](c2ccccc2)c2ccccc2)cc1)c1ccc(F)cc1F]

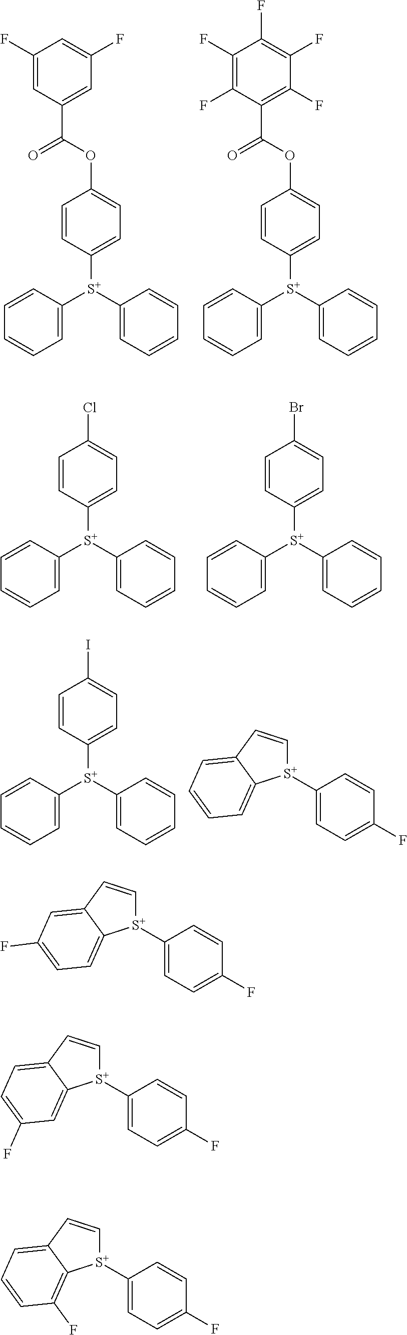

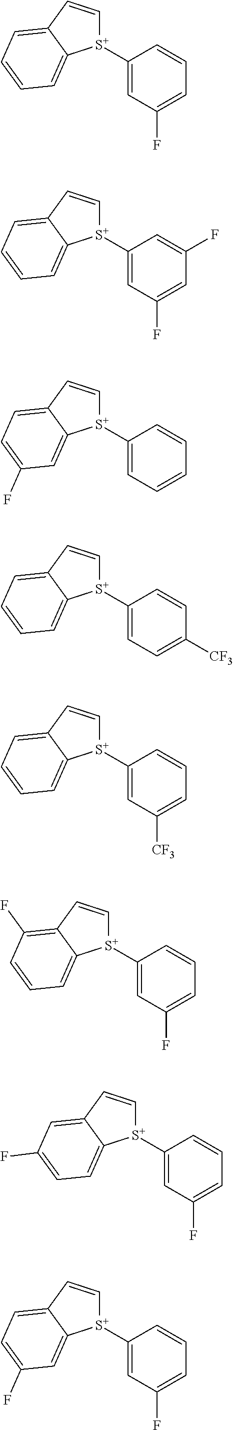

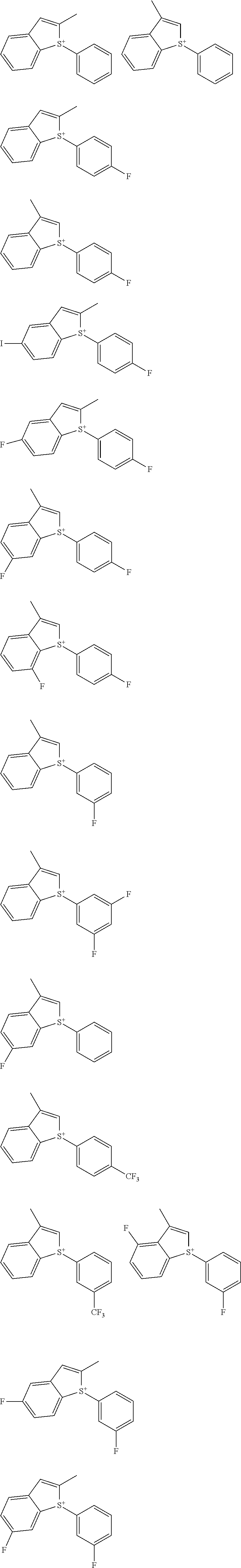

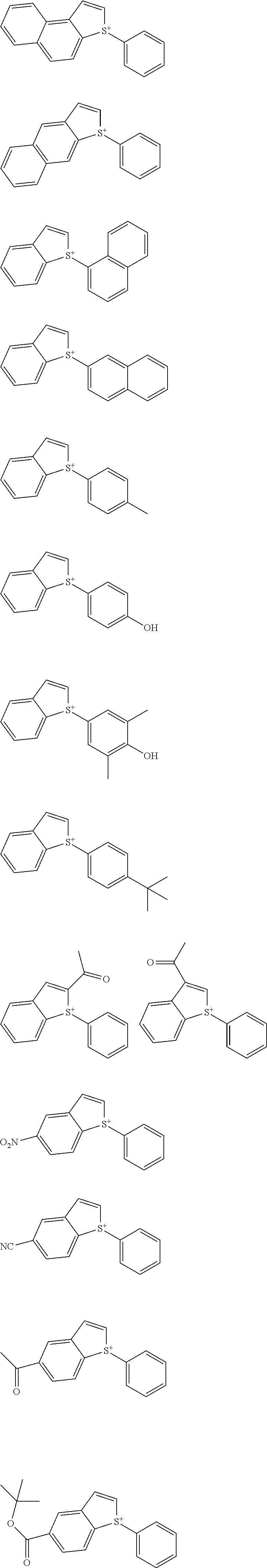

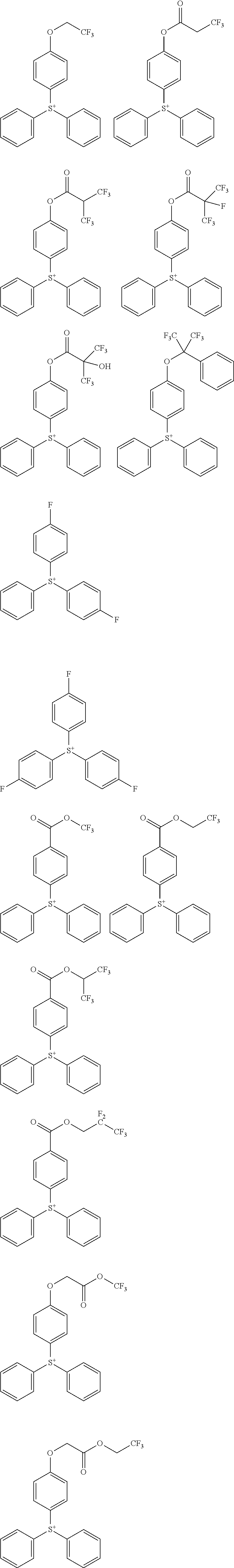

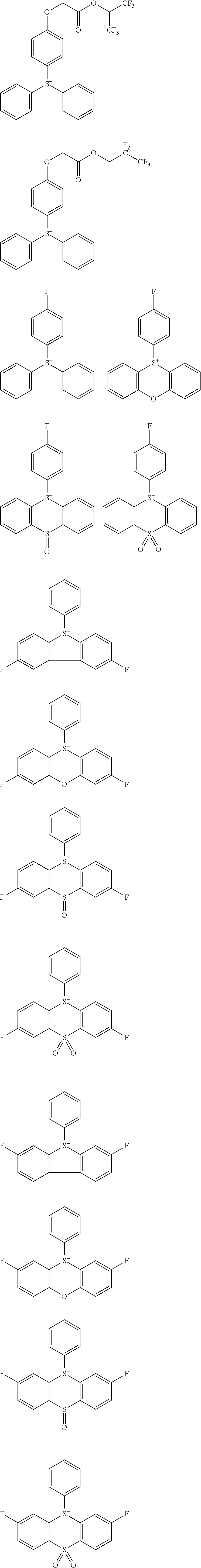

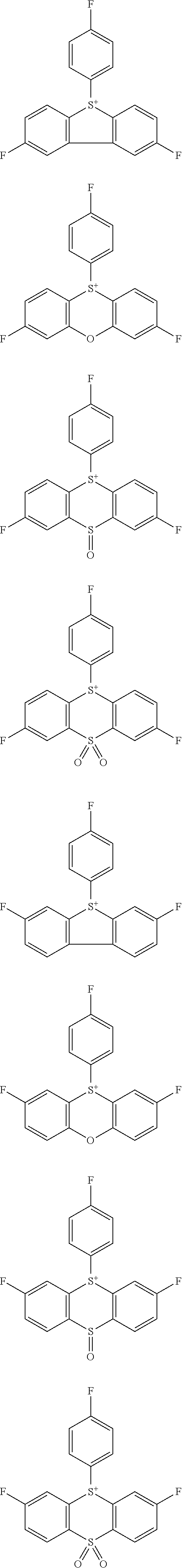

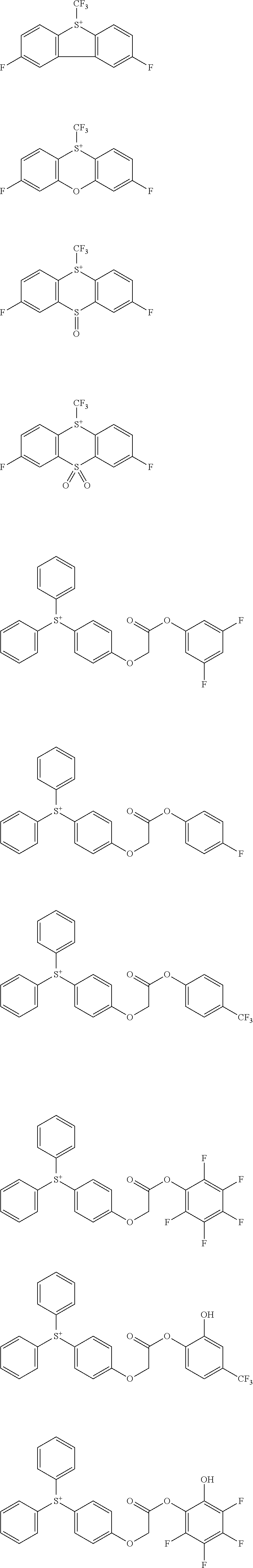

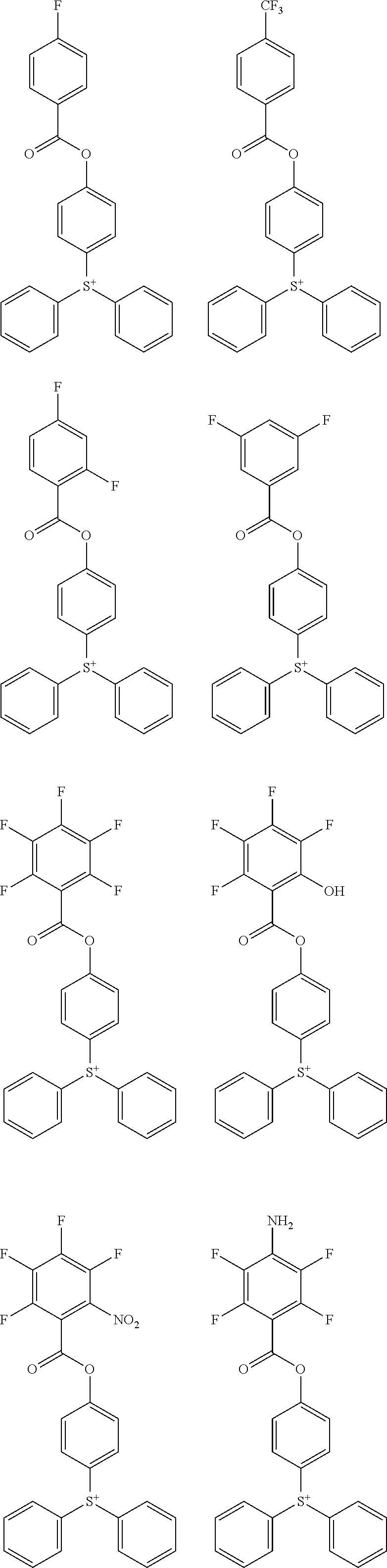

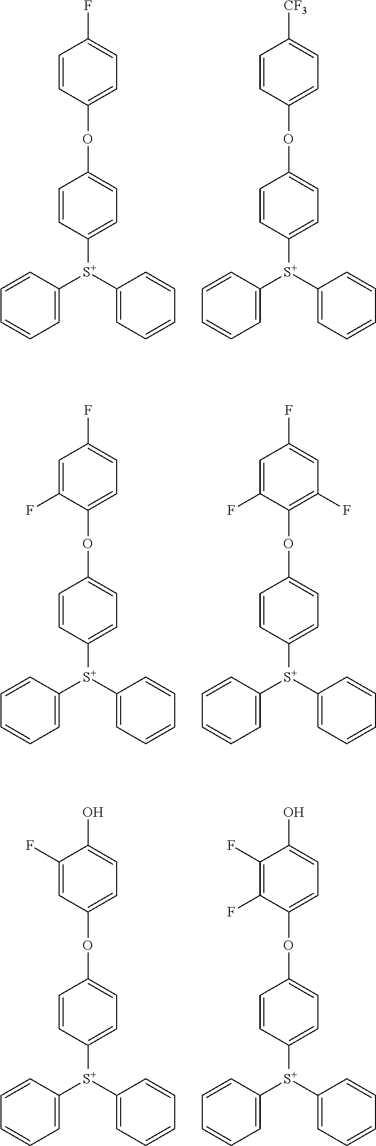 [SMILES: Fc1ccc(Oc2ccc([S+](c3ccccc3)c3ccccc3)cc2)cc1 ; FC(F)(F)c1ccc(Oc2ccc([S+](c3ccccc3)c3ccccc3)cc2)cc1 ; Fc1ccc(Oc2ccc([S+](c3ccccc3)c3ccccc3)cc2)c(F)c1 ; Fc1cc(F)c(Oc2ccc([S+](c3ccccc3)c3ccccc3)cc2)c(F)c1 ; Oc1ccc(Oc2ccc([S+](c3ccccc3)c3ccccc3)cc2)cc1F ; Oc1ccc(Oc2ccc([S+](c3ccccc3)c3ccccc3)cc2)c(F)c1F]

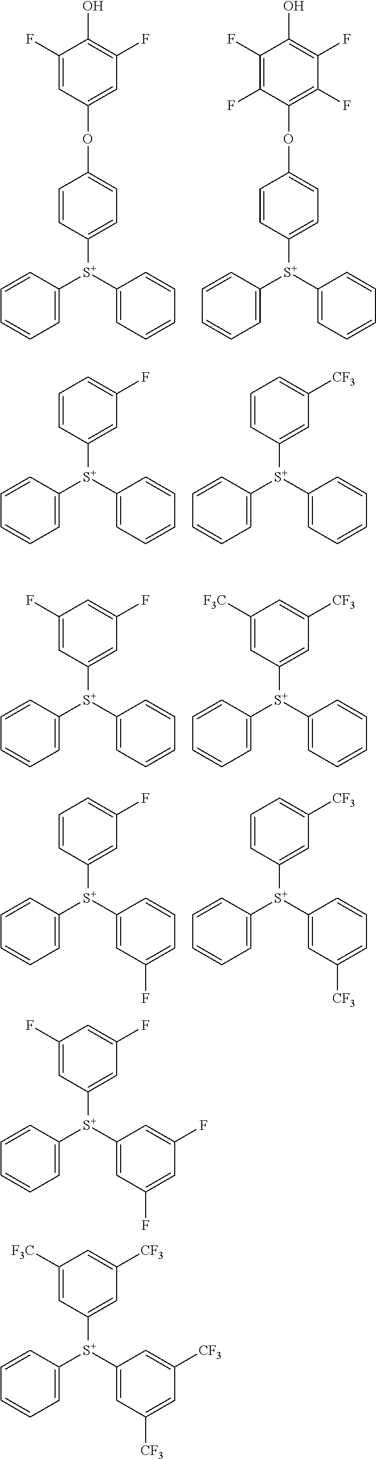

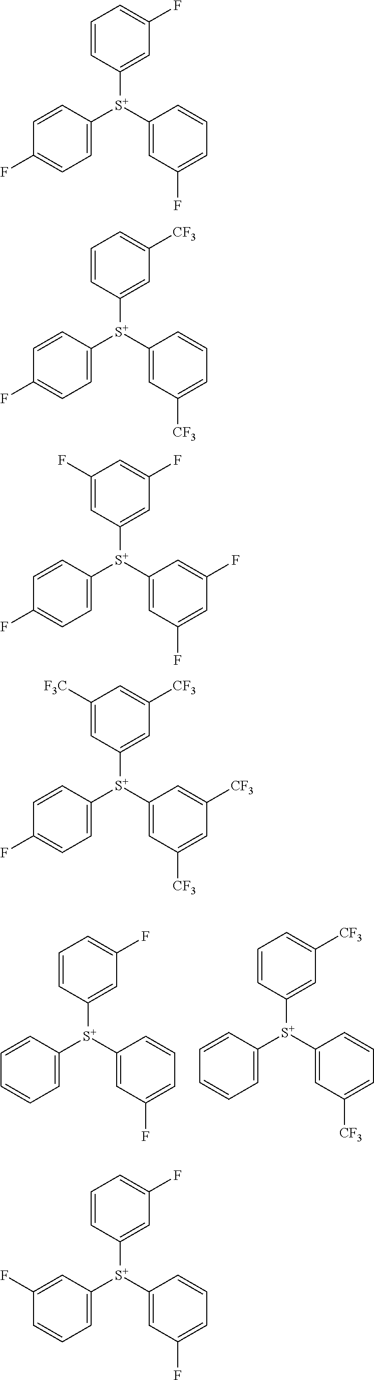

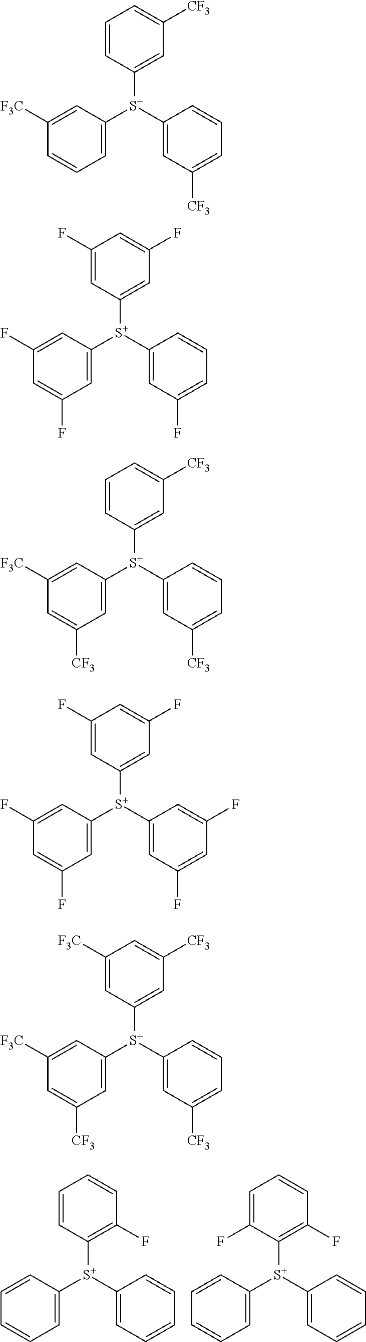

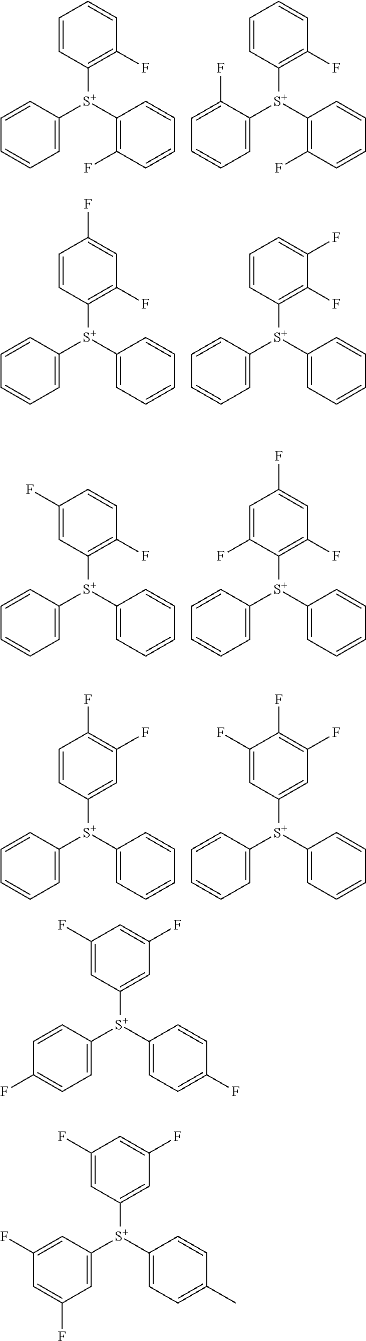

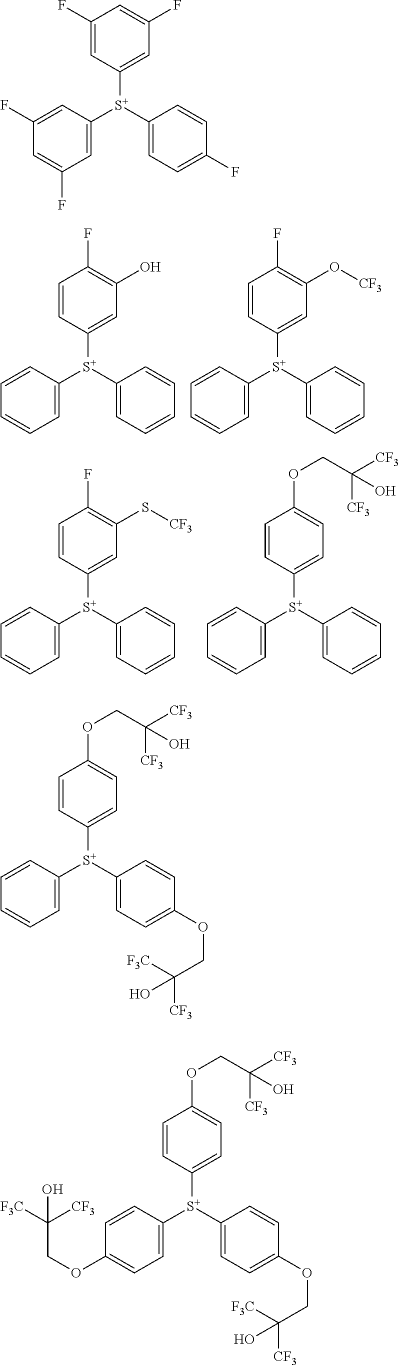

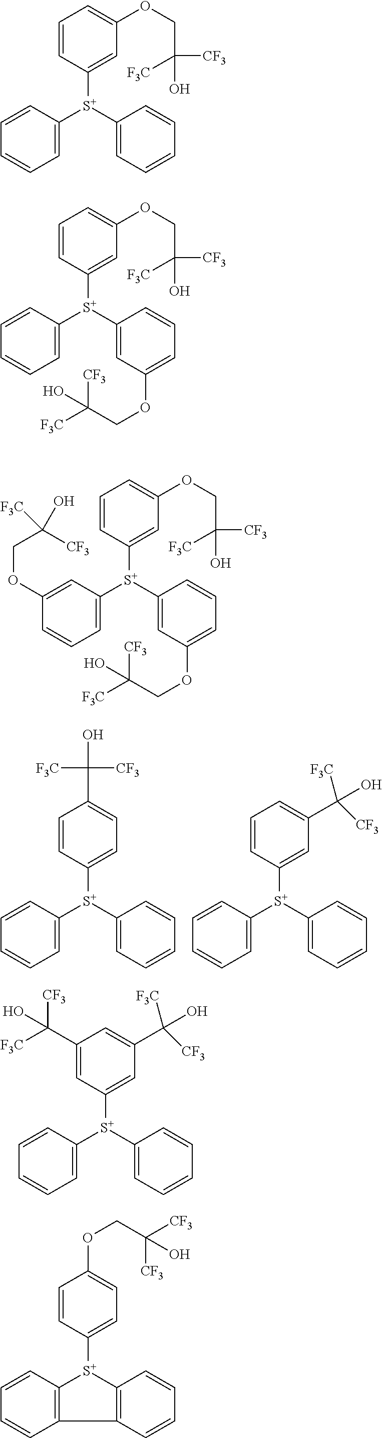

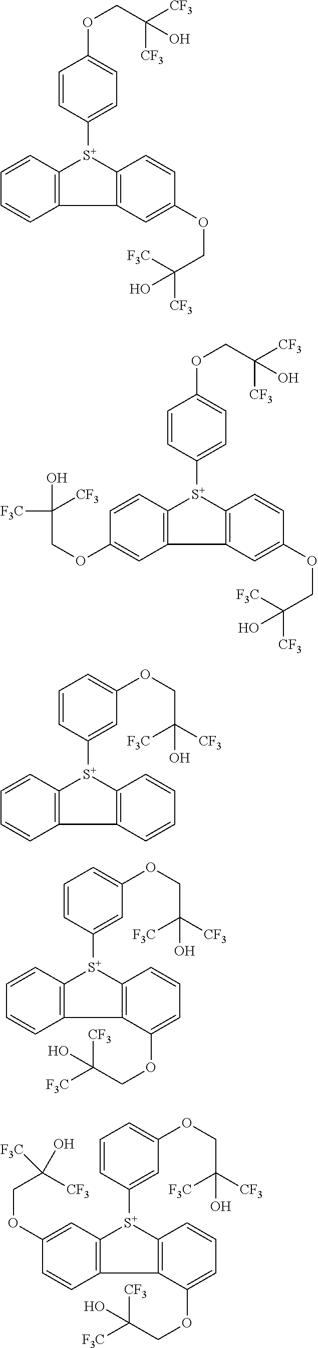

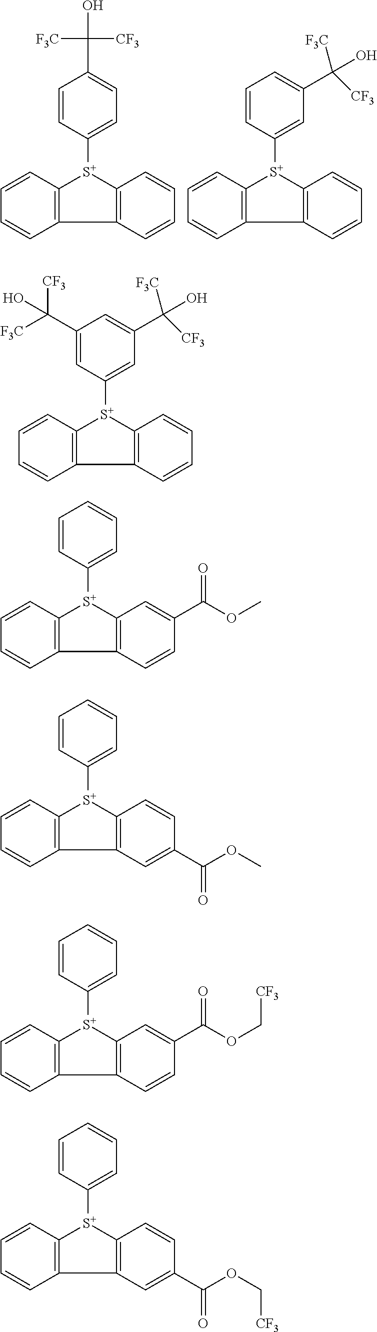

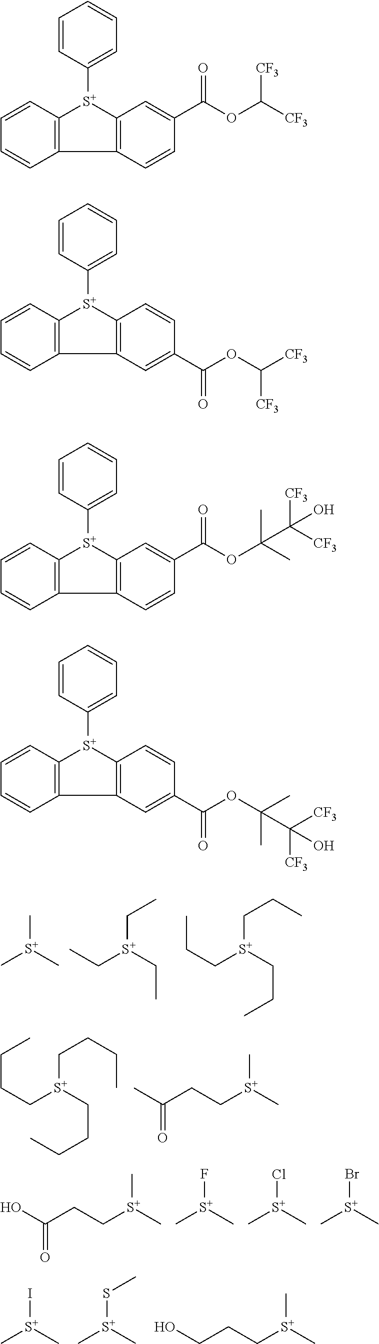

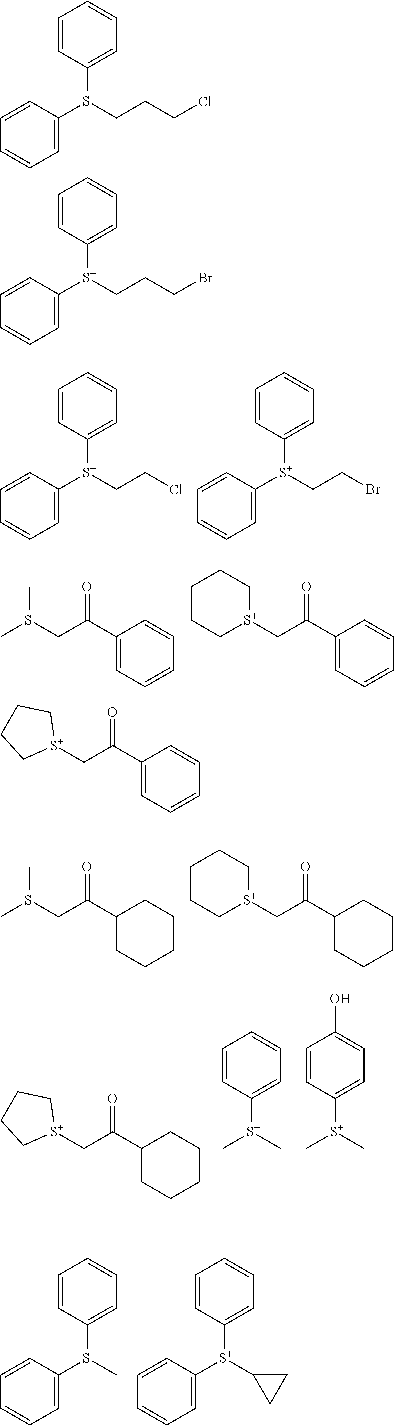

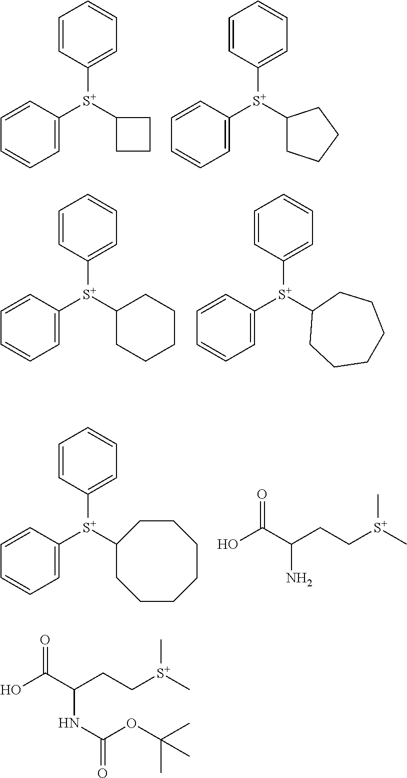

Examples of the iodonium cation represented by the general formula (Cation-2) include those shown below, but are not limited thereto.

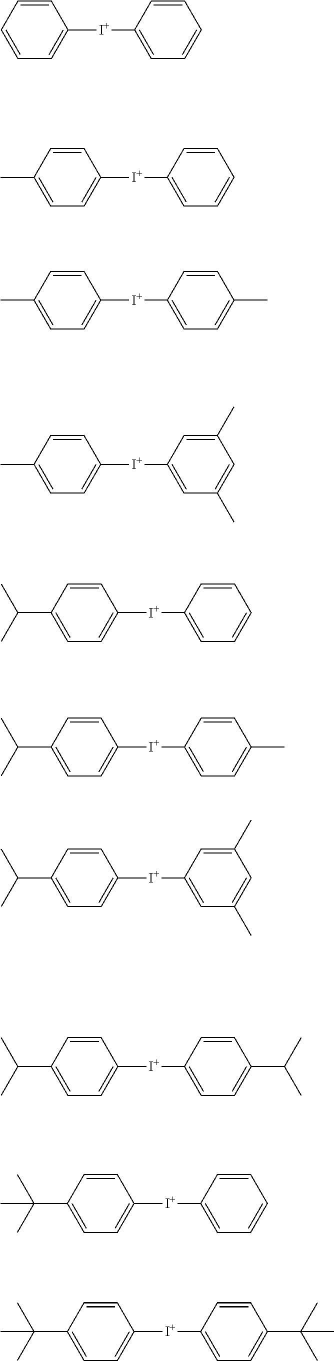

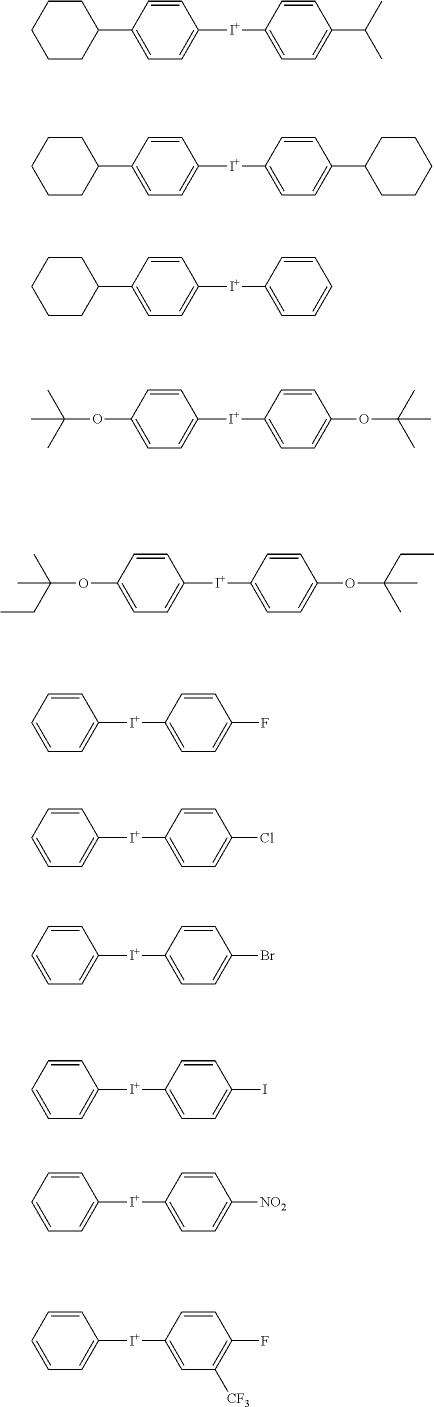

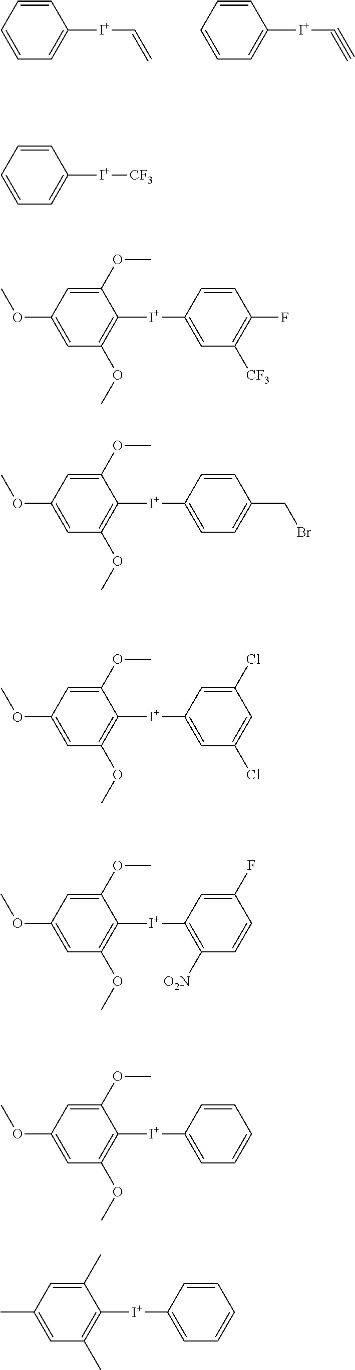

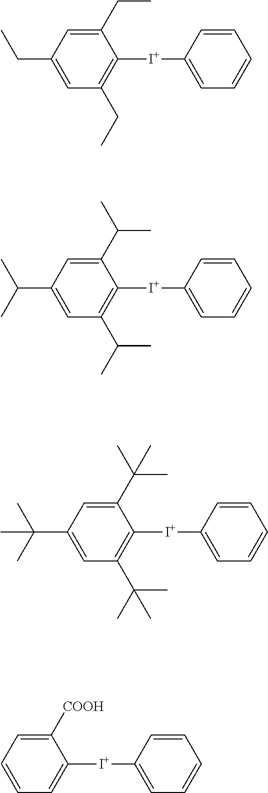

Examples of the ammonium cation represented by the general formula (Cation-3) include the following, but are not limited thereto.

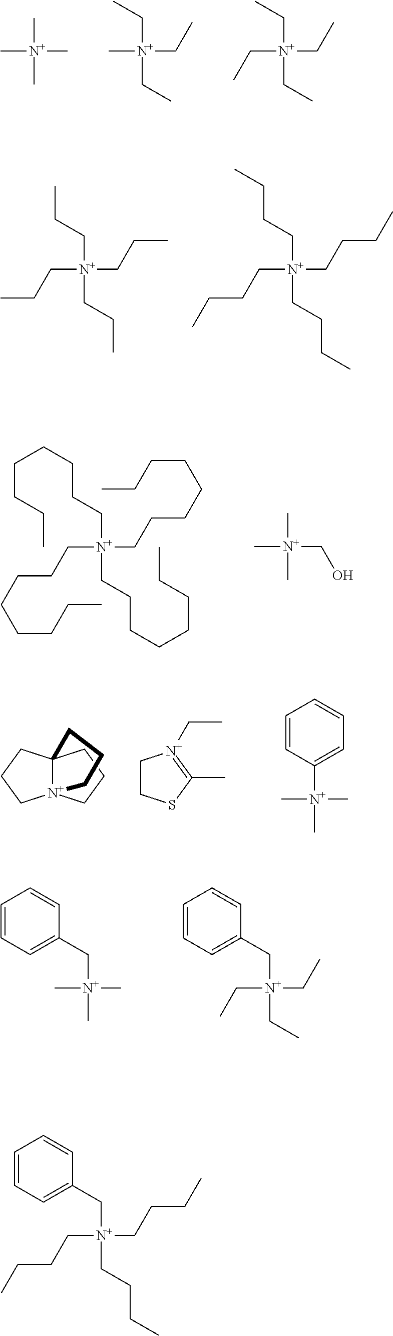

Examples of specific structures of the inventive onium salt include all combinations of the above-described anions and cations.

The inventive onium salt can be synthesized by, for example, subjecting a hydrochloride or a carbonate having an onium cation to ion exchange by using a corresponding aromatic carboxylic acid anion.

When the inventive onium salt and an onium salt that generates a strong acid, such as a sulfonic acid, an imidic acid, or a methide acid (hereinafter, these acids are defined collectively as strong acids), are both present, the corresponding carboxylic acid and strong acid are generated by irradiation with light. Meanwhile, a large amount of onium salt that has not been decomposed is present in portions where the exposure dose is small. A strong acid functions as a catalyst to cause a deprotection reaction of the base polymer, but the inventive onium salt hardly causes a deprotection reaction. The strong acid undergoes ion exchange with the remaining carboxylic acid sulfonium salt to form an onium salt of a strong acid, and instead, the carboxylic acid is released. In other words, through the ion exchange, the strong acid is neutralized by the carboxylic acid onium salt. That is, the inventive onium salt functions as a quencher (acid diffusion controller). This onium salt quencher tends to generally allow low LWR of a resist pattern compared with a quencher containing an amine compound.

Salt exchange between the strong acid and the carboxylic acid onium salt is repeated countless times. The place where the strong acid is generated at the end of exposure to light is different from the place where the strong-acid-generating onium salt is originally present. It is assumed that a repeated cycle of photo acid generation and salt exchange averages acid generation points, resulting in a smaller LWR of a resist pattern after development.

A structural characteristic of the inventive onium salt is that the onium salt has a carbonylamide structure or a sulfonylamide structure in the anion. The carbonylamide structure and the sulfonylamide structure have hardly any basicity on the nitrogen atom due to an electron-withdrawing effect of the carbonyl group and the sulfonyl group adjacent to the NH in the structure, and the bonded hydrogen atom is slightly acidic. A plurality of heteroatoms are present in the carbonylamide structure and the sulfonylamide structure, and the structures have a plurality of lone pairs. Therefore, excessive diffusion of generated acid to an unexposed portion can be suppressed by electrostatic interaction between protons of the generated acid and the lone pairs. Contrarily, along with the quenching of the generated acid and generation of the carboxylic acid in an exposed portion, affinity with an alkaline developer is improved by the slightly acidic hydrogen atom bonded to the nitrogen atom in the carbonylamide structure and the sulfonylamide structure. Thus, it is possible to achieve excellent contrast between exposed portions and unexposed portions and suppress development defects.

### [Resist Composition]

The present invention provides a resist composition containing an acid diffusion controller including the above-described onium salt. The resist composition may contain a base polymer, an acid generator, an organic solvent, and other components. In the following, each component will be described.

### [Acid Diffusion Controller]

The present invention provides an acid diffusion controller including the above-described onium salt.

Since the inventive onium salt is contained, the onium salt functions as an acid diffusion controller of a resist composition, and the acid diffusion controller including the inventive onium salt is preferably contained in a resist composition.

As explained above, due to the structural characteristics of the inventive onium salt, excessive diffusion of a generated acid to an unexposed portion can be suppressed in an unexposed portion, while the generated acid is quenched and a carboxylic acid is generated in an exposed portion, improving affinity with an alkaline developer. Because of the action of this onium salt, the inventive resist composition, which contains the above-described onium salt as an acid diffusion controller, has excellent contrast between exposed and unexposed portions and can suppress development defects.

The amount of the inventive onium salt (acid diffusion controller) contained in the resist composition is preferably 0.001 to 50 parts by mass, more preferably 0.01 to 40 parts by mass based on 100 parts by mass of the base polymer described below. One kind of the inventive onium salt may be used, or two or more kinds thereof may be used in combination.

The inventive acid diffusion controller may also be combined with one or more kinds of acid diffusion controller (blend quencher) other than that of the present invention in any proportion, as described below. The blend quencher may be a known acid diffusion controller, and is not particularly limited. The total amount of the combined acid diffusion controllers to be contained is preferably 0.001 to 50 parts by mass, more preferably 0.01 to 40 parts by mass based on 100 parts by mass of the base polymer.

### [Base Polymer]

The inventive resist composition may contain a base polymer. In the case of a positive resist composition, the base polymer contains a repeating unit containing an acid-labile group. As the repeating unit containing an acid-labile group, a repeating unit represented by the following general formula (a1) (hereinafter, also referred to as a repeating unit-a1) and/or a repeating unit represented by the following general formula (a2) (hereinafter, also referred to as a repeating unit-a2) is preferable.

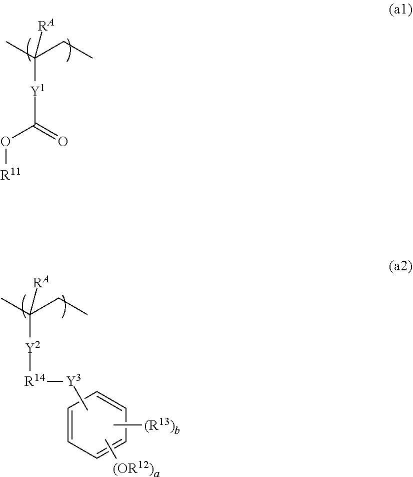

In the general formulae (a1) and (a2), each R^{A} independently represents a hydrogen atom or a methyl group. Y¹ represents a single bond or a linking group having 1 to 12 carbon atoms containing at least one selected from a phenylene group, a naphthylene group, an ester bond, and a lactone ring. Y² represents a single bond or an ester bond. Y³ represents a single bond, an ether bond, or an ester bond. RI′ and R¹² each independently represent an acid-labile group. Note that when the base polymer contains both the repeating unit-a1 and the repeating unit-a2, R¹¹ and R¹² may be identical to or different from one another. R¹² represents a fluorine atom, a trifluoromethyl group, a cyano group, or a saturated hydrocarbyl group having 1 to 6 carbon atoms. R¹⁴ represents a single bond or an alkanediyl group having 1 to 6 carbon atoms, and some of the carbon atoms are optionally substituted with an ether bond or an ester bond. “a” represents 1 or 2 and “b” represents an integer of 0 to 4, provided that 1≤a+b≤5.

Examples of a monomer to give the repeating unit-a1 include those shown below, but are not limited thereto. Incidentally, in the following formulae, R^{A} and R¹¹ are the same as above.

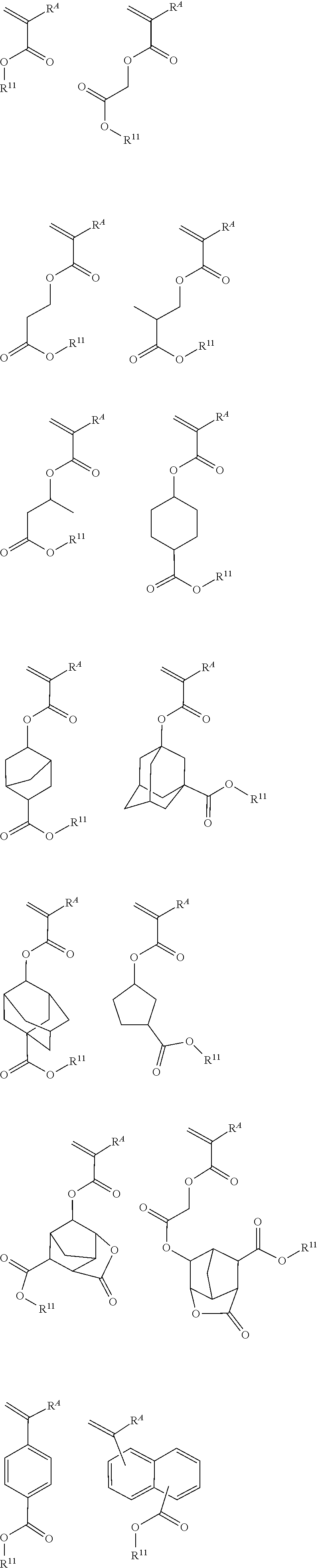

Examples of a monomer to give the repeating unit-a2 include those shown below, but are not limited thereto. Incidentally, in the following formulae, R^{A} and R¹² are the same as above.

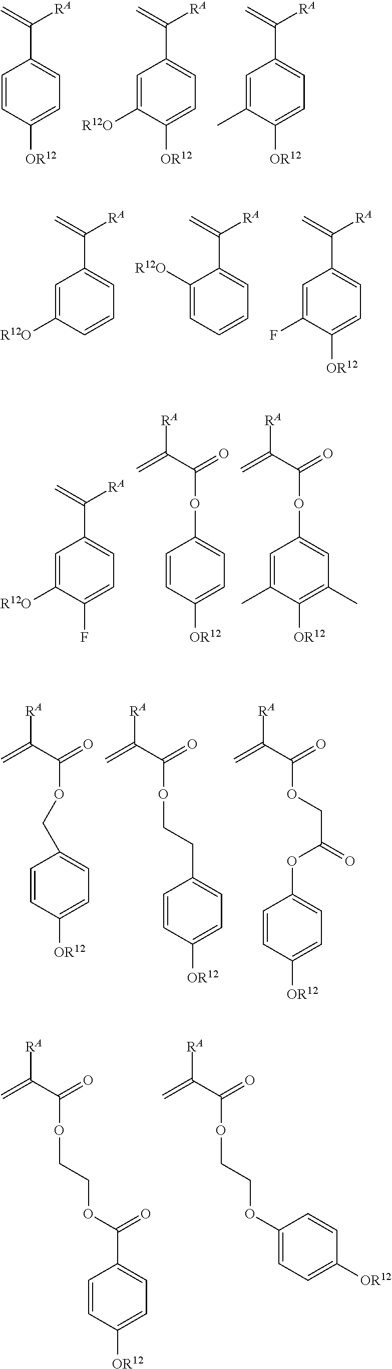

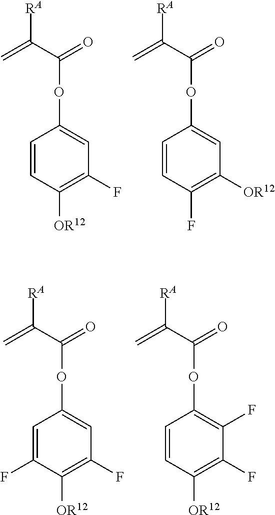

Examples of the acid-labile groups represented by R¹¹ and R¹² in the general formulae (a1) and (a2) include those disclosed in JP 2013-80033 A and JP 2013-83821 A.

Typical examples of the acid-labile groups include those represented by the following formulae (AL-1) to (AL-3).

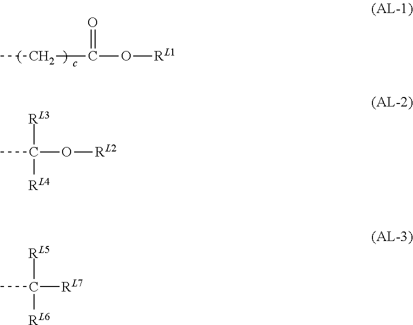

In the formulae, a broken line represents an attachment point.

In the general formulae (AL-1) and (AL-2), R^{L1} and R^{L2} each independently represent a hydrocarbyl group having 1 to 40 carbon atoms, and optionally contain a heteroatom such as an oxygen atom, a sulfur atom, a nitrogen atom, or a fluorine atom. The hydrocarbyl group may be saturated or unsaturated, and may be linear, branched, or cyclic. As the hydrocarbyl group, a saturated hydrocarbyl group having 1 to 40 carbon atoms is preferable, and a saturated hydrocarbyl group having 1 to 20 carbon atoms is more preferable.

In the general formula (AL-1), “c” represents an integer of 0 to 10, preferably an integer of 1 to 5.

In the general formula (AL-2), R^{L3} and R^{L4} each independently represent a hydrogen atom or a hydrocarbyl group having 1 to 20 carbon atoms, and optionally contain a heteroatom such as an oxygen atom, a sulfur atom, a nitrogen atom, or a fluorine atom. The hydrocarbyl group may be saturated or unsaturated, and may be linear, branched, or cyclic. As the hydrocarbyl group, a saturated hydrocarbyl group having 1 to 20 carbon atoms is preferable. Furthermore, any two of R^{L2}, R^{L3}, and R^{L4} may bond with each other to form a ring having 3 to 20 carbon atoms together with a carbon atom bonded therewith, or together with the carbon atom and an oxygen atom. As the ring, a ring having 4 to 16 carbon atoms is preferable, and an aliphatic ring is particularly preferable.

In the formula (AL-3), R^{L5}, R^{L6}, and R^{L7} each independently represent a hydrocarbyl group having 1 to 20 carbon atoms, and optionally contain a heteroatom such as an oxygen atom, a sulfur atom, a nitrogen atom, or a fluorine atom. The hydrocarbyl group may be saturated or unsaturated, and may be linear, branched, or cyclic. As the hydrocarbyl group, a saturated hydrocarbyl group having 1 to 20 carbon atoms is preferable. Furthermore, any two of R^{L5}, R^{L6}, and R^{L7} may bond with each other to form a ring having 3 to 20 carbon atoms together with a carbon atom bonded therewith. As the ring, a ring having 4 to 16 carbon atoms is preferable, and an aliphatic ring is particularly preferable.

When the base polymer of the resist composition contains the repeating unit-a1 and/or -a2, the resist composition may be a chemically amplified positive resist composition.

Cases where the base polymer of the resist composition does not contain an acid-labile group are also favorable, and in such cases, the resist composition may be a chemically amplified negative resist composition.

The base polymer may also contain, as an adhesive group, a repeating unit-b containing a phenolic hydroxy group. Examples of a monomer to give the repeating unit-b include those shown below, but are not limited thereto. Incidentally, in the following formulae, R^{A} is as defined above.

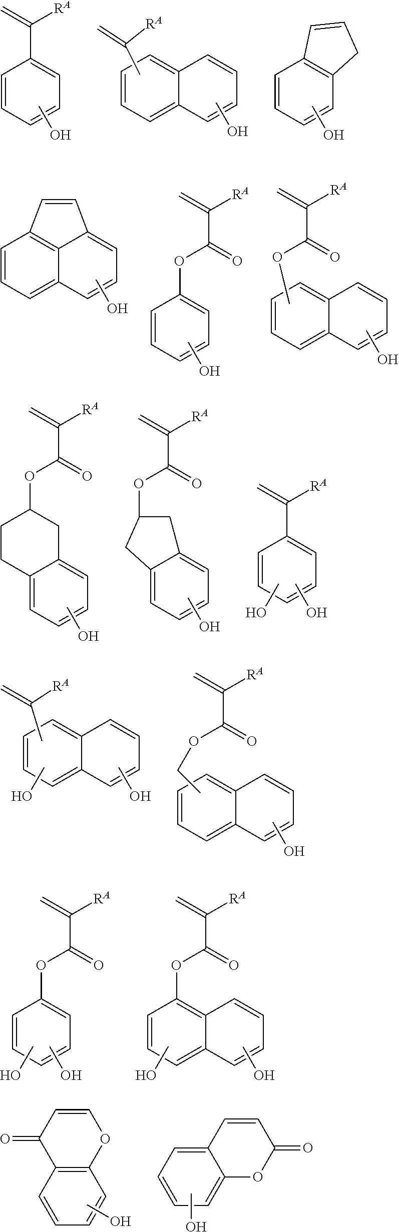

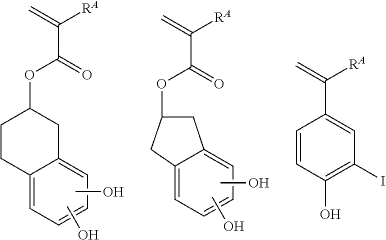

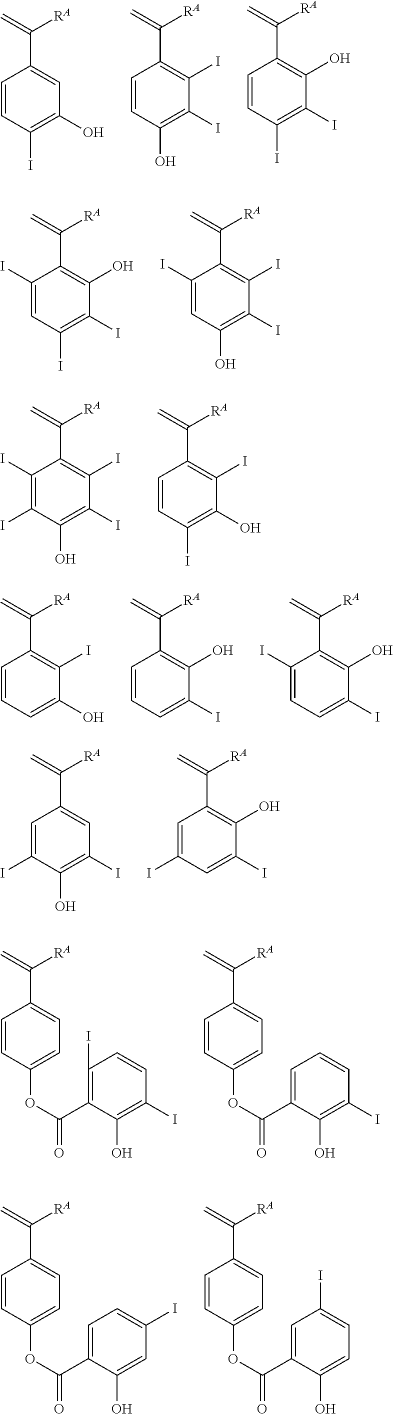

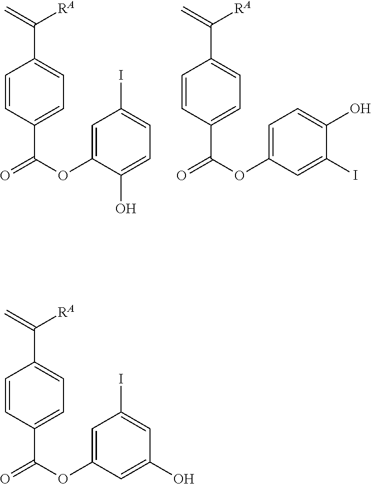

The base polymer may also contain, as a different adhesive group, a repeating unit-c containing a group other than a phenolic hydroxy group, that is, any of a hydroxy group, a lactone ring, a sultone ring, an ether bond, an ester bond, a sulfonic acid ester bond, a carbonyl group, a sulfonyl group, a cyano group, and a carboxy group. Examples of a monomer to give the repeating unit-c include those shown below, but are not limited thereto. Incidentally, in the following formulae, R^{A} is as defined above.

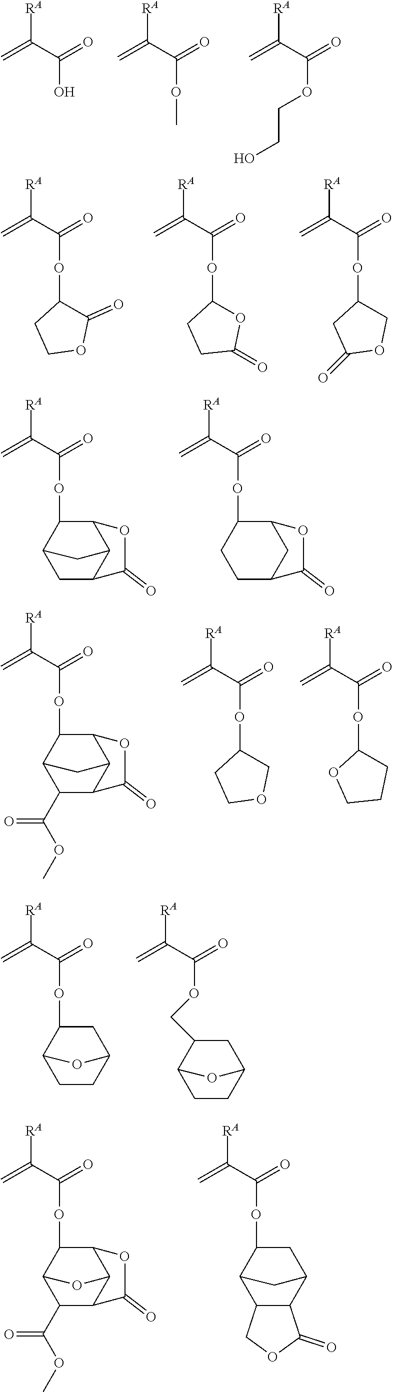

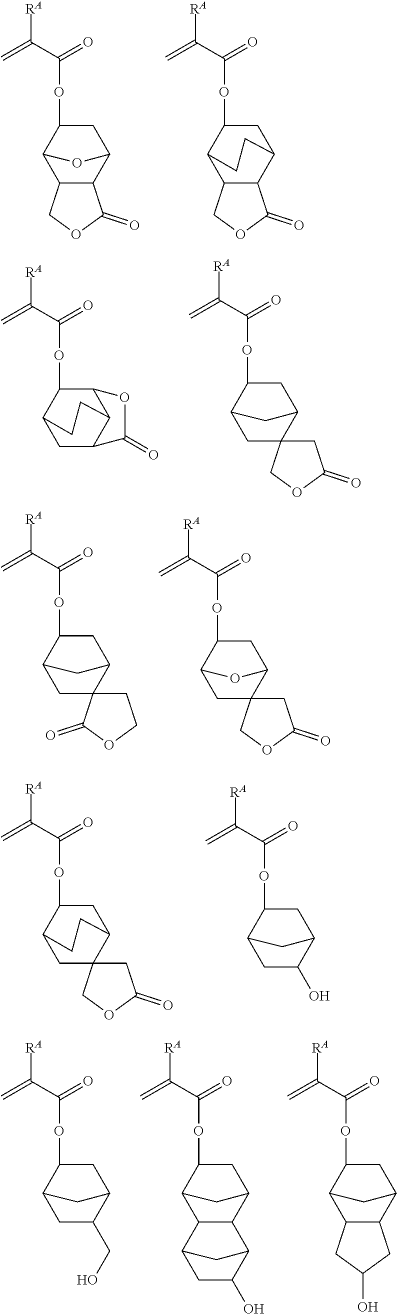

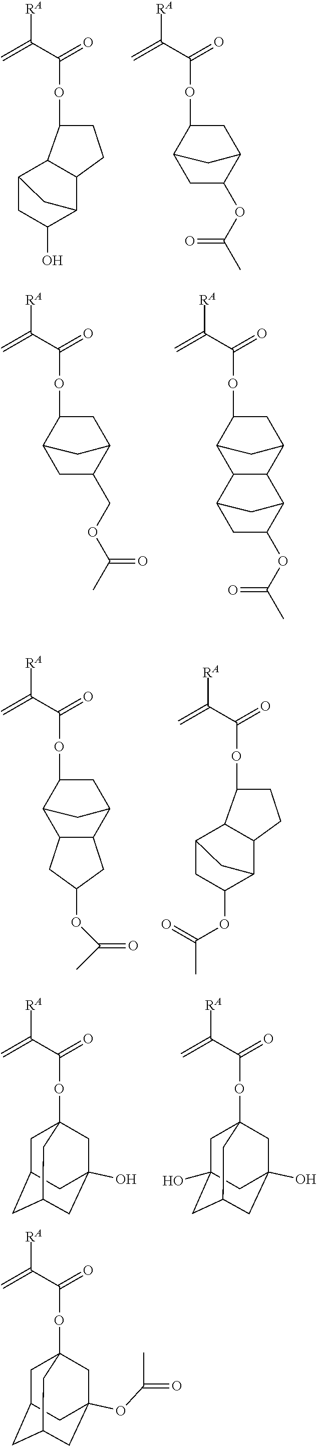

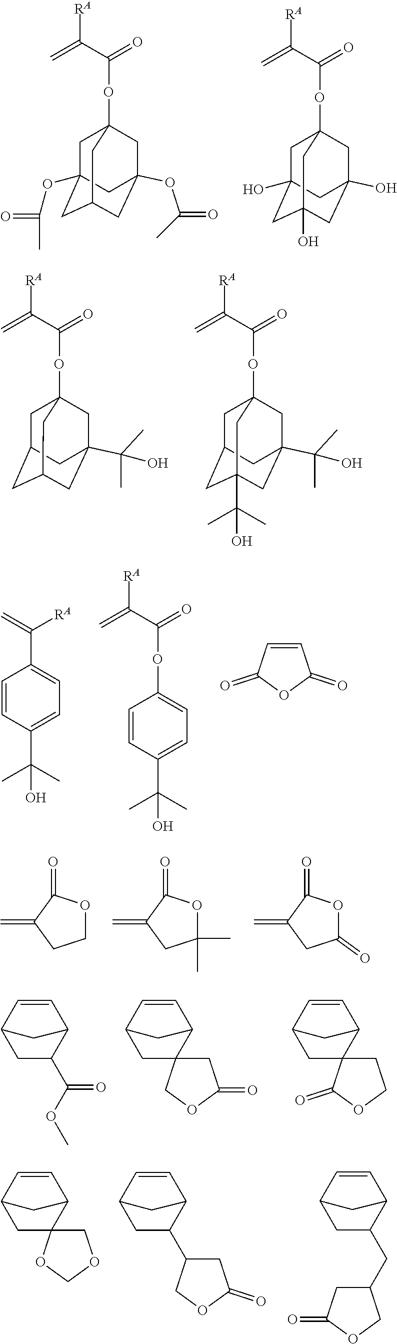

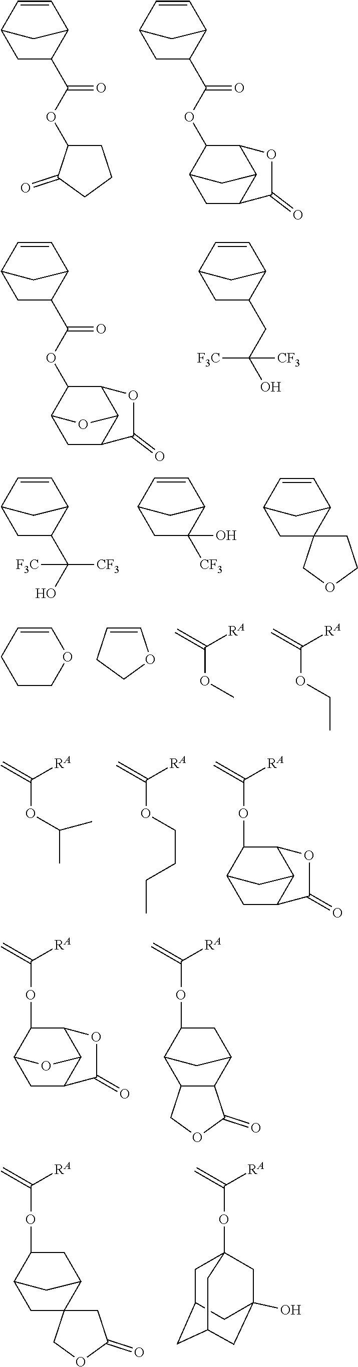

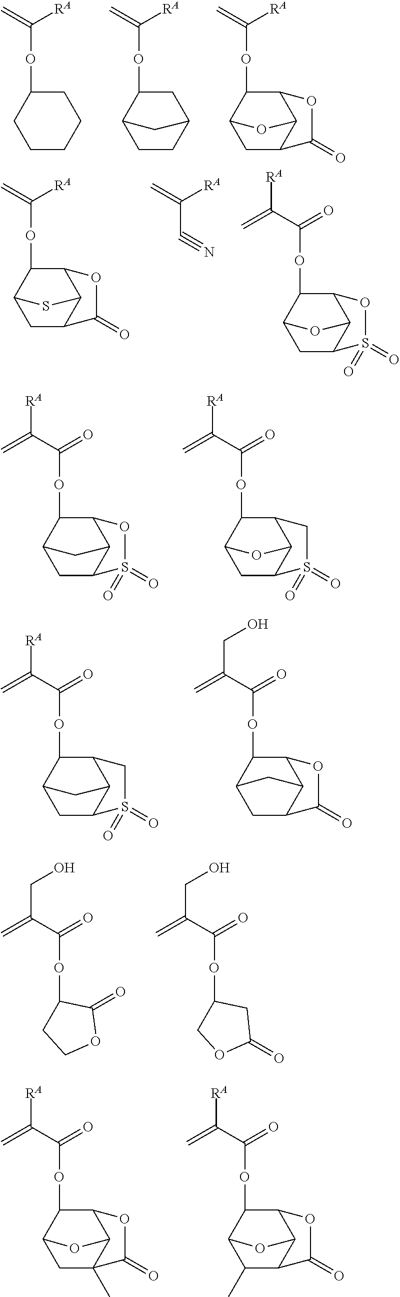

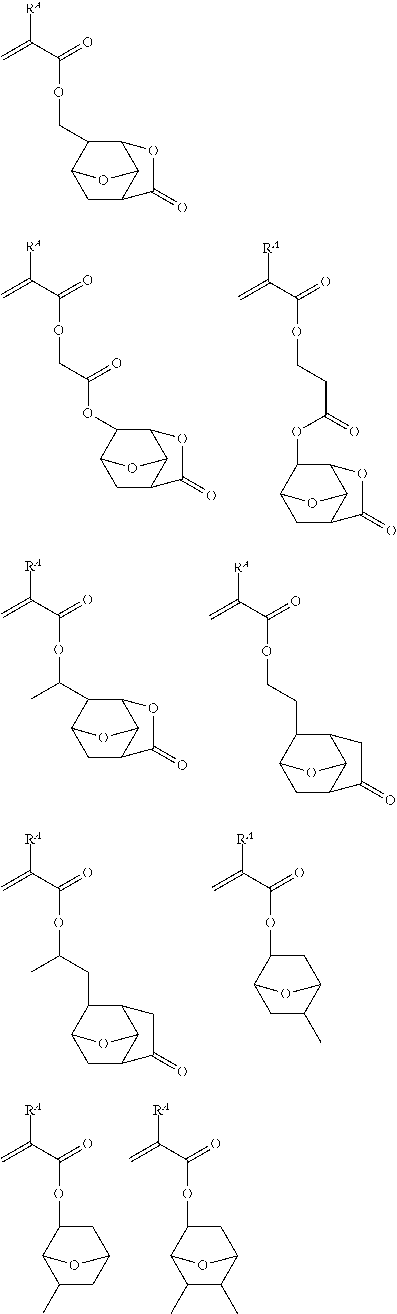

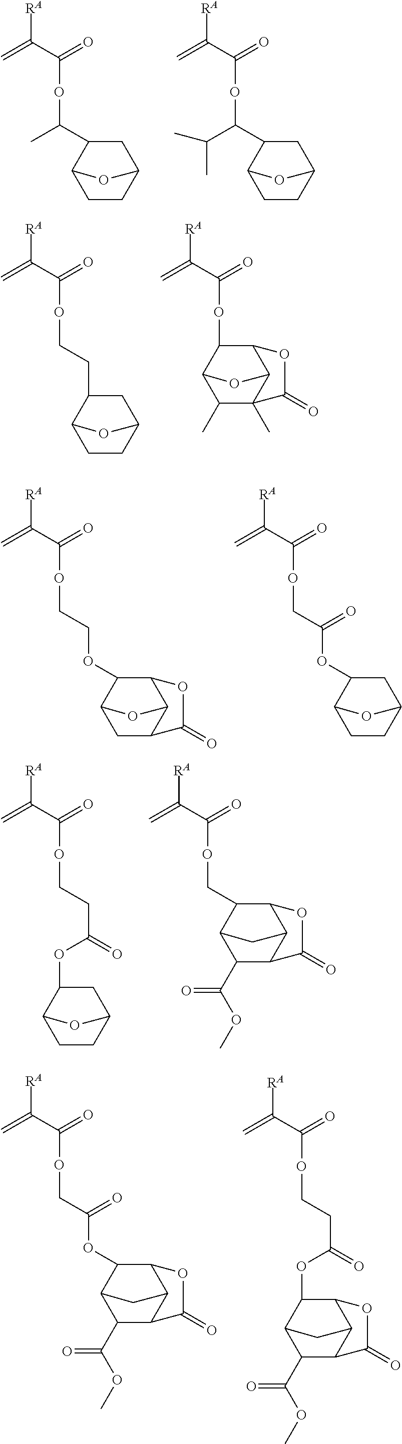

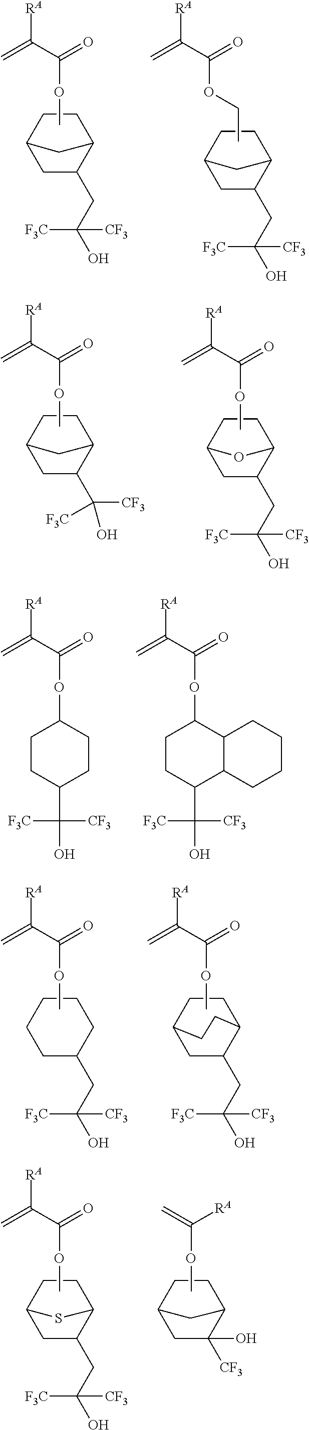

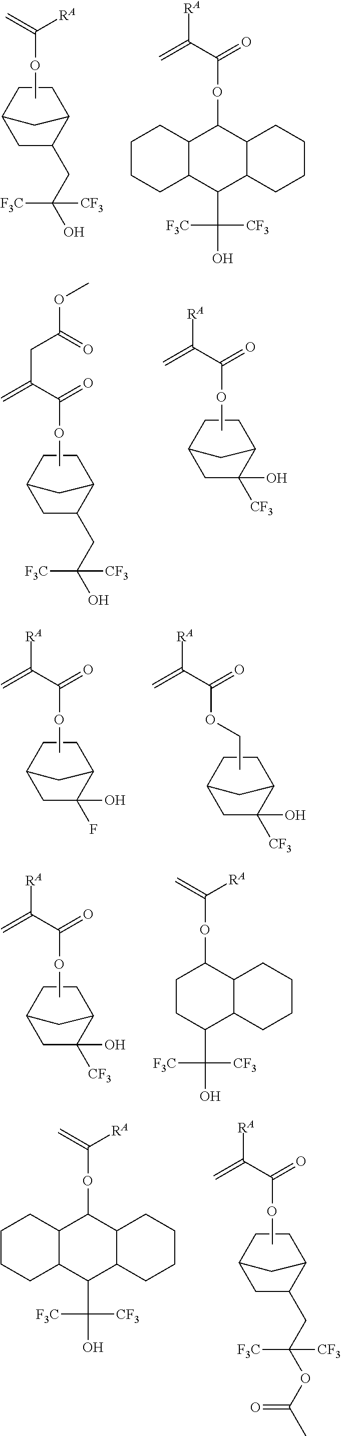

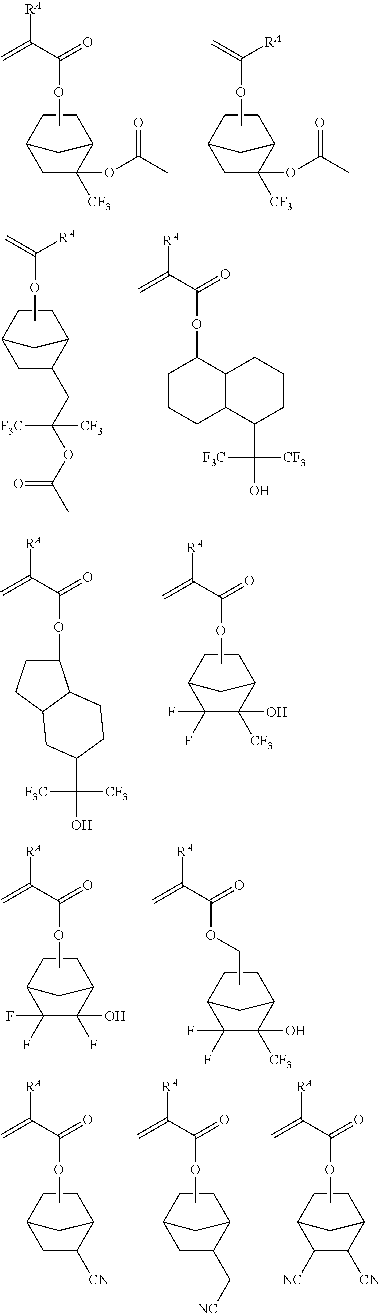

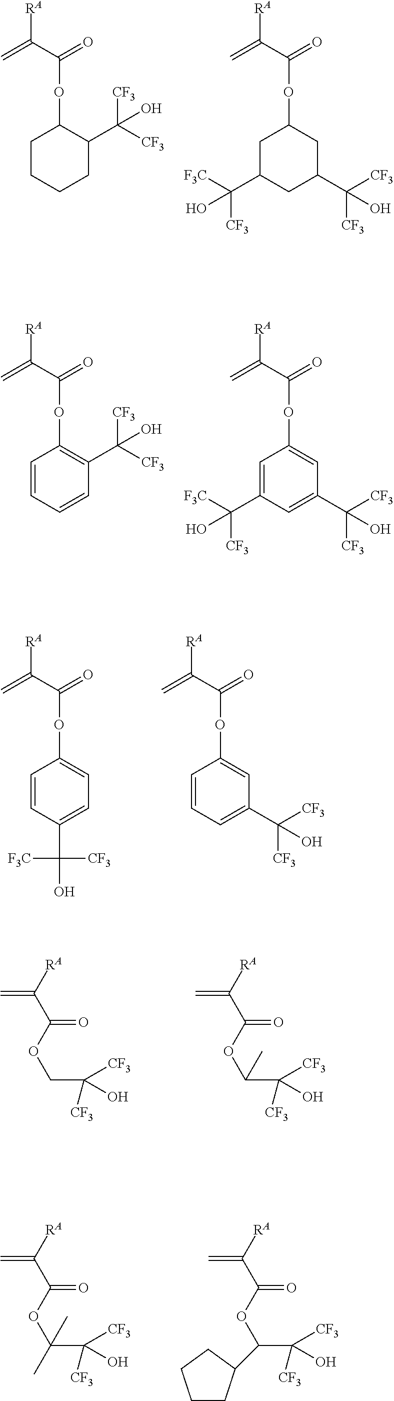

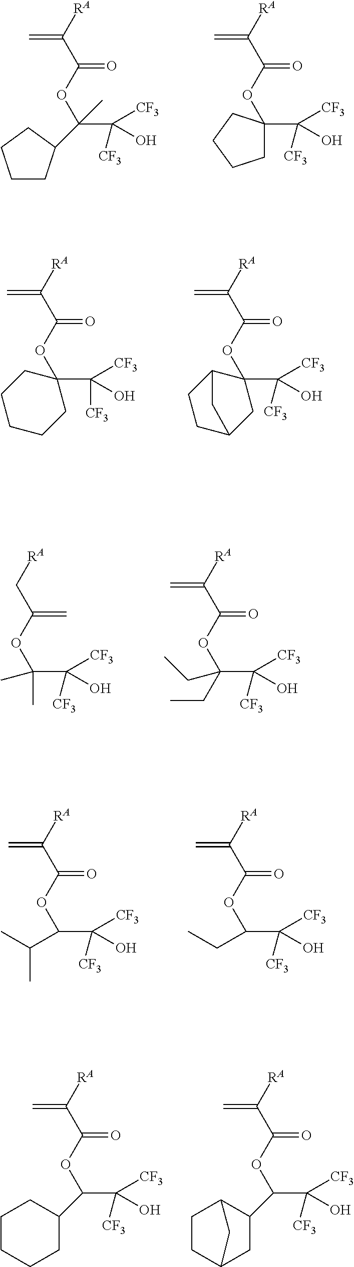

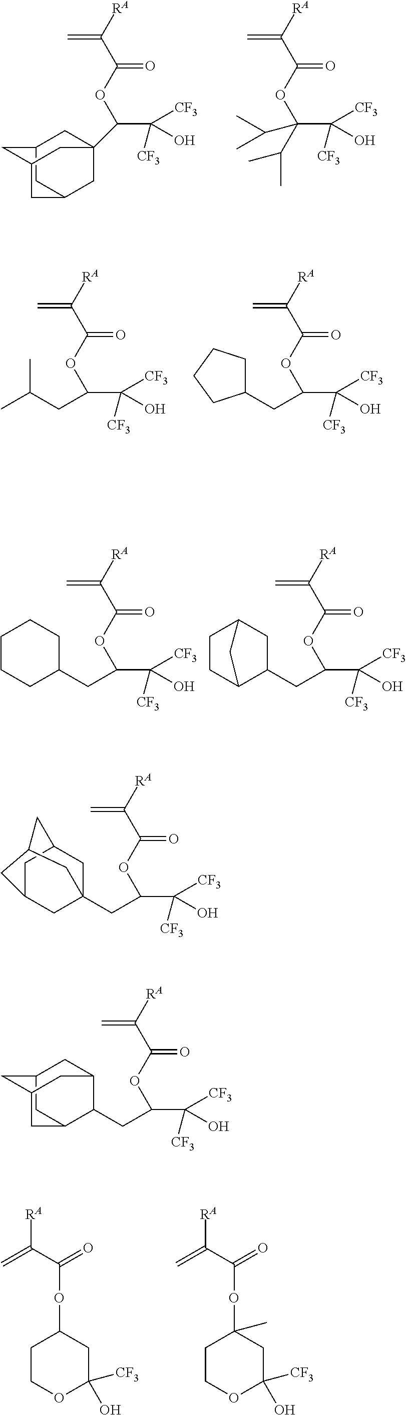

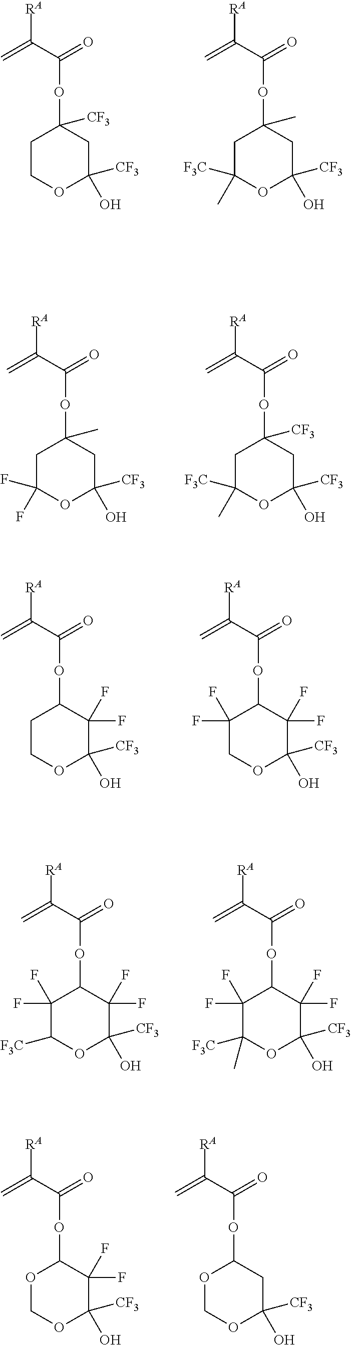

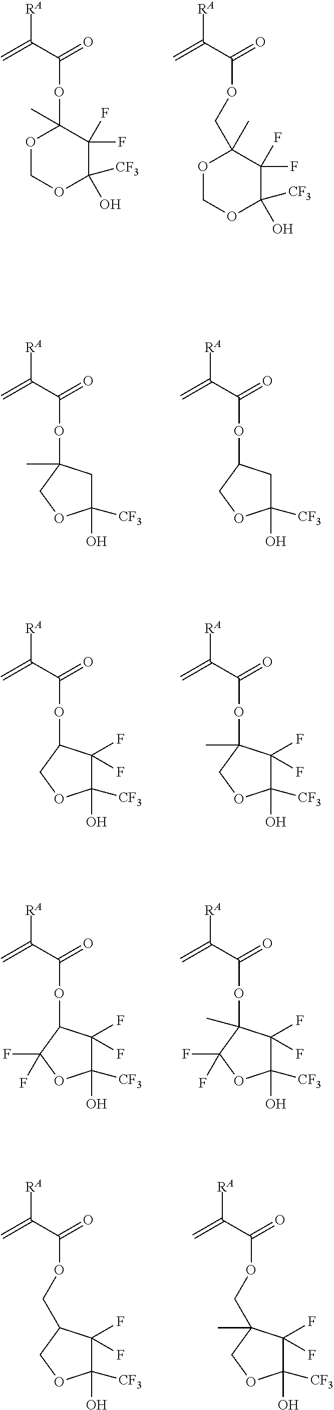

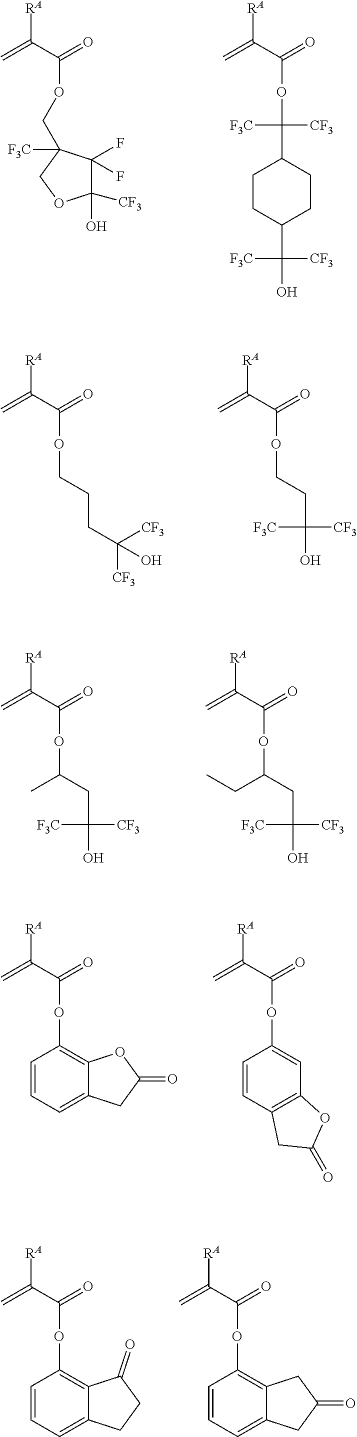

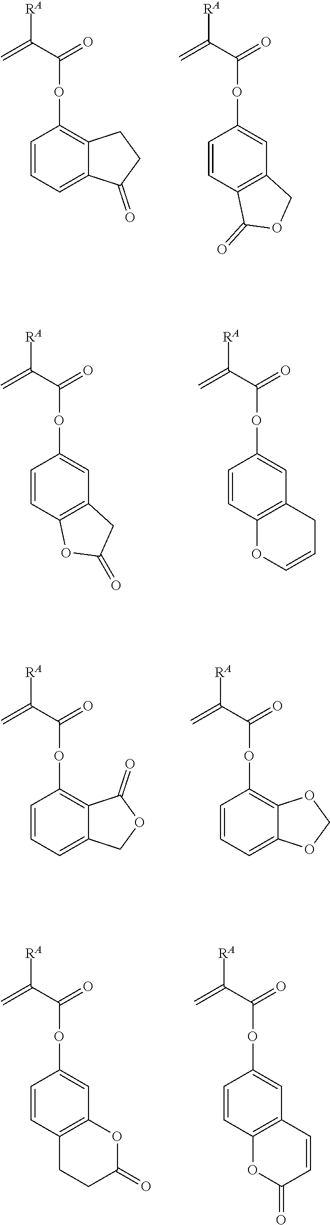

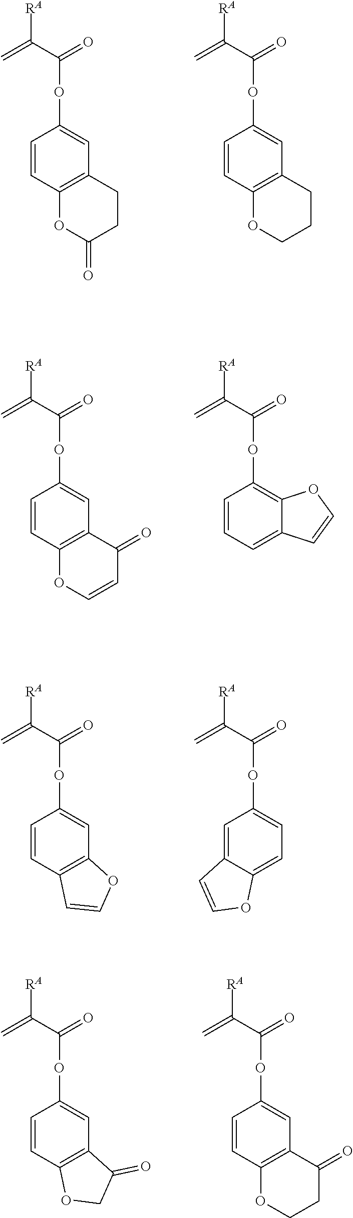

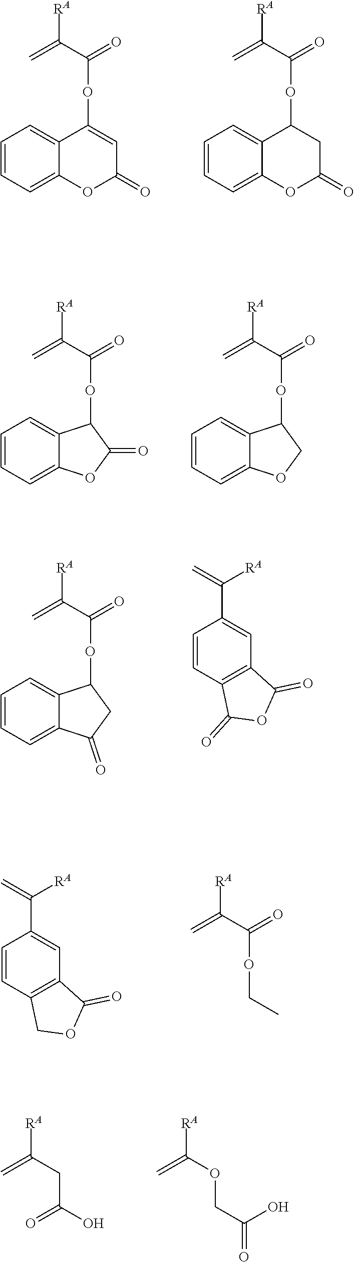

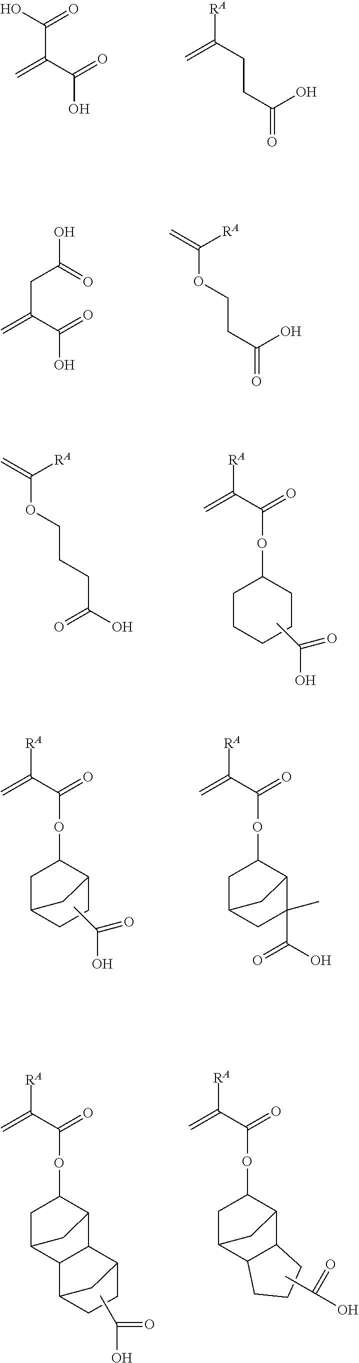

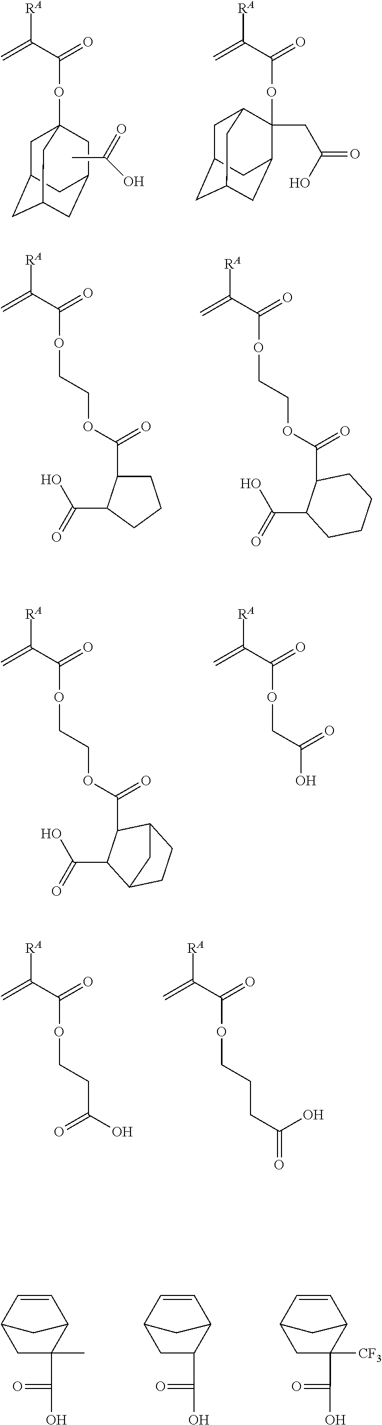

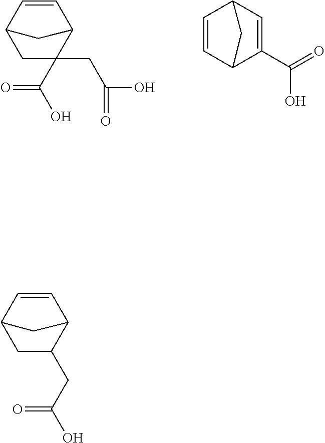

The base polymer may also contain a repeating unit-d derived from indene, benzofuran, benzothiophene, acenaphthylene, chromone, coumarin, norbornadiene, or a derivative thereof. Examples of a monomer to give the repeating unit-d include those shown below, but are not limited thereto.

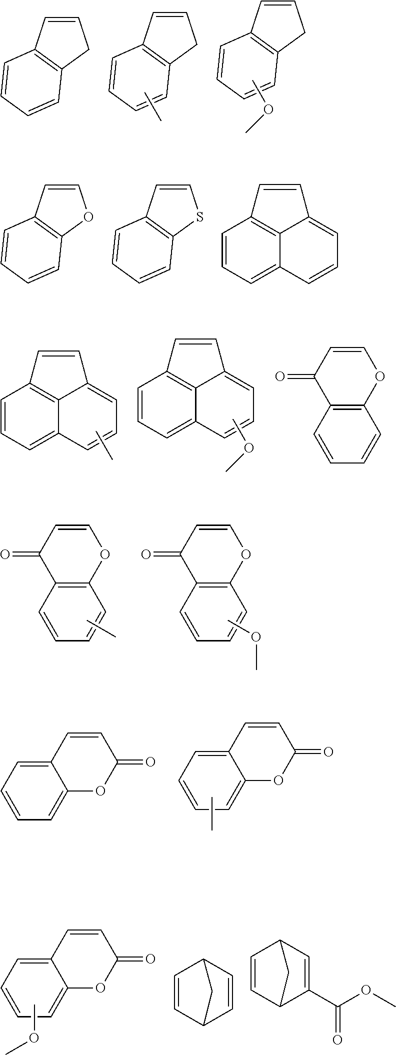

The base polymer may also contain a repeating unit-e derived from styrene, vinylnaphthalene, vinylanthracene, vinylpyrene, methyleneindane, vinylpyridine, or vinylcarbazole.

The base polymer may also contain a repeating unit-f derived from an onium salt including a polymerizable unsaturated bond. Preferable examples of the repeating unit-f include a repeating unit represented by the following general formula (f1) (hereinafter, also referred to as a repeating unit-f1), a repeating unit represented by the following general formula (f2) (hereinafter, also referred to as a repeating unit-f2), and a repeating unit represented by the following general formula (f3) (hereinafter, also referred to as a repeating unit-f3). Note that one of the repeating units-f1 to -f3 may be used, or a combination of two or more kinds thereof may be used.

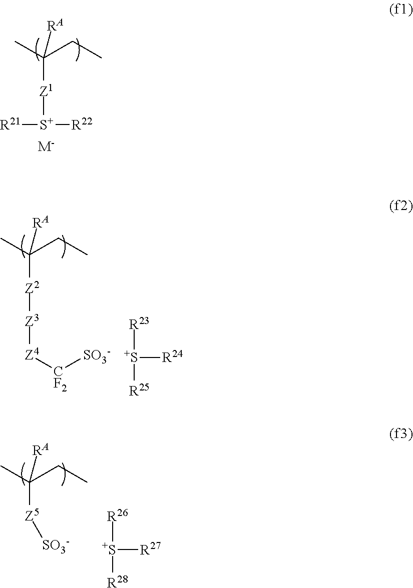

In the general formulae (f1) to (f3), each R^{A} independently represents a hydrogen atom or a methyl group. Z¹ represents a single bond, an aliphatic hydrocarbylene group having 1 to 6 carbon atoms, a phenylene group, a naphthylene group, an ester bond, a group having 7 to 18 carbon atoms derived from a combination of these groups, —O—Z¹¹—, —C(═O)—O—Z¹¹—, or —C(═O)—NH—Z¹¹—. Z¹¹ represents an aliphatic hydrocarbylene group having 1 to 6 carbon atoms, a phenylene group, a naphthylene group, or a group having 7 to 18 carbon atoms derived from a combination of these groups, Z¹¹ optionally containing a carbonyl group, an ester bond, an ether bond, or a hydroxy group. Z² represents a single bond or an ester bond. Z³ represents a single bond, —Z³¹ —C(═O)—O—, —Z³¹ —O—, or —Z³¹ —O—C(═O)—. Z³¹ represents a hydrocarbylene group having 1 to 12 carbon atoms, a phenylene group, or a group having 7 to 18 carbon atoms derived from a combination of these groups, Z³¹ optionally containing a carbonyl group, an ester bond, an ether bond, an iodine atom, or a bromine atom. Z⁴ represents a methylene group, a 2, 2,2-trifluoro-1,1-ethanediyl group, or a carbonyl group. Z⁵ represents a single bond, a methylene group, an ethylene group, a phenylene group, a fluorinated phenylene group, a phenylene group substituted with a trifluoromethyl group, —O—Z⁵¹ —, —C(═O)—O—Z⁵¹ —, or —C(═O)—NH—Z⁵¹ —. Z⁵¹ represents an aliphatic hydrocarbylene group having 1 to 6 carbon atoms, a phenylene group, a fluorinated phenylene group, a phenylene group substituted with a trifluoromethyl group, or a combination of these groups, Z⁵¹ optionally containing any of a carbonyl group, an ester bond, an ether bond, a halogen atom, and a hydroxy group.

In the general formulae (f1) to (f3), R²¹ to R²⁸ each independently represent a hydrocarbyl group having 1 to 20 carbon atoms and optionally containing a heteroatom such as a halogen atom. The hydrocarbyl group may be saturated or unsaturated, and may be linear, branched, or cyclic. Specific examples thereof include the hydrocarbyl groups given as examples of the hydrocarbyl groups represented by R^{11′} to R^{19′} in the description of the general formulae (Cation-1) to (Cation-3). Part or all of the hydrogen atoms of the hydrocarbyl group may be substituted with a group containing a heteroatom, such as an oxygen atom, a sulfur atom, a nitrogen atom, or a halogen atom, and part of the carbon atoms of these groups may be substituted with a group containing a heteroatom such as an oxygen atom, a sulfur atom, or a nitrogen atom. Thus, the resulting hydrocarbyl group may contain a hydroxy group, a fluorine atom, a chlorine atom, a bromine atom, an iodine atom, a cyano group, a nitro group, a carbonyl group, an ether bond, an ester bond, a sulfonic acid ester bond, a carbonate bond, a lactone ring, a sultone ring, a carboxylic anhydride, a haloalkyl group, etc. R²³ and R²⁴ or R²⁶ and R²⁷ may also be bonded to each other to form a ring together with the sulfur atom bonded to R²³ and R²⁴ or R²⁶ and R²⁷. In this event, examples of the ring include those given in the description of the general formula (Cation-1) as examples of the ring that can be formed by R^{11′} and R^{12′} being bonded to one another together with the sulfur atom bonded to R^{11′} and R^{12′}.

In the general formula (f1), M⁻ represents a non-nucleophilic counter ion. Examples of the non-nucleophilic counter ion include halide ions such as chloride ion and bromide ion; fluoroalkylsulfonate ions such as triflate ion, 1,1,1-trifluoroethanesulfonate ion, and nonafluorobutanesulfonate ion; arylsulfonate ions such as tosylate ion, benzenesulfonate ion, 4-fluorobenzenesulfonate ion, and 1,2,3,4,5-pentafluorobenzenesulfonate ion; alkylsulfonate ions such as mesylate ion and butanesulfonate ion; imide ions such as bis(trifluoromethylsulfonyl)imide ion, bis(perfluoroethylsulfonyl)imide ion, and bis(perfluorobutylsulfonyl)imide ion; and methide ions such as tris(trifluoromethylsulfonyl)methide ion and tris(perfluoroethylsulfonyl)methide ion.

Other examples of the non-nucleophilic counter ion include sulfonate ions having a fluorine atom substituted at a position as shown by the following general formula (f1-1), sulfonate ions having a fluorine atom substituted at a position and having a trifluoromethyl group substituted at β, position as shown by the following general formula (f1-2), etc.

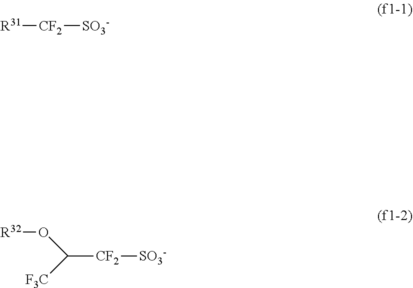

In the general formula (f1-1), R³¹ represents a hydrogen atom or a hydrocarbyl group having 1 to 20 carbon atoms, the hydrocarbyl group optionally containing an ether bond, an ester bond, a carbonyl group, a lactone ring, or a fluorine atom. The hydrocarbyl group may be saturated or unsaturated, and may be linear, branched, or cyclic. Specific examples thereof include groups given as examples of a hydrocarbyl group represented by R¹¹¹ in the formula (3A′) described below.

In the general formula (f1-2), R³² represents a hydrogen atom, a hydrocarbyl group having 1 to 30 carbon atoms, or a hydrocarbylcarbonyl group having 6 to 20 carbon atoms, the hydrocarbyl group and hydrocarbylcarbonyl group optionally containing an ether bond, an ester bond, a carbonyl group, or a lactone ring. The hydrocarbyl moiety of the hydrocarbyl group and the hydrocarbylcarbonyl group may be saturated or unsaturated, and may be linear, branched, or cyclic. Specific examples thereof include the groups given as examples of the hydrocarbyl group represented by R¹¹¹ in the formula (3A′) described below.

Examples of a cation of a monomer to give the repeating unit-f1 include those shown below, but are not limited thereto. Note that in the following formulae, R^{A} is as defined above.

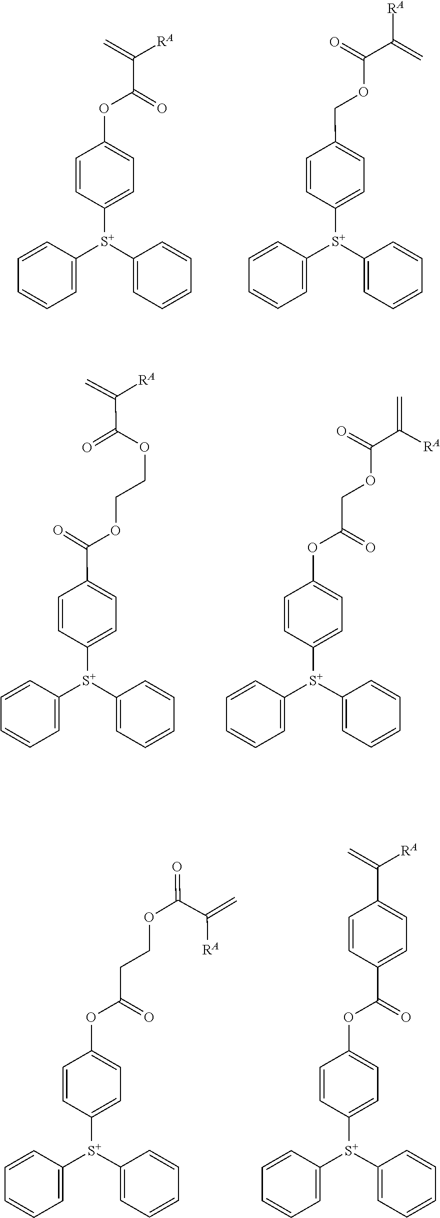

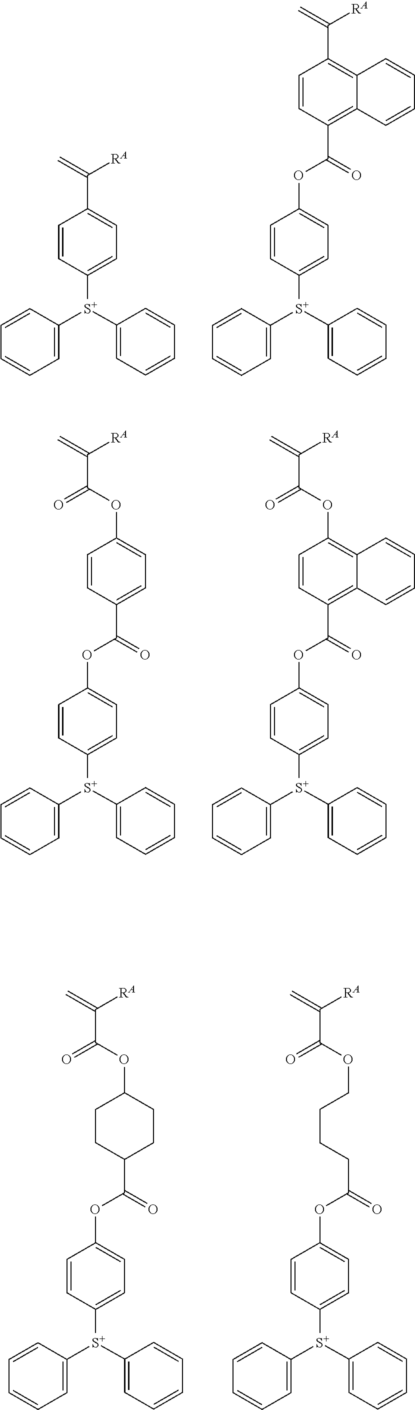

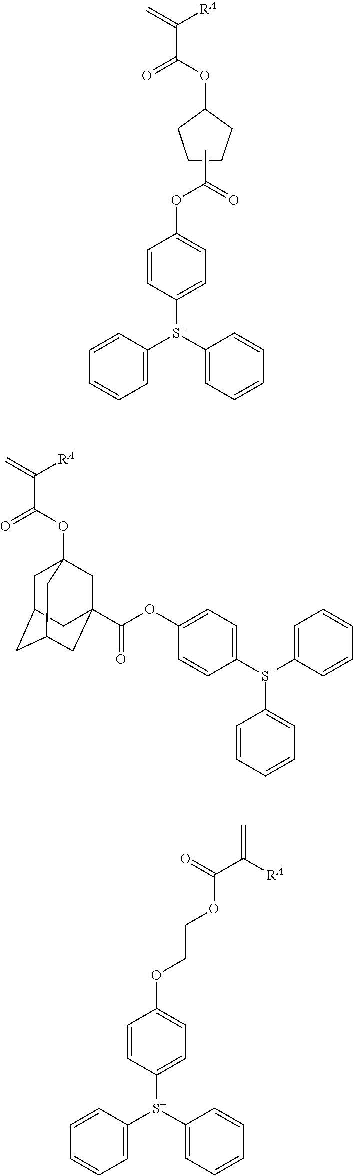

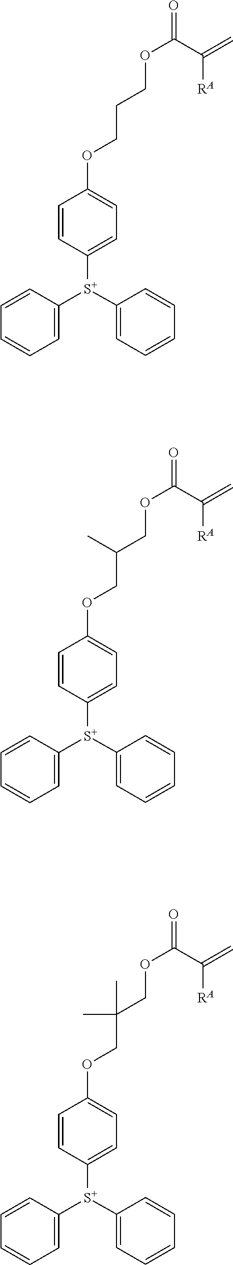

Specific examples of a cation of a monomer to give the repeating unit-f2 or -f3 include the cations given as examples of the cation of a sulfonium salt represented by the formula (Cation-1).

Examples of an anion of a monomer to give the repeating unit-f2 include those shown below, but are not limited thereto. Note that in the following formulae, R^{A} is as defined above.

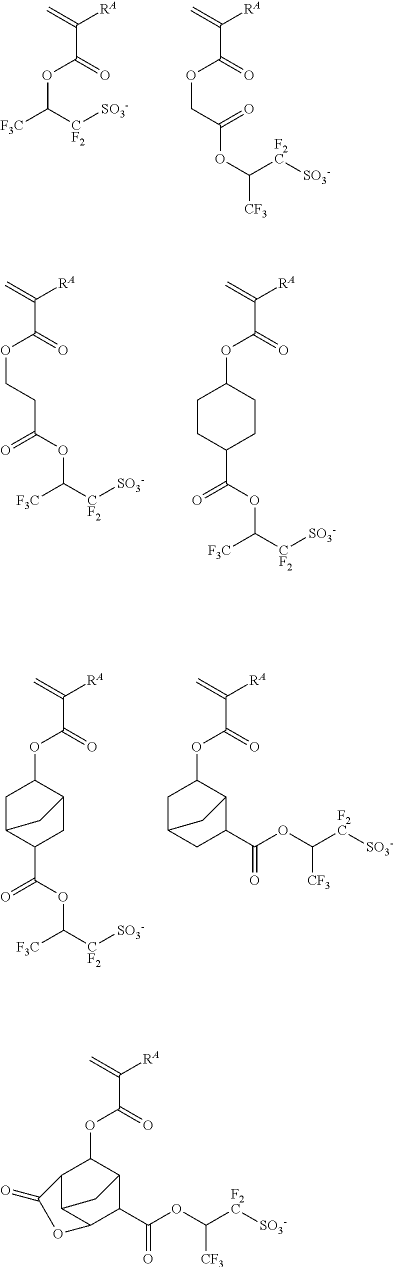

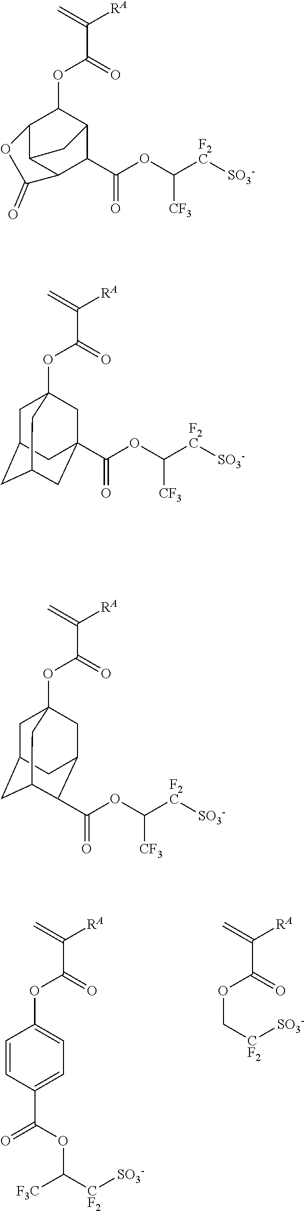

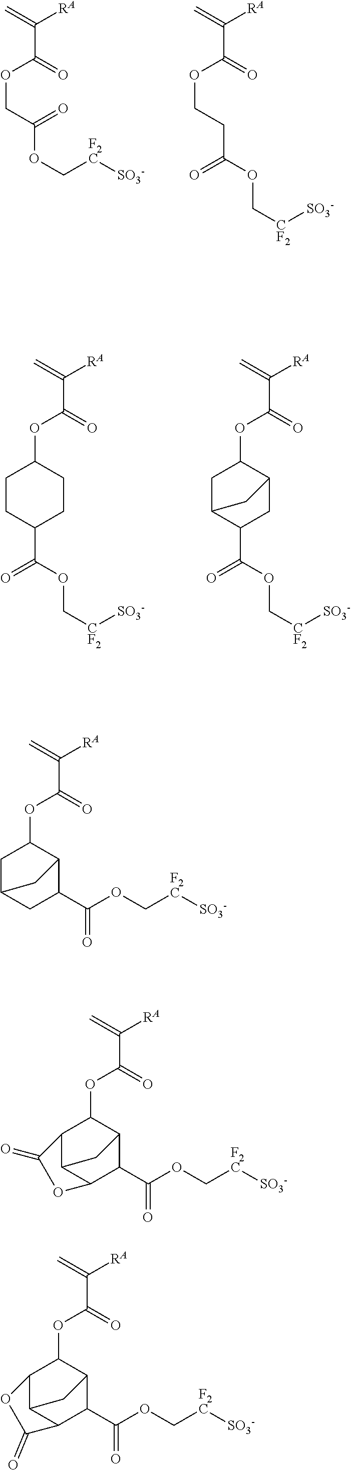

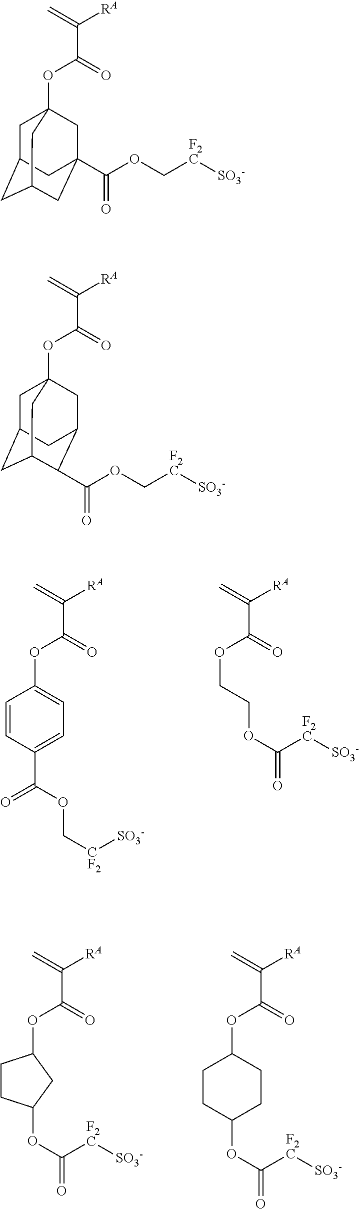

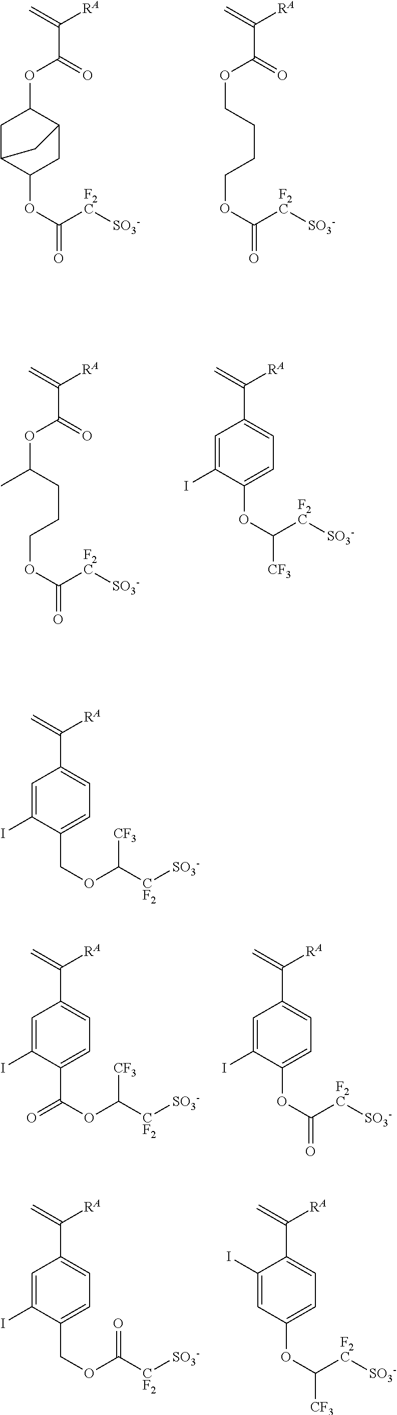

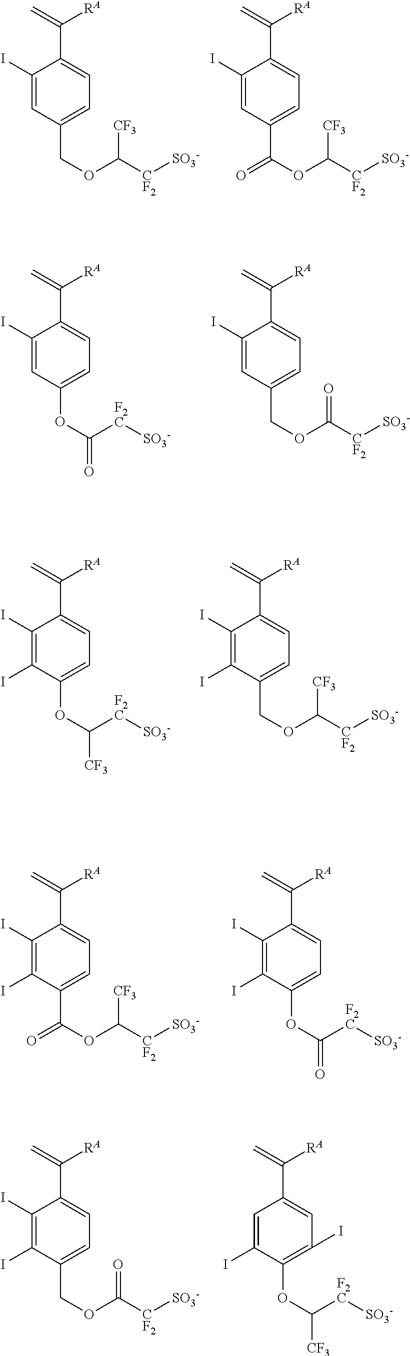

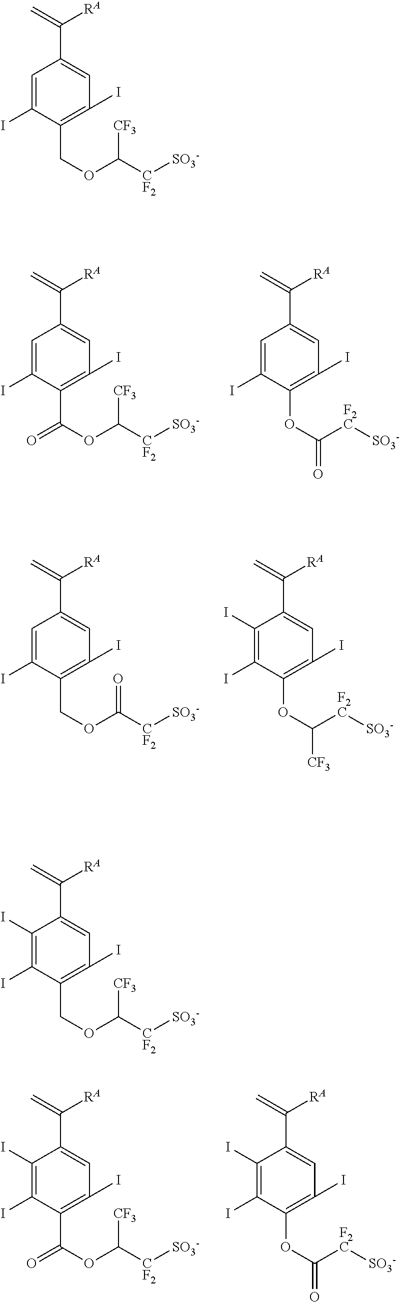

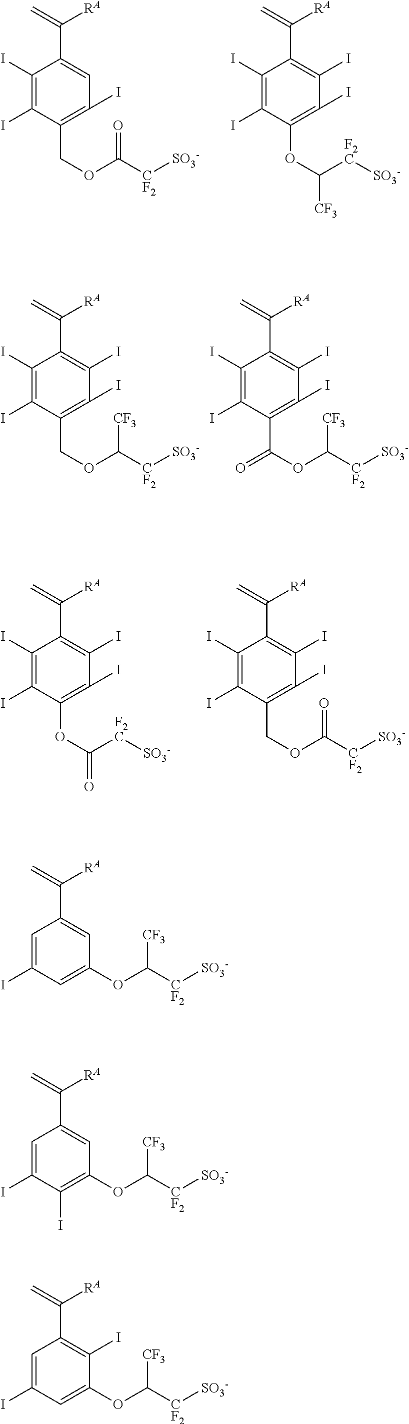

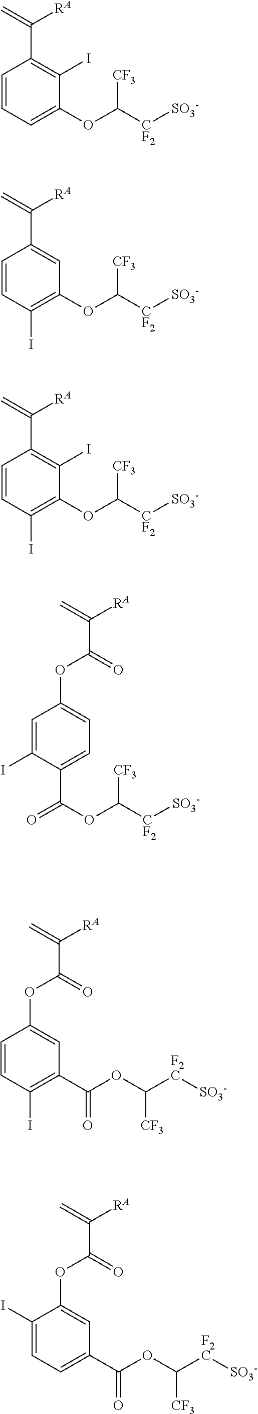

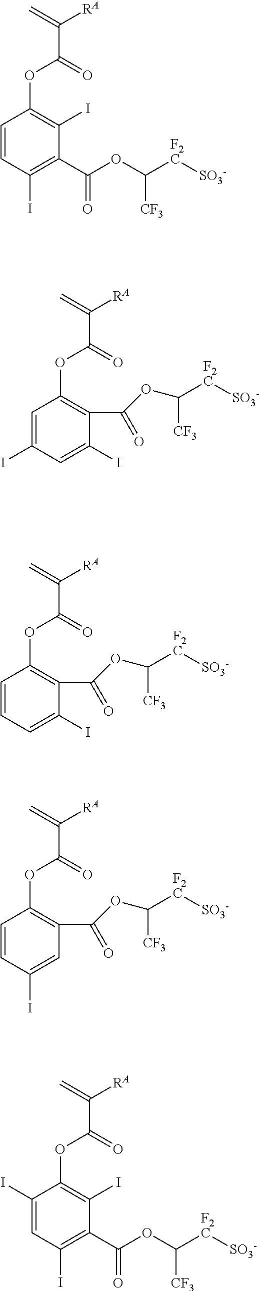

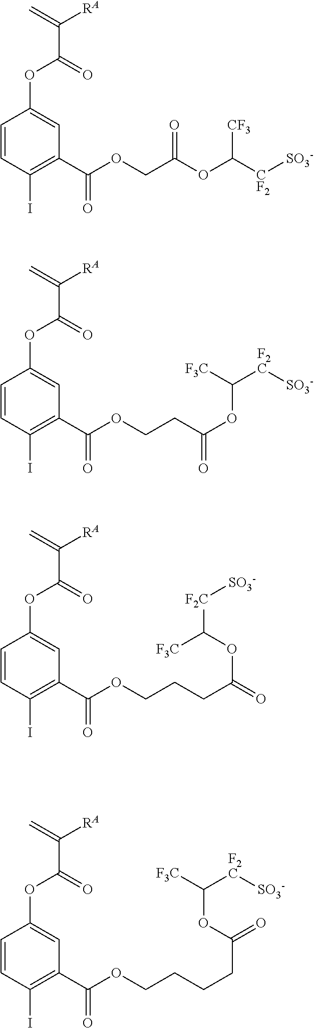

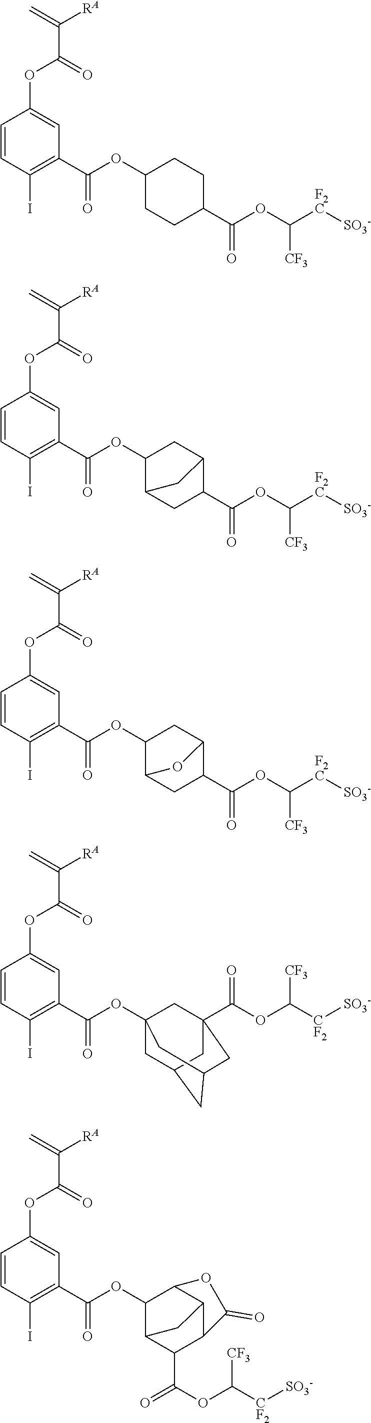

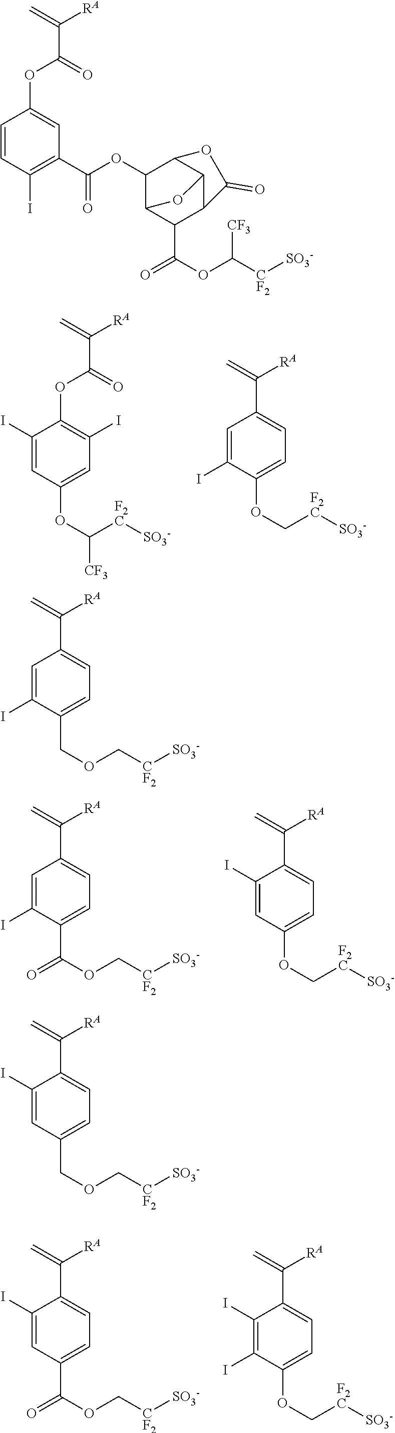

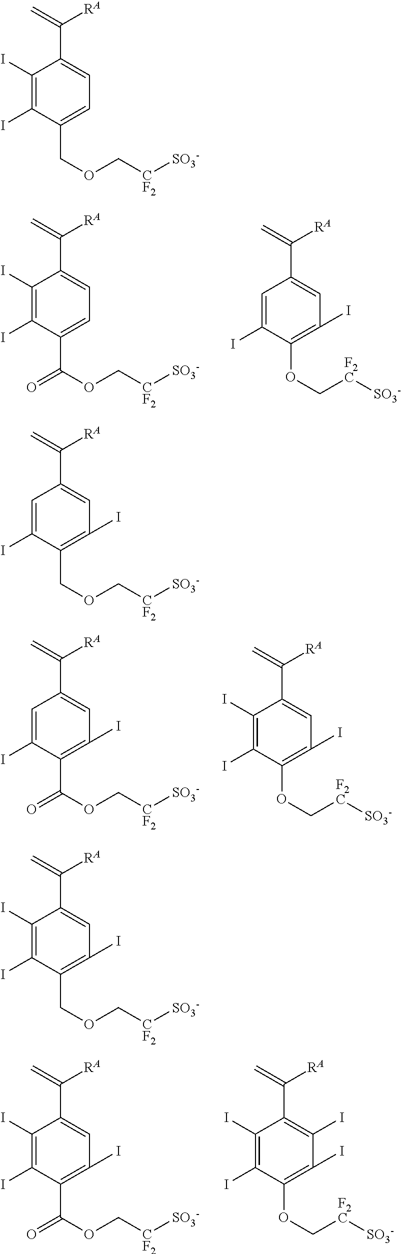

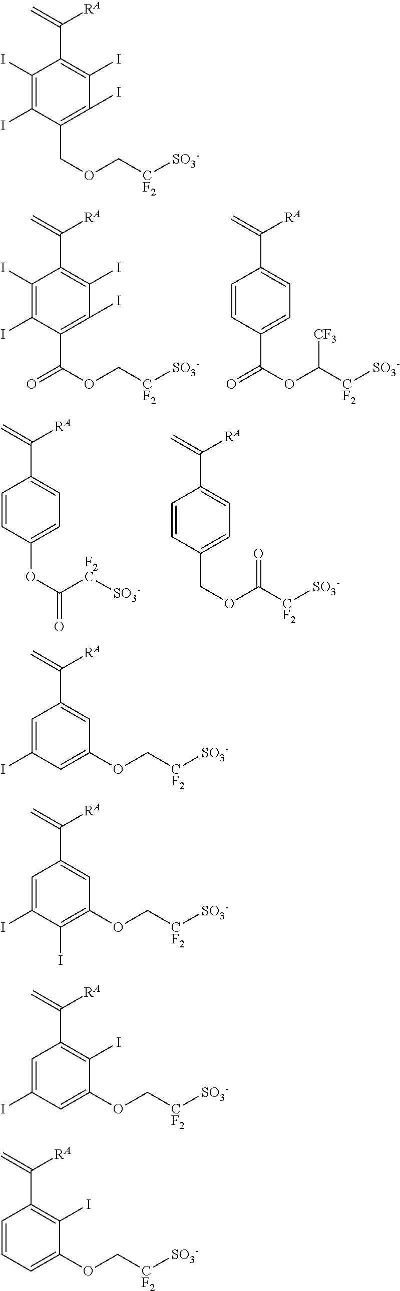

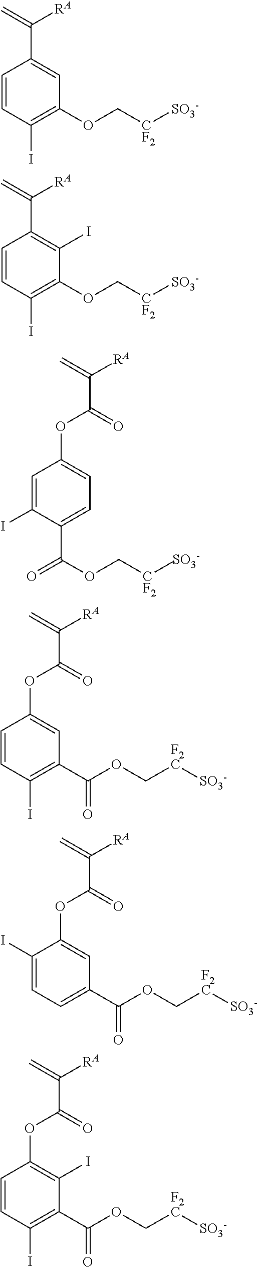

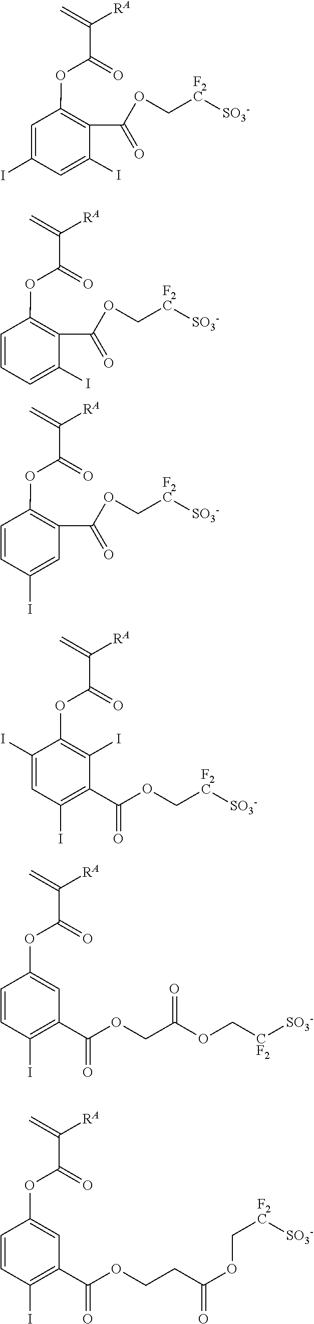

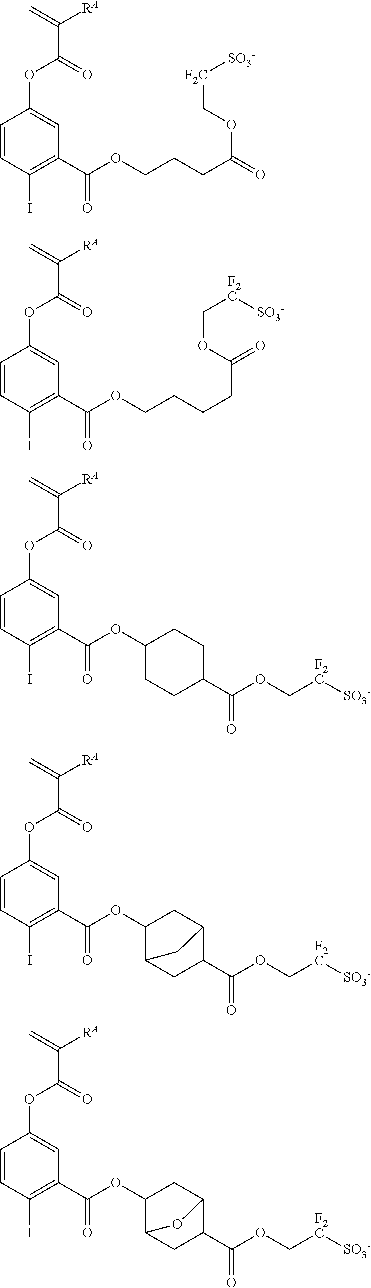

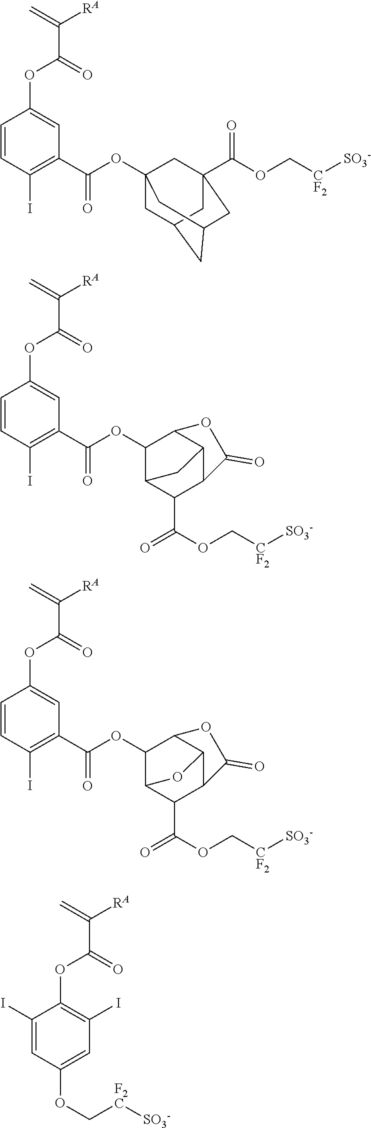

Examples of an anion of a monomer to give the repeating unit-f3 include those shown below, but are not limited thereto. Note that in the following formulae, R^{A} is as defined above.

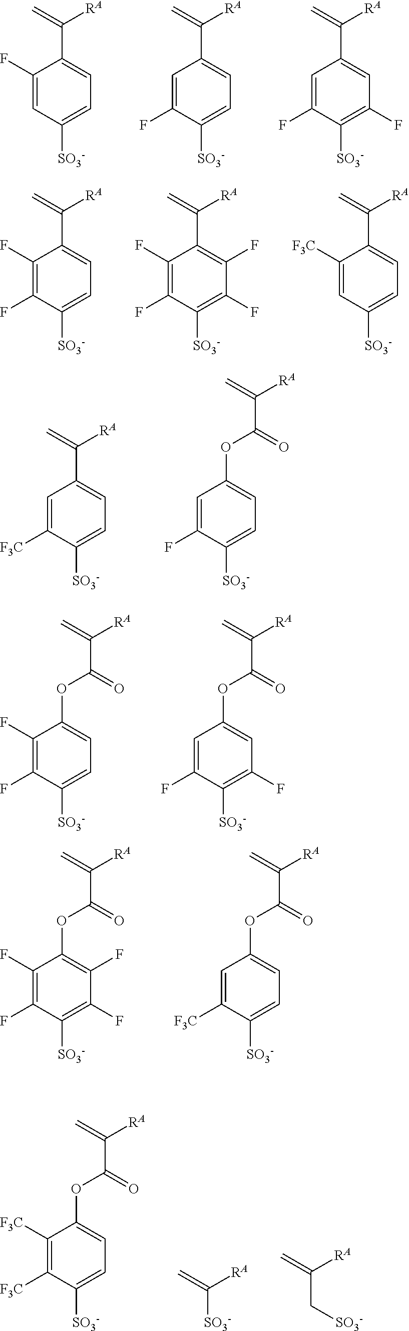

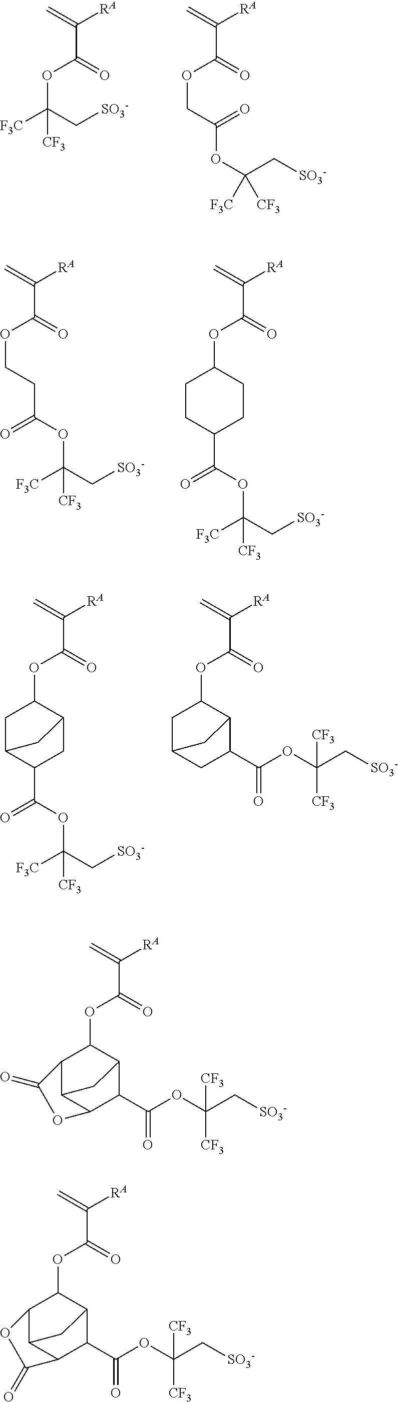

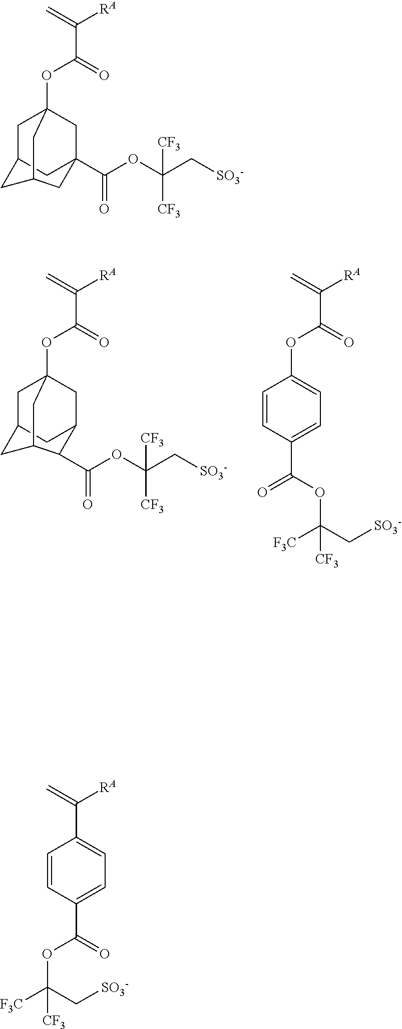

The repeating units-f1 to -f3 each function as an acid generator. By making an acid generator bond to a polymer main chain, acid diffusion can be reduced. Thus, degradation of resolution due to blurring by acid diffusion can be prevented. In addition, LWR and CDU can be improved by the uniform dispersion of the acid generator. Note that when a base polymer containing the repeating unit-f is used, blending of the additive-type acid generator described below may be omitted.

In the base polymer, the content ratios of the repeating units −a1, −a2, −b, −c, −d, −e, −f1, −f2, and −f3 are preferably 0≤a1≤0.9, 0≤a2≤0.9, 0≤a1+a2≤0.9, 0≤b≤0.9, 0≤c≤0.9, 0≤d≤0.5, 0≤e≤0.5, 0≤f1≤0.5, 0≤f2≤0.5, 0≤f3≤0.5, and 0≤f1+f2+f3≤0.5; more preferably 0≤a1≤0.8, 0≤a2≤0.8, 0≤a1+a2≤0.8, 0≤b≤0.8, 0≤c≤0.8, 0≤d≤0.4, 0≤e≤0.4, 0≤f1≤0.4, 0≤f2≤0.4, 0≤f3≤0.4, and 0≤f1+f2+f3≤0.4; and further preferably 0≤a1≤0.7, 0≤a2≤0.7, 0≤a1+a2≤0.7, 0≤b≤0.7, 0≤c≤0.7, 0≤d≤0.3, 0≤e≤0.3, 0≤f1≤0.3, 0≤f2≤0.3, 0≤f3≤0.3, and 0≤f1+f2+f3≤0.3, provided that a1+a2+b+c+d+f1+f2+f3+e=1.0.

The base polymer may be synthesized, for example, by subjecting the monomers to give the repeating units described above to heat polymerization in an organic solvent to which a radical polymerization initiator has been added.

Examples of the organic solvent used in the polymerization include toluene, benzene, tetrahydrofuran (THF), diethyl ether, dioxane, etc. Examples of the polymerization initiator include 2,2′-azobisisobutyronitrile (AIBN), 2,2′-azobis(2,4-dimethylvaleronitrile), dimethyl-2,2-azobis(2-methylpropionate), benzoyl peroxide, lauroyl peroxide, etc. The temperature during the polymerization is preferably 50 to 80° C. The reaction time is preferably 2 to 100 hours, more preferably 5 to 20 hours.

In the case where the monomer containing a hydroxy group is copolymerized, the process may include: substituting the hydroxy group with an acetal group susceptible to deprotection with acid, such as an ethoxyethoxy group, prior to the polymerization; and performing the deprotection with weak acid and water after the polymerization. Alternatively, the process may include: substituting the hydroxy group with an acyl group such as an acetyl group, a formyl group and a pivaloyl group, prior to the polymerization; and performing alkaline hydrolysis after the polymerization.

In a case where hydroxystyrene or hydroxyvinylnaphthalene is copolymerized, at first, acetoxystyrene or acetoxyvinylnaphthalene may be used in place of hydroxystyrene or hydroxyvinylnaphthalene; after the polymerization, the acetoxy group may be deprotected by the alkaline hydrolysis to convert the acetoxystyrene or acetoxyvinylnaphthalene to hydroxystyrene or hydroxyvinylnaphthalene.

In the alkaline hydrolysis, a base such as ammonia water or triethylamine is usable. The reaction temperature is preferably −20 to 100° C., more preferably 0 to 60° C. The reaction time is preferably 0.2 to 100 hours, more preferably 0.5 to 20 hours.

The base polymer has a polystyrene-based weight-average molecular weight (Mw) of preferably 1,000 to 500,000, more preferably 2,000 to 30,000, determined by gel permeation chromatography (GPC) using THF as an eluent. When the Mw is within such a range, the obtained resist film has excellent heat resistance and solubility to an alkaline developer.

Further, when the base polymer has a sufficiently narrow molecular weight distribution (Mw/Mn), there is no low-molecular-weight or high-molecular-weight polymer. Accordingly, there is no risk of foreign matters being observed on the pattern after the exposure or the pattern profile being degraded. The finer the pattern rule, the stronger the influences of Mw and Mw/Mn. Hence, in order to obtain a resist composition suitably used for finer pattern dimensions, the base polymer preferably has a narrow dispersity Mw/Mn of 1.0 to 2.0, particularly preferably 1.0 to 1.5. The molecular weight distribution can be measured along with the weight-average molecular weight.

The base polymer may contain two or more kinds of polymers that differ in composition ratio, Mw, and Mw/Mn.

### [Acid Generator]

The inventive resist composition may contain an acid generator that generates an acid (hereinafter, also referred to as additive-type acid generator). The generated acid is preferably a strong acid. Here, the term strong acid means, in the case of a chemically amplified positive resist composition, a compound that has sufficient acidity to cause a deprotection reaction of the acid-labile group of the base polymer; and in the case of a chemically amplified negative resist composition, a compound that has sufficient acidity to cause a polarity change reaction or a crosslinking reaction by acid. When such an acid generator is contained, the above-described onium salt functions as a quencher, so that the inventive resist composition can function as a chemically amplified positive resist composition or a chemically amplified negative resist composition.

Examples of the acid generator include compounds that generate acids in response to actinic light or radiation (photo-acid generators). The photo-acid generator can be any photo-acid generator as long as the compound generates an acid upon high-energy beam irradiation. Preferably, the photo-acid generator generates a sulfonic acid, imide acid, or methide acid. Suitable photo-acid generators include sulfonium salt, iodonium salt, sulfonyldiazomethane, N-sulfonyloxyimide, oxime-O-sulfonate type acid generators, etc. Specific examples of the photo-acid generator include ones disclosed in paragraphs to of JP 2008-111103 A.

Moreover, a sulfonium salt shown by the following general formula (3-1) and an iodonium salt shown by the following general formula (3-2) can also be used suitably as photo-acid generators.

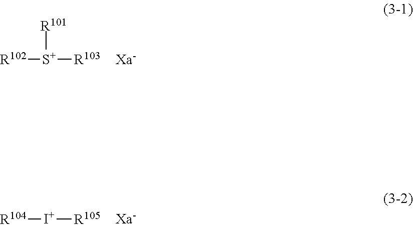

In the general formulae (3-1) and (3-2), R un to R¹⁰⁵ each independently represent a hydrocarbyl group having 1 to 20 carbon atoms and optionally containing a heteroatom such as a halogen atom. The hydrocarbyl group may be saturated or unsaturated, and may be linear, branched, or cyclic. Specific examples thereof include those given as examples of the hydrocarbyl groups represented by R^{11′} to R^{19′} in the description of the formulae (Cation-1) to (Cation-3). R¹⁰¹ and R¹⁰² may also be bonded to each other to form a ring together with the sulfur atom bonded to R¹⁰¹ and R¹⁰². In this event, examples of the ring include those given as examples of rings that can be formed by R^{11′} and R^{12′} being bonded to each other together with the sulfur atom bonded to R^{11′} and R^{12′} in the description of the formula (Cation-1).

Examples of the cation of the sulfonium salt represented by the general formula (3-1) include the cations given as examples of the cation of the sulfonium salt represented by the formula (Cation-1), but are not limited thereto.

Examples of the cation of the iodonium salt represented by the general formula (3-2) include the cations given as examples of the cation of the iodonium salt represented by the formula (Cation-2), but are not limited thereto.

In the general formulae (3-1) and (3-2), Xa⁻ represents an anion selected from the following formulae (3A) to (3D).

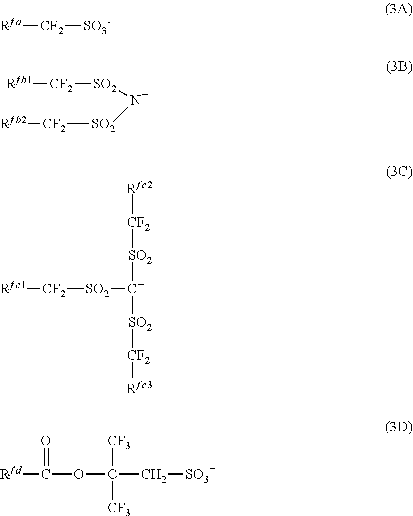

In the general formula (3A), R^{fa} represents a fluorine atom or a hydrocarbyl group having 1 to 40 carbon atoms and optionally containing a heteroatom. The hydrocarbyl group may be saturated or unsaturated, and may be linear, branched, or cyclic. Specific examples thereof include groups given as examples of a hydrocarbyl group represented by R¹¹¹ in the formula (3A′) described below.

As the anion represented by the formula (3A), an anion represented by the following general formula (3A′) is preferable.

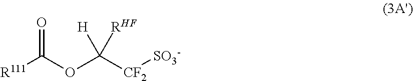

In the general formula (3A′), R^{HF} represents a hydrogen atom or a trifluoromethyl group, preferably a trifluoromethyl group. R¹¹¹ represents a hydrocarbyl group having 1 to 38 carbon atoms and optionally containing a heteroatom. The heteroatom is preferably an oxygen atom, a nitrogen atom, a sulfur atom, a halogen atom, or the like, more preferably an oxygen atom. The hydrocarbyl group particularly preferably has 6 to 30 carbon atoms from the viewpoint of achieving high resolution in fine pattern formation.

The hydrocarbyl group represented by R¹¹¹ may be saturated or unsaturated, and may be linear, branched, or cyclic. Specific examples thereof include alkyl groups having 1 to 38 carbon atoms, such as a methyl group, an ethyl group, a propyl group, an isopropyl group, a butyl group, an isobutyl group, a sec-butyl group, a tert-butyl group, a pentyl group, a neopentyl group, a hexyl group, a heptyl group, a 2-ethylhexyl group, a nonyl group, an undecyl group, a tridecyl group, a pentadecyl group, a heptadecyl group, and an icosanyl group; cyclic saturated hydrocarbyl groups having 3 to 38 carbon atoms, such as a cyclopentyl group, a cyclohexyl group, a 1-adamantyl group, a 2-adamantyl group, a 1-adamantylmethyl group, a norbornyl group, a norbornylmethyl group, a tricyclodecanyl group, a tetracyclododecanyl group, a tetracyclododecanylmethyl group, and a dicyclohexylmethyl group; unsaturated aliphatic hydrocarbyl groups having 2 to 38 carbon atoms, such as an allyl group and a 3-cyclohexenyl group; aryl groups having 6 to 38 carbon atoms, such as a phenyl group, a 1-naphthyl group, and a 2-naphthyl group; aralkyl groups having 7 to 38 carbon atoms, such as a benzyl group and a diphenylmethyl group; and groups derived from combinations of these groups.

Furthermore, part or all of the hydrogen atoms of these groups may be substituted with a group containing a heteroatom, such as an oxygen atom, a sulfur atom, a nitrogen atom, or a halogen atom, and part of the carbon atoms of these groups may be substituted with a group containing a heteroatom such as an oxygen atom, a sulfur atom, or a nitrogen atom. The resulting hydrocarbyl group may contain a hydroxy group, a fluorine atom, a chlorine atom, a bromine atom, an iodine atom, a cyano group, a nitro group, a carbonyl group, an ether bond, an ester bond, a sulfonic acid ester bond, a carbonate bond, a lactone ring, a sultone ring, a carboxylic anhydride, a haloalkyl group, etc. Examples of the hydrocarbyl group containing a heteroatom include a tetrahydrofuryl group, a methoxymethyl group, an ethoxymethyl group, a methylthiomethyl group, an acetamidomethyl group, a trifluoroethyl group, a (2-methoxyethoxy)methyl group, an acetoxymethyl group, a 2-carboxy-1-cyclohexyl group, a 2-oxopropyl group, a 4-oxo-1-adamantyl group, a 3-oxocyclohexyl group, etc.

The synthesis of the sulfonium salt containing the anion shown by the general formula (3A′) is described in detail in JP 2007-145797 A, JP 2008-106045 A, JP 2009-7327 A, JP 2009-258695 A, etc. In addition, sulfonium salts disclosed in JP 2010-215608 A, JP 2012-41320 A, JP 2012-106986 A, JP 2012-153644 A, etc. are also suitably used.

Examples of the anion represented by the general formula (3A) include those given as examples of the anion represented by a formula (1A) in JP 2018-197853 A.

In the general formula (3B), R^{fb1} and R^{fb2} each independently represent a fluorine atom or a hydrocarbyl group having 1 to 40 carbon atoms and optionally containing a heteroatom. The hydrocarbyl group may be saturated or unsaturated, and may be linear, branched, or cyclic. Specific examples thereof include those given as examples of the hydrocarbyl group represented by R¹¹¹ in the general formula (3A′). R^{fb1} and R^{fb2} are preferably a fluorine atom or a linear fluorinated alkyl group having 1 to 4 carbon atoms. Alternatively, R^{fb1} and R^{fb2} may bond with each other to form a ring together with a group (—CF₂—SO₂—N⁻—SO₂—CF₂—) bonded therewith. In this event, the group obtained by R^{fb1} and R^{fb2} being bonded to each other is preferably a fluorinated ethylene group or a fluorinated propylene group.

In the general formula (3C), R^{fc1}, R^{fc2}, and R^{fc3} each independently represent a fluorine atom or a hydrocarbyl group having 1 to 40 carbon atoms and optionally containing a heteroatom. The hydrocarbyl group may be saturated or unsaturated, and may be linear, branched, or cyclic. Specific examples thereof include those given as examples of the hydrocarbyl group represented by R¹¹¹ in the general formula (3A′). R^{fc1}, R^{fc2}, and R^{fc3} are preferably a fluorine atom or a linear fluorinated alkyl group having 1 to 4 carbon atoms. Alternatively, R^{fc1} and R^{fc2} may bond with each other to form a ring together with a group (—CF₂—SO₂—C⁻—SO₂—CF₂—) bonded therewith. In this event, the group obtained by R^{fc1} and R^{fc2} being bonded with each other is preferably a fluorinated ethylene group or a fluorinated propylene group.

In the general formula (3D), R^{fd} represents a hydrocarbyl group having 1 to 40 carbon atoms and optionally containing a heteroatom. The hydrocarbyl group may be saturated or unsaturated, and may be linear, branched, or cyclic. Specific examples thereof include those given as examples of the hydrocarbyl group represented by R¹¹¹ in the formula (3A′).

The synthesis of the sulfonium salt containing the anion shown by the general formula (3D) is described in detail in JP 2010-215608 A and JP 2014-133723 A.

Examples of the anion represented by the general formula (3D) include those given as examples of the anion represented by a formula (1D) in JP 2018-197853 A.

Note that the photo-acid generator containing the anion shown by the general formula (3D) does not have a fluorine atom at α position of the sulfo group, but has two trifluoromethyl groups at β position, thereby providing sufficient acidity to cut the acid-labile group in the base polymer. Thus, this photo-acid generator is utilizable.

One shown by the following general formula (4) can also be used suitably as a photo-acid generator.

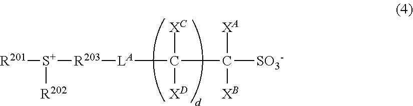

In the general formula (4), R²⁰¹ and R²⁰² each independently represent a hydrocarbyl group having 1 to 30 carbon atoms and optionally containing a heteroatom such as a halogen atom. R²⁰³ represents a hydrocarbylene group having 1 to 30 carbon atoms and optionally containing a heteroatom. In addition, any two of R²⁰¹, R²⁰², and R²⁰³ may be bonded with each other to form a ring together with a sulfur atom bonded therewith. In this event, examples of the ring include those given as examples of rings that can be formed by R^{11′} and R^{12′} being bonded to each other together with the sulfur atom bonded thereto in the description of the general formula (Cation-1).

The hydrocarbyl groups represented by R²⁰¹ and R²⁰² may be saturated or unsaturated, and may be linear, branched, or cyclic. Specific examples thereof include: alkyl groups having 1 to 30 carbon atoms, such as a methyl group, an ethyl group, a propyl group, an isopropyl group, an n-butyl group, a sec-butyl group, a tert-butyl group, an n-pentyl group, a tert-pentyl group, an n-hexyl group, an n-octyl group, a 2-ethylhexyl group, an n-nonyl group, and an n-decyl group; cyclic saturated hydrocarbyl groups having 3 to 30 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, an oxanorbornyl group, a tricyclo[5.2.1.0^{2,6}]decanyl group, and an adamantyl group; aryl groups having 6 to 30 carbon atoms, such as a phenyl group, a methylphenyl group, an ethylphenyl group, an n-propylphenyl group, an isopropylphenyl group, an n-butylphenyl group, an isobutylphenyl group, a sec-butylphenyl group, a tert-butylphenyl group, a naphthyl group, a methylnaphthyl group, an ethylnaphthyl group, an n-propylnaphthyl group, an isopropylnaphthyl group, an n-butylnaphthyl group, an isobutylnaphthyl group, a sec-butylnaphthyl group, a tert-butylnaphthyl group, and an anthracenyl group; and groups derived from combinations of these groups. Furthermore, part or all of the hydrogen atoms of these groups may be substituted with a group containing a heteroatom, such as an oxygen atom, a sulfur atom, a nitrogen atom, or a halogen atom, and part of the carbon atoms of these groups may be substituted with a group containing a heteroatom such as an oxygen atom, a sulfur atom, or a nitrogen atom. The resulting hydrocarbyl group may contain a hydroxy group, a fluorine atom, a chlorine atom, a bromine atom, an iodine atom, a cyano group, a nitro group, a carbonyl group, an ether bond, an ester bond, a sulfonic acid ester bond, a carbonate bond, a lactone ring, a sultone ring, a carboxylic anhydride, a haloalkyl group, etc.

The hydrocarbylene group represented by R²⁰³ may be saturated or unsaturated, and may be linear, branched, or cyclic. Specific examples thereof include alkanediyl groups having 1 to 30 carbon atoms, such as a methanediyl group, an ethane-1,1-diyl group, an ethane-1,2-diyl group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, and a heptadecane-1,17-diyl group; cyclic saturated hydrocarbylene groups having 3 to 30 carbon atoms, such as a cyclopentanediyl group, a cyclohexanediyl group, a norbornanediyl group, and an adamantanediyl group; arylene groups having 6 to 30 carbon atoms, such as a phenylene group, a methylphenylene group, an ethylphenylene group, an n-propylphenylene group, an isopropylphenylene group, an n-butylphenylene group, an isobutylphenylene group, a sec-butylphenylene group, a tert-butylphenylene group, a naphthylene group, a methylnaphthylene group, an ethylnaphthylene group, an n-propylnaphthylene group, an isopropylnaphthylene group, an n-butylnaphthylene group, an isobutylnaphthylene group, a sec-butylnaphthylene group, and a tert-butylnaphthylene group; and groups derived from combinations of these groups. Furthermore, part or all of the hydrogen atoms of these groups may be substituted with a group containing a heteroatom, such as an oxygen atom, a sulfur atom, a nitrogen atom, or a halogen atom, and part of the carbon atoms of these groups may be substituted with a group containing a heteroatom such as an oxygen atom, a sulfur atom, or a nitrogen atom. The resulting hydrocarbylene group may contain a hydroxy group, a fluorine atom, a chlorine atom, a bromine atom, an iodine atom, a cyano group, a nitro group, a carbonyl group, an ether bond, an ester bond, a sulfonic acid ester bond, a carbonate bond, a lactone ring, a sultone ring, a carboxylic anhydride, a haloalkyl group, etc. As the heteroatom, an oxygen atom is preferable.

In the general formula (4), LA represents a single bond, an ether bond, or a hydrocarbylene group having 1 to 20 carbon atoms and optionally containing a heteroatom. The hydrocarbylene group may be saturated or unsaturated, and may be linear, branched, or cyclic. Specific examples thereof include those given as examples of the hydrocarbylene group represented by R²⁰³.

In the general formula (4), X^{A}, X^{B}, X^{C}, and X^{D} each independently represent a hydrogen atom, a fluorine atom, or a trifluoromethyl group, provided that at least one of X^{A}, X^{B}, X_{C}, and X^{D} is a fluorine atom or a trifluoromethyl group.

In the general formula (4), “d” represents an integer of 0 to 3.

As the photo-acid generator shown by the general formula (4), those shown by the following general formula (4′) are preferable.

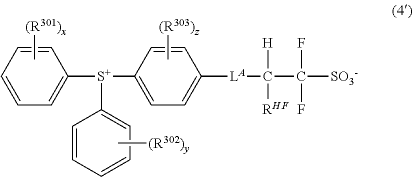

In the general formula (4′), LA is as defined above. R^{HF} represents a hydrogen atom or a trifluoromethyl group, preferably a trifluoromethyl group. R³⁰¹, R³⁰², and R³⁰³ each independently represent a hydrogen atom or a hydrocarbyl group having 1 to 20 carbon atoms and optionally containing a heteroatom. The hydrocarbyl group may be saturated or unsaturated, and may be linear, branched, or cyclic. Specific examples thereof include those given as examples of the hydrocarbyl group represented by R¹¹¹ in the formula (3A′). “x” and “y” each independently represent an integer of 0 to 5, and “z” represents an integer of 0 to 4.

Examples of the photo-acid generator represented by the general formula (4) include those given as examples of a photo-acid generator represented by a formula (2) in JP 2017-026980 A.

The photo-acid generators containing the anion shown by the general formula (3A′) or (3D) are particularly preferable because of small acid diffusion and excellent solubility to a solvent. A photo-acid generator shown by the formula (4′) is also particularly preferable because the acid diffusion is quite small.

As the photo-acid generator, it is also possible to use a sulfonium salt or an iodonium salt having an anion having an aromatic ring substituted with an iodine atom or a bromine atom. Examples of such salts include those represented by the following general formula (5-1) or (5-2).

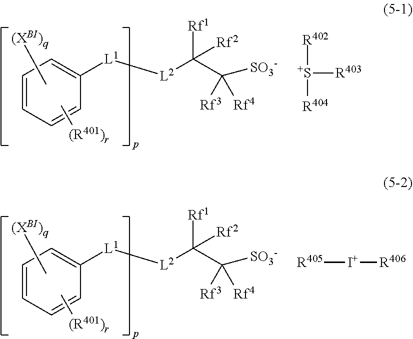

In the general formulae (5-1) and (5-2), “p” represents an integer that satisfies 1≤p≤3. “q” and “r” represent integers that satisfy 1≤q≤5, 0≤r≤3, and 1≤q+r≤5. “q” is preferably an integer that satisfies 1≤q≤3, and is more preferably 2 or 3. “r” is preferably an integer that satisfies 0≤r≤2.

In the general formulae (5-1) and (5-2), X^{BI} represents an iodine atom or a bromine atom, and when “p” and/or “q” is 2 or more, the X^{BI}s may be identical to or different from one another.

In the general formulae (5-1) and (5-2), L¹ represents a single bond, an ether bond, an ester bond, or a saturated hydrocarbylene group having 1 to 6 carbon atoms and optionally including an ether bond or an ester bond. The saturated hydrocarbylene group may be linear, branched, or cyclic.

In the general formulae (5-1) and (5-2), L² represents a single bond or a divalent linking group having 1 to 20 carbon atoms when “p” is 1, and represents a linking group having a valency of (p+1) and having 1 to 20 carbon atoms when “p” is 2 or 3, the linking group optionally containing an oxygen atom, a sulfur atom, or a nitrogen atom.

In the general formulae (5-1) and (5-2), R⁴⁰¹ represents a hydroxy group, a carboxy group, a fluorine atom, a chlorine atom, a bromine atom, an amino group, a hydrocarbyl group having 1 to 20 carbon atoms, a hydrocarbyloxy group having 1 to 20 carbon atoms, a hydrocarbylcarbonyl group having 2 to 20 carbon atoms, a hydrocarbyloxycarbonyl group having 2 to 20 carbon atoms, a hydrocarbylcarbonyloxy group having 2 to 20 carbon atoms, a hydrocarbylsulfonyloxy group having 1 to 20 carbon atoms, —N(R^{401A}) (R^{401B}), —N(R^{401C})—C(═O) —R^{401D}, or —N(R^{401C})—C(═O)—O—R^{401D}. The hydrocarbyl group, the hydrocarbyloxy group, the hydrocarbylcarbonyl group, the hydrocarbyloxycarbonyl group, the hydrocarbylcarbonyloxy group, and the hydrocarbylsulfonyloxy group optionally have a fluorine atom, a chlorine atom, a bromine atom, a hydroxy group, an amino group, an ether bond, an ester bond, or an amide bond. R^{401A} and R^{401B} each independently represent a hydrogen atom or a saturated hydrocarbyl group having 1 to 6 carbon atoms. R^{401C} represents a hydrogen atom or a saturated hydrocarbyl group having 1 to 6 carbon atoms, and optionally contains a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. R^{401D} represents an aliphatic hydrocarbyl group having 1 to 16 carbon atoms, an aryl group having 6 to 14 carbon atoms, or an aralkyl group having 7 to 15 carbon atoms, and optionally contains a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. The aliphatic hydrocarbyl group may be saturated or unsaturated, and may be linear, branched, or cyclic. The saturated hydrocarbyl group, the saturated hydrocarbyloxy group, the saturated hydrocarbyloxycarbonyl group, the saturated hydrocarbylcarbonyl group, and the saturated hydrocarbylcarbonyloxy group may be linear, branched, or cyclic. When “p” and/or “r” is 2 or more, the multiple R⁴⁰¹ may be identical to or different from one another.

In particular, a hydroxy group, —N(R^{401C})—C(═O)—R^{401D}, —N(R^{401C}) —C(═O) —O—R^{401D}, a fluorine atom, a chlorine atom, a bromine atom, a methyl group, a methoxy group, etc. are preferable as R⁴⁰¹.

In the general formulae (5-1) and (5-2), R^{f1} to R^{f4} each independently represent a hydrogen atom, a fluorine atom, or a trifluoromethyl group, provided that at least one of R^{f1} to R^{f4} is a fluorine atom or a trifluoromethyl group. R^{f1} and R^{f2} may also be combined to form a carbonyl group. In particular, R^{f2} and R^{f4} are preferably both a fluorine atom.

In the general formulae (5-1) and (5-2), R⁴⁰² to R⁴⁰⁶ each independently represent a hydrocarbyl group having 1 to 20 carbon atoms and optionally containing a heteroatom such as a halogen atom. The hydrocarbyl group may be saturated or unsaturated, and may be linear, branched, or cyclic. Specific examples thereof include those given as examples of the hydrocarbyl groups represented by R^{11′} to R^{19′} in the description of the general formula (Cation-1). Furthermore, part or all of the hydrogen atoms of these groups may be substituted with a hydroxy group, a carboxy group, a halogen atom, a cyano group, a nitro group, a mercapto group, a sultone ring, a sulfone group, or a sulfonium salt-containing group; and part of the carbon atoms of the groups may be substituted with an ether bond, an ester bond, a carbonyl group, an amide bond, a carbonate bond, or a sulfonic acid ester bond. Furthermore, R⁴⁰² and R⁴⁰³ may be bonded to each other to form a ring together with the sulfur atom bonded thereto. In this event, examples of the ring include those given as examples of rings that can be formed by R^{11′} and R^{12′} being bonded to each other together with the sulfur atom bonded thereto in the description of the general formula (Cation-1).

Examples of the cation of the sulfonium salt represented by the general formula (5-1) include those given as examples of the cation of a sulfonium salt represented by the general formula (Cation-1).

Meanwhile, examples of the cation of the iodonium salt represented by the formula (5-2) include those given as examples of the cation of an iodonium salt represented by the general formula (Cation-2).

Examples of the anion of the onium salt represented by the general formula (5-1) or (5-2) include those shown below, but are not limited thereto. Incidentally, in the following formulae, X^{BI} is as defined above.

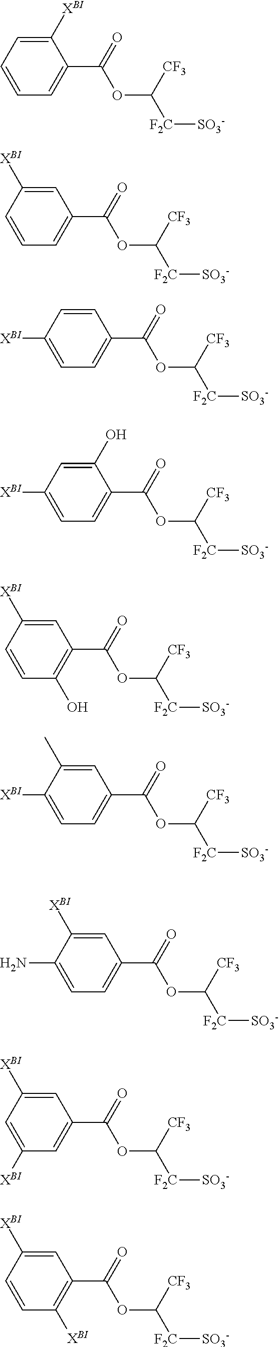

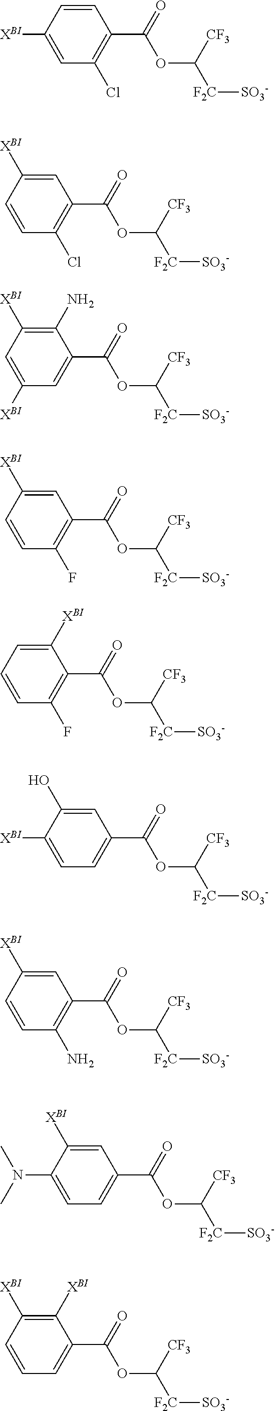

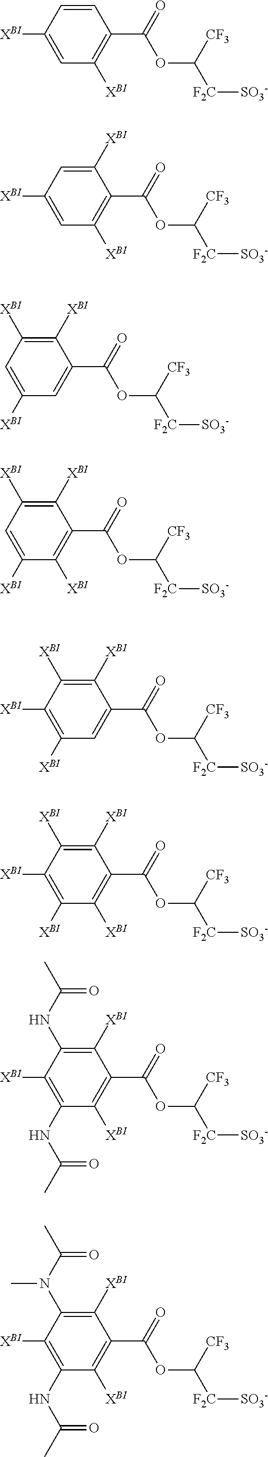

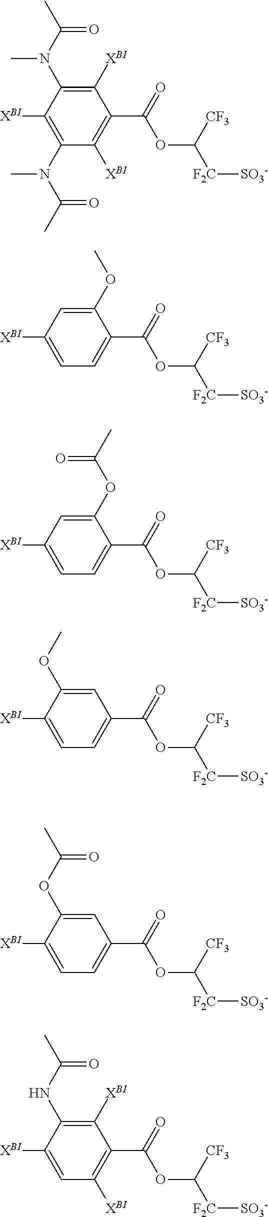

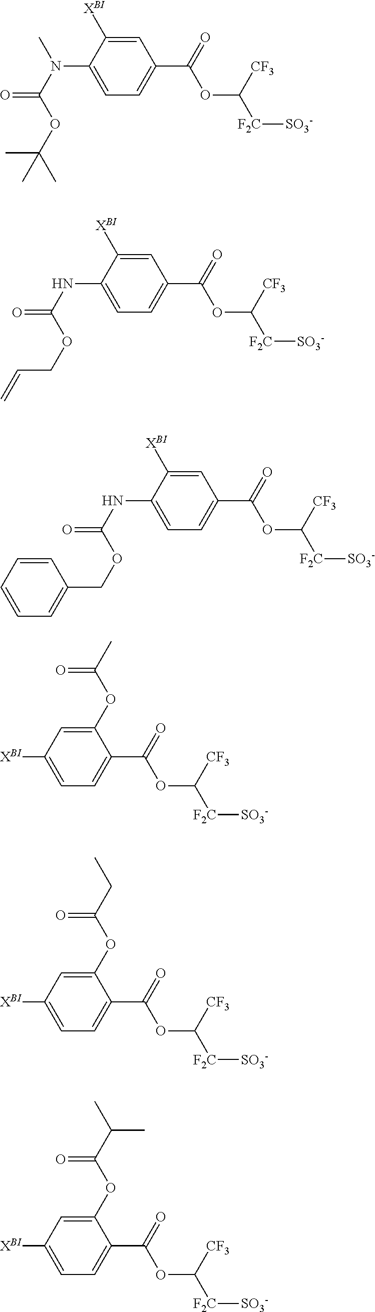

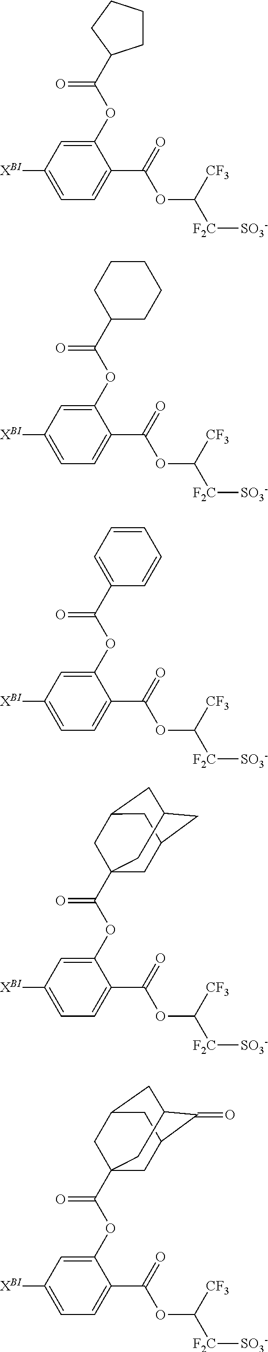

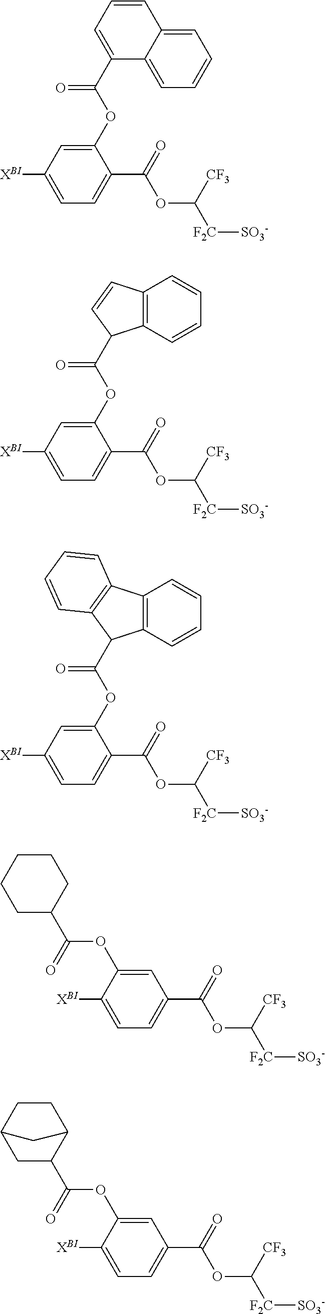

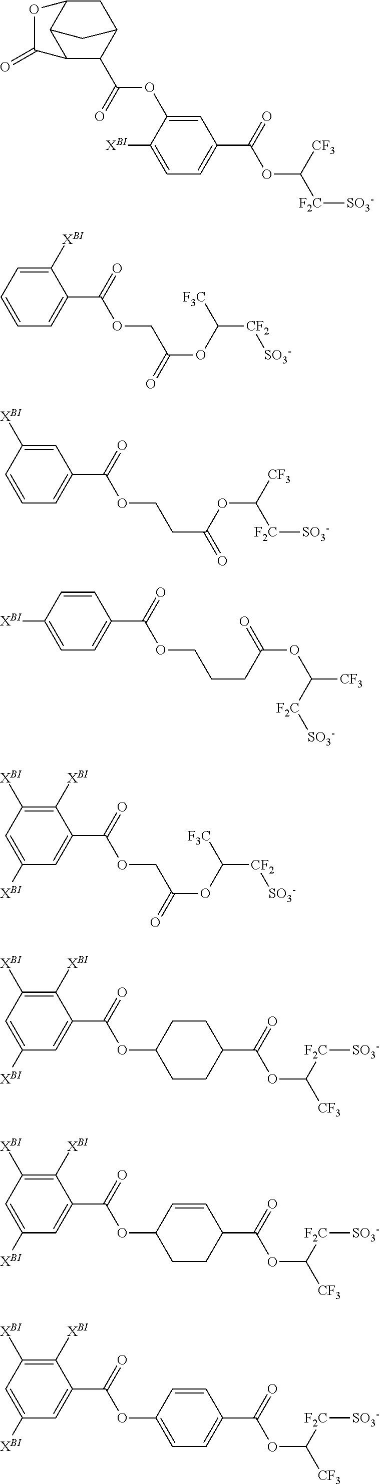

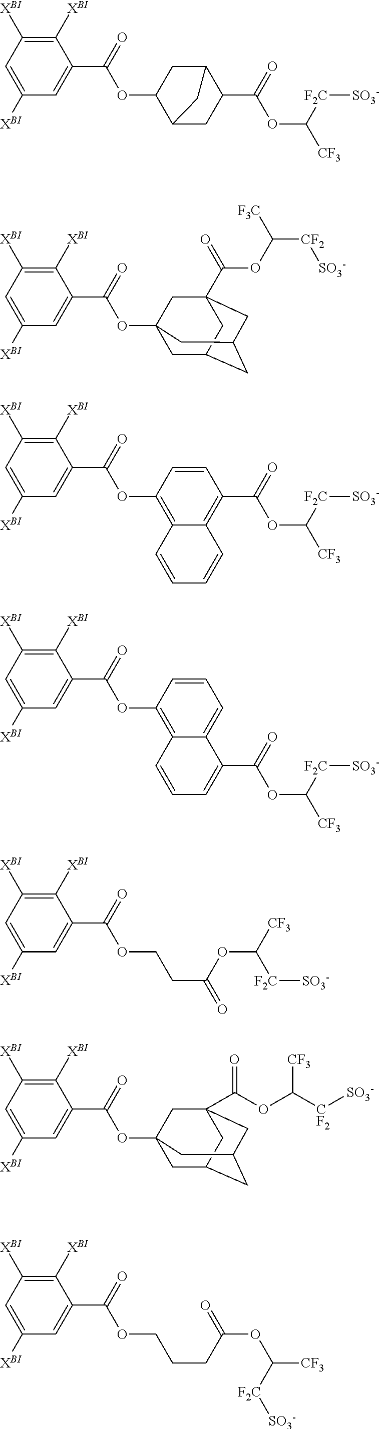

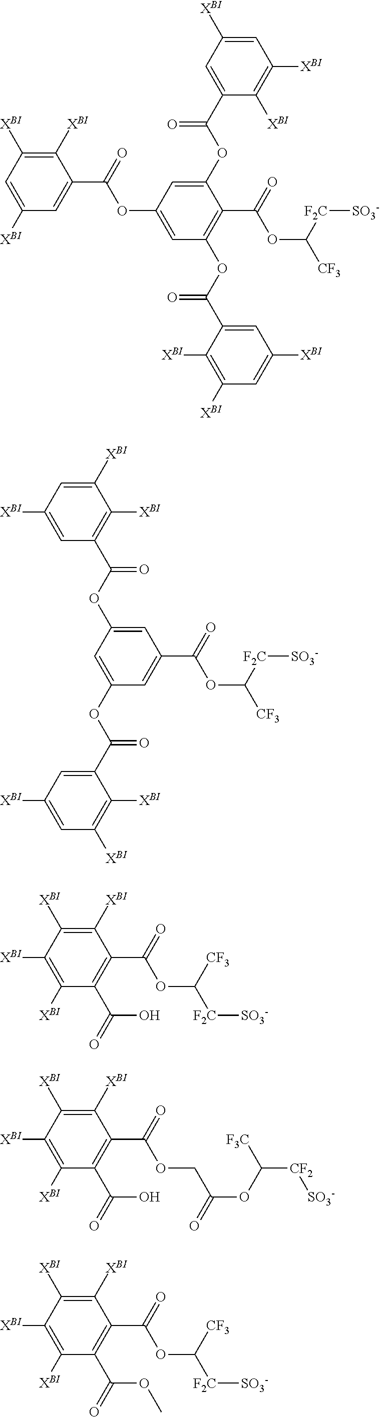

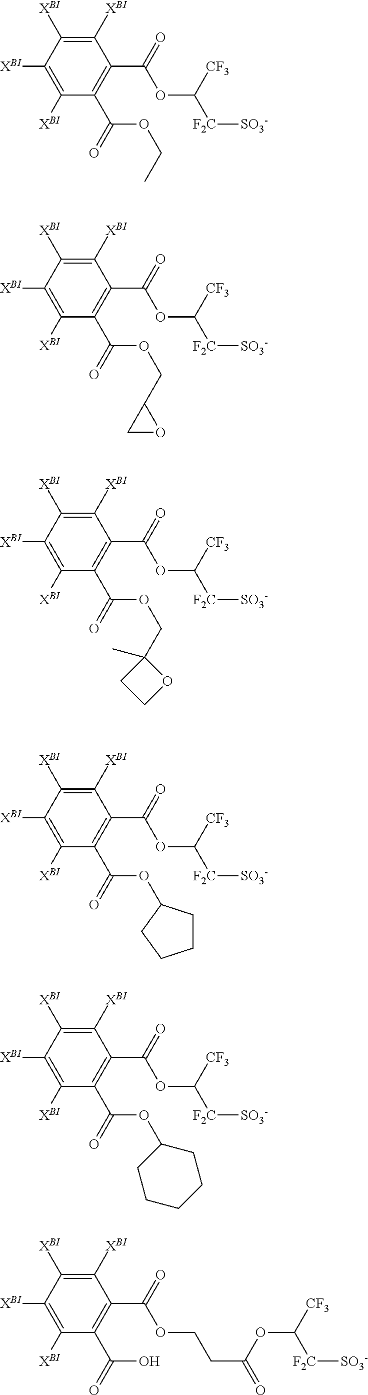

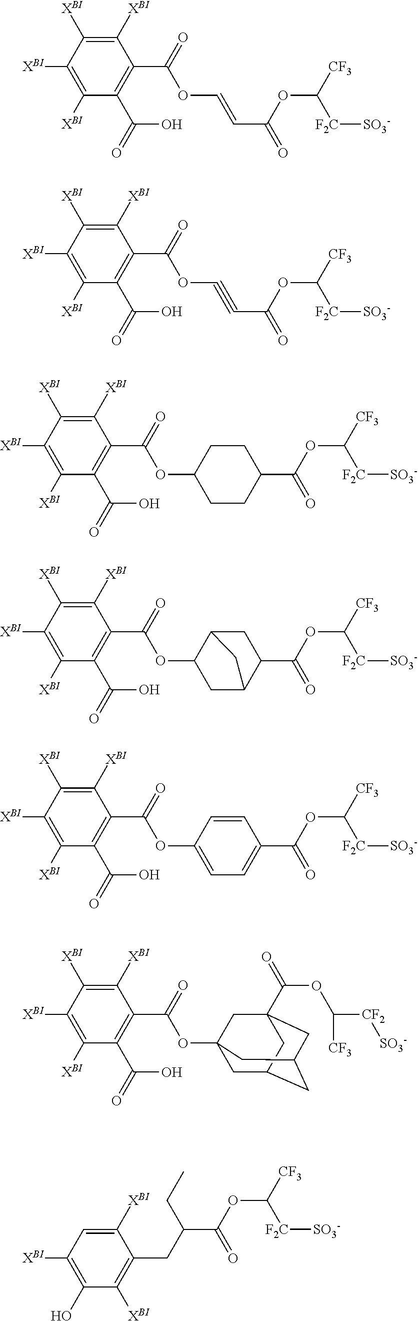

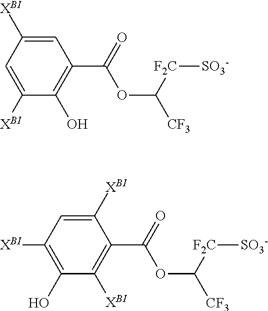

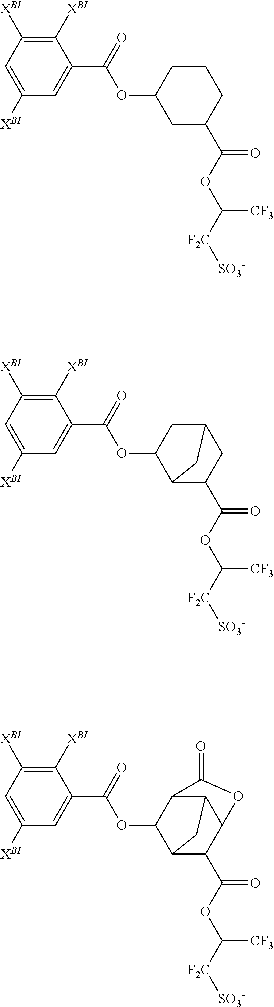

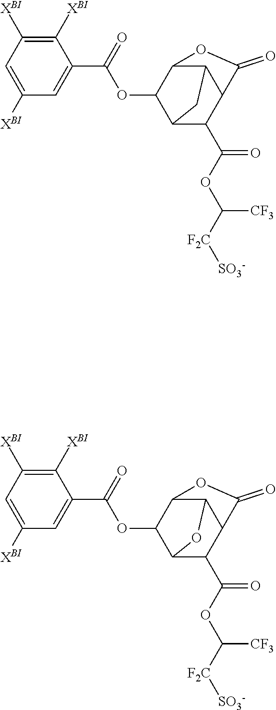

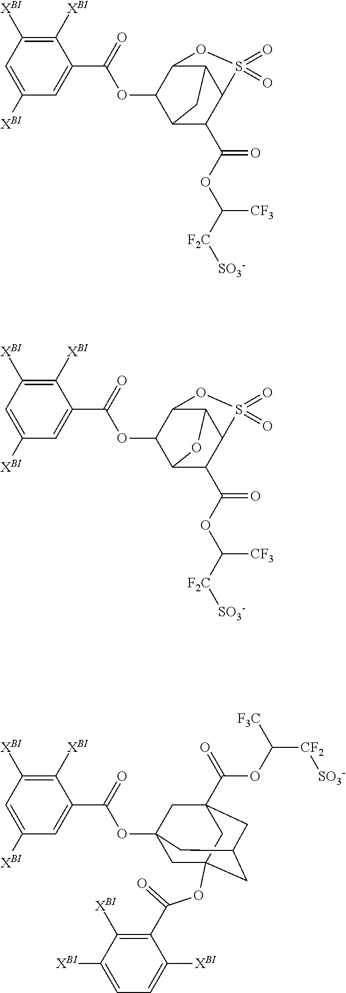

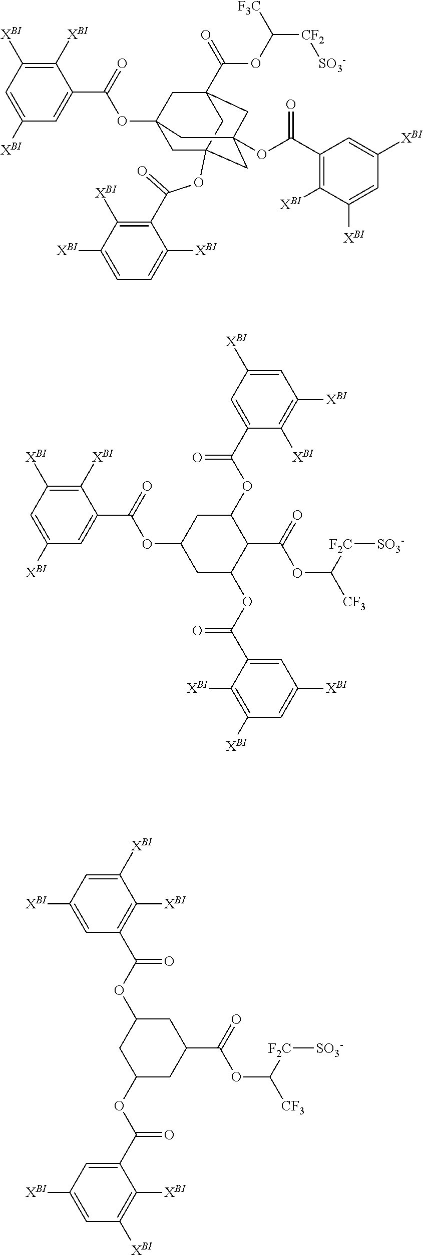

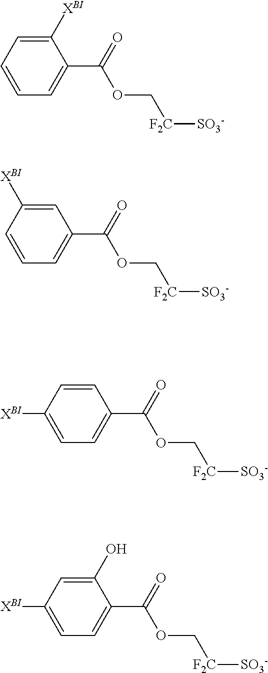

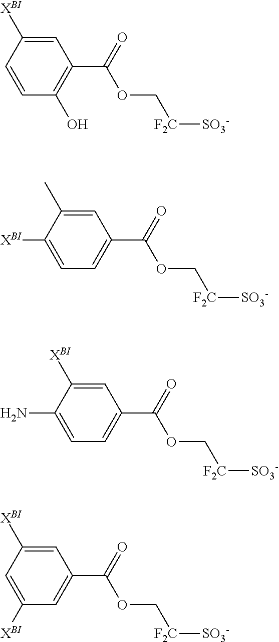

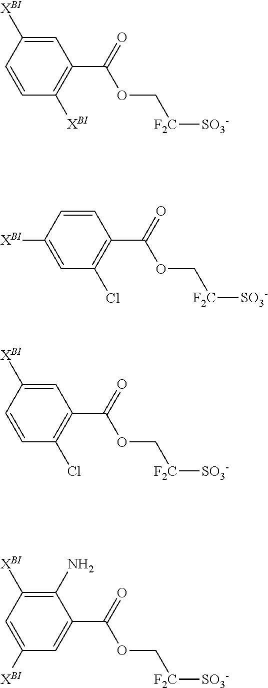

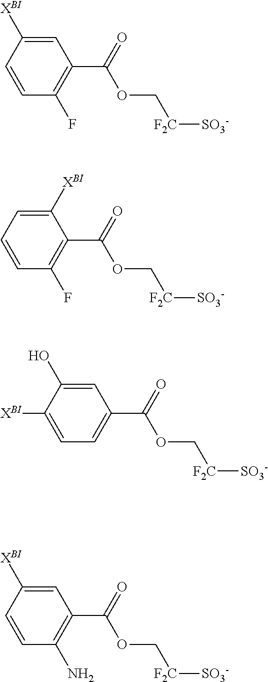

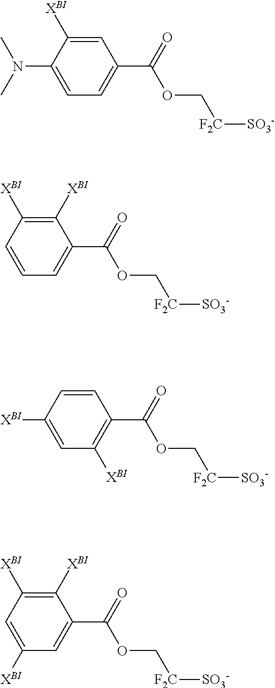

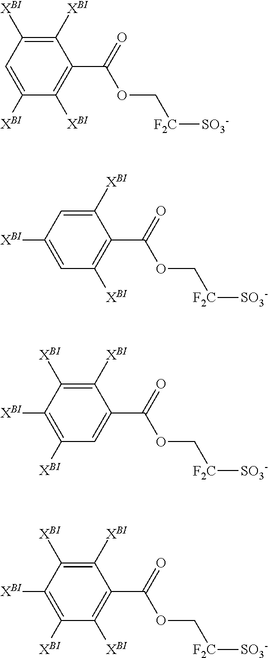

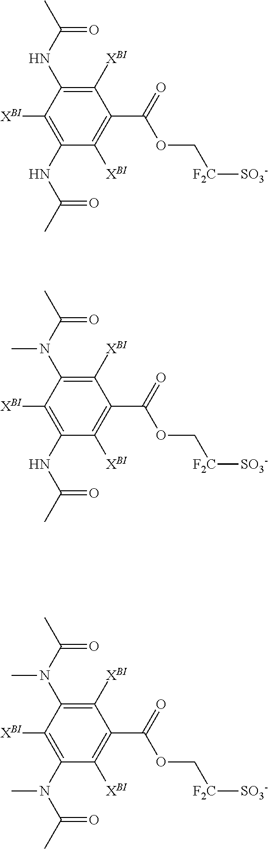

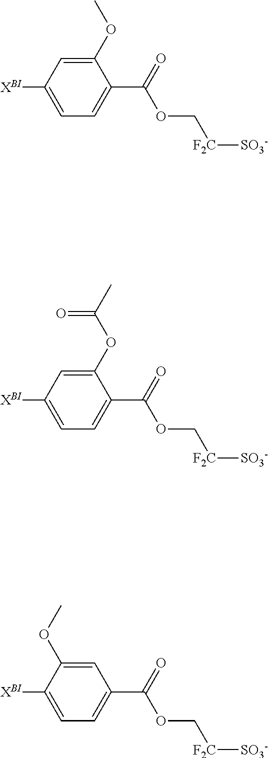

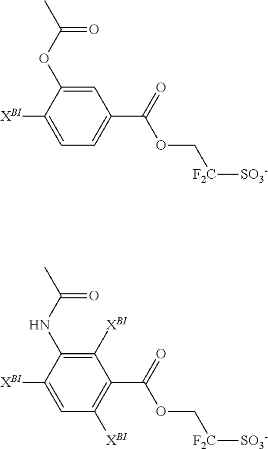

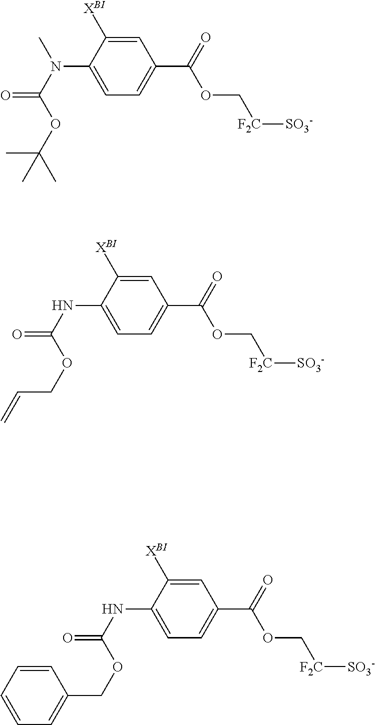

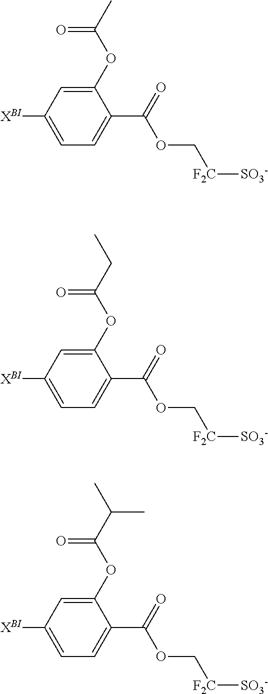

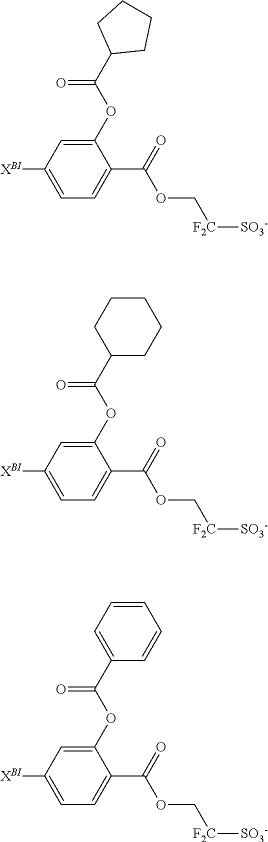

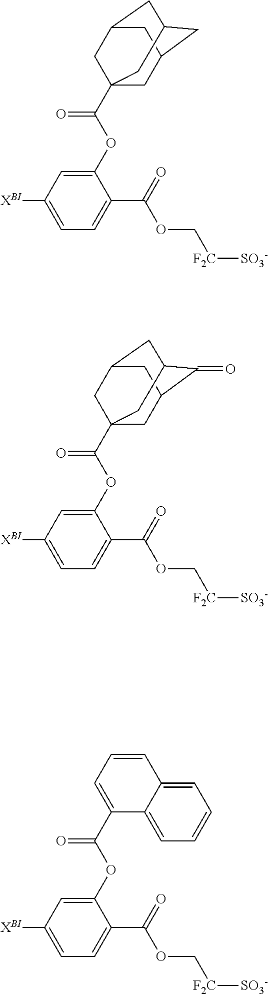

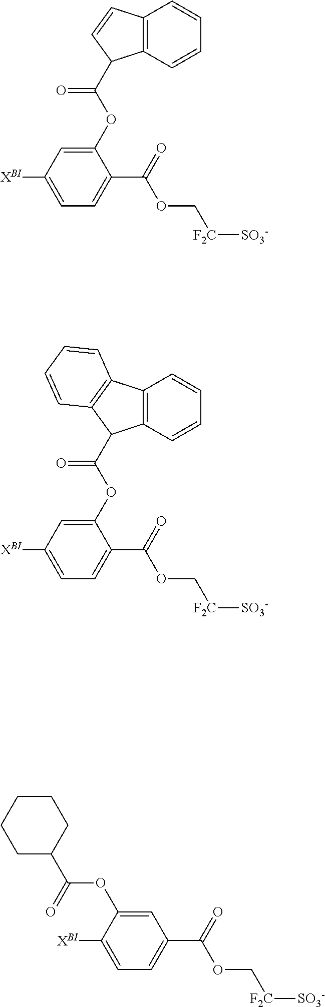

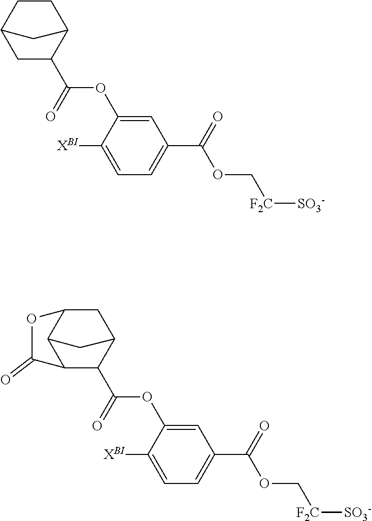

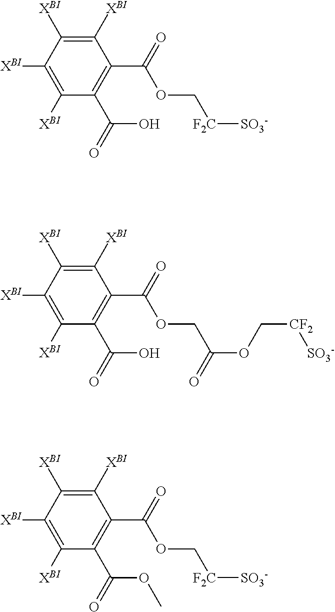

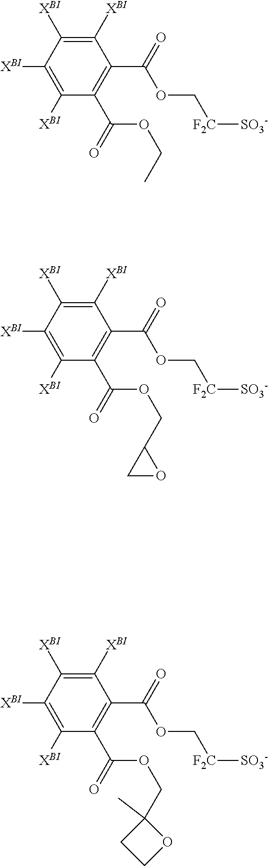

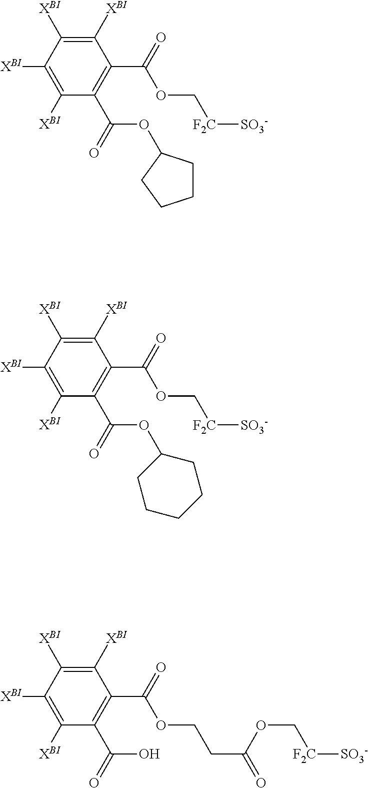

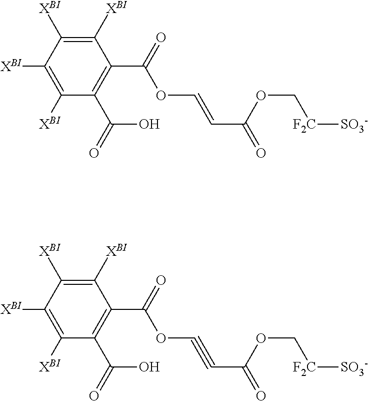

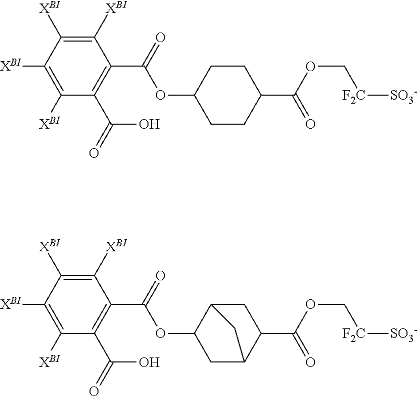

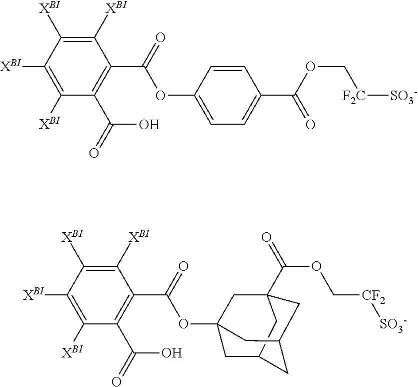

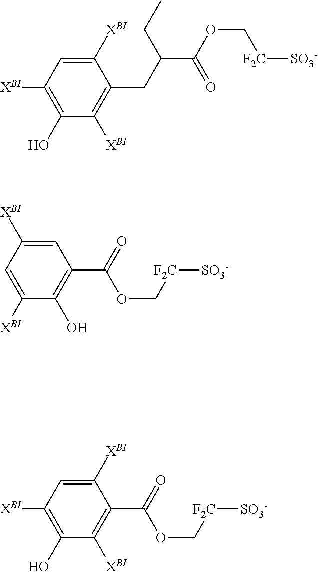

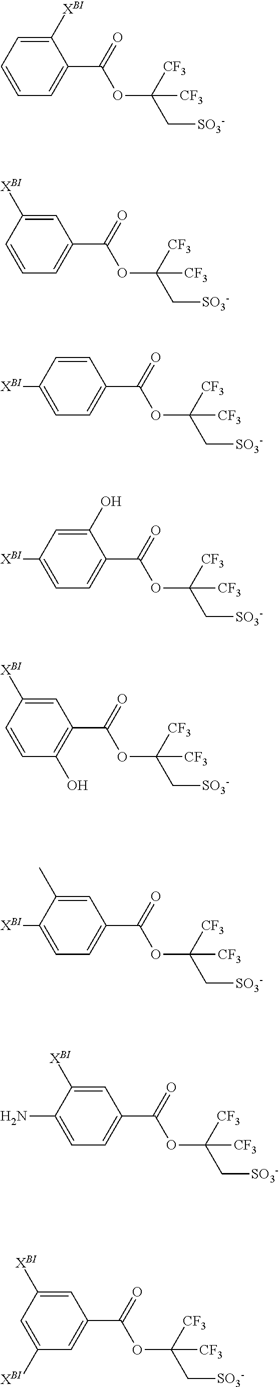

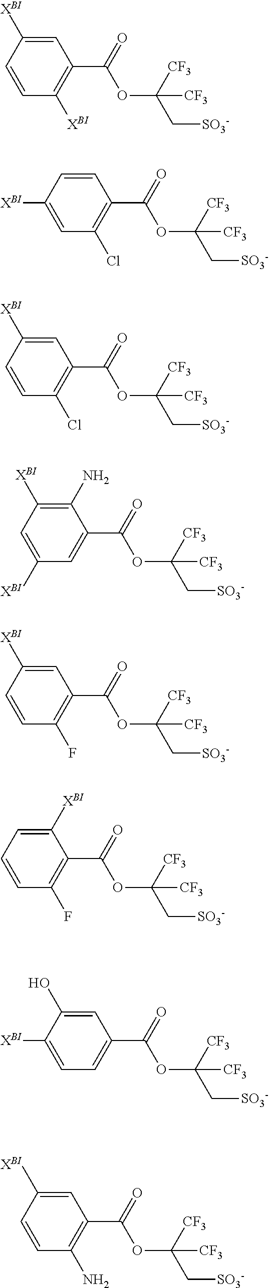

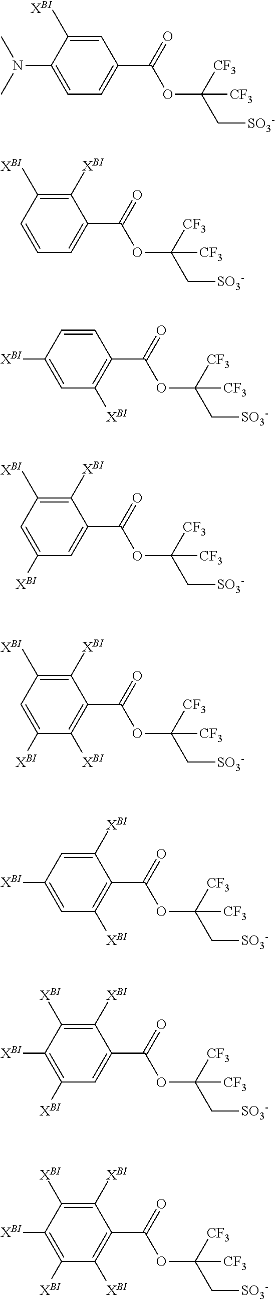

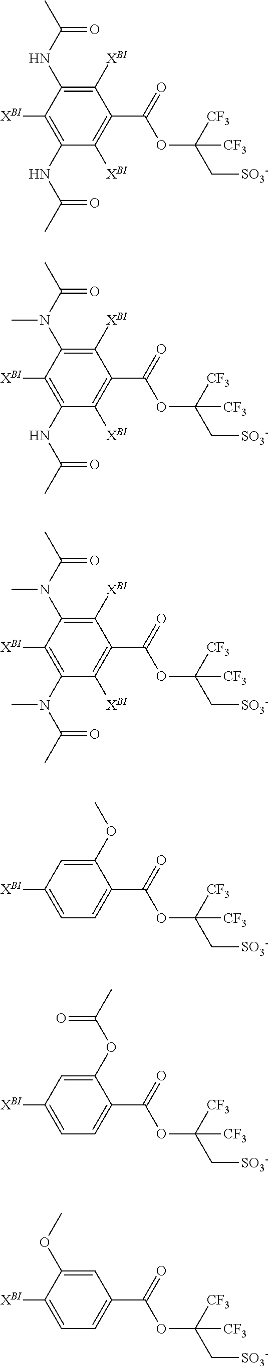

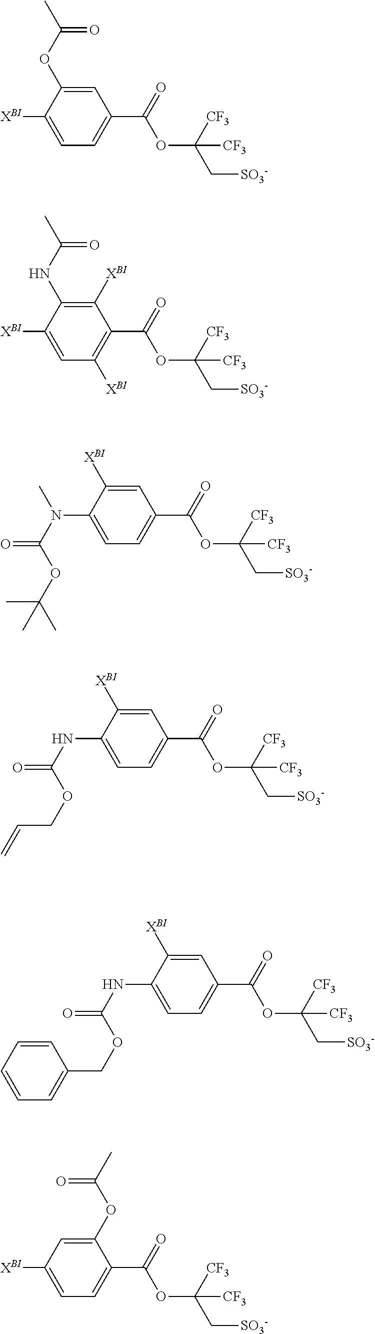

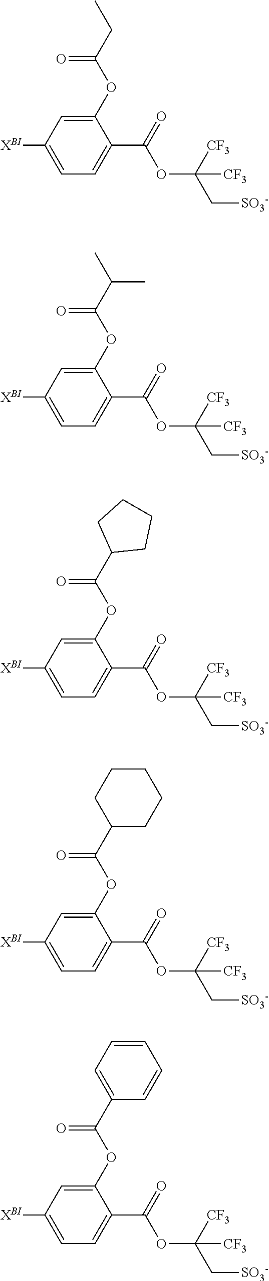

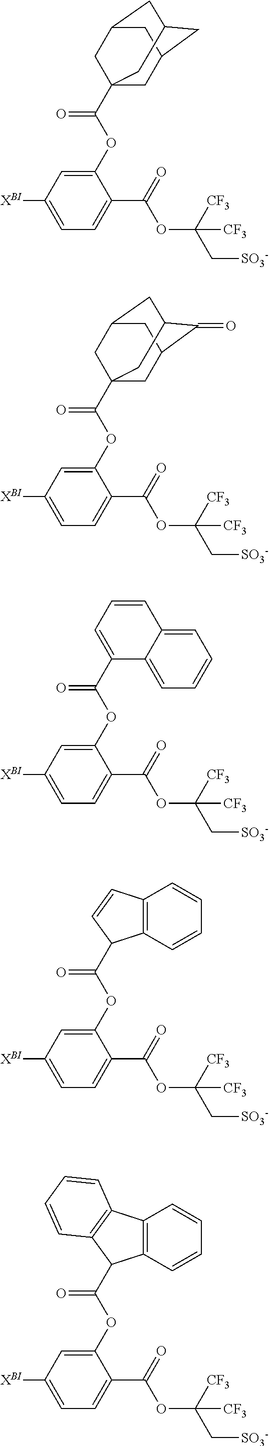

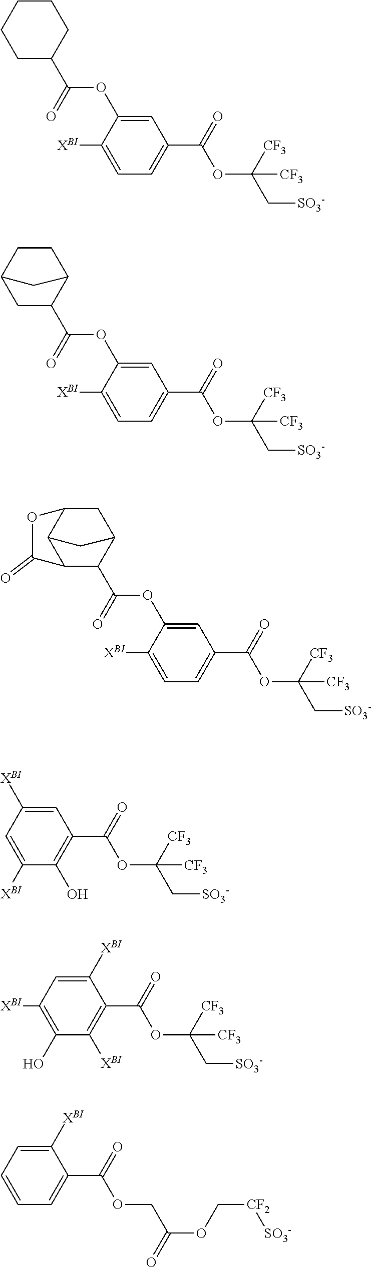

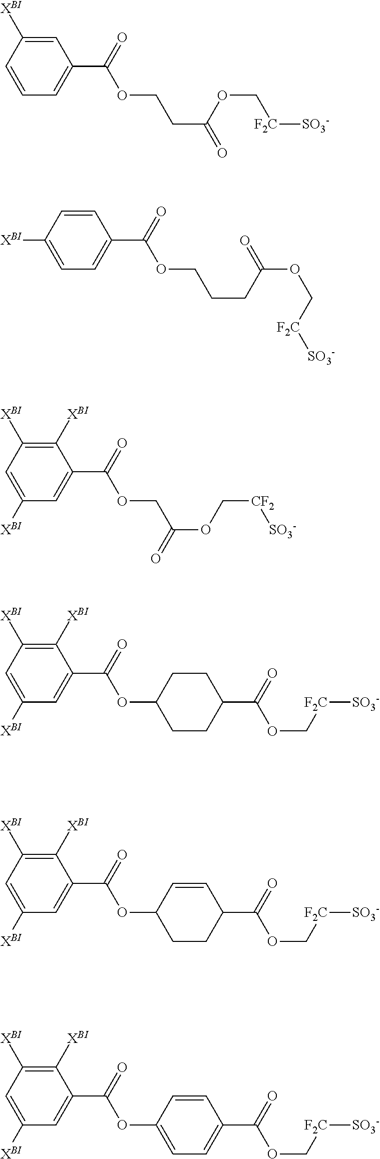

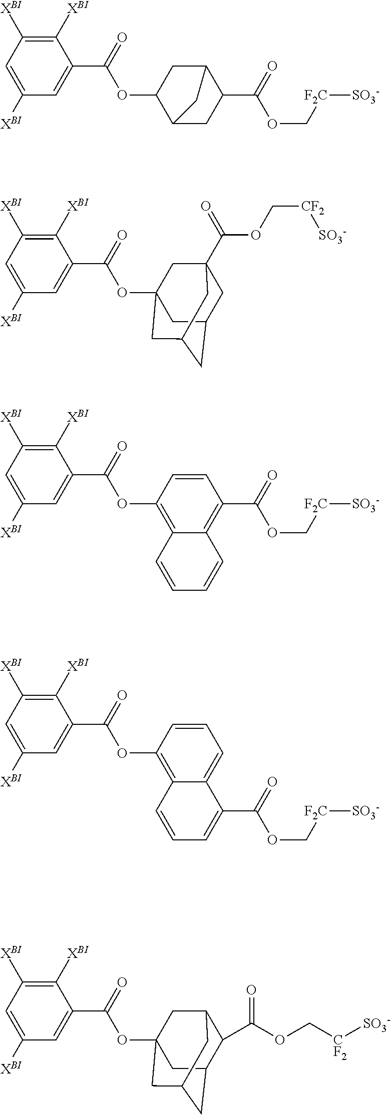

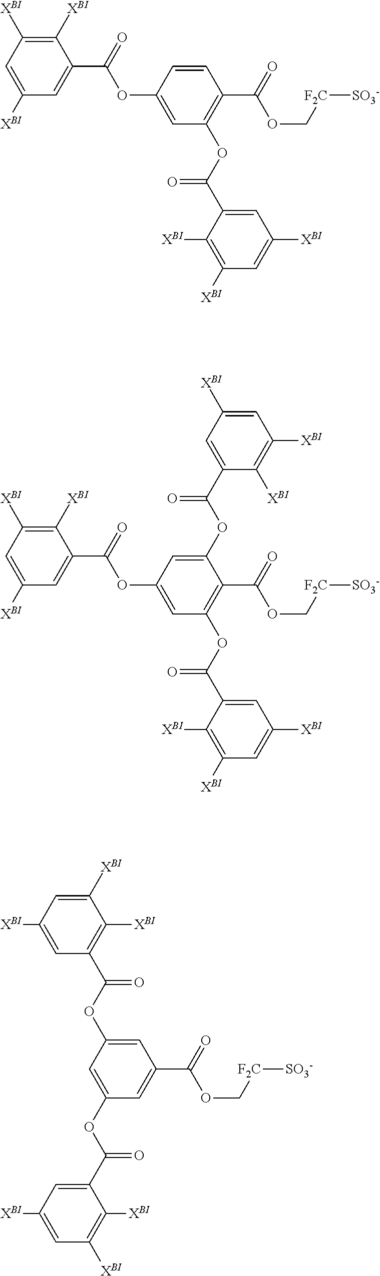

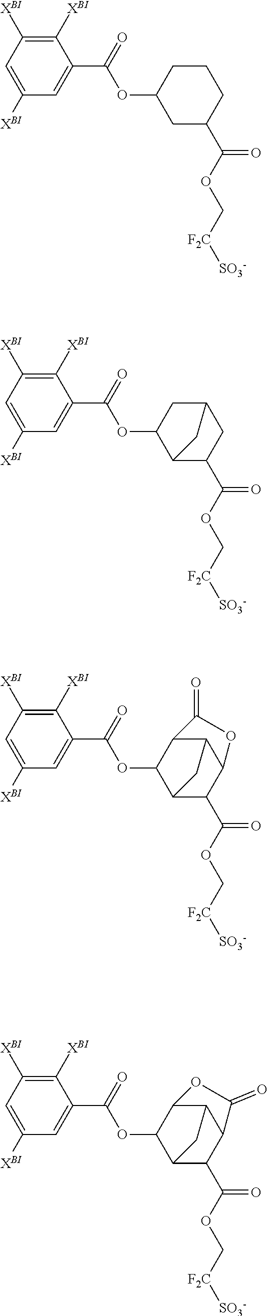

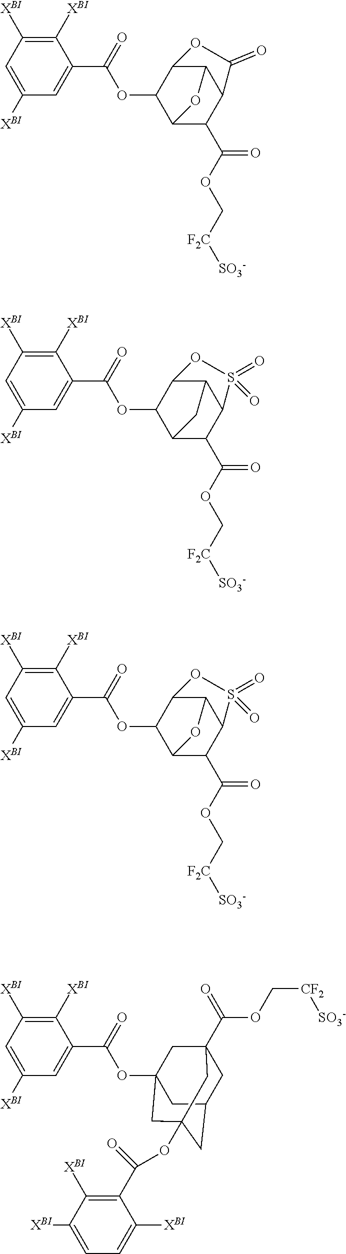

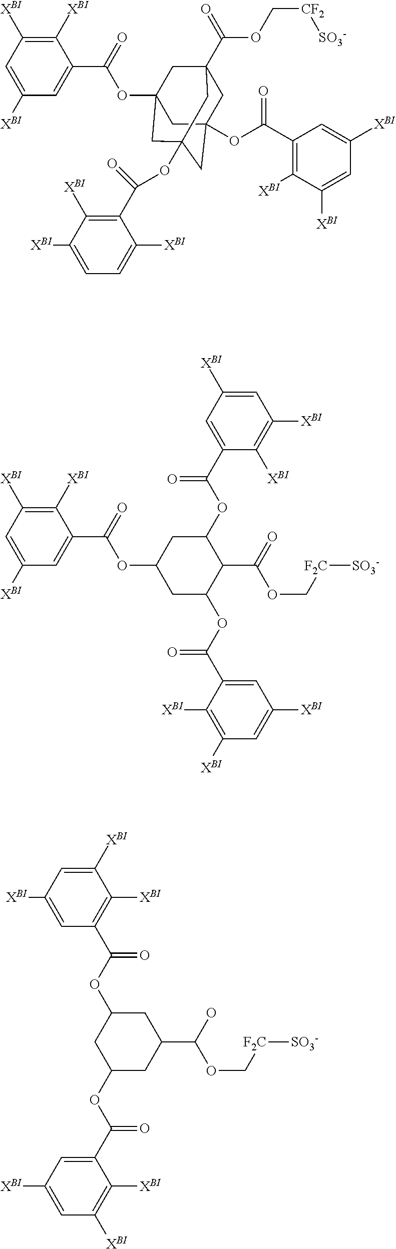

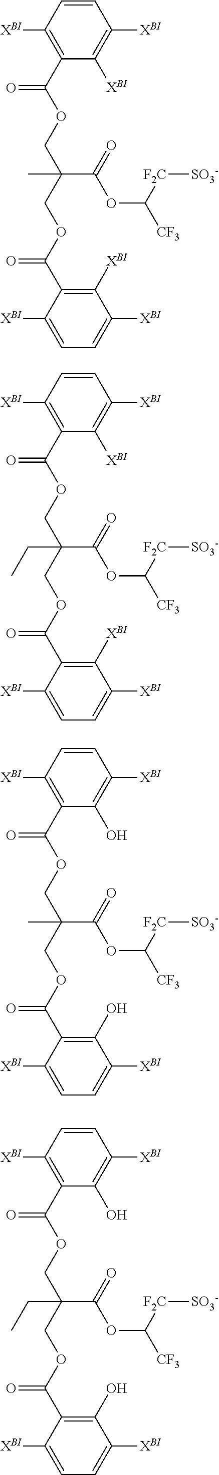

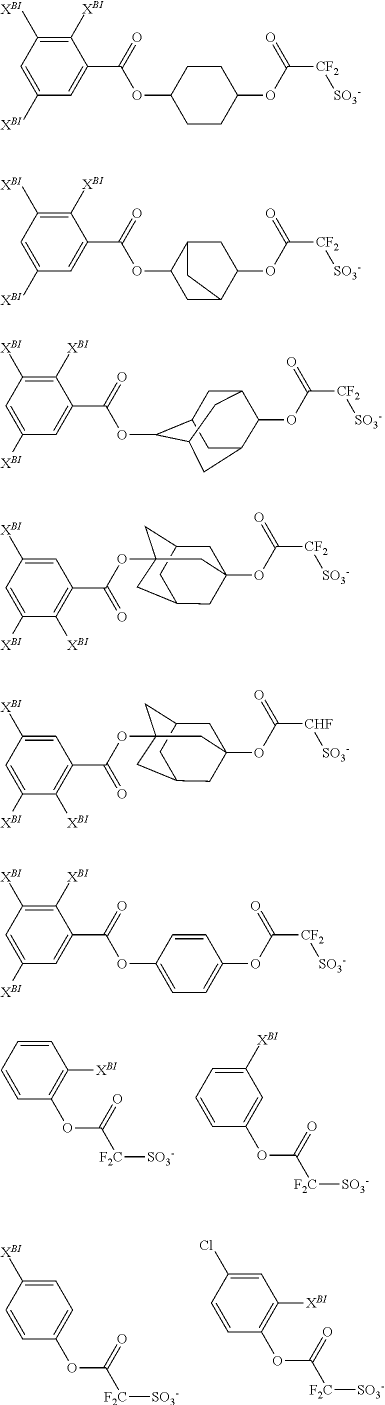

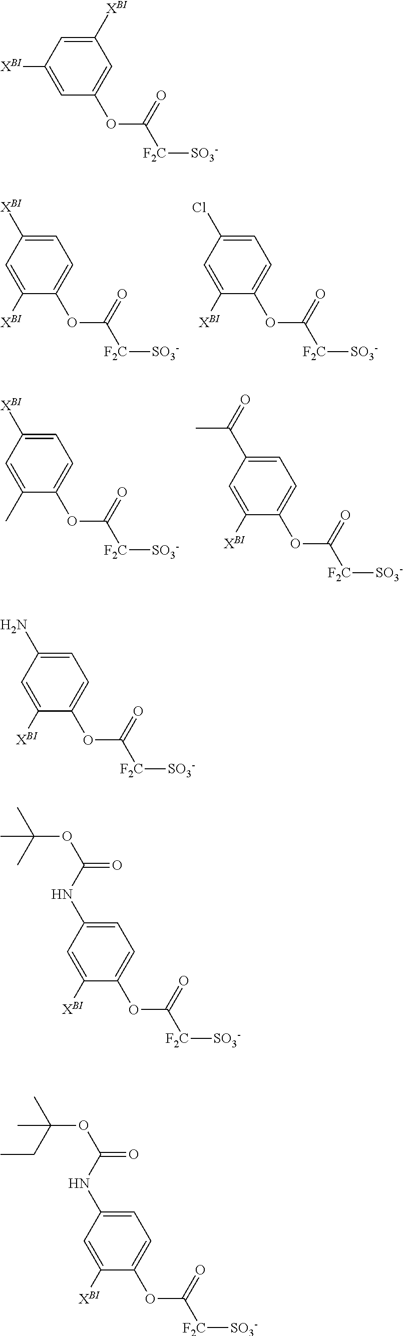

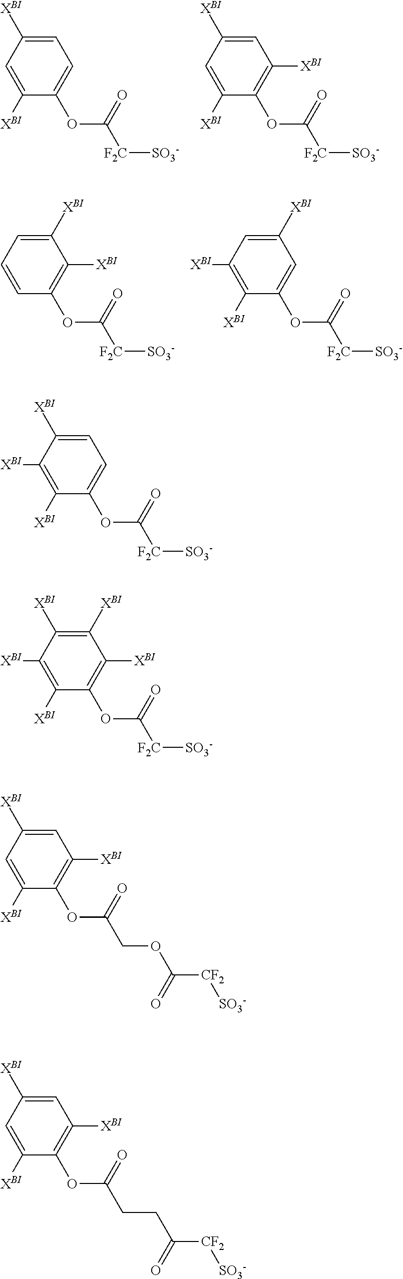

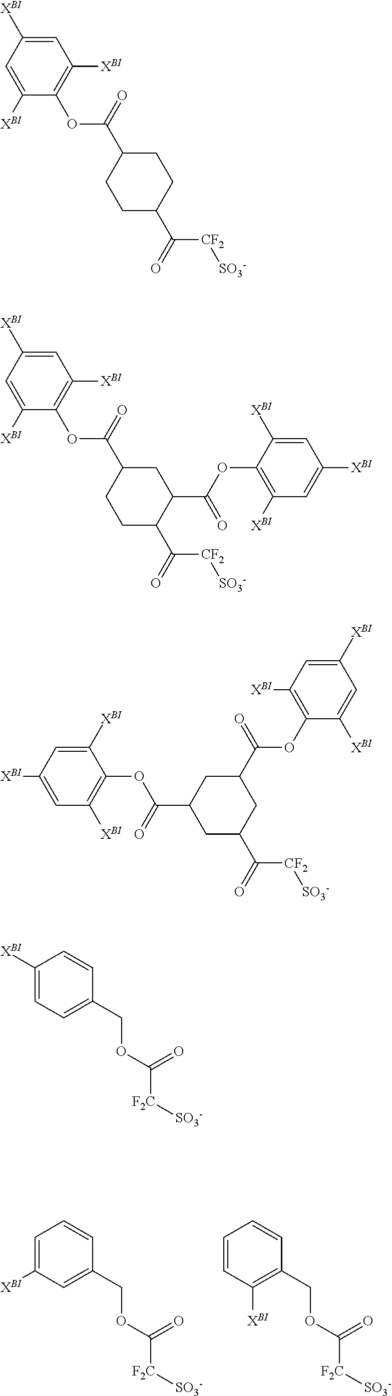

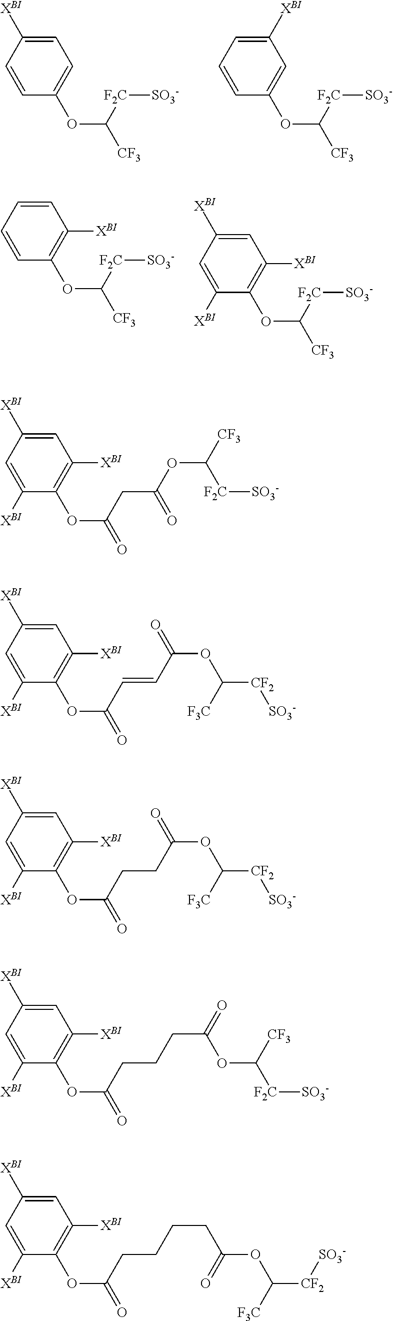

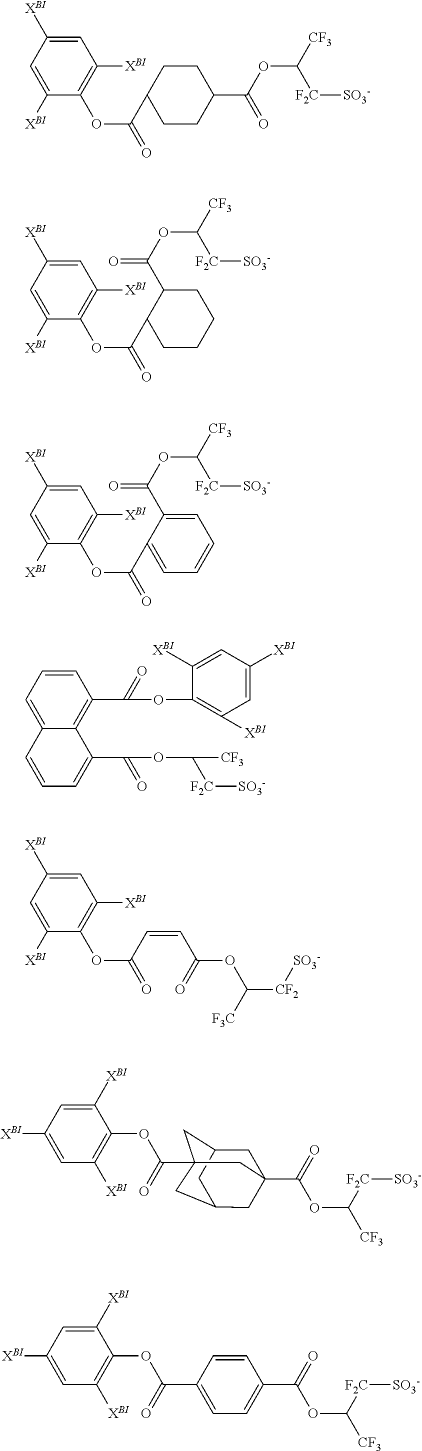

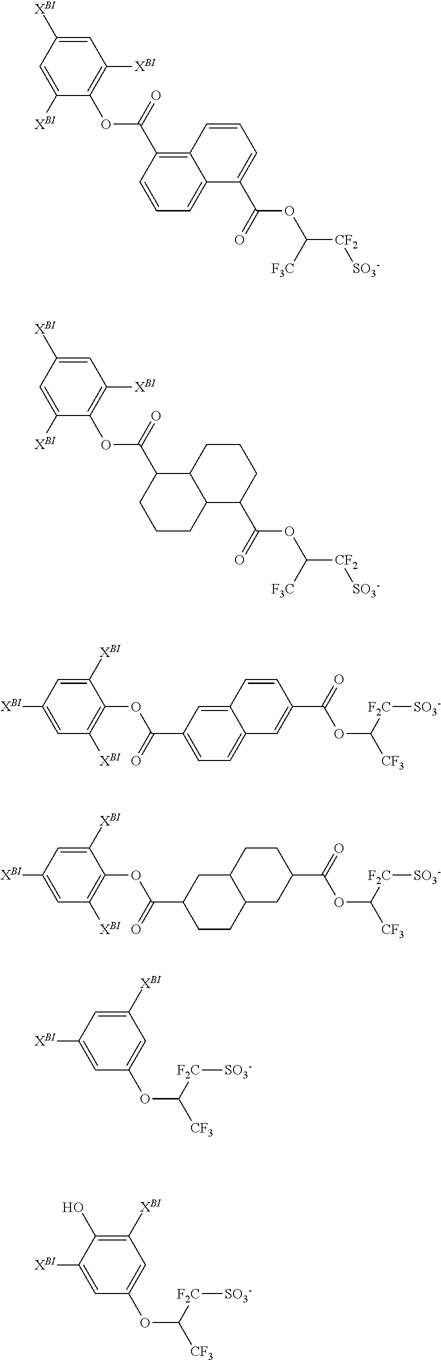

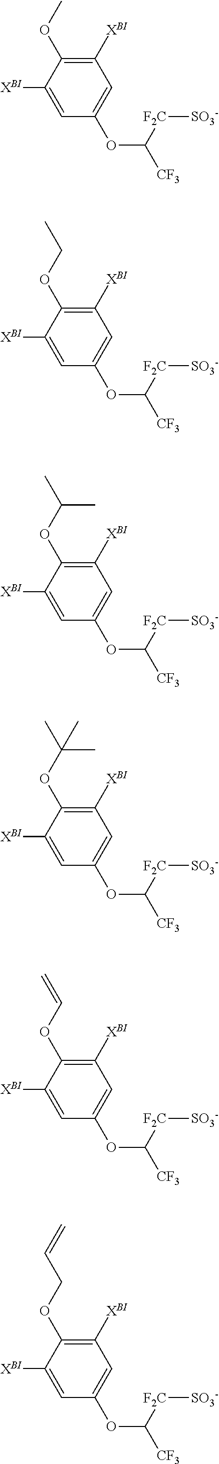

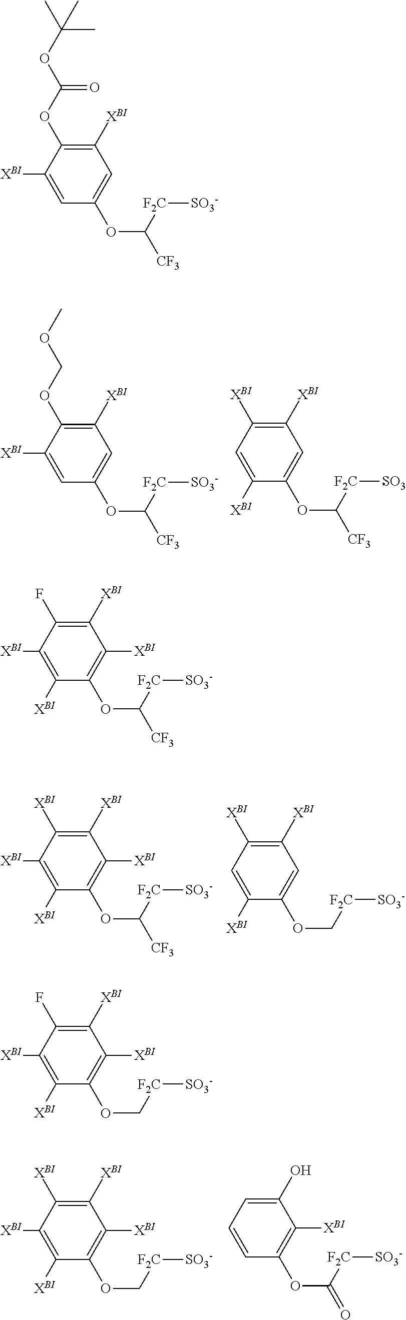

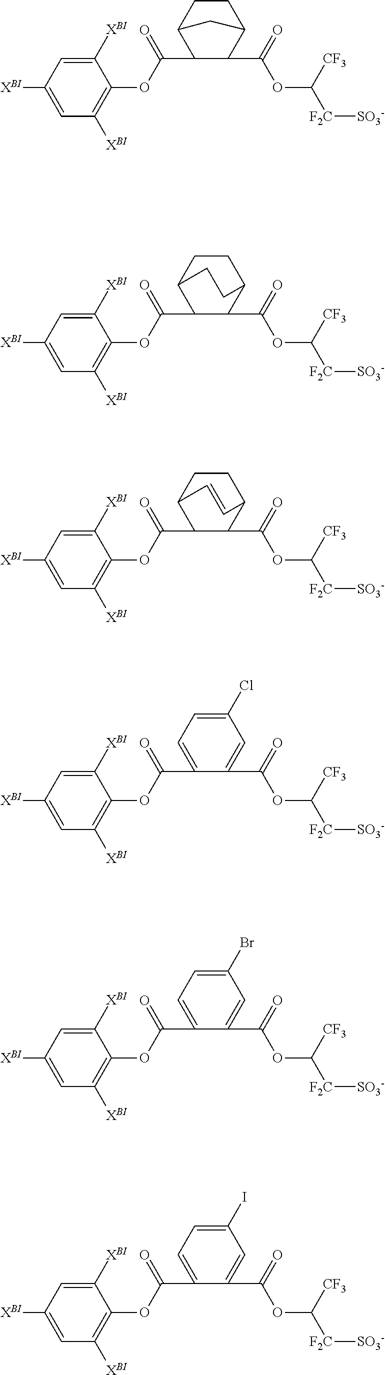

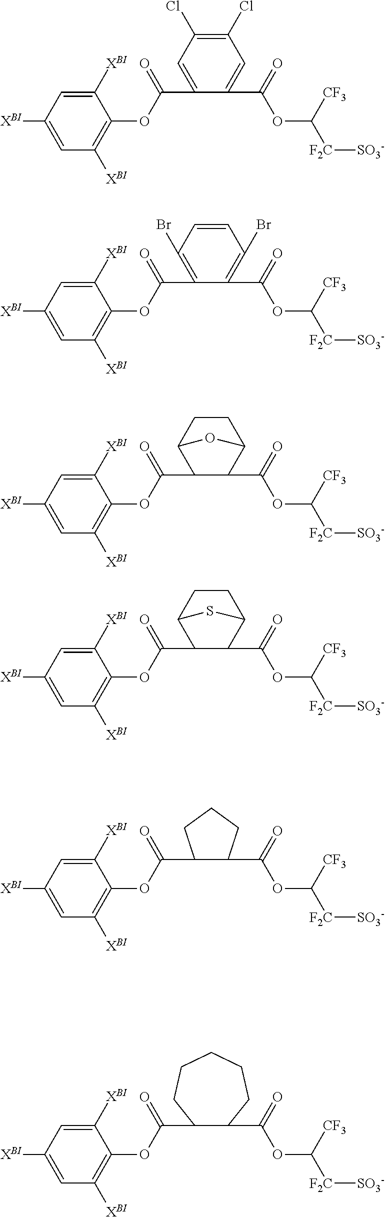

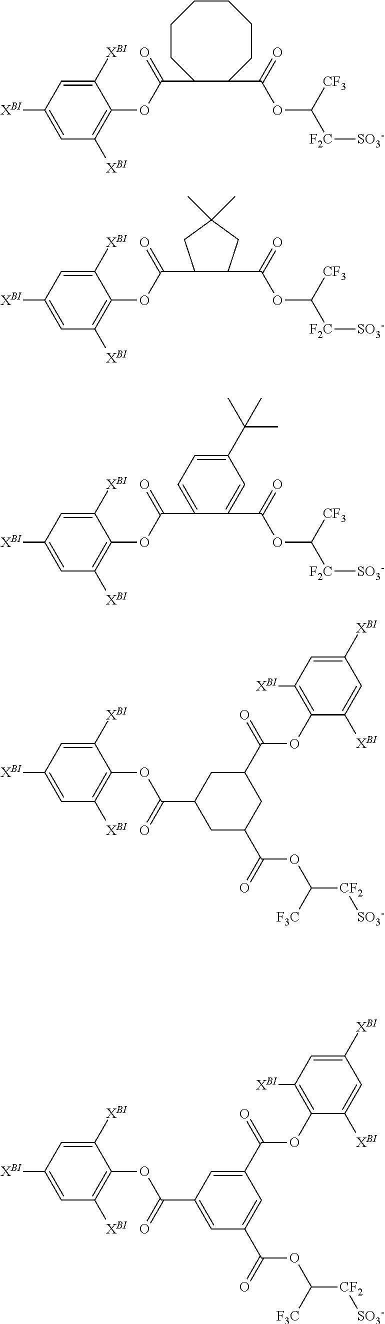

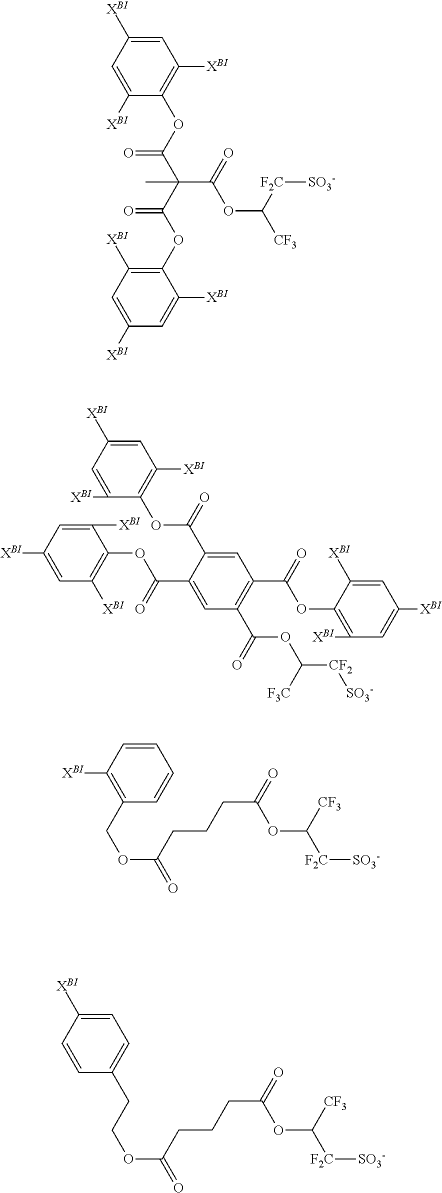

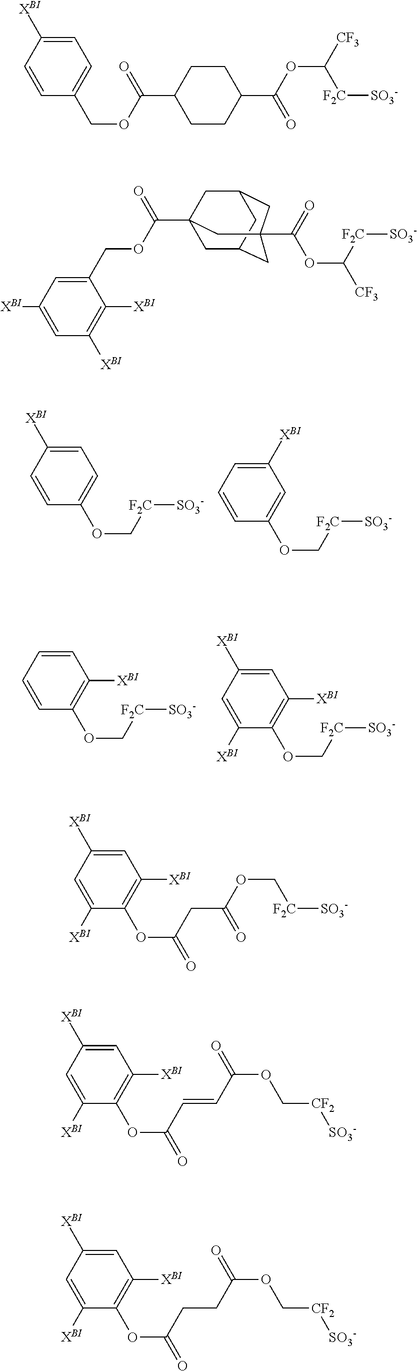

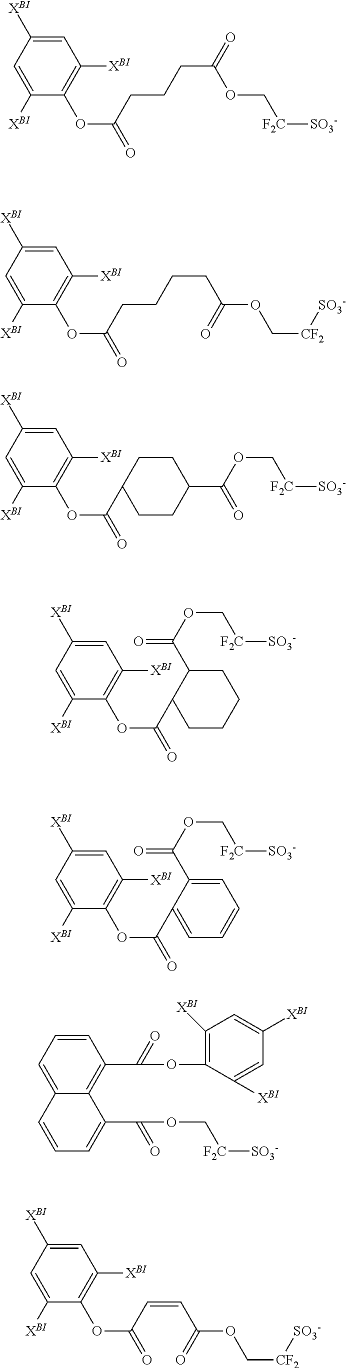

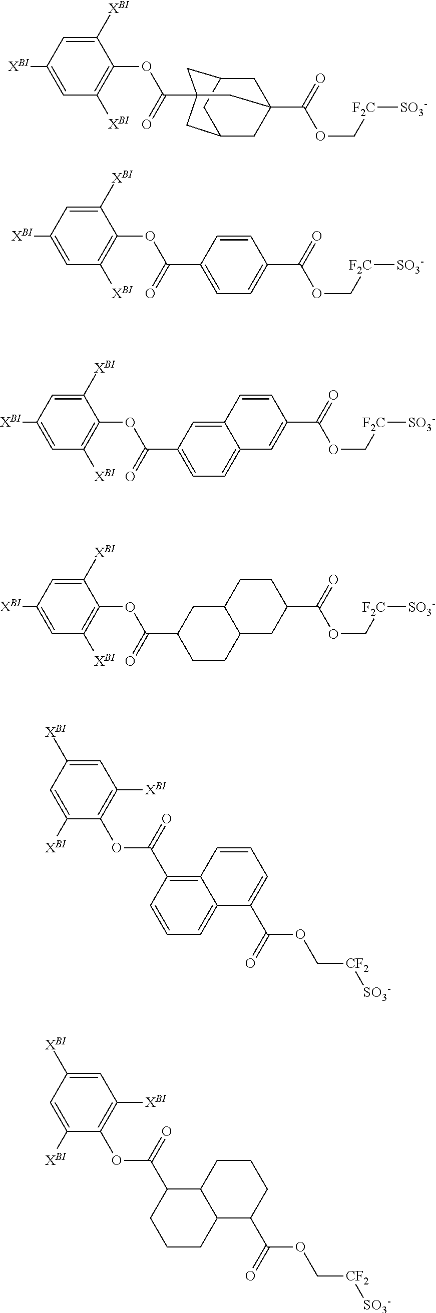

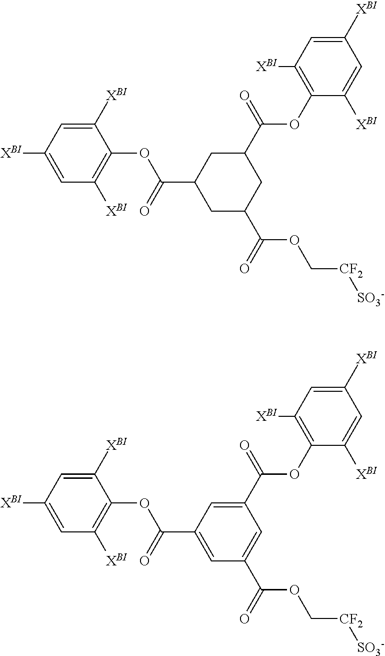

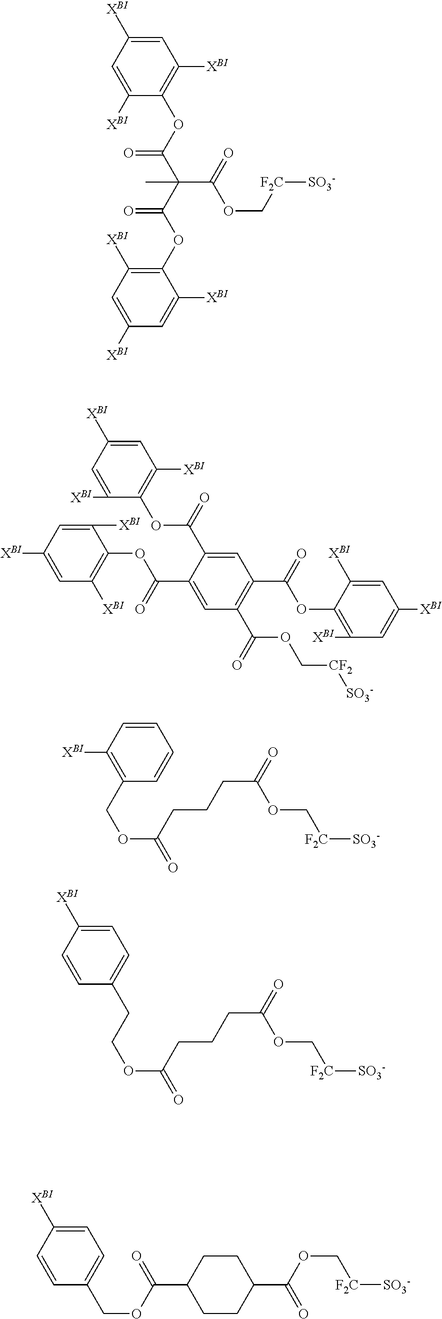

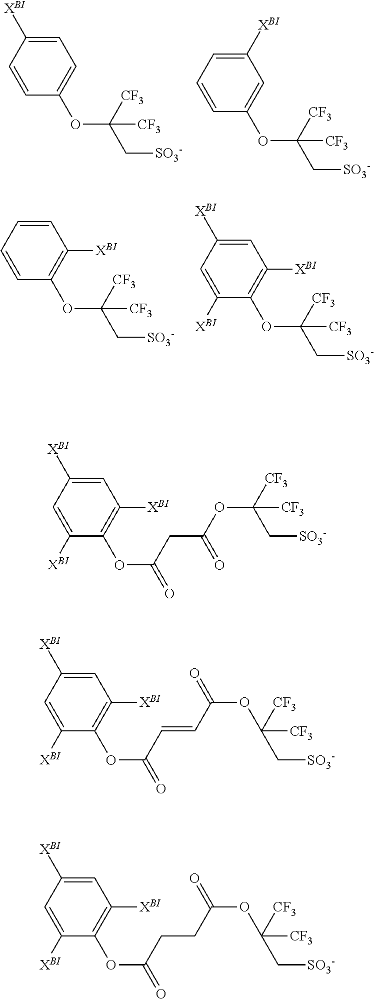

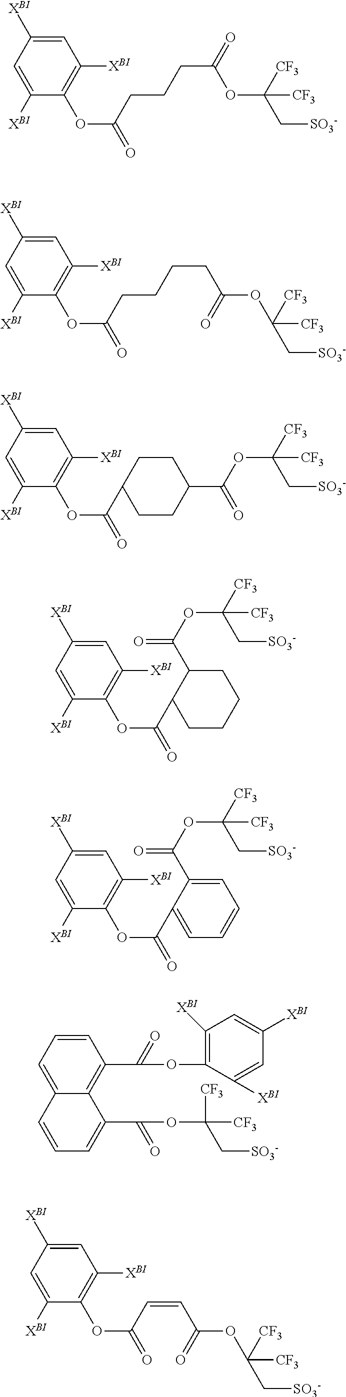

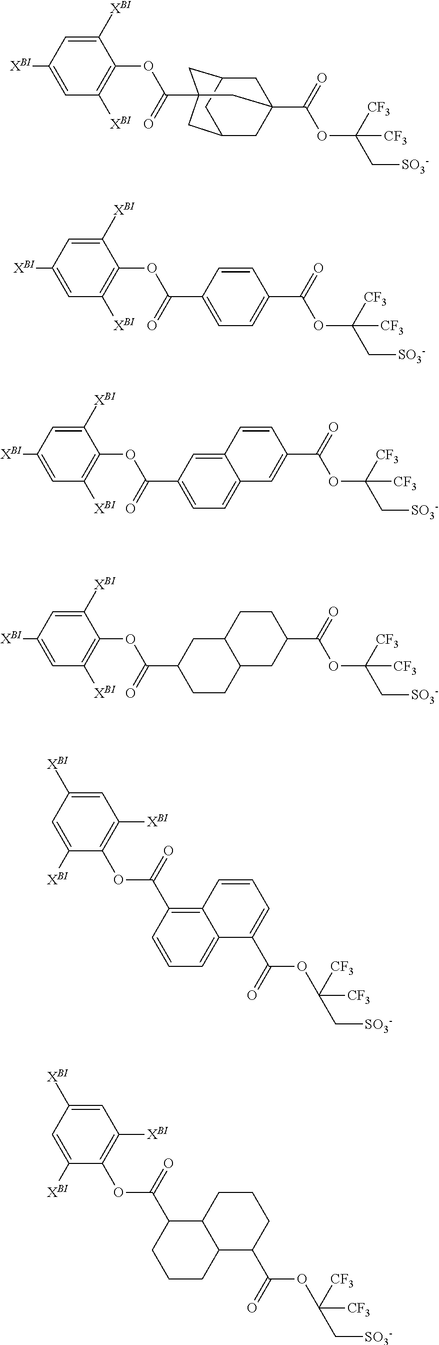

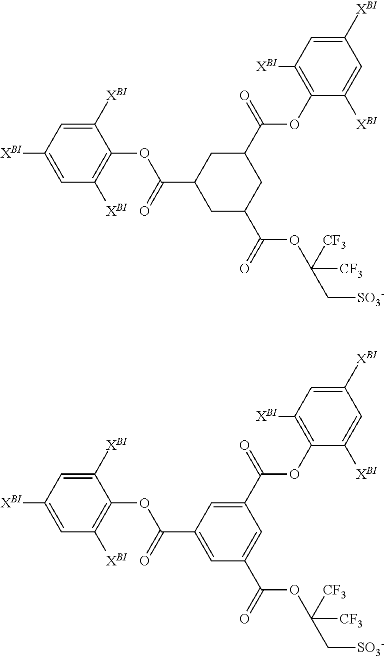

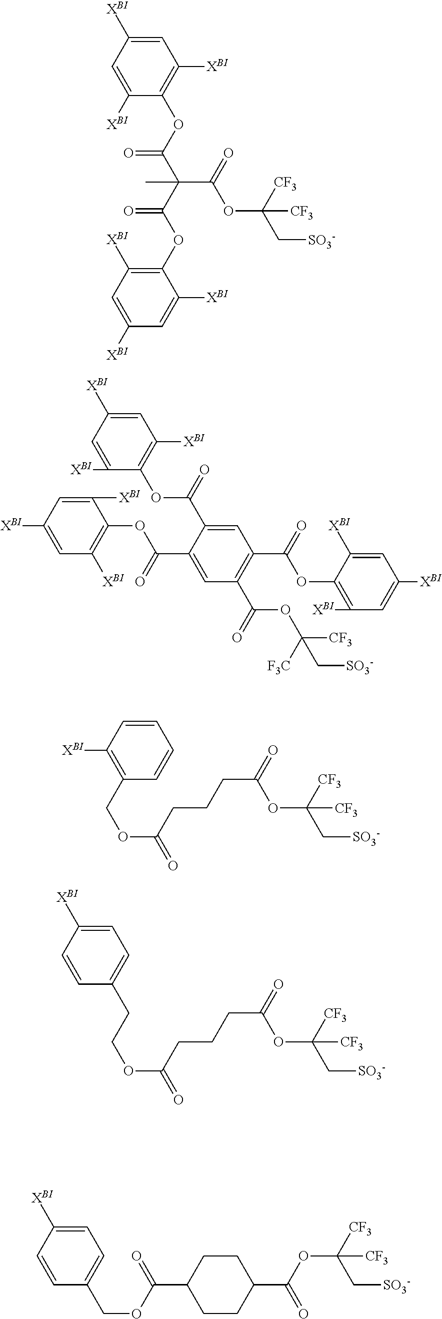

When the inventive resist composition contains an additive-type acid generator, the additive-type acid generator is preferably contained in an amount of 0.1 to 50 parts by mass, more preferably 1 to 40 parts by mass based on 100 parts by mass of the base polymer. Incorporating any of the repeating units-f1 to -f3 into the base polymer and/or incorporating the additive-type acid generator enables the inventive resist composition to function as a chemically amplified resist composition.

### [Organic Solvent]

The inventive resist composition may contain an organic solvent. The organic solvent is not particularly limited as long as it is capable of dissolving the above-described components and the components described below. Examples of such an organic solvent include ones disclosed in paragraphs [144] and [145] of JP 2008-111103 A: ketones, such as cyclohexanone, cyclopentanone, methyl-2-n-pentyl ketone, and 2-heptanone; alcohols, such as 3-methoxybutanol, 3-methyl-3-methoxybutanol, 1-methoxy-2-propanol, 1-ethoxy-2-propanol, and diacetone alcohol; ethers, such as propylene glycol monomethyl ether, ethylene glycol monomethyl ether, propylene glycol monoethyl ether, ethylene glycol monoethyl ether, propylene glycol dimethyl ether, and diethylene glycol dimethyl ether; esters, such as propylene glycol monomethyl ether acetate, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones, such as γ-butyrolactone; etc.

The inventive resist composition preferably contains the organic solvent in an amount of 100 to 10,000 parts by mass, more preferably 200 to 8,000 parts by mass based on 100 parts by mass of the base polymer. One kind of the organic solvent may be contained, or two or more kinds thereof may be contained in mixture.

### [Other Components]

In addition to the above-described components, the inventive resist composition may also contain a surfactant, a dissolution inhibitor, a crosslinking agent, a quencher other than the inventive onium salt (hereinafter, referred to as other quenchers), a water-repellency enhancer, an acetylene alcohol, etc.

Examples of the surfactant include ones disclosed in paragraphs [0165] and [0166] of JP 2008-111103 A.

Adding a surfactant can further enhance or control the coatability of the resist composition. When the inventive resist composition contains the surfactant, the contained amount is preferably 0.0001 to 10 parts by mass based on 100 parts by mass of the base polymer. One kind of the surfactant may be used, or two or more kinds thereof may be used in combination.

When the inventive resist composition is a positive resist composition, blending a dissolution inhibitor can further increase the difference in dissolution rate between exposed and unexposed areas, and further enhance the resolution. Examples of the dissolution inhibitor include compounds: the compounds each have a molecular weight of preferably 100 to 1,000, more preferably 150 to 800; and which contains two or more phenolic hydroxy groups per molecule, and in which 0 to 100 mol % of all the hydrogen atoms of the phenolic hydroxy groups are substituted with acid-labile groups; or a compound which contains a carboxy group in a molecule, and in which 50 to 100 mol % of all the hydrogen atoms of such carboxy groups are substituted with acid-labile groups on average. Specific examples include compounds obtained by substituting acid-labile groups for hydrogen atoms of hydroxy groups or carboxy groups of bisphenol A, trisphenol, phenolphthalein, cresol novolak, naphthalenecarboxylic acid, adamantanecarboxylic acid, cholic acid; etc. Examples of such compounds are disclosed in paragraphs to of JP 2008-122932 A.

When the inventive resist composition is a positive type and contains the above-described dissolution inhibitor, the contained amount is preferably 0 to 50 parts by mass, more preferably 5 to 40 parts by mass based on 100 parts by mass of the base polymer. One kind of the dissolution inhibitor may be used, or two or more kinds thereof may be used in combination.

Meanwhile, when the inventive resist composition is a negative resist composition, the dissolution rate of exposed areas can be decreased by adding a crosslinking agent, and thus, a negative pattern can be obtained. Examples of the crosslinking agent include epoxy compounds, melamine compounds, guanamine compounds, glycoluryl compounds, or urea compounds each of which is substituted with at least one group selected from a methylol group, an alkoxymethyl group, and an acyloxymethyl group; isocyanate compounds; azide compounds; compounds having a double bond such as an alkenyloxy group, etc. These compounds may be used as an additive, or introduced into a polymer side chain as a pendant group. In addition, compounds containing a hydroxy group can also be used as a crosslinking agent.

Examples of the epoxy compounds include tris(2,3-epoxypropyl)isocyanurate, trimethylolmethane triglycidyl ether, trimethylolpropane triglycidyl ether, triethylolethane triglycidyl ether, etc.

Examples of the melamine compounds include hexamethylolmelamine, hexamethoxymethylmelamine, such compounds as hexamethylolmelamine having 1 to 6 methylol groups methoxymethylated, and mixtures thereof; and hexamethoxyethylmelamine, hexaacyloxymethylmelamine, such compounds as hexamethylolmelamine having 1 to 6 methylol groups acyloxymethylated, and mixtures thereof.

Examples of the guanamine compounds include tetramethylolguanamine, tetramethoxymethylguanamine, such compounds as tetramethylolguanamine having 1 to 4 methylol groups methoxymethylated, and mixtures thereof; and tetramethoxyethylguanamine, tetraacyloxyguanamine, such compounds as tetramethylolguanamine having 1 to 4 methylol groups acyloxymethylated, and mixtures thereof.

Examples of the glycoluril compounds include tetramethylolglycoluril, tetramethoxyglycoluril, tetramethoxymethylglycoluril, such compounds as tetramethylolglycoluril having 1 to 4 methylol groups methoxymethylated, and mixtures thereof; and such compounds as tetramethylolglycoluril having 1 to 4 methylol groups acyloxymethylated, and mixtures thereof.

Examples of the urea compounds include tetramethylol urea, tetramethoxymethyl urea, tetramethoxyethyl urea, such compounds as tetramethylol urea having 1 to 4 methylol groups methoxymethylated, mixtures thereof, and the like.

Examples of the isocyanate compounds include tolylene diisocyanate, diphenylmethane diisocyanate, hexamethylene diisocyanate, cyclohexane diisocyanate, etc.

Examples of the azide compounds include 1,1′-biphenyl-4,4′-bisazide, 4,4′-methylidene bisazide, 4,4′-oxybisazide, etc.

Examples of the compounds containing an alkenyloxy group include ethylene glycol divinyl ether, triethylene glycol divinyl ether, 1,2-propanediol divinyl ether, 1,4-butanediol divinyl ether, tetramethylene glycol divinyl ether, neopentyl glycol divinyl ether, trimethylolpropane trivinyl ether, hexanediol divinyl ether, 1,4-cyclohexanediol divinyl ether, pentaerythritol trivinyl ether, pentaerythritol tetravinyl ether, sorbitol tetravinyl ether, sorbitol pentavinyl ether, etc.

When the inventive resist composition is a negative type and contains the above-described crosslinking agent, the contained amount is preferably 0.1 to 50 parts by mass, more preferably 1 to 40 parts by mass based on 100 parts by mass of the base polymer. One kind of the crosslinking agent may be used, or two or more kinds thereof may be used in combination.

Examples of the other quenchers include conventional basic compounds. Examples of the conventional basic compounds include primary, secondary, and tertiary aliphatic amines, mixed amines, aromatic amines, heterocyclic amines, nitrogen-containing compounds having a carboxy group, nitrogen-containing compounds having a sulfonyl group, nitrogen-containing compounds having a hydroxy group, nitrogen-containing compounds having a hydroxyphenyl group, alcoholic nitrogen-containing compounds, amides, imides, carbamates, etc. Particularly preferable are primary, secondary, and tertiary amine compounds disclosed in paragraphs [0146] to [0164] of JP 2008-111103 A; in particular, amine compounds having a hydroxy group, an ether bond, an ester bond, a lactone ring, a cyano group, or a sulfonic acid ester group; compounds having a carbamate group disclosed in JP 3790649 B; etc. Adding such a basic compound can, for example, further suppress the acid diffusion rate in the resist film and correct the shape.

Other examples of the other quenchers include onium salts, such as sulfonium salts, iodonium salts, and ammonium salts, of carboxylic acids and sulfonic acids which are not fluorinated at α position as disclosed in JP 2008-158339 A. While α-fluorinated sulfonic acid, imide acid, or methide acid is necessary to deprotect the acid-labile group of carboxylic acid ester, a carboxylic acid or sulfonic acid not fluorinated at a position is released by salt exchange with the onium salt not fluorinated at α position. Such carboxylic acid and sulfonic acid not fluorinated at α position hardly induce deprotection reaction, and thus function as quenchers.

Other examples of the other quenchers further include a polymeric quencher disclosed in JP 2008-239918 A. This quencher is oriented on the surface of a resist film, and enhances the rectangularity of a resist pattern. The polymeric quencher also has effects of preventing rounding of pattern top and film thickness loss of pattern when a top coat for immersion exposure is applied.

When the inventive resist composition contains other quenchers, the contained amount is preferably 0 to 5 parts by mass, more preferably 0 to 4 parts by mass based on 100 parts by mass of the base polymer. One kind of the other quenchers may be used, or two or more kinds thereof may be used in combination.

The water-repellency enhancer enhances the water repellency of the resist film surface, and can be employed in immersion lithography with no top coat. The water-repellency enhancer is preferably a polymer containing a fluorinated alkyl group, a polymer containing a 1,1,1,3,3,3-hexafluoro-2-propanol residue with a particular structure, etc., more preferably ones exemplified in JP 2007-297590 A, JP 2008-111103 A, etc. The water-repellency enhancer needs to be dissolved in an alkaline developer or an organic solvent developer. The particular water-repellency enhancer having a 1,1,1,3,3,3-hexafluoro-2-propanol residue mentioned above has favorable solubility to developers. A polymer containing a repeating unit with an amino group or amine salt as a water-repellency enhancer exhibits high effects of preventing acid evaporation during post-exposure baking (PEB) and opening failure of a hole pattern after development. When the inventive resist composition contains the water-repellency enhancer, the contained amount is preferably 0 to 20 parts by mass, more preferably 0.5 to 10 parts by mass based on 100 parts by mass of the base polymer. One kind of the water-repellency enhancer may be used, or two or more kinds thereof may be used in combination.

Examples of the acetylene alcohol include ones disclosed in paragraphs to of JP 2008-122932 A. When the inventive resist composition contains the acetylene alcohol, the contained amount is preferably 0 to 5 parts by mass based on 100 parts by mass of the base polymer. One kind of the acetylene alcohol may be used, or two or more kinds thereof may be used in combination.

The inventive resist composition allows excellent contrast and improvement of LWR and CDU. This is because of the effect of suppressing acid diffusion achieved by the amide structure or sulfonamide structure of the inventive onium salt and because of improved affinity with alkaline developers.

### [Patterning Process]

When the inventive resist composition is used for manufacturing various integrated circuits, known lithography techniques are applicable. Examples of a patterning process include a method including the steps of:
forming a resist film on a substrate by using the above-described resist composition;
exposing the resist film to a high-energy beam; and
developing the exposed resist film by using a developer.

Firstly, the inventive resist composition is applied onto a substrate (such as Si, SiO₂, SiN, SiON, TiN, WSi, BPSG, SOG, and organic antireflective film) for manufacturing an integrated circuit or a substrate (such as Cr, CrO, CrON, MoSi₂, and SiO₂) for manufacturing a mask circuit by an appropriate coating process such as spin coating, roll coating, flow coating, dip coating, spray coating, or doctor coating so that the coating film can have a thickness of 0.01 to 2 μm. The resultant is prebaked on a hot plate preferably at 60 to 150° C. for 10 seconds to 30 minutes, more preferably at 80 to 120° C. for 30 seconds to 20 minutes. In this manner, a resist film is formed.

Then, the resist film is exposed using a high-energy beam. Examples of the high-energy beam include ultraviolet ray, deep ultraviolet ray, EB (electron beam), EUV (extreme ultraviolet ray) at a wavelength of 3 to 15 nm, X-ray, soft X-ray, excimer laser beam, g-ray, synchrotron radiation, etc. When ultraviolet ray, deep ultraviolet ray, EUV, X-ray, soft X-ray, excimer laser beam, γ-ray, synchrotron radiation, or the like is employed as the high-energy beam, the irradiation is performed directly or using a mask for forming a target pattern at an exposure dose of preferably about 1 to 200 mJ/cm², more preferably about 10 to 100 mJ/cm². When EB is employed as the high-energy beam, the exposure dose is preferably about 0.1 to 300 ρC/cm², more preferably about 0.5 to 200 ρC/cm², and the writing is performed directly or using a mask for forming a target pattern. Note that the inventive resist composition is particularly suitable for fine patterning with a KrF excimer laser beam, an ArF excimer laser beam, an EB, an EUV, X-ray, soft X-ray, γ-ray, or synchrotron radiation among the high-energy beams. In particular, a KrF excimer laser beam, an ArF excimer laser beam, an EB, or an EUV having a wavelength of 3 to 15 nm is suitably used. The inventive resist composition is particularly suitable for fine patterning with an EB or an EUV.

The exposure may or may not be followed by PEB on a hot plate or in an oven preferably at 30 to 150° C. for 10 seconds to 30 minutes, more preferably at 50 to 120° C. for 30 seconds to 20 minutes.

After the exposure or PEB, the exposed resist film is developed using a developer of 0.1 to 10 mass %, preferably 2 to 5 mass % aqueous alkaline solution such as tetramethylammonium hydroxide (TMAH), tetraethylammonium hydroxide, tetrapropylammonium hydroxide, or tetrabutylammonium hydroxide for 3 seconds to 3 minutes, preferably 5 seconds to 2 minutes by a conventional technique, such as a dip, puddle, or spray method. Thus, the target pattern is formed. In the case of a positive resist composition, the portion irradiated with the light is dissolved by the developer, while the unexposed portion remains undissolved. In this way, the target positive pattern is formed on the substrate. A negative resist composition is the reverse of the positive resist composition. That is, the portion irradiated with the light is made insoluble to the developer, while the unexposed portion dissolves.

The positive resist composition containing a base polymer that contains an acid-labile group can also be used to obtain a negative pattern by organic solvent development. Examples of the developer used in this event include 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, phenylmethyl acetate, benzyl formate, phenylethyl formate, methyl 3-phenylpropionate, benzyl propionate, phenylethyl acetate, 2-phenylethyl acetate, etc. One of these organic solvents can be used, or two or more thereof can be used in mixture.

When the development is completed, rinsing can be performed. The rinsing liquid is preferably a solvent that is miscible with the developer but does not dissolve the resist film. As such a solvent, it is preferable to use an alcohol having 3 to 10 carbon atoms, an ether compound having 8 to 12 carbon atoms, and an alkane, alkene, alkyne and aromatic solvent, each having 6 to 12 carbon atoms.

Examples of the alcohol having 3 to 10 carbon atoms include n-propyl alcohol, isopropyl alcohol, 1-butyl alcohol, 2-butyl alcohol, isobutyl alcohol, tert-butyl alcohol, 1-pentanol, 2-pentanol, 3-pentanol, tert-pentyl alcohol, neopentyl alcohol, 2-methyl-1-butanol, 3-methyl-1-butanol, 3-methyl-3-pentanol, cyclopentanol, 1-hexanol, 2-hexanol, 3-hexanol, 2,3-dimethyl-2-butanol, 3,3-dimethyl-1-butanol, 3,3-dimethyl-2-butanol, 2-ethyl-1-butanol, 2-methyl-1-pentanol, 2-methyl-2-pentanol, 2-methyl-3-pentanol, 3-methyl-1-pentanol, 3-methyl-2-pentanol, 3-methyl-3-pentanol, 4-methyl-1-pentanol, 4-methyl-2-pentanol, 4-methyl-3-pentanol, cyclohexanol, 1-octanol, etc.

Examples of the ether compound having 8 to 12 carbon atoms include di-n-butyl ether, diisobutyl ether, di-sec-butyl ether, di-n-pentyl ether, diisopentyl ether, di-sec-pentyl ether, di-tert-pentyl ether, di-n-hexyl ether, etc.

Examples of the alkane having 6 to 12 carbon atoms include hexane, heptane, octane, nonane, decane, undecane, dodecane, methylcyclopentane, dimethylcyclopentane, cyclohexane, methylcyclohexane, dimethylcyclohexane, cycloheptane, cyclooctane, cyclononane, etc. Examples of the alkene having 6 to 12 carbon atoms include hexene, heptene, octene, cyclohexene, methylcyclohexene, dimethylcyclohexene, cycloheptene, cyclooctene, etc. Examples of the alkyne having 6 to 12 carbon atoms include hexyne, heptyne, octyne, etc.

Examples of the aromatic solvent include toluene, xylene, ethylbenzene, isopropylbenzene, tert-butylbenzene, mesitylene, etc.

The rinsing can reduce resist pattern collapse and defect formation. Meanwhile, the rinsing is not necessarily essential, and the amount of the solvent used can be reduced by not performing the rinsing.

After the development, a hole pattern or trench pattern can be shrunk by thermal flow, RELACS process, or DSA process. A shrink agent is applied onto the hole pattern, and the shrink agent undergoes crosslinking on the resist film surface by diffusion of the acid catalyst from the resist film during baking, so that the shrink agent is attached to sidewalls of the hole pattern. The baking temperature is preferably 70 to 180° C., more preferably 80 to 170° C. The baking time is preferably 10 to 300 seconds. The extra shrink agent is removed, and thus the hole pattern is shrunk.

### EXAMPLE

Hereinafter, the present invention will be described specifically with reference to Synthesis Examples, Examples, and Comparative Examples. However, the present invention is not limited to the following Examples. Incidentally, the following apparatus was used.

MALDI TOF-MS: 53000, manufactured by JEOL Ltd.

### [1] Synthesis of Onium Salt

### [Example 1-1] Synthesis of SQ-1

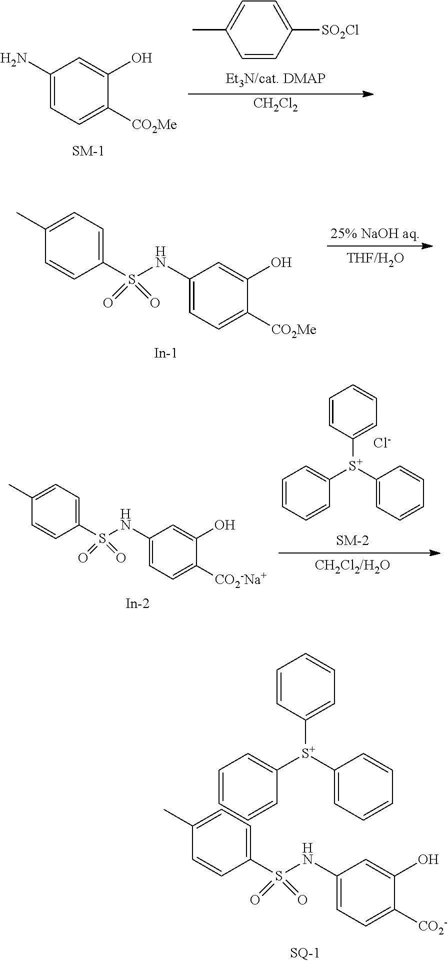

### (1) Synthesis of Intermediate In-1

Under a nitrogen atmosphere, a starting material SM-1 (16.7 g), p-toluenesulfonyl chloride (22.9 g), and 4-dimethylaminopyridine (1.2 g) were dissolved in methylene chloride (150 g). The reaction solution was cooled in an ice bath, and while maintaining the inner temperature at 20° C. or lower, triethylamine (13.2 g) was added dropwise. After the addition, the inner temperature was raised to room temperature, and the reaction solution was aged for 12 hours. After the aging, the reaction solution was cooled, and water (80 g) was dropped thereto to terminate the reaction. After that, the reaction solution was separated, a common aqueous work-up was performed, the solvent was evaporated, and then a product was recrystallized with hexane to obtain 30.8 g of an intermediate In-1 as a white crystal (96% yield).

### (2) Synthesis of Intermediate In-2

Under a nitrogen atmosphere, the intermediate In-1 (30.8 g) was dissolved in THF (100 g). The reaction solution was cooled in an ice bath, and while maintaining the inner temperature at 20° C. or lower, a 25 mass % aqueous solution of sodium hydroxide (16.1 g) was added dropwise. After the addition, the inner temperature was raised to room temperature, and the reaction solution was aged for 12 hours. After the aging, the reaction solution was concentrated, and the precipitated solid was subjected to solid-liquid washing with diisopropyl ether. The solid was filtered and dried to obtain 28.8 g of an intermediate In-2 as a white crystal (91% yield).

### (3) Synthesis of Onium Salt SQ-1

Under a nitrogen atmosphere, the intermediate In-2 (9.9 g) and a starting material SM-2 (10.8 g) were dissolved in methylene chloride (50 g) and water (30 g), and the mixture was stirred for 20 minutes. The reaction solution was separated, the organic layer was collected, a common aqueous work-up was performed, and the solvent was evaporated to obtain 15.4 g of an onium salt SQ-1 as a colorless oil (90% yield).

The TOF-MS results of the onium salt SQ-1 are shown below.

MALDI TOF-MS: POSITIVE M⁺263 (C₁₈H₁₅S⁺ equivalent)
NEGATIVE M⁻265 (C₁₄H₁₂NO₅S⁻ equivalent)

### [Examples 1-2 to 1-9] Syntheses of SQ-2 to SQ-9

Various onium salts were synthesized by organic synthesis reactions. The structures of the onium salts contained in resist compositions (chemically amplified resist compositions) are shown below.

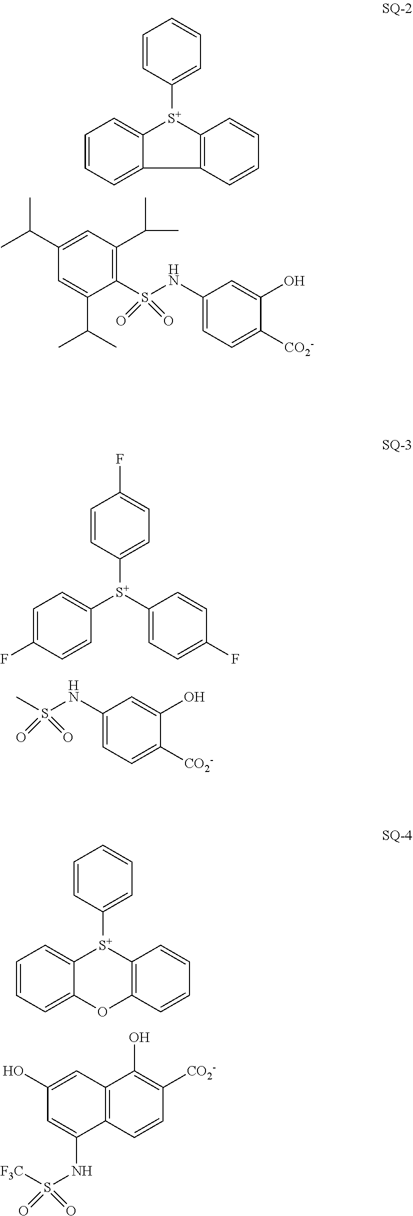

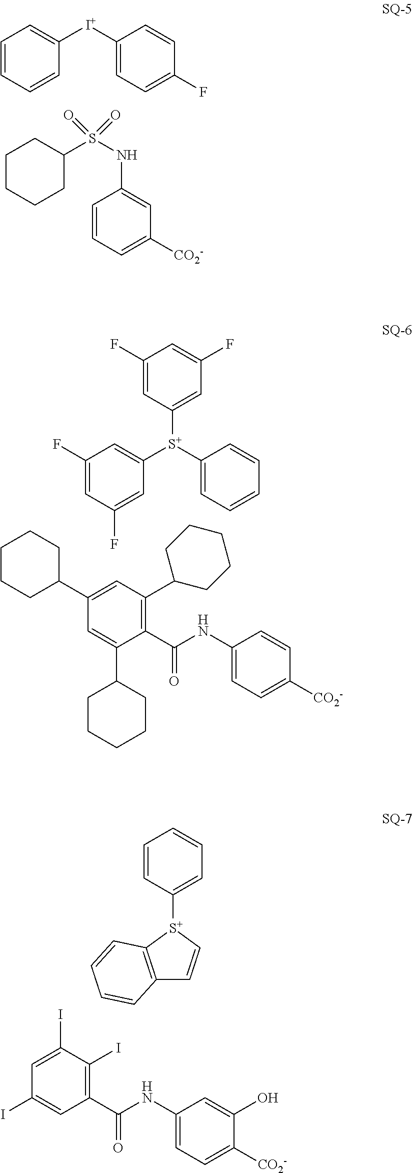

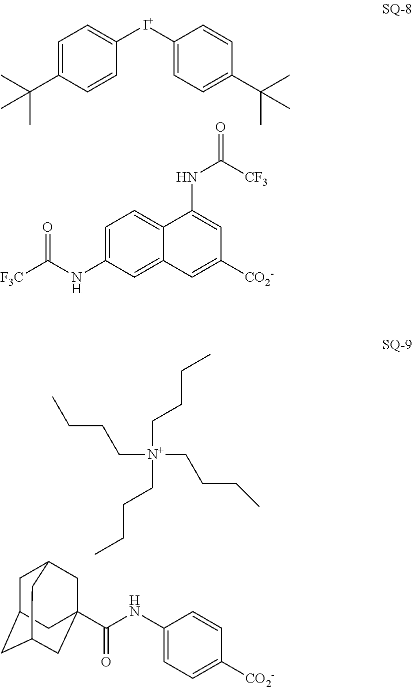

### [2] [Synthesis Example] Syntheses of Base Polymers (P-1 to P-5)

The monomers were combined to perform a copolymerization reaction in a solvent THF. A crystal was precipitated in methanol, furthermore, repeatedly washed with hexane, then isolated and dried to obtain a base polymer (P-1 to P-5) of the composition shown below. The composition of the obtained base polymer was identified by ¹H-NMR, and the Mw and Mw/Mn were identified by GPC (eluent: THF, standard: polystyrene).

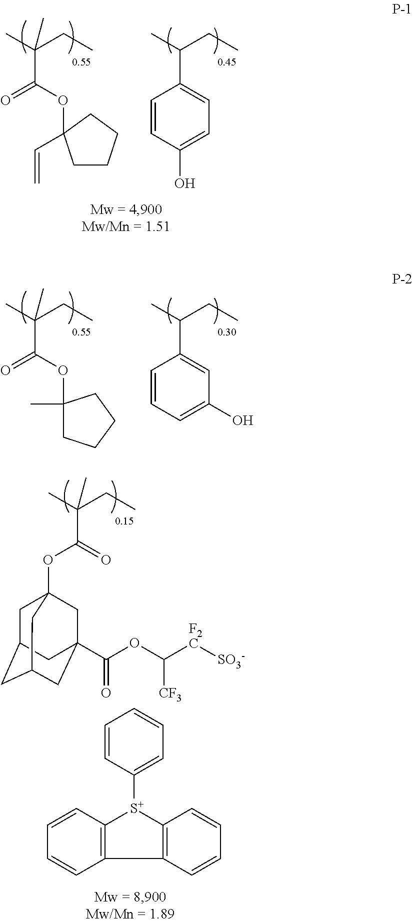

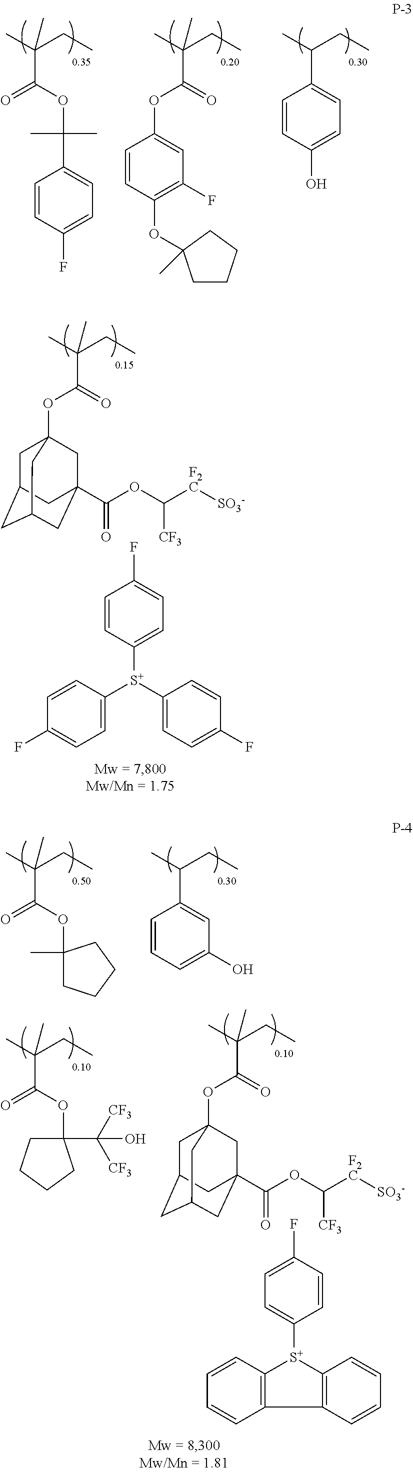

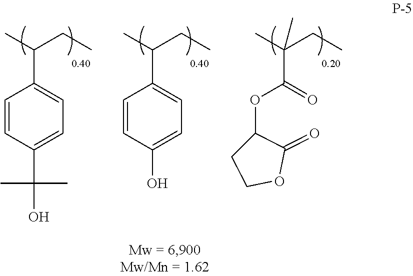

### [3] [Examples 2-1 to 2-20 and Comparative Examples 1-1 to 1-12] Preparation of Resist Composition

### (1) Preparation of Resist Composition

A solution obtained by dissolving the components in accordance with the composition shown in Tables 1 and 2 was filtered through a filter having a pore size of 0.2 μm. Thus, resist compositions were prepared. The resist compositions of Examples 2-1 to 2-18 and Comparative Examples 1-1 to 1-10 were positive resist compositions, and the resist compositions of Examples 2-19 and 2-20 and Comparative Examples 1-11 and 1-12 were negative resist compositions.

The components in Table 1 are as follows.
Organic Solvents:
   PGMEA (propylene glycol monomethyl ether acetate)
   DAA (diacetone alcohol)
Photo-acid generators: PAG-1 to PAG-5

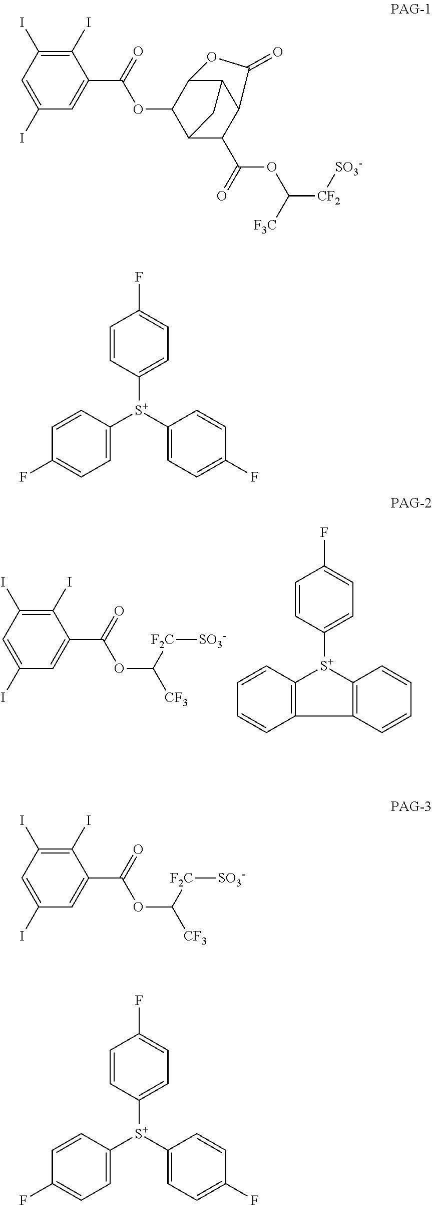

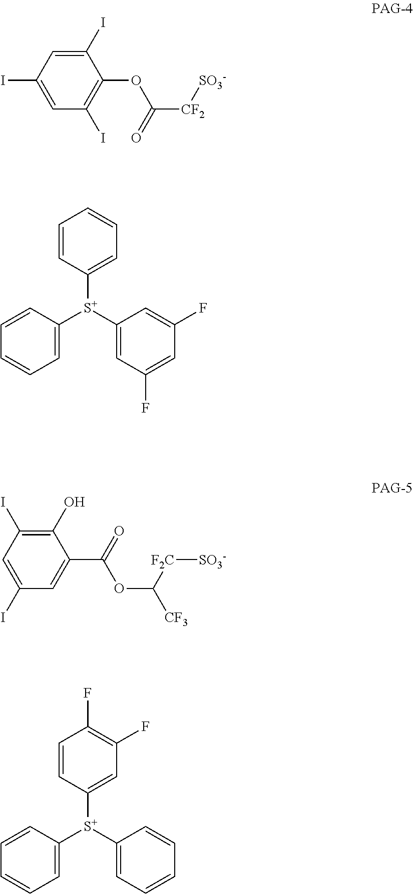

Blend quenchers: bQ-1 and bQ-2

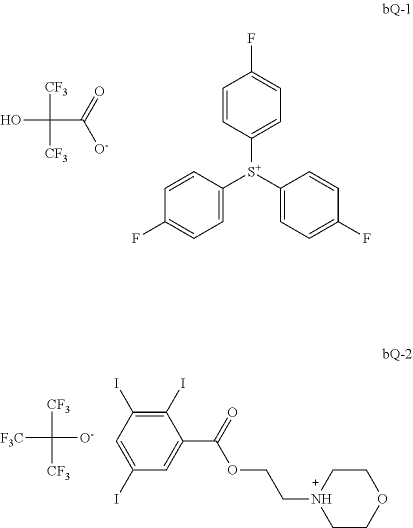 [SMILES: O=C([O-])C(O)(C(F)(F)F)C(F)(F)F ; Fc1ccc([S+](c2ccc(F)cc2)c2ccc(F)cc2)cc1 ; [O-]C(C(F)(F)F)(C(F)(F)F)C(F)(F)F ; O=C(OCC[NH+]1CCOCC1)c1cc(I)cc(I)c1I]

Comparative quenchers: cSQ-1 to cSQ-4

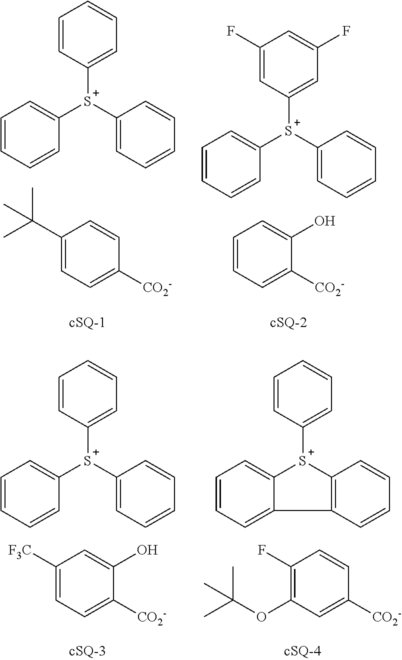

TABLE 1

| | | | Photo-acid | | Organic | Organic |
|---|---|---|---|---|---|---|
| | | Base polymer | generator | Quencher | solvent 1 | solvent 2 |
| | Resist | (parts by | (parts by | (parts by | (parts by | (parts by |
| | composition | mass) | mass) | mass) | mass) | mass) |
| Example | R-1 | P-1 (100) | PAG-1 (30.2) | SQ-1 (4.6) | PGMEA | DAA |
| 2-1 | | | | | (3,000) | (500) |
| Example | R-2 | P-1 (100) | PAG-2 (24.8) | SQ-2 (4.5) | PGMEA | DAA |
| 2-2 | | | | | (3,000) | (500) |
| Example | R-3 | P-1 (100) | PAG-2 (24.8) | SQ-3 (4.7) | PGMEA | DAA |
| 2-3 | | | | | (3,000) | (500) |
| Example | R-4 | P-1 (100) | PAG-2 (24.6) | SQ-4 (4.5) | PGMEA | DAA |
| 2-4 | | | | | (3,000) | (500) |
| Example | R-5 | P-1 (100) | PAG-2 (24.4) | SQ-5 (4.8) | PGMEA | DAA |
| 2-5 | | | | | (3,000) | (500) |
| Example | R-6 | P-1 (100) | PAG-3 (25.2) | SQ-6 (5.1) | PGMEA | DAA |
| 2-6 | | | | | (3,000) | (500) |
| Example | R-7 | P-1 (100) | PAG-4 (25.3) | SQ-7 (4.8) | PGMEA | DAA |
| 2-7 | | | | | (3,000) | (500) |
| Example | R-8 | P-1 (100) | PAG-3 (25.2) | SQ-8 (4.6) | PGMEA | DAA |
| 2-8 | | | | | (3,000) | (500) |
| Example | R-9 | P-1 (100) | PAG-1 (30.4) | SQ-9 (2.4) | PGMEA | DAA |
| 2-9 | | | | bQ-1 (2.4) | (3,000) | (500) |
| Example | R-10 | P-2 (100) | — | SQ-1 (4.6) | PGMEA | DAA |
| 2-10 | | | | | (3,000) | (500) |
| Example | R-11 | P-2 (100) | — | SQ-2 (4.9) | PGMEA | DAA |
| 2-11 | | | | | (3,000) | (500) |
| Example | R-12 | P-2 (100) | — | SQ-3 (4.8) | PGMEA | DAA |
| 2-12 | | | | | (3,000) | (500) |
| Example | R-13 | P-3 (100) | PAG-3 (10.5) | SQ-4 (4.8) | PGMEA | DAA |
| 2-13 | | | | | (3,000) | (500) |
| Example | R-14 | P-3 (100) | — | SQ-6 (4.7) | PGMEA | DAA |
| 2-14 | | | | | (2,200) | (900) |
| Example | R-15 | P-3 (100) | — | SQ-8 (4.6) | PGMEA | DAA |
| 2-15 | | | | | (2,200) | (900) |
| Example | R-16 | P-4 (100) | — | SQ-1 (2.3) | PGMEA | DAA |
| 2-16 | | | | bQ-2 (2.3) | (2,200) | (900) |
| Example | R-17 | P-4 (100) | — | SQ-5 (4.7) | PGMEA | DAA |
| 2-17 | | | | | (2,200) | (900) |
| Example | R-18 | P-4 (100) | — | SQ-2 (4.7) | PGMEA | DAA |
| 2-18 | | | | | (2,200) | (900) |
| Example | R-19 | P-5 (100) | PAG-1 (30.2) | SQ-1 (4.5) | PGMEA | DAA |
| 2-19 | | | | | (2,200) | (900) |
| Example | R-20 | P-5 (100) | PAG-5 (25.2) | SQ-7 (4.6) | PGMEA | DAA |
| 2-20 | | | | | (2,200) | (900) |

TABLE 2

| | | Base | Photo-acid | | Organic | Organic |
|---|---|---|---|---|---|---|
| | Resist | polymer | generator | Quencher | solvent 1 | solvent 2 |
| | com- | (parts by | (parts by | (parts by | (parts by | (parts by |
| | position | mass) | mass) | mass) | mass) | mass) |
| Comparative | CR-1 | P-1(100) | PAG-1(30.1) | cSQ-1(4.6) | PGMEA | DAA |
| Example 1-1 | | | | | (3,000) | (500) |
| Comparative | CR-2 | P-1(100) | PAG-2(24.7) | cSQ-2(4.6) | PGMEA | DAA |
| Example 1-2 | | | | | (3,000) | (500) |
| Comparative | CR-3 | P-1(100) | PAG-3(25.3) | cSQ-3(4.5) | PGMEA | DAA |
| Example 1-3 | | | | | (3,000) | (500) |
| Comparative | CR-4 | P-1(100) | PAG-4(25.1) | cSQ-4(4.7) | PGMEA | DAA |
| Example 1-4 | | | | | (3,000) | (500) |
| Comparative | CR-5 | P-2(100) | — | cSQ-4(4.6) | PGMEA | DAA |
| Example 1-5 | | | | | (3,000) | (500) |
| Comparative | CR-6 | P-2(100) | — | cSQ-2(4.6) | PGMEA | DAA |
| Example 1-6 | | | | | (3,000) | (500) |
| Comparative | CR-7 | P-3(100) | — | cSQ-1(4.5) | PGMEA | DAA |
| Example 1-7 | | | | | (3,000) | (500) |
| Comparative | CR-8 | P-3(100) | PAG-3(10.6) | cSQ-2(4.6) | PGMEA | DAA |
| Example 1-8 | | | | | (3,000) | (500) |
| Comparative | CR-9 | P-4(100) | — | cSQ-3(4.4) | PGMEA | DAA |
| Example 1-9 | | | | | (3,000) | (500) |
| Comparative | CR-10 | P-4(100) | — | cSQ-3(4.7) | PGMEA | DAA |
| Example 1-10 | | | | | (3,000) | (500) |
| Comparative | CR-11 | P-5(100) | PAG-1(30.2) | cSQ-2(4.6) | PGMEA | DAA |
| Example 1-11 | | | | | (3,000) | (500) |
| Comparative | CR-12 | P-5(100) | PAG-5(24.8) | cSQ-4(4.5) | PGMEA | DAA |
| Example 1-12 | | | | | (3,000) | (500) |

### [4] EUV Lithography Evaluation (1)

### Examples 3-1 to 3-20 and Comparative Examples 2-1 to 2-12

Each of the chemically amplified resist compositions (R-1 to R-20 and CR-1 to CR-12) shown in Tables 1 and 2 was spin-applied on a Si substrate on which a silicon-containing spin-on hard mask SHB-A940, manufactured by Shin-Etsu Chemical Co., Ltd. (silicon content of 43 mass %), was formed with 20 nm in film thickness. Then, the substrate was prebaked at 100° C. for 60 seconds by using a hot plate to produce a resist film with 50 nm in film thickness. This resist film was exposed using an EUV scanner NXE3300 (NA 0.33, σ 0.9/0.6, dipole illumination), manufactured by ASML Holding N.V. The exposure was performed with an LS pattern with an on-wafer size of 18 nm and a pitch of 36 nm, and with changing an exposure dose and focus (exposure dose pitch: 1 mJ/cm², focus pitch: 0.020 μm). After the exposure, PEB was performed at a temperature shown in Tables 3 and 4 for 60 seconds. Thereafter, puddle development with a 2.38 mass % aqueous TMAH solution for 30 seconds, rinse with a surfactant-containing rinse material, and spin-drying were performed to obtain a positive pattern in Examples 3-1 to 3-18 and Comparative Examples 2-1 to 2-10. In Examples 3-19 and 3-20 and Comparative Examples 2-11 and 2-12, a negative pattern was obtained.

The obtained LS pattern was observed with a length-measurement SEM (CG6300), manufactured by Hitachi High-Tech Corporation, to evaluate sensitivity, exposure latitude (EL), LWR, depth of focus (DOF), and collapse limit in accordance with the following methods. Tables 3 and 4 show the results.

### [Sensitivity Evaluation]

An optimum exposure dose Eₒₚ (mJ/cm²) to yield the LS pattern with 18 nm in line width and 36 nm in pitch was determined to specify this value as sensitivity. The smaller this value, the higher the sensitivity.

### [EL Evaluation]

From exposure doses to form the LS pattern within a range of ±10% of 18 nm space width (16.2 to 19.8 nm), EL (unit: %) was determined by the following equation. The larger the EL value, the better the performance.

EL (%)=(|E₁−E₂|/Eₒₚ)×100
E₁: An optimum exposure dose to yield an LS pattern with 16.2 nm in line width and 36 nm in pitch.
E₂: An optimum exposure dose to yield an LS pattern with 19.8 nm in line width and 36 nm in pitch.
Eₒₚ: An optimum exposure dose to yield the LS pattern with 18 nm in line width and 36 nm in pitch.

### [Lwr Evaluation]

In the LS pattern obtained by irradiation at Eₒₚ, sizes at 10 positions in the longitudinal direction of the line were measured. From the results, a tripled value (3a) of a standard variation (a) was determined as LWR. A smaller LWR value can yield a pattern with smaller roughness and uniform line width.

### [DOF Evaluation]

As evaluation of depth of focus, determined was a focus range to form the LS pattern within a range of ±10% of 18 nm size (16.2 to 19.8 nm). A larger DOF value indicates wider depth of focus.

### [Evaluation of Collapse Limit of Line Pattern]

Line sizes of the LS patterns at each exposure dose with the optimum focus were measured at 10 positions in the longitudinal direction. A narrowest line size obtained without collapse was specified as a collapse limit size. A smaller limit size value indicates a better collapse limit.

TABLE 3

| | | PEB | Optimum | | | | |
|---|---|---|---|---|---|---|---|
| | Resist | temper- | exposure | | | | Collapse |
| | com- | ature | dose | EL | LWR | DOF | limit |
| | position | (º C.) | (mJ/cm²) | (%) | (nm) | (nm) | (nm) |
| Example 3-1 | R-1 | 105 | 31 | 18 | 2.7 | 120 | 10.4 |
| Example 3-2 | R-2 | 100 | 32 | 17 | 2.8 | 120 | 10.3 |
| Example 3-3 | R-3 | 105 | 32 | 18 | 2.9 | 100 | 10.3 |
| Example 3-4 | R-4 | 100 | 31 | 17 | 2.8 | 110 | 10.2 |
| Example 3-5 | R-5 | 100 | 33 | 17 | 2.7 | 110 | 10.6 |
| Example 3-6 | R-6 | 100 | 32 | 17 | 2.8 | 110 | 11 |
| Example 3-7 | R-7 | 100 | 33 | 17 | 2.9 | 100 | 10.9 |
| Example 3-8 | R-8 | 105 | 32 | 18 | 2.7 | 110 | 11.1 |
| Example 3-9 | R-9 | 100 | 33 | 18 | 3 | 110 | 11.3 |
| Example 3-10 | R-10 | 100 | 27 | 18 | 2.9 | 120 | 11.2 |
| Example 3-11 | R-11 | 105 | 28 | 17 | 2.7 | 120 | 11.4 |
| Example 3-12 | R-12 | 100 | 28 | 18 | 2.8 | 110 | 10.9 |
| Example 3-13 | R-13 | 95 | 27 | 17 | 2.8 | 100 | 10.2 |
| Example 3-14 | R-14 | 100 | 29 | 19 | 2.7 | 110 | 10.8 |
| Example 3-15 | R-15 | 100 | 27 | 17 | 2.8 | 120 | 11.3 |
| Example 3-16 | R-16 | 100 | 28 | 18 | 2.8 | 120 | 11.5 |
| Example 3-17 | R-17 | 105 | 27 | 18 | 2.8 | 110 | 10.8 |
| Example 3-18 | R-18 | 105 | 28 | 18 | 2.8 | 120 | 11.4 |
| Example 3-19 | R-19 | 110 | 36 | 17 | 3 | 120 | 11.4 |
| Example 3-20 | R-20 | 110 | 36 | 17 | 2.9 | 120 | 11.5 |

TABLE 4

| | | PEB | Optimum | | | | |
|---|---|---|---|---|---|---|---|
| | Resist | temper- | exposure | | | | Collapse |
| | com- | ature | dose | EL | LWR | DOF | limit |
| | position | (° C.) | (mJ/cm²) | (%) | (nm) | (nm) | (nm) |
| Comparative | CR-1 | 105 | 41 | 14 | 3.6 | 90 | 14.3 |
| Example 2-1 | | | | | | | |
| Comparative | CR-2 | 100 | 41 | 15 | 3.5 | 80 | 13.4 |
| Example 2-2 | | | | | | | |
| Comparative | CR-3 | 105 | 40 | 14 | 3.5 | 70 | 14.2 |
| Example 2-3 | | | | | | | |
| Comparative | CR-4 | 100 | 39 | 15 | 3.4 | 80 | 13.4 |
| Example 2-4 | | | | | | | |
| Comparative | CR-5 | 100 | 34 | 14 | 3.3 | 80 | 14.2 |
| Example 2-5 | | | | | | | |
| Comparative | CR-6 | 105 | 35 | 15 | 3.5 | 80 | 13.4 |
| Example 2-6 | | | | | | | |
| Comparative | CR-7 | 100 | 34 | 16 | 3.5 | 90 | 12.6 |
| Example 2-7 | | | | | | | |
| Comparative | CR-8 | 100 | 35 | 15 | 3.3 | 80 | 12.9 |
| Example 2-8 | | | | | | | |
| Comparative | CR-9 | 105 | 34 | 14 | 3.5 | 80 | 13.4 |
| Example 2-9 | | | | | | | |
| Comparative | CR-10 | 105 | 33 | 16 | 3.6 | 90 | 13.2 |
| Example 2-10 | | | | | | | |
| Comparative | CR-11 | 110 | 43 | 16 | 3.6 | 90 | 12.9 |
| Example 2-11 | | | | | | | |
| Comparative | CR-12 | 105 | 41 | 15 | 3.6 | 100 | 13.5 |
| Example 2-12 | | | | | | | |

From the results shown in Tables 3 and 4, the chemically amplified resist compositions containing a quencher of the present invention were found to have good sensitivity and excellent EL, LWR, and DOF in the cases of both positive and negative resist compositions. In addition, the composition had a small collapse limit value, and was also confirmed to hardly cause pattern collapse even in fine pattern formation.

### [5] EUV Lithography Evaluation (2)

### Examples 4-1 to 4-20 and Comparative Examples 3-1 to 3-12

A silicon substrate with a silicon-containing spin-on hard mask SHB-A940 (silicon content: 43 mass %) manufactured by Shin-Etsu Chemical Co., Ltd. formed to have a film thickness of 20 nm was respectively spin-coated with each resist composition shown in Tables 1 and 2. The resultant was prebaked using a hot plate at 100° C. for 60 seconds to prepare a resist film having a film thickness of 60 nm. Subsequently, the resist film was exposed using an EUV scanner NXE3400 (NA: 0.33, σ: 0.9/0.6, quadrupole illumination, with a mask having a hole pattern with a pitch of 44 nm and +20% bias (on-wafer size)) manufactured by ASML, followed by PEB on the hot plate at a temperature shown in Tables 5 and 6 for 60 seconds, and development with a 2.38 mass % aqueous TMAH solution for 30 seconds to obtain a hole pattern with a size of 22 nm in Examples 4-1 to 4-18 and Comparative Examples 3-1 to 3-10, and a dot pattern with a size of 22 nm in Examples 4-19 and 4-20 and Comparative Examples 3-11 and 3-12.

Using a length-measurement SEM (CG6300), manufactured by Hitachi High-Tech Corporation, an exposure dose at which the hole or dot size of 22 nm was formed was measured and determined as sensitivity. In addition, the dimensions of 50 holes or dots in this event were measured, and the tripled value (3a) of a standard variation (a) calculated from the measurement results was obtained as the CDU. Tables 5 and 6 show the results.

TABLE 5

| | | | Optimum | |
|---|---|---|---|---|
| | | PEB | exposure | |
| | Resist | temperature | dose | CDU |
| | composition | (° C.) | (mJ/cm²) | (nm) |
| Example 4-1 | R-1 | 85 | 31 | 3.4 |
| Example 4-2 | R-2 | 85 | 33 | 3.1 |
| Example 4-3 | R-3 | 80 | 31 | 3.3 |
| Example 4-4 | R-4 | 80 | 30 | 3.1 |
| Example 4-5 | R-5 | 85 | 31 | 3.3 |
| Example 4-6 | R-6 | 85 | 33 | 3.1 |
| Example 4-7 | R-7 | 90 | 32 | 3.2 |
| Example 4-8 | R-8 | 80 | 31 | 3 |
| Example 4-9 | R-9 | 85 | 33 | 3.3 |
| Example 4-10 | R-10 | 85 | 28 | 3.2 |
| Example 4-11 | R-11 | 85 | 27 | 3.1 |
| Example 4-12 | R-12 | 85 | 28 | 3.1 |
| Example 4-13 | R-13 | 80 | 28 | 3.3 |
| Example 4-14 | R-14 | 85 | 27 | 3.2 |
| Example 4-15 | R-15 | 85 | 27 | 3.2 |
| Example 4-16 | R-16 | 80 | 25 | 3.2 |
| Example 4-17 | R-17 | 85 | 27 | 3.1 |
| Example 4-18 | R-18 | 85 | 27 | 3 |
| Example 4-19 | R-19 | 105 | 32 | 3.4 |
| Example 4-20 | R-20 | 110 | 33 | 3.3 |

TABLE 6

| | | | Optimum | |
|---|---|---|---|---|
| | | PEB | exposure | |
| | Resist | temperature | dose | CDU |
| | composition | (° C.) | (mJ/cm²) | (nm) |
| Comparative | CR-1 | 80 | 34 | 3.4 |
| Example 3-1 | | | | |
| Comparative | CR-2 | 85 | 35 | 3.5 |
| Example 3-2 | | | | |
| Comparative | CR-3 | 85 | 37 | 3.6 |
| Example 3-3 | | | | |
| Comparative | CR-4 | 80 | 35 | 3.3 |
| Example 3-4 | | | | |
| Comparative | CR-5 | 80 | 35 | 3.5 |
| Example 3-5 | | | | |
| Comparative | CR-6 | 80 | 36 | 3.6 |
| Example 3-6 | | | | |
| Comparative | CR-7 | 85 | 33 | 3.7 |
| Example 3-7 | | | | |
| Comparative | CR-8 | 80 | 34 | 3.5 |
| Example 3-8 | | | | |
| Comparative | CR-9 | 85 | 35 | 3.6 |
| Example 3-9 | | | | |
| Comparative | CR-10 | 80 | 37 | 3.8 |
| Example 3-10 | | | | |
| Comparative | CR-11 | 105 | 42 | 4.3 |
| Example 3-11 | | | | |
| Comparative | CR-12 | 110 | 40 | 4.5 |
| Example 3-12 | | | | |

From the results shown in Tables 5 and 6, the chemically amplified resist compositions containing a quencher of the present invention were found to have good sensitivity and excellent CDU in the cases of both positive and negative resist compositions.

The present description includes the following embodiments.

[1]: An onium salt represented by the following general formula (1),

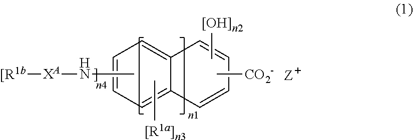

wherein n1 represents an integer of 0 or 1; n2 represents an integer of 0 to 3; R^{1a} represents a hydrocarbyl group having 1 to 20 carbon atoms and optionally containing a heteroatom; n3 represents an integer of 0 to 3; R^{1b} represents a hydrocarbyl group having 1 to 36 carbon atoms and optionally containing a heteroatom; X^{A} represents a carbonyl group that forms an amide bond together with an adjacent —NH or represents a sulfonyl group that forms a sulfonamide bond together with the adjacent —NH; n4 represents an integer of 1 or 2; and Z⁺ represents an onium cation.

[2]: The onium salt of the above [1], wherein the general formula (1) is represented by the following formula (1-A),

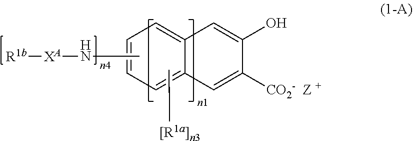

wherein R^{1a}, R^{1b}, X^{A}, n1, n3, n4, and Z⁺ are as defined above.
[3]: The onium salt of the above [2], wherein the general formula (1) is represented by the following formula (1-B),

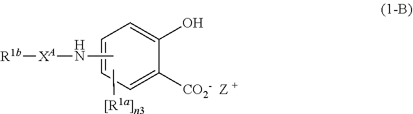

wherein R^{1a}, R^{1b}, X^{A}, n3, and Z⁺ are as defined above.
[4]: The onium salt of any one of the above [1] to [3], wherein the Z⁺ in the general formula (1) further represents an onium cation represented by one of the following general formulae (Cation-1) to (Cation-3),

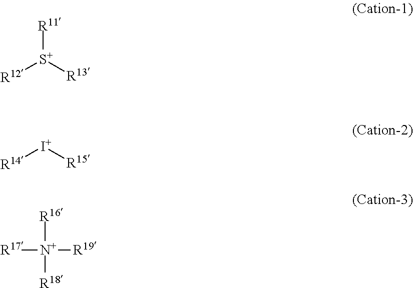

wherein R^{11′} to R^{19′} each independently represent a saturated or unsaturated, linear, branched, or cyclic hydrocarbyl group having 1 to 30 carbon atoms and optionally containing a heteroatom.
[5]: An acid diffusion controller comprising the onium salt of any one of the above [1] to [4].
[6]: A resist composition comprising the acid diffusion controller of the above [5].
[7]: The resist composition of the above [6], further comprising an acid generator to generate an acid.
[8]: The resist composition of the above [7], wherein the acid generator generates a sulfonic acid, imide acid, or methide acid.
[9]: The resist composition of any one of the above [6] to [8], further comprising an organic solvent.
[10]: The resist composition of any one of the above [6] to [9], further comprising a base polymer.
[11]: The resist composition of the above [10], wherein the base polymer contains a repeating unit represented by the following general formula (a1) and/or a repeating unit represented by the following general formula (a2),

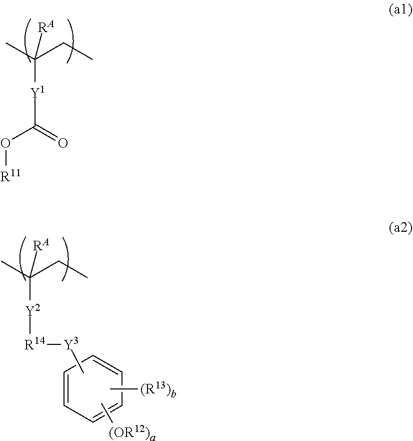

wherein each R^{A} independently represents a hydrogen atom or a methyl group; Y¹ represents a single bond or a linking group having 1 to 12 carbon atoms containing at least one selected from a phenylene group, a naphthylene group, an ester bond, and a lactone ring; Y² represents a single bond or an ester bond; Y³ represents a single bond, an ether bond, or an ester bond; R¹¹ and R¹² each independently represent an acid-labile group; R¹² represents a fluorine atom, a trifluoromethyl group, a cyano group, or a saturated hydrocarbyl group having 1 to 6 carbon atoms; R¹⁴ represents a single bond or an alkanediyl group having 1 to 6 carbon atoms, and some of the carbon atoms are optionally substituted with an ether bond or an ester bond; and “a” represents 1 or 2 and “b” represents an integer of 0 to 4, provided that 1≤a+b≤5.
[12]: The resist composition of the above [11], being a chemically amplified positive resist composition.
[13]: The resist composition of the above [10], wherein the base polymer contains no acid-labile group.
[14]: The resist composition of the above [13], being a chemically amplified negative resist composition.
[15]: The resist composition of the above [10], wherein the base polymer further contains at least one repeating unit selected from repeating units represented by the following general formulae (f1) to (f3),

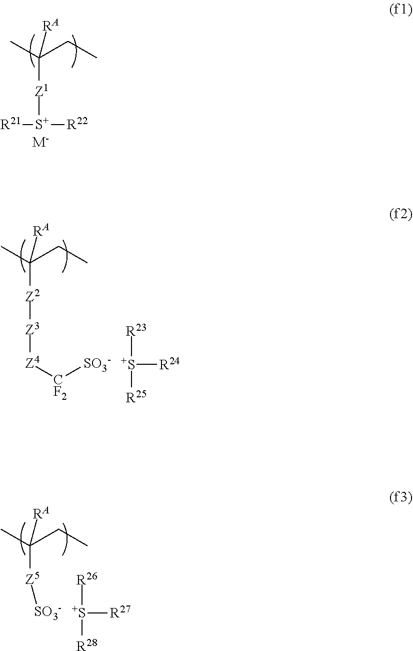

wherein each R^{A} independently represents a hydrogen atom or a methyl group; Z¹ represents a single bond, an aliphatic hydrocarbylene group having 1 to 6 carbon atoms, a phenylene group, a naphthylene group, an ester bond, a group having 7 to 18 carbon atoms derived from a combination of these groups, —O—Z¹¹—, —C(═O)—O—Z¹¹—, or —C(═O)—NH—Z¹¹—; Z″ represents an aliphatic hydrocarbylene group having 1 to 6 carbon atoms, a phenylene group, a naphthylene group, or a group having 7 to 18 carbon atoms derived from a combination of these groups, Z¹¹ optionally containing a carbonyl group, an ester bond, an ether bond, or a hydroxy group; Z² represents a single bond or an ester bond; Z³ represents a single bond, —Z³¹ —C(═O)—O—, —Z³¹ —O—, or —Z³¹ —O—C(═O)—; Z³¹ represents a hydrocarbylene group having 1 to 12 carbon atoms, a phenylene group, or a group having 7 to 18 carbon atoms derived from a combination of these groups, Z³¹ optionally containing a carbonyl group, an ester bond, an ether bond, an iodine atom, or a bromine atom; Z⁴ represents a methylene group, a 2,2,2-trifluoro-1,1-ethanediyl group, or a carbonyl group; Z⁵ represents a single bond, a methylene group, an ethylene group, a phenylene group, a fluorinated phenylene group, a phenylene group substituted with a trifluoromethyl group, —O—Z⁵¹ —, —C(═O) —O—Z⁵¹ —, or —C(═O) —NH—Z⁵¹—; Z⁵¹ represents an aliphatic hydrocarbylene group having 1 to 6 carbon atoms, a phenylene group, a fluorinated phenylene group, a phenylene group substituted with a trifluoromethyl group, or a combination of these groups, Z⁵¹ optionally containing any of a carbonyl group, an ester bond, an ether bond, a halogen atom, and a hydroxy group; R²¹ to R²⁸ each independently represent a hydrocarbyl group having 1 to 20 carbon atoms and optionally containing a heteroatom; R²³ and R²⁴ or R²⁶ and R²⁷ are optionally bonded to each other to form a ring together with a sulfur atom bonded to R²³ and R²⁴ or R²⁶ and R²⁷; and M⁻ represents a non-nucleophilic counterion.
[16]: The resist composition of any one of the above [6] to [15], further comprising a surfactant.
[17]: A patterning process comprising the steps of:
forming a resist film on a substrate by using the resist composition of any one of the above [6] to [16];
exposing the resist film to a high-energy beam; and
developing the exposed resist film by using a developer.
   [18]: The patterning process of the above [17], wherein the high-energy beam is a KrF excimer laser beam, an ArF excimer laser beam, an electron beam, or an extreme ultraviolet ray having a wavelength of 3 to 15 nm.

It should be noted that the present invention is not limited to the above-described embodiments. The embodiments are just examples, and any examples that have substantially the same feature and demonstrate the same functions and effects as those in the technical concept disclosed in claims of the present invention are included in the technical scope of the present invention.

1. An onium salt represented by the following general formula (1), 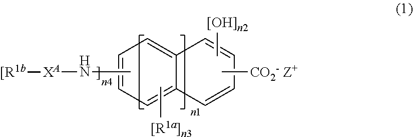
wherein n1 represents an integer of 0 or 1; n2 represents an integer of 0 to 3; R^{1a} represents a hydrocarbyl group having 1 to 20 carbon atoms and optionally containing a heteroatom; n3 represents an integer of 0 to 3; R^{1b} represents a hydrocarbyl group having 1 to 36 carbon atoms and optionally containing a heteroatom; X^{A} represents a carbonyl group that forms an amide bond together with an adjacent —NH or represents a sulfonyl group that forms a sulfonamide bond together with the adjacent —NH; n4 represents an integer of 1 or 2; and Z⁺ represents an onium cation.

2. The onium salt according to claim 1, wherein the general formula (1) is represented by the following formula (1-A), 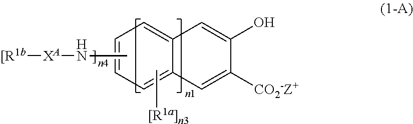
wherein R^{1a}, R^{1b}, X^{A}, n1, n3, n4, and Z⁺ are as defined above.

3. The onium salt according to claim 2, wherein the general formula (1) is represented by the following formula (1-B), 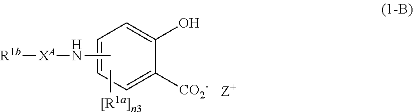
wherein R^{1a}, R^{1b}, X^{A}, n3, and Z⁺ are as defined above.

4. The onium salt according to claim 1, wherein the Z⁺ in the general formula (1) further represents an onium cation represented by one of the following general formulae (Cation-1) to (Cation-3), 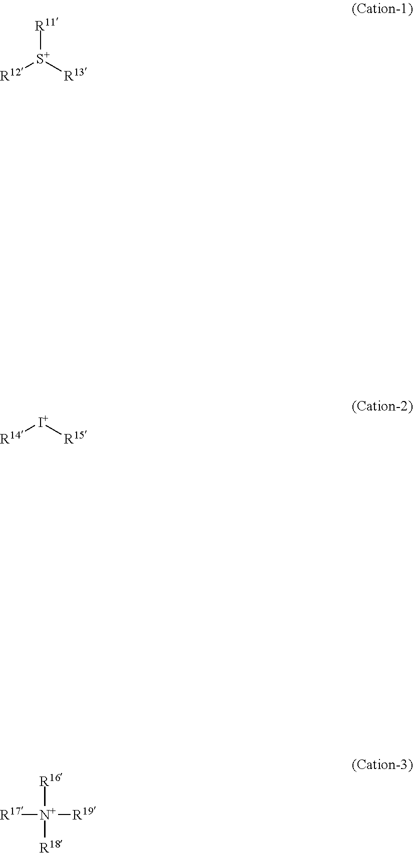
wherein R^{11′} to R^{19′} each independently represent a saturated or unsaturated, linear, branched, or cyclic hydrocarbyl group having 1 to 30 carbon atoms and optionally containing a heteroatom.

5. An acid diffusion controller comprising the onium salt according to claim 1.

6. A resist composition comprising the acid diffusion controller according to claim 5.

7. The resist composition according to claim 6, further comprising an acid generator to generate an acid.

8. The resist composition according to claim 7, wherein the acid generator generates a sulfonic acid, imide acid, or methide acid.

9. The resist composition according to claim 6, further comprising an organic solvent.

10. The resist composition according to claim 6, further comprising a base polymer.

11. The resist composition according to claim 10, wherein the base polymer contains a repeating unit represented by the following general formula (a1) and/or a repeating unit represented by the following general formula (a2), 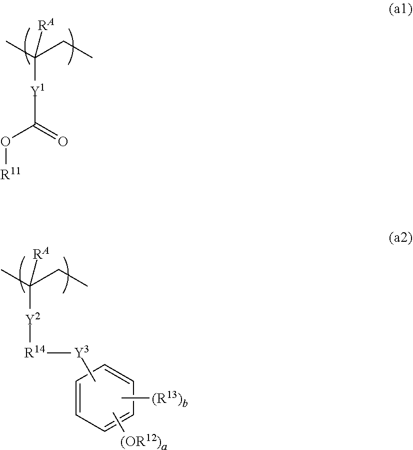
wherein each R^{A} independently represents a hydrogen atom or a methyl group; Y¹ represents a single bond or a linking group having 1 to 12 carbon atoms containing at least one selected from a phenylene group, a naphthylene group, an ester bond, and a lactone ring; Y² represents a single bond or an ester bond; Y³ represents a single bond, an ether bond, or an ester bond; R¹¹ and R¹² each independently represent an acid-labile group; R¹³ represents a fluorine atom, a trifluoromethyl group, a cyano group, or a saturated hydrocarbyl group having 1 to 6 carbon atoms; R¹⁴ represents a single bond or an alkanediyl group having 1 to 6 carbon atoms, and some of the carbon atoms are optionally substituted with an ether bond or an ester bond; and “a” represents 1 or 2 and “b” represents an integer of 0 to 4, provided that 1≤a+b≤5.

12. The resist composition according to claim 11, being a chemically amplified positive resist composition.

13. The resist composition according to claim 10, wherein the base polymer contains no acid-labile group.

14. The resist composition according to claim 13, being a chemically amplified negative resist composition.

15. The resist composition according to claim 10, wherein the base polymer further contains at least one repeating unit selected from repeating units represented by the following general formulae (f1) to (f3), 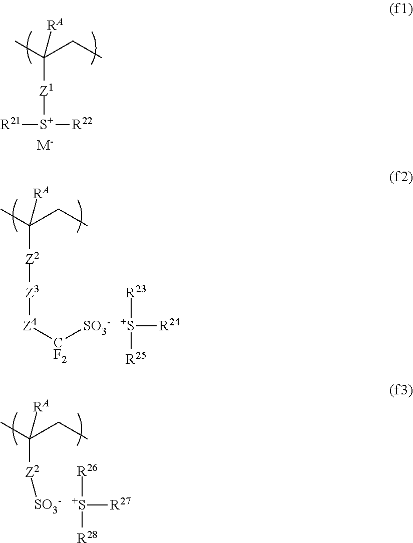
wherein each R^{A} independently represents a hydrogen atom or a methyl group; Z¹ represents a single bond, an aliphatic hydrocarbylene group having 1 to 6 carbon atoms, a phenylene group, a naphthylene group, an ester bond, a group having 7 to 18 carbon atoms derived from a combination of these groups, —O—Z¹¹—, —C(═O)—O—Z¹¹—, or —C(═O)—NH—Z¹¹—; Z¹¹ represents an aliphatic hydrocarbylene group having 1 to 6 carbon atoms, a phenylene group, a naphthylene group, or a group having 7 to 18 carbon atoms derived from a combination of these groups, Z¹¹ optionally containing a carbonyl group, an ester bond, an ether bond, or a hydroxy group; Z² represents a single bond or an ester bond; Z³ represents a single bond, —Z³¹ —C(═O)—O—, —Z³¹ —O—, or —Z³¹ —O—C(═O)—; Z³¹ represents a hydrocarbylene group having 1 to 12 carbon atoms, a phenylene group, or a group having 7 to 18 carbon atoms derived from a combination of these groups, Z³¹ optionally containing a carbonyl group, an ester bond, an ether bond, an iodine atom, or a bromine atom; Z⁴ represents a methylene group, a 2,2,2-trifluoro-1,1-ethanediyl group, or a carbonyl group; Z⁵ represents a single bond, a methylene group, an ethylene group, a phenylene group, a fluorinated phenylene group, a phenylene group substituted with a trifluoromethyl group, —O—Z⁵¹ —, —C(═O)—O—Z⁵¹ —, or —C(═O)—NH—Z⁵¹ —; Z⁵¹ represents an aliphatic hydrocarbylene group having 1 to 6 carbon atoms, a phenylene group, a fluorinated phenylene group, a phenylene group substituted with a trifluoromethyl group, or a combination of these groups, Z⁵¹ optionally containing any of a carbonyl group, an ester bond, an ether bond, a halogen atom, and a hydroxy group; R²¹ to R²⁸ each independently represent a hydrocarbyl group having 1 to 20 carbon atoms and optionally containing a heteroatom; R²³ and R²⁴ or R²⁶ and R²⁷ are optionally bonded to each other to form a ring together with a sulfur atom bonded to R²³ and R²⁴ or R²⁶ and R²⁷; and M⁻ represents a non-nucleophilic counterion.

16. The resist composition according to claim 6, further comprising a surfactant.

17. A patterning process comprising the steps of:
forming a resist film on a substrate by using the resist composition according to claim 6;
exposing the resist film to a high-energy beam; and
developing the exposed resist film by using a developer.

18. The patterning process according to claim 17, wherein the high-energy beam is a KrF excimer laser beam, an ArF excimer laser beam, an electron beam, or an extreme ultraviolet ray having a wavelength of 3 to 15 nm.
